(19) United States
(12) Patent Application Publication
(10) Pub. No.: US 2023/0250172 A1
(43) Pub. Date: Aug. 10, 2023

(54) IMPLANTABLE SCAFFOLDS AND USES THEREOF FOR IMMUNOTHERAPY AND OTHER USES

(71) Applicant: The Regents of the University of California, Oakland, CA (US)

(72) Inventors: Manish J. BUTTE, Los Angeles, CA (US); Mohammad Mahdi HASANI-SADRABADI, Los Angeles, CA (US); Fatemeh S. MAJEDI, Los Angeles, CA (US)

(73) Assignee: The Regents of the University of California, Oakland, CA (US)

(21) Appl. No.: 17/933,817

(22) Filed: Sep. 20, 2022

Related U.S. Application Data
(63) Continuation of application No. 17/638,519, filed on Feb. 25, 2022.
(60) Provisional application No. 62/902,346, filed on Sep. 18, 2019.

(51) Int. Cl. C07K 16/28; A61P 35/00; C07K 16/24

(52) U.S. Cl. CPC C07K 16/2809; A61P 35/00; C07K 16/246; C07K 16/247; C07K 16/248; C07K 16/2818

(57) ABSTRACT

An implantable or injectable scaffold comprising immunostimulatory compounds and a suppressor of regulatory T cell induction is provided for use in immunotherapy treatments, including the treatment of cancers and other tumors, in particular solid tumors including inoperable tumors, as well as for other applications of immune enhancement and/or suppression.

### CROSS-REFERENCE TO RELATED APPLICATIONS

This application is a continuation-in-part of U.S. patent application Ser. No. 17/638,519, which is the U.S. national stage application of PCT/US2020/51363, filed Sep. 18, 2020, which claims priority to U.S. Provisional Patent Application Ser. No. 62/902,346, filed Sep. 18, 2019. These applications are all incorporated by reference herein in their entirety.

### STATEMENT OF GOVERNMENT INTEREST

This invention was made with government support under Grant Numbers GM110482 and DE029157, awarded by the National Institutes of Health. The government has certain rights in the invention.

### FIELD OF INTEREST

This disclosure relates to implantable scaffolds comprising a T cell immunoregulatory compound, such as a T cell immunostimulatory compound, a compound that suppresses induction of regulatory T cells, or a T cell immunosuppression compound. These implantable scaffolds may be used for the controlled release of a cytokine within a localized environment of a tumor, e.g., as part of a therapeutic treatment of cancer, or for localized treatment at a focus of interest of an autoimmune disease or an allergic reaction or hypersensitivity reaction, a localized site of an infection or infectious disease, a localized site of an injury or other damage, a transplant or other surgical site, or a blood clot. They may also be used for the controlled release of a cytokine for the regulation of immunity in general and for other therapeutic uses.

### BACKGROUND OF THE INVENTION

Therapeutic modulation of immunity has made significant headway in the fight against cancer. However, global immunomodulation results in systemic adverse effects including severe inflammation, provocation of autoimmunity, and susceptibility to infection.

Cytokines influence the proliferation and differentiation of cultured, primary T cells. Augmentation and engineering of immune responses have major applications in combating cancers, including solid tumor cancers.

Activation of cytotoxic T cells for cancer immunotherapy has significant potential for patients with tumors or following tumor resection, but obstacles persist in available procedures. Transforming growth factor-beta (TGF-β) is a potent component of the tumor microenvironment, which promotes cancer growth and metastasis and promotes the induction of regulatory T cells (Tregs; T regulatory cells) from the helper T cells drawn to the tumor. TGF-β also potently inhibits cytotoxic T cells in the tumor microenvironment. TGF-β has, therefore, become a target in the enhancement of immunotherapy. However, systemic TGF-β inhibition in preclinical models has shown major adverse effects on the cardiovascular, gastrointestinal, and skeletal systems, owing to the pleotropic effects that TGF-β plays across the body. Similarly, IL-2 is a cytokine that plays the major role in activation and expansion of helper and cytotoxic T cells (CTLs) to fight infections and cancer. IL-2 also helps activate natural killer cells for fighting viruses and cancer. Unfortunately, systemic delivery of IL-2 has been shown to be inefficient and has additional limitations including continuous secretion eliciting non-specific immune response.

A tumor, whether benign or malignant, is caused by abnormal growth of cells or a tissue. Cancer is an abnormal and malignant state in which uncontrolled proliferation of one or more cell populations interferes with normal biological functioning. Standard treatments for cancer include surgery, chemotherapy, and radiation therapy. T cell immunotherapy is a promising approach for cancer. However, significant challenges hamper its therapeutic potential, including insufficient activation, delivery, and clonal expansion of T cells into the tumor environment. Even non-cancerous tumors may pose significant health challenges, such as when they are located at treatment site that is difficult to access or when they chronically recur. 91% of deaths from cancer occur due to solid tumors, over 1000 deaths per day, highlighting a profound unmet need for new therapies. Solid tumors elude clearance by T cells due to a variety of immunosuppressive features of the tumor microenvironment. TGF-β made in the tumor milieu promotes development of regulatory T cells, which suppress cytotoxic responses, but TGF-β cannot easily be suppressed globally because of autoimmune and other side effects. Cytotoxic effector functions of intratumoral T cells are weakly activated, but global T cell activation cannot be pursued due to adverse effects like cytokine storm.

Some infectious and non-infectious medical conditions exist, at least initially, in localized environments within the body. For example, these types of diseases and conditions are often difficult to treat without systemic exposure to therapeutic agents, which may have significant side effects. Some autoimmune diseases (e.g., rheumatoid arthritis, juvenile dermatomyositis, psoriasis, psoriatic arthritis, sarcoidosis, lupus, Crohn's disease, eczema, vasculitis, ulcerative colitis, multiple sclerosis) may present with at least some localized symptoms or symptoms in a particular system of the body, but treatment options may leave the patient having to choose between alleviating one or more symptoms (e.g., use of a non-steroidal anti-inflammatory drug [NSAID] or an antihistamine or a dermatological ointment or cream providing limited relief of a given symptom) or systemic exposure of the entire body to a more aggressive treatment (e.g., methotrexate) with a concomitant increase in potentially dangerous side effects. Likewise, some infectious diseases (e.g., shingles) or initially localized infections (e.g., methicillin-resistant Staphylococcus aureus [MRSA] infection) may have few treatment options or may require the use of more aggressive systemic treatments. In addition, traumatic injury, chronic damage (e.g., osteoarthritis), surgery, or a blood clot may necessitate the use of more aggressive systemic treatments, notwithstanding the limited location of the injury or surgical site. Furthermore, the concern over potential rejection of a transplant (e.g., a transplanted organ) necessitates aggressive systemic treatments with immune suppression drugs, often with significant side effects, also notwithstanding the limited location of the transplant site.

Despite recent successes in cancer immunotherapies that emphasize high therapeutic potency in treating patients with progressive tumors, significant challenges, including insufficient activation and eventual exhaustion of effector T cells as well as suppression of their effector responses in the tumor microenvironment; and inadequate ability to expand tumor-specific T cell ex vivo hinder the potential of T cell therapies, especially in solid tumors. Most of the current immunotherapy approaches aim to facilitate T cells to fight tumors and provoke their infiltration. Some of the commonly used strategies include blocking inhibitory receptors such as anti PD-1 and anti CTLA-4 while others include evoking cytotoxic T lymphocyte (CTL) responses such as chimeric antigen receptor (CAR)-T cell therapies and adoptive cell transfer (ACT) approaches. Despite their revolutionary approaches for hematopoietic cancers, potency of these methods and the need for expanding tumor-specific T cells is a need not yet satisfactorily met for solid tumor therapy. One of the major flaws associated with checkpoint inhibitor therapies (CPI) and chemokine therapies such as IL-2 or IL-12 that hampers their clinical translation is their administration route and all the immune-related adverse events that are affiliated with it. In this regard few attempts have been made towards making the deliveries more targeted. Nanogel “backpacks” have demonstrated the release of cytokines to T cells. However, the continual release of cytokines risks systemic exposure, side effects, and compromise of a limited supply of cytokine. Collagen-binding domain fused to IL-12 is another example that emphasizes the impact of tumor targeting and prolongation of cytokine release in the tumor stroma. Though, the IV administration in this case especially puts patients with cardiovascular disease at risk. The other matter in these systems is that the rate of release of cytokines is not well controlled. It has been shown that the rate at which cytokines are delivered to CD8+ T cells impacts their differentiation and effector functionality. To tackle the issues related to ACT and improve ex vivo activation and expansion of tumor-reactive T cells, antigen-presenting cell (APC) mimetic scaffolds have been developed that show polyclonal expansion of T cells. Yet they lack the ability to manipulate tumor microenvironment so that it favors formation of tumor fighting T cells.

Another challenge that tumors face is the presence of T regulatory cells (Tregs). Transforming growth factor β (TGF-β) is known to be a key factor in the induction of Tregs from helper T cells drawn to the tumor, which then promotes cancer growth and metastasis. TGF-β also potently inhibits cytotoxic T cells in the tumor microenvironment and has, therefore, become an exciting target in the enhancement of immunotherapy. However, systemic TGF-β inhibition in preclinical models has shown major adverse effects on the cardiovascular, gastrointestinal, and skeletal systems, owing to the pleiotropic effects that TGF-β plays across the body. The release of TGF-β inhibitors by injected nanoliposomes has been shown to reduce metastases but has not shown a local impact in regulatory T cells. Moreover, mechanical stiffness of the niche in which T cells home and face antigens makes a difference on their fate.

Thus, there remains an unmet need for compositions and methods of treatment of cancers and other tumors, for example, but not limited to, treatment of benign or malignant solid tumors. A major gap in treatment exists, wherein there is an inability to provide local factors where most needed in the treatment of solid tumors, while avoiding systemic exposure to immunomodulatory agents.

Similarly, there remains an unmet need for compositions and methods of treatment of localized conditions or symptoms of, for example, but not limited to, infectious and non-infectious medical conditions, injuries, damage, surgery, and transplant. A major gap in treatment exists, wherein there is an inability to provide local factors and other treatments where most needed in the treatment of localized conditions or symptoms, while avoiding systemic exposure to immunomodulatory agents.

Accordingly, there is a need for improving the effectiveness of immunotherapy.

### SUMMARY OF THE INVENTION

To facilitate the immune response against solid tumors, provided herein is a multifunctional biomaterial that is placed adjacent to a tumor and which attracts and potentiates cytotoxic T cells and suppresses local regulatory T cells. Together these activities allow for the much sought after materials and methods for overcoming the immunosuppressive effects of the microenvironment of solid tumors.

Additionally, provided herein is a multifunctional biomaterial placed in a treatment area to deliver compositions treating localized symptoms of, for example, but not limited to, infectious and non-infectious medical conditions, injuries, damage, surgery, and transplant, where most needed in the treatment of localized conditions or symptoms, while avoiding systemic exposure to immunomodulatory agents.

In some aspects, a porous scaffold is provided comprising at least one compound that regulates T cell immune response; and at least one compound that regulates induction of regulatory T cells (Tregs).

In some embodiments, the compound that regulates T cell immune response comprises a T cell immunostimulatory compound or a T cell immunosuppression compound. In some embodiments, the compound that suppresses induction of Tregs comprises a TGF-β inhibitor. In some embodiments, the TGF-β inhibitor is a TGF-β receptor inhibitor. In some embodiments, the TGF-β inhibitor is galinusertib (LY2157299) or SB505124. In other embodiments, the at least one compound that regulates induction of Tregs comprises a compound that induces Tregs. In some embodiments, the compound that induces Tregs is a TGF-β or an activator thereof.

Compounds that suppression induction of Tregs include, but are not limited to, inhibitors of transforming growth factor-beta (TGF-β), such as an inhibitor of the TGF-β receptor. Non-limiting examples of TGF-β receptor inhibitors include galinusertib (LY2157299), SB505124, small molecule inhibitors, antibodies, chemokines, apoptosis signals (e.g., cytotoxic T-lymphocyte-associated protein 4/programmed cell death protein 1 (CTLA-4/PD-1); Granzyme; tumor necrosis factor (TNF)-related apoptosis-inducing ligand (TRAIL); Fas/Fas-L, Galectin-9/transmembrane immunoglobulin and mucin domain 3 (TIM-3)). Compounds that induce Tregs include TGF-β and activators thereof (e.g., SB 431542, A 83-01, RepSox, LY 364947, D 4476, SB 525334, GW 788388, SD 208, R 268712, IN 1130, SM 16, A 77-01, AZ 12799734).

In some embodiments, the at least one compound that regulates T cell immune response comprises a T cell immunostimulatory compound and the at least one compound that regulates induction of Tregs comprises a compound that suppresses induction of Tregs. In some embodiments, the T cell immunostimulatory compound is a T cell activator, a T cell attractant or a T cell adhesion compound. In some embodiments, the T cell immunostimulatory compound comprises a cytokine, a therapeutic or diagnostic protein, a growth factor, a chemokine, a therapeutic or diagnostic antibody or fragment thereof, an antigen-binding protein, a Fc fusion protein, an anticoagulant, an enzyme, a hormone, a thrombolytic, a peptide, an oligonucleotide, a nucleic acid, a chemokine ligand, or an anti-cluster of differentiation (anti-CD) antibody or fragment thereof. In some embodiments, the cytokine comprises an interleukin (IL). In some embodiments, the T cell immunostimulatory compound comprises interleukin-2 (IL-2), interleukin-4 (IL-4), interleukin-6 (IL-6), interleukin-7 (IL-7), interleukin-10 (IL-10), interleukin-12 (IL-12), interleukin-15 (IL-15), IL-2 superkine, chemokine (C-C motif) ligand 19 (CCL19), chemokine (C-C motif) ligand 21 (CCL21), anti-cluster of differentiation 3 (anti-CD3), or anti-cluster of differentiation 28 (anti-CD28), or any combination thereof. In some embodiments, the IL-2 superkine comprises the sequence as set forth in SEQ ID NO: 3. In some embodiments, the immunostimulatory compound is a CpG oligonucleotide (including toll-like receptor 9 (TLR9) agonist adjuvants), poly (I:C), monophosphoryl lipid A (MPLA), imiquimod, or a cyclic dinucleotide.

In some embodiments, the at least one compound that regulates T cell immune response comprises a T cell immunosuppression compound and the at least one compound that regulates induction of Tregs comprises a compound that induces Tregs. In some embodiments, the T cell immunosuppression compound comprises stromal cell-derived factor 1a (SDF-1a). In some embodiments, the growth factor comprises transforming growth factor-beta (TGF-β), vascular endothelial growth factor (VEGF), or bone morphogenetic protein-2 (BMP-2). In some embodiments, the scaffolds comprises IL-2, IL-4 and TGF-β.

In some embodiments, the at least one compound that regulates induction of regulatory T cells is released slowly from the scaffold.

In some embodiments, the at least one compound that regulates induction of regulatory T cells comprises a compound that suppresses induction of regulatory T cells or a compound that induces regulatory T cells.

In some embodiments, the compound that suppresses induction of regulatory T cells is an inhibitor of transforming growth factor-beta (TGF-β), such as a TGF-β receptor inhibitor. In some embodiments, the inhibitor is galinusertib (LY2157299) or SB505124.

In another related aspect, one or more of the compounds comprises a therapeutic or diagnostic protein. In another related aspect, one or more of the compounds comprises a cytokine, a chemokine, a therapeutic or diagnostic antibody or fragment thereof, an antigen-binding protein, a Fc fusion protein, an anticoagulant, an enzyme, a hormone, or a thrombolytic. In another related aspect, the cytokine comprises an interleukin. In another related aspect, the interleukin comprises an IL-2, IL-4, IL-6, IL-7, IL-10, an IL-12, an IL-15, or an IL-2 superkine. In yet another related aspect, a cytokine may include a human cytokine. In still another related aspect, an IL-2 cytokine comprises an IL-2 superkine. In some embodiments, the IL-2 superkine comprises the sequence as set forth in SEQ ID NO: 3.

In some embodiments, the at least one compound that regulates T cell immune response is bound to heparin. In some embodiments, the heparin is bound to one or more microparticles embedded in the scaffold. In some embodiments, the one or more microparticles comprise one or more silica microparticles. In some embodiments, the heparin is provided at about 2 nanomols per milligram (nmol/mg) of silica. In some embodiments, the one or more silica microparticles are about 3 microns (μm) to about 25 microns (μm). In some embodiments, the silica is mesoporous silica. In some embodiments, the loading of the one or more silica microparticles by the at least one compound that regulates T cell immune response is increased by the bound heparin. In some embodiments, the release of the at least one compound that regulates T cell immune response from the one or more silica microparticles is reduced by the bound heparin. In some embodiments, the silica microparticles persist in vivo for at least 15-20 days.

In some embodiments, the porous scaffold further comprises one or more nanoparticles. In some embodiments, the nanoparticles and/or microparticles comprise poly(lactic-co-glycolic acid) (PLGA). In some embodiments, the nanoparticles or microparticles are loaded with at least one compound that regulates induction of regulatory T cells.

In some embodiments, the scaffold is biocompatible or biodegradable. In some embodiments, the scaffold comprises a polymer selected from alginate, hyaluronic acid and chitosan, PLGA, or any combination thereof. In some embodiments, the polymer comprises an arginine-glycine-aspartate (RGD) peptide. In some embodiments, the porous scaffold comprises pores of from about 10 to about 300 microns.

In some embodiments, the scaffold is provided to be surgically implantable or injectable or administrable through a catheter. In some embodiments, the scaffold further comprises one or more immune cells. In some embodiments, the one or more immune cells are T cells. In some embodiments, the T cells comprise transgenic and wild-type, murine and human CD4+ and CD8+ T cells. In some embodiments, the T cells are chimeric antigen receptor T cells (CAR-T cells). In some embodiments, anti-CD3 or anti-CD28 antibodies are covalently bound to the polymer.

In some embodiments, the porous scaffold comprises an alginate-RGD polymer comprising silica-heparin microparticles bound to IL-2, anti-CD3 and anti-CD28, PLGA nanoparticles or microparticles comprising a TGF-β inhibitor, and anti-CD3 and anti-CD28 antibodies covalently bound to the alginate-RGD polymer.

In some aspects, a method is provided of regulating an immune response to a disease or medical condition or symptoms thereof, at a focus of interest in a subject in need, the method comprising providing a porous scaffold at a site at or near a site of the focus of interest, the porous scaffold comprising at least one compound that regulates T cell immune response and at least one compound that regulates induction of regulatory T cells (Tregs).

In some embodiments, the disease or medical condition comprises a tumor, a suspected tumor, or a resected tumor and the porous scaffold is provided at or adjacent to a focus of interest comprising the tumor, suspected tumor, or resected tumor.

In some embodiments, the tumor is a solid tumor. In some embodiments, the tumor, suspected tumor, or resected tumor comprises a cancerous, pre-cancerous, or non-cancerous tumor. In some embodiments, the tumor comprises a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor (for example, a triple-negative breast cancer), an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma, esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non-small cell lung cancer (NSCLC), squamous cell non-small cell lung cancer, breast cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16;16)(p13.1;q22); CBFB-MYH11; adult acute myeloid leukemia with t(8;21)(q22;q22); RUNX1-RUNX1T1; adult acute myeloid leukemia with t(9;11)(p22;q23); MLLT3-MLL; adult acute promyelocytic leukemia with t(15;17)(q22;q12); PML-RARA; alkylating agent-related acute myeloid leukemia, chronic lymphocytic leukemia, Richter's syndrome; Waldenstrom's macroglobulinemia, adult glioblastoma; adult gliosarcoma, recurrent glioblastoma, recurrent childhood rhabdomyosarcoma, recurrent Ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent Merkel cell carcinoma; stage III Merkel cell carcinoma; stage IV Merkel cell carcinoma, myelodysplastic syndrome and recurrent mycosis fungoides and Sezary syndrome. In some embodiments, at the site, T cells are stimulated to target the tumor, suspected tumor, or resected tumor, and the induction of Tregs is suppressed.

In some embodiments, said treating reduces the size of the tumor, eliminates the tumor, slows the growth or regrowth of the tumor, slows the growth or regrowth of a secondary tumor, or prolongs survival of said subject or any combination thereof.

In some embodiments, at the site, T cells are stimulated to target the focus of interest, and the induction of Tregs is suppressed. In other embodiments, at the site, T cells are suppressed at or near the focus of interest, and Tregs are induced.

In some embodiments, the disease or medical condition comprises an autoimmune disease, and the porous scaffold is provided at or adjacent to a focus of interest comprising an autoimmune-targeted or symptomatic focus of said autoimmune disease; the disease or medical condition comprises an allergic reaction or hypersensitivity reaction, and the porous scaffold is provided at or adjacent to a focus of interest comprising a reactive focus of said allergic reaction or hypersensitivity reaction; the disease or medical condition comprises a localized infection or an infectious disease, and the porous scaffold is provided at or adjacent to a focus of interest comprising a focus of infection or symptoms; the disease or medical condition comprises an injury or a site of chronic damage, and the porous scaffold is provided at or adjacent to a focus of interest comprising the injury or the site of chronic damage; the disease or medical condition comprises a surgical site, and the porous scaffold is provided at or adjacent to a focus of interest comprising the surgical site; the disease or medical condition comprises a transplanted organ, tissue, or cell, and the porous scaffold is provided at or adjacent to a focus of interest comprising a transplant site; or the disease or medical condition comprises a blood clot causing or at risk for causing a myocardial infarction, an ischemic stroke, or a pulmonary embolism, and the porous scaffold is provided at or adjacent to a focus of interest comprising the site of the blood clot. In some embodiments, said treating reduces or eliminates inflammation or another symptom of said autoimmune-targeted or symptomatic focus of said autoimmune disease, prolongs survival of said subject, or any combination thereof; reduces or eliminates inflammation or another symptom of allergic reaction or hypersensitivity reaction at said reactive focus of said allergic reaction or hypersensitivity reaction, prolongs survival of said subject, or any combination thereof; reduces or eliminates infection or symptoms at said focus of infection or symptoms of said localized infection or infectious disease, prolongs survival of said subject, or any combination thereof; reduces, eliminates, inhibits or prevents structural, organ, tissue, or cell damage, inflammation, infection, or another symptom at said site of injury or said site of chronic damage, improves structural, organ, tissue, or cell function at said site of injury or said site of chronic damage, improves mobility of said subject, prolongs survival of said subject, or any combination thereof; reduces, eliminates, inhibits, or prevents structural, organ, tissue, or cell damage, inflammation, infection, or another symptom at said surgical site, improves structural, organ, tissue, or cell function at said surgical site, improves mobility of said subject, prolongs survival of said subject, or any combination thereof; reduces, eliminates, inhibits or prevents transplanted organ, tissue, or cell damage or rejection, inflammation, infection or another symptom at said transplant site, improves mobility of said subject, prolongs survival of said transplanted organ, tissue, or cell, prolongs survival of said subject, or any combination thereof; or reduces or eliminates said blood clot causing or at risk for causing said myocardial infarction, said ischemic stroke, or said pulmonary embolism in said subject, improves function or survival of a heart, brain, or lung organ, tissue, or cell in said subject, reduces damage to a heart, brain, or lung organ, tissue, or cell in said subject, prolongs survival of a heart, brain, or lung organ, tissue, or cell in said subject, prolongs survival of said subject, or any combination thereof. In some embodiments, the disease or medical condition comprises a blood clot causing or at risk for causing a myocardial infarction, an ischemic stroke, or a pulmonary embolism, and the porous scaffold is provided at or adjacent to a focus of interest comprising the site of the blood clot together with angioplasty or another clot removal treatment.

In some aspects, a method is provided for stimulating T cells to target a solid tumor and for suppressing the induction of Tregs in a patient comprising providing the porous scaffold described herein at a site at or near a solid tumor, a suspected solid tumor or a resected solid tumor, the porous scaffold comprising at least one response cell immunostimulatory compound and at least one compound that suppresses induction of regulatory T cells (Tregs). In one embodiment, the tumor is an inoperable tumor.

In some aspects, a method is provided for regulating an immune response at a focus of interest in a subject in need, said method comprising providing a porous scaffold to the subject, at or near a site of the focus of interest, the porous scaffold comprising at least one compound that regulates T cell immune response; and at least one compound that regulates induction of regulatory T cells (Tregs), wherein regulating the immune response comprises increasing or decreasing proliferation of cytotoxic T cells; increasing or decreasing proliferation of helper T cells; maintaining, increasing, or decreasing the population of helper T cells at the site of said focus of interest; activating or suppressing cytotoxic T cells at the site of said focus of interest; or any combination thereof. In some aspects, a method is provided for treating a disease or medical condition, or alleviating symptoms thereof, at a focus of interest in a subject in need, said method comprising providing a porous scaffold at a site at or near a focus of interest, the porous scaffold comprising: at least one compound that regulates T cell immune response; and at least one compound that regulates induction of regulatory T cells (Tregs).

In another aspect, a method is provided herein for making a porous biocompatible or biodegradable scaffold for regulating an immune response at a focus of interest in a subject in need, the method comprising: providing a porous scaffold comprising a polymer: embedding in the scaffold one or more microparticles or one or more nanoparticles, the one or more microparticles bound to heparin, and the heparin bound to at least one compound that regulates T cell immune response; or the one or more nanoparticles or microparticles are bound to or encapsulate at least one compound that regulates induction of regulatory T cells (Tregs). In some embodiments, the porous biocompatible or biodegradable scaffold comprising a polymer comprising alginate, hyaluronic acid, chitosan, PLGA, or a combination thereof, or an arginine-glycine-aspartate (RGD) peptide, or an alginate-RGD polymer; the one or more microparticles comprising silica-heparin or poly(lactic-co-glycolic acid) (PLGA); or the nanoparticles comprising poly(lactic-co-glycolic acid) (PLGA). In some embodiments, the porous biocompatible or biodegradable scaffold further comprising one or more immune cells. In some embodiments, the porous biocompatible or biodegradable scaffold further comprising anti-CD3 or anti-CD28 antibodies covalently bound to the polymer. In some embodiments, the at least one compound that regulates T cell immune response comprising a T cell immunostimulatory compound comprising a cytokine, a therapeutic or diagnostic protein, a growth factor, a chemokine, a therapeutic or diagnostic antibody or fragment thereof, an antigen-binding protein, a Fc fusion protein, an anticoagulant, an enzyme, a hormone, a thrombolytic, a peptide, an oligonucleotide, a nucleic acid, a chemokine ligand, or an anti-cluster of differentiation (anti-CD) antibody or fragment thereof, interleukin-2 (IL-2), interleukin-4 (IL-4), interleukin-6 (IL-6), interleukin-7 (IL-7), interleukin-10 (IL-10), interleukin-12 (IL-12), interleukin-15 (IL-15), IL-2 superkine, chemokine (C-C motif) ligand 21 (CCL21), anti-CD3 or anti-CD28, a CpG oligonucleotide (including toll-like receptor 9 (TLR9) agonist adjuvants), poly (I:C), monophosphoryl lipid A (MPLA), imiquimod, or a cyclic dinucleotide or any combination thereof; or the at least one compound that regulates induction of regulatory T cells (Tregs) comprising a compound that suppresses induction of Tregs comprising galinusertib (LY2157299), SB505124, or another transforming growth factor-beta (TGF-β) inhibitor.

In one embodiment, the present disclosure provides a porous biocompatible or biodegradable scaffold for regulating an immune response in a subject in need thereof, the scaffold comprising (i) one or more microparticles, (ii) one or more nanoparticles, and (iii) a polymer comprising one or more of alginate, hyaluronic acid and chitosan, wherein the one or more microparticles comprise heparin, and the heparin is bound to at least one compound that regulates T cell immune response, wherein the one or more nanoparticles or microparticles comprise at least one compound that regulates induction of regulatory T cells (Tregs). In one embodiment, the alginate comprises one or more arginine-glycine-aspartate (RGD) peptides. In one embodiment, the one or more microparticles comprise one or more silica microparticles. In one embodiment, the one or more nanoparticles or microparticles comprise poly(lactic-co-glycolic acid) (PLGA).

In some embodiments of the above porous scaffold, the at least one compound that regulates T cell immune response comprises a cytokine, a growth factor, a chemokine, or an antibody or fragment thereof. In one embodiment, the compound that regulates T cell immune response comprises one or more of interleukin-2 (IL-2), interleukin-4 (IL-4), interleukin-6 (IL-6), interleukin-7 (IL-7), interleukin-10 (IL-10), interleukin-12 (IL-12), interleukin-15 (IL-15), IL-2 superkine, chemokine (C-C motif) ligand 21 (CCL21), anti-CD3 antibodies, and anti-CD28 antibodies. In some embodiments, the antibodies are covalently bound to the polymer. In some embodiments, the at least one compound that regulates induction of regulatory T cells is an inhibitor of transforming growth factor-beta (TGF-β). In one embodiment, the inhibitor of TGF-β is galinusertib (LY2157299) or SB505124.

In some embodiments, the above porous scaffold comprises one or more silica-heparin microparticles bound to IL-2; one or more PLGA nanoparticles or microparticles comprising a TGF-β inhibitor; and anti-CD3 and anti-CD28 antibodies covalently bound to an alginate-RGD polymer.

In some embodiments, the above porous scaffold further comprises one or more immune cells. In some embodiments, the immune cells comprise wild-type or transgenic T cells, murine or human T cells, CD4+ or CD8+ T cells, or chimeric antigen receptor T cells (CAR-T cells).

In another embodiment, the present disclosure provides a method of treating a first tumor in a subject in need thereof, the method comprising providing the porous scaffold disclosed herein at a site at or near the first tumor, thereby treating the first tumor in the subject. In one embodiment, the porous scaffold is surgically implanted or inserted at a site at or near the first tumor. In one embodiment, the first tumor is a solid tumor. In one embodiment, the above porous scaffold comprises T cells that are stimulated to target the first tumor, and induction of Tregs is suppressed. In some embodiments, the above method of treatment reduces or eliminates the first tumor, slows the growth or regrowth of the first tumor, prolongs survival of the subject, or any combination thereof. In other embodiments, the above method of treatment slows or reduces metastasis of the first tumor. In other embodiments, the above method of treatment induces immune memory that can protect against recurrence of the first tumor. In one embodiment, the immune memory comprises inducing central memory (CD44+CD62L+CD8+) T cells. In another embodiment, the above method of treatment induces immune responses against a second tumor at a site away from the site of the first tumor. In one embodiment, the first tumor is breast cancer, for example, a triple-negative breast cancer. In another embodiment, the first tumor is melanoma. In some embodiments, the second tumor is a metastasis of the first tumor. In some embodiments, the metastasis is metastatic breast cancer or metastatic melanoma.

In another embodiment, the present disclosure provides a method of preventing metastasis of a tumor in a subject in need thereof, the method comprising providing the porous scaffold disclosed herein at a site at or near the tumor, thereby preventing metastasis of the tumor in the subject. In some embodiments, the porous scaffold is surgically implanted or inserted at a site at or near the tumor. In one embodiment, the tumor is a solid tumor. In some embodiments, the above porous scaffold comprises T cells that are stimulated to target the tumor, and induction of Tregs is suppressed. In one embodiment, the above method of treatment prolongs survival of the subject. In some embodiments, the tumor is breast cancer, for example, a triple-negative breast cancer. In some embodiments, the tumor is melanoma. In some embodiments, the metastasis is metastatic breast cancer or metastatic melanoma.

### BRIEF DESCRIPTION OF THE FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1F describe the formation and characterization of the silica-heparin microparticles. FIG. 1A is a schematic depicting the chemical modification of the microparticle surface with heparin. FIG. 1B shows a scanning electron micrograph (SEM) synthesized mesoporous microparticles having the diameter of the resulting particles at 3-25 um with pore size 1-7 nm. FIG. 1C is a graph depicting the degree of heparin-conjugation of silica microparticles with various initial amounts of heparin in the reaction mixture. FIG. 1D is a graph demonstrating the binding efficiency of IL-2 (interleukin-2) to the microparticles with a greater than 10-fold improved loading of IL-2 (ug/mg IL-2-bound microparticles) of heparin-modified microparticles (closed circles) compared with unmodified microparticles (open circles). FIG. 1E is a graph demonstrating cumulative release of IL-2 from heparin-functionalized and unmodified silica microparticles at 37 C, showing the delayed kinetics of IL-2 release by heparin-modified microparticles (closed circles) compared with the kinetics of IL-2 release by unmodified microparticles (open circles). FIG. 1F is a graph demonstrating in vitro degradation of silica-based microparticles over time, comparing the degradation of heparin-modified silica microparticles (closed circles) vs. unmodified microparticles (open circles), with the inset bar graph depicting the average decay rate per day for heparin-modified silica microparticles (solid bar) vs. unmodified microparticles (open bar). Calculated diffusion coefficients are shown (inset).

FIG. 2 shows a graph depicting the encapsulation efficiency of IL-2 in unmodified (open circles) compared to heparin-functionalized (closed circles) silica microparticles as a function of initial IL-2 concentration.

FIG. 3 shows a graph demonstrating that incubation of naive CD8+ T cells in presence of silica-based antigen presenting cells (APCs) can induce activation of T cells, measured by tracking the proliferation of T cells as a function of time using carboxyfluorescein succinimidyl ester (CFSE) dilution assay.

FIGS. 4A-4C are graphs and tables demonstrating how T cell activation is modulated by artificial antigen presenting cells (aAPCs). Naive CD4+ and CD8+ T cells were co-cultured with various formulations of aAPCs in two dimensions (2D) (blue closed triangle=IL-2+, anti-CD3/anti-CD28+ antibodies (aCD3/aCD28+); open triangle=IL-2−, aCD3/aCD28+; blue closed square=IL-2+, aCD3/aCD28−; open square=IL-2−, aCD3/aCD28−; black closed square=DYNABEAD® control). FIG. 4A shows flow cytometry analysis of cell division (as a function of CFSE dilution) and percentage of T cells with high expression of CD44 and of T cells upregulating CD25 assayed three days post-stimulation. FIG. 4B shows the percentage of T cells expressing the effector cytokines IL-2, IFN-γ (interferon-gamma), or TNF-α (tumor necrosis factor-alpha). Each dot represents one experiment. FIG. 4C shows fluorescence-activated cell sorting (FACS) quantification of CD8-to-CD4 ratio of T cells cultured with varying formulations of particles, compared to DYNABEADS® (THERMOFISHER SCIENTIFIC™). The starting ratio for all conditions was 0.5.

FIG. 5 shows flow cytometry analysis of cell division (CFSE dilution) in x-axis and CD25 expression in y-axis assayed on day 3 post-stimulation of naive CD8+ T cells with different formulations of developed aAPCs in two-dimensional (2D) culture. Left to right: plain silica microparticles; heparin-modified silica microparticles with IL-2; silica microparticles with anti-CD3/anti-CD28; and heparin-modified silica microparticles with IL-2 and anti-CD3/anti-CD28.

FIGS. 6A-6C show scanning electron micrographs (SEMs) and three-dimensional (3D) activation of the scaffolds. FIG. 6A shows SEM images of macroporous 3D scaffolds. Images were taken from a region within the bulk of the scaffold. Scale bar is 200 μm. FIG. 6B shows Colored SEM images demonstrating association of T cells with the alginate-based scaffolds. Images were taken from a pore wall of the scaffold where T cells were aligned. Scale bar is 10 μm. FIG. 6C depicts flow cytometry analysis of cell division (CFSE dilution) in x-axis and CD25 expression in y-axis assayed three days post-stimulation of naive T cells with different formulations in a three-dimensional (3D) scaffold on the horizontal axis). Left to right: plain 3D scaffold (alginate-RGD [Arg-Gly-Asp] 20 mM); plain 3D scaffold (alginate-RGD 20 mM)+aCD3/aCD28 silica (heparin+IL-2); 3D* (aCD3/aCD28 post-modified) (alginate-RGD 20 mM); 3D* (aCD3/aCD28 post-modified) (alginate-RGD 20 mM)+aCD3/aCD28 silica (no IL-2); 3D* (aCD3/aCD28 post-modified) (alginate-RGD 20 mM)+aCD3/aCD28 silica (heparin+IL-2).

FIG. 7 shows SEM images demonstrating associates of T cells with the alginate-based scaffolds. Images were taken from a region within the pores of the scaffold where T cells engaged. Scale bars are indicated in each panel.

FIGS. 8A-8C show a series of graphs demonstrating how T cell activation is modulated by aAPCs-loaded 3D scaffolds. Naive CD4+ and CD8+ T cells were co-cultured with various formulations of 3D scaffolds (blue closed circle=aAPC+, aCD3/aCD28+; open circle=aAPC+, aCD3/aCD28−; open square=control microparticle (UP; particles that load and release IL-2 but they don't present aCD3/aCD28) [aAPC−, aCD3/aCD28−]). FIG. 8A shows flow cytometry analysis of cell division (CFSE dilution) and CD25/CD44 expression assayed three days post-stimulation with the percentage of cells that divided at least once and the percentage of T cells with high expression of CD44 and percentage of T cells upregulating CD25. FIG. 8B shows the percentage of T cells expressing the effector cytokines IL-2, IFN-γ, or TNF-α. Each dot represents one experiment. FIG. 8C shows FACS quantification of CD8-to-CD4 ratio of T cells cultured with varying formulations of particles, compared to DYNABEADS®. The starting ratio for all conditions was 0.5.

FIG. 9 is a graph demonstrating release of IL-2 encapsulated within aAPCs in an alginate-based 3D scaffold. The released IL-2 was measured using an enzyme-linked immunosorbent assay (ELISA) kit over time under gentle shaking (50 rpm) at 37° C.

FIG. 10 is a graph depicting how porous scaffolds support robust expansion of T cells. Absolute counts of viable T cells in scaffolds fabricated with different formulations are shown (blue line=aAPC+, aCD3/aCD28+; red line=aAPC+, aCD3/aCD28−; gray line=control UP [aAPC−, aCD3/aCD28−]).

FIGS. 11A-11C show a series of graphs demonstrating mechanical characteristics of the 3D hydrogels. FIG. 11A depicts the changes in mechanical properties of freeze-dried scaffolds in the absence or presence of silica-based aAPCs with or without post-conjugation with anti-CD3/anti-CD28 antibodies (open triangle=blank; open circle=with aAPCs; blue closed circle=with aAPCs and aCD3/aCD28 conjugated). For shelf-life evaluation of scaffolds, freeze-dried hydrogel batches were stored at 4° C. for different durations up to six months and changes in (FIG. 11B) mechanical properties and (FIG. 11C) level of activated CD8 T cells were used to assess their shelf-life stability (blue closed circle=with aAPCs and aCD3/aCD28 conjugated; aqua closed circle=with aAPCs and aCD3/aCD28 conjugated after being stored for different periods_). The individual data are presented (n=5). The results were statistically analyzed using one-way analysis of variance (ANOVA) with post-hoc analysis. For all the tests, the threshold was set to P<0.05 for statistically significant. Results showed no statistically significant (p>0.05) change in elastic modulus or T cell activation.

FIGS. 12A-12B show graphs demonstrating the mechanical properties (FIG. 12A) and T cell activation (FIG. 12B) of scaffolds after 1 or 5 cycles of X-ray irradiation at 25 kGy dose compared to freshly prepared samples. The individual data are presented (n=5). The results were statistically analyzed using one-way ANOVA with post-hoc analysis. For all the tests, the threshold was set to P<0.05 for statistically significant. Results showed no statistically significant (p>0.05) changes in elastic modulus or T cell activation.

FIGS. 13A-13B show the chemical structure, molecular weight and reported IC50 values of the two tested transforming growth factor beta (TGFβ) inhibitors, TGF-beta receptor 1 inhibitor (TGF-β receptor 1 inhibitor [TβRI]; Galunisertib LY2157299; FIG. 13A) and TGF-beta receptor (TGF-β receptor inhibitor [TβR]; SB505124; FIG. 13B). In the graphs on the right, in vitro inhibition assays were used to determine effectiveness of these two molecules to inhibit formation of regulatory T cells (Treg) as a function of the geometric mean of Foxp3 expression (top) and the percentage of Foxp3+ cells (bottom) (closed circle=LY2157299; open circle=SB505124). Foxp3+CD25+CD4+ T cells are known as Tregs which their presence suppresses the immunotherapy and help the tumor to growth faster. Suppression of Tregs is known as one of the most effective approaches against cancers.

FIGS. 14A-14D show the results of studies of suppression of Treg formation. FIG. 14A depicts dynamic light scattering (DLS) showing monodisperse formation of TGF-beta inhibitor (TGFbi)-loaded poly(lactic-co-glycolic acid (PLGA) nanoparticles. The inset shows stable suspension of formed nanoparticles in water 24 hours (h) after dispersion. FIG. 14B is a graph depicting release of TGFbi from nanoparticles over time at 37° C. The chemical structure of selected TGFbi, LY2157299, is also shown. FIG. 14C depicts 2D activation and Treg formation using aAPCs in the presence of soluble TGFb. Inhibition using Soluble TGFbi (10 uM) or PLGA NPs loaded with equivalent amounts of TGFbi. FIG. 14D is a graph depicting quantified percentages of formed Tregs in 2D.

FIGS. 15A-15D show the results of studies of 3D Treg inhibition. FIG. 15A is an SEM depicting encapsulation of TGFbi-loaded PLGA nanoparticles in a 3D scaffold. FIG. 15B is a graph depicting release of TGFbi from scaffolds as a function of time at 37° C. FIG. 15C depicts 3D activation and Treg formation using antigen-presenting scaffolds in the presence of soluble TGFb. Inhibition using Soluble TGFbi (10 uM) or PLGA NPs loaded with equivalent amounts of TGFbi. FIG. 15D is a graph depicting quantified percentages of formed Tregs in 3D.

FIG. 16 is a series of graphs depicting the assessment of CCL21 chemotaxis in recruitment of naive and activated CD4+ and CD8+ T cells in vitro. 5×10⁵ (5×105) naive or activated T cells were loaded on the top filter of the transwell chamber (upper left=naïve CD4+; upper right=naïve CD8+; lower left=activated CD4+; lower right=activated CD8+). Hydrogels containing various concentrations of CCL21 were placed in the bottom wells at the indicated concentrations. Viable cells migrating to the lower chamber after 4 h were quantified after digesting the scaffold. Chemotactic Index: fold migration over background (empty scaffolds).

FIG. 17 is a graph depicting the assessment of CCL21 chemotaxis in recruitment of B16F10-OVA tumor cells. 5×105 cells were loaded on the top filter of the transwell chamber. Hydrogels containing various concentrations of CCL21 were placed in the bottom wells at the indicated concentrations. Viable cells migrating to the lower chamber after 8 h were quantified after digesting the scaffold. Chemotactic Index: fold migration over background (empty scaffolds). 5 μm pore size was selected for transwell migration assay.

FIG. 18 is a schematic representation of proposed in vivo mechanism of action. Sustained release of CCL21 helps recruitment of endogenous T cells while presentation of surface conjugated activation cues (anti-CD3 and anti-CD28) and sustained release of IL-2 will activate recruited T cells. Sustained release of TGFb inhibitor will prevent formation of Tregs both in scaffolds and tumors.

FIG. 19 is a series of schematics and photographs depicting an implementation approach as follows: The schematics of the top panel show timing of tumor inoculation and follow up surgical implantation of the biomaterial scaffold. The engineered device is surgically implanted in a B16-F10-ova bearing mouse (available, e.g., ATCC® CRL-6475™), as shown in the photographs in the bottom panel (inset SEM of the scaffold structure).

FIG. 20 is a bright field micrograph of hematoxylin and eosin (H&E) staining of the cross sections of the subcutaneously implanted scaffolds that originated from the alginate biopolymer, 7 days after implantation.

FIG. 21 is a series of photographs depicting clearance of melanoma tumors. Representative images of tumors in situ (top) and subsequently extracted (bottom) from wild-type mice 22 days after tumor inoculation with phosphate buffered saline (PBS; left), control scaffold (center), or full scaffold (right). Local recruitments and activation of endogenous T cells plus Treg suppression via the implanted alginate-based scaffold successfully eliminated the aggressive melanoma tumor in mice. Full scaffold: Alginate-RGD scaffolds, loaded with aAPCs and CCL21, and post-conjugated with anti-CD3 and anti-CD28. Control Scaffold: Alginate-RGD scaffolds.

FIG. 22 shows graphs depicting melanoma (B16-F10-Ova) tumor growth (left) and final tumor mass (right) in wild-type mice implanted with either full (n=7; blue closed circles) or control scaffolds (n=4; red closed circles) compared to PBS (n=4; open circles). Each point represents one mouse.

FIGS. 23A-23B show graphs depicting flow cytometry analysis and FACS quantification (red=control scaffold; blue=full scaffold). FIG. 23A shows flow cytometry analysis of CD4+ and CD8+ T cells recruited and expanded in the scaffolds 17 days after subcutaneous implantation of cell-free scaffolds. FIG. 23B shows FACS quantification of CD8-to-CD4 ratio of recruited T cells extracted from full and control scaffolds. Full scaffold: Alginate-RGD scaffolds, loaded with aAPCs and CCL21, and post-conjugated with anti-CD3 and anti-CD28. Control Scaffold: Alginate-RGD scaffolds.

FIGS. 24A-24D show graphs depicting activation of recruited T cells inside scaffolds. Flow cytometry analysis of T cell activation is studied 17 days after subcutaneous implantation of scaffolds. Activation of recruited CD8+ T cells was monitored by measuring surface expression of CD44 as well as intracellular measurement of Granzyme B (GZMB) expression (red=control scaffold; blue=full scaffold). FIG. 24A show the percentage of T cells with high expression of CD44 and mean fluorescence intensity (MFI) of T cells upregulating CD44 were plotted alongside with representative flow cytometry graphs. FIG. 24B show the percentage of T cells with high intracellular expression of GZMB and MFI of GZMB secreting T cells were plotted. Representative flow cytometry graphs also presented. FIG. 24C show the percentage of T cells with high expression of CD44 activation marker and GZMB effector cytokine were plotted. Representative flow cytometry graphs also presented. FIG. 24D shows the percentage of PD-1 expressing T cells and their MFIs gated on PD-1+ T cells were plotted. Representative flow cytometry graphs also presented. Full scaffold: Alginate-RGD scaffolds, loaded with aAPCs and CCL21, and post-conjugated with anti-CD3 and anti-CD28. Control Scaffold: Alginate-RGD scaffolds.

FIG. 25 shows graphs depicting status of recruited endogenous OTI T Cells (available, e.g., at Charles River Laboratories; C57BL/6-Tg(TcraTcrb)1100Mjb/Crl; OT-1) in scaffolds (red=control scaffold; blue=full scaffold) and demonstrating the results of flow cytometry analysis of OTI CD8+ T cells recruitment and expansion in scaffolds 17 days after subcutaneous implantation of cell-free full and control scaffolds.

FIGS. 26A-26B show graphs depicting the presence of CD8+ T cells and OTI T Cells in tumors (white=PBS; red=control scaffold; blue=full scaffold) and demonstrating the results of flow cytometry analysis of the percentage of (FIG. 26A) CD8+ and (FIG. 26B) OTI CD8+ T cells in tumor 22 days after subcutaneous injection of B16F10-ova cells.

FIGS. 27A-27C show graphs depicting the presence of activated CD8+ T cells inside tumors (white/black=PBS; red=control scaffold; blue=full scaffold) using flow cytometry analysis of T cell activation is studied 22 days after inoculation of tumor cells. Activated CD8+ T cells in the tumor microenvironment were monitored by measuring their surface CD44 expression as well as Granzyme B (GZMB) intracellular expression. FIG. 27A shows the percentage of T cells with high intracellular expression of GZMB and mean fluorescence intensity (MFI) of T cells upregulating GZMB were plotted alongside with representative flow cytometry graphs.

FIG. 27B shows the percentage of T cells with high expression of CD44 activation marker and GZMB effector cytokine were plotted. Representative flow cytometry graphs are also presented. FIG. 27C shows the percentage of PD-1 expressing T cells and their MFIs gated on PD-1+ T cells were plotted. Representative flow cytometry graphs also presented.

FIG. 28 shows graphs depicting the frequency of Foxp3+CD25+CD4+ Tregs in tumor bearing mice. Representative flow cytometry graphs are shown for mice treated with full scaffolds (blue), control scaffolds (red), and PBS (white/black).

FIGS. 29A-29B show graphs depicting the presence of CD8+ T cells and OTI T cells in tumor draining lymph nodes and demonstrating the results of flow cytometry analysis of percentage of (FIG. 29A) CD8+ and (FIG. 29B) OTI CD8+ T cells in tumor draining lymph nodes 22 days after subcutaneous injection of B16F10-ova cells in mice receiving different treatment (white/black=PBS; red=control scaffold; blue=full scaffold).

FIGS. 30A-30C show graphs depicting the presence of activated CD8+ T cells in tumors draining lymph nodes (white/black=PBS; red=control scaffold; blue=full scaffold) and demonstrating the results of flow cytometry analysis of T cell activation is studied 22 days after inoculation of tumor cells. Activation of CD8+ T cells in the tumor draining lymph nodes was monitored by measuring their surface CD44 expression as well as Granzyme B (GZMB) intracellular expression. FIG. 30A shows the percentage of T cells with high intracellular expression of GZMB and mean fluorescence intensity (MFI) of T cells upregulating GZMB were plotted alongside with representative flow cytometry graphs. FIG. 30B shows the percentage of T cells with high expression of CD44 activation marker and GZMB effector cytokine were plotted. Representative flow cytometry graphs also presented. FIG. 30C shows the percentage of PD-1 expressing T cells and their MFIs gated on PD-1+ T cells were plotted. Representative flow cytometry graphs also presented.

FIG. 31 shows a series of graphs depicting the frequency of Foxp3+CD25+CD4+ Tregs in tumor draining lymph nodes. Representative flow cytometry graphs are shown for mice treated with full scaffolds (blue), control scaffolds (red), and PBS (white/black).

FIGS. 32A-32B shows a series of graphs depicting the presence of CD8+ T cells and OTI T cells in spleen (white/black=PBS; red=control scaffold; blue=full scaffold) and demonstrating the results of flow cytometry analysis of the percentage of (FIG. 32A) CD8+ and (FIG. 32B) OTI CD8+ T cells in the spleen of tumor-bearing mice 22 days after subcutaneous injection of B16F10-ova cells for mice with different treatments.

FIGS. 33A-33C show a series of graphs depicting the presence of activated CD8+ T cells in spleen (white/black=PBS; red=control scaffold; blue=full scaffold) and demonstrating the results of flow cytometry analysis of T cell activation is studied 22 days after inoculation of tumor cells. The percentage of GZMB+CD44+ T cells was similar in treated vs untreated conditions accompanied with their FACS representatives (FIG. 33A). The percentage of T cells with high intracellular expression of GZMB and mean fluorescence intensity (MFI) of T cells upregulating GZMB were plotted alongside with representative flow cytometry graphs (FIG. 33B). The percentage of PD-1 expressing T cells and their MFIs gated on PD-1+ T cells were plotted (FIG. 33C). Representative flow cytometry graphs are also presented.

FIG. 34 shows a series of graphs demonstrating that engineered scaffolds can suppress growth of melanoma tumors via recruitment of endogenous T cells (white/black=PBS; red=control scaffold; blue=CCL21 full scaffold; pink=SDF-1a full scaffold). Melanoma (B16-F10-Ova) tumor growth in wild-type mice with full, control scaffolds or PBS treatment (n=4-7) was studied. Here, the therapeutic effects of two chemokines (CCL21 [blue] and SDF-1a [pink]) were studied. Each point represents a mouse.

FIG. 35 shows a series of graphs demonstrating that engineered scaffolds can suppress growth of melanoma tumors via recruitment of endogenous T cells (white/black=PBS; red=control scaffold; blue=CCL21 full scaffold; pink=SDF-1a full scaffold). Melanoma (B16-F10-Ova) tumor masses were measured 22 days after tumor inoculation in wild-type mice treated with Full or control scaffolds or PBS treatment (n=4-7). Here the therapeutic effects of two chemokines (CCL21 [blue] and SDF-1a [pink]) was studied. Each point represents a mouse.

FIG. 36 shows a series of graphs depicting the status of recruited T cells in scaffolds and demonstrating the results of flow cytometry analysis of CD4+ and CD8+ T cells recruited by the scaffolds 17 days after subcutaneous implantation of cell-free (full) scaffolds releasing either CCL21 (blue) or SDF-1a (pink) chemokines (n=4). FACS quantification of CD8-to-CD4 ratio of recruited T cells extracted from full and control scaffolds.

FIG. 37 shows a series of graphs depicting the frequency of activated CD44+CD8+ and GZMB+CD8+ in scaffolds after treating mice with full scaffolds releasing either CCL21 (blue) or SDF-1a (pink) chemokines (n=4). Representative flow cytometry data were provided.

FIGS. 38A-38C show schematics and graphs demonstrating that engineered scaffolds can preserve their therapeutic function several months after fabrication. FIG. 38A shows schematics demonstrating the timing of tumor inoculation and follow up surgical implantation of cells-free scaffolds. Melanoma (B16-F10-Ova) tumor growth (FIG. 38B) and final tumor masses (FIG. 38C) were measured 22 days after tumor inoculation in wild-type mice treated with fresh (blue) or 6-month old (pink) full scaffolds (n=4-7). Each point represents a mouse. Full scaffold: Alginate-RGD scaffolds, loaded with aAPCs and CCL21, and post-conjugated with anti-CD3 and anti-CD28. Control Scaffold: Alginate-RGD scaffolds.

FIGS. 39A-39F show a series of graphs depicting recruitment and activation of endogenous CD8+ and CD4+ T cells in freshly prepared (blue) and 6-month old (pink) scaffolds and demonstrating flow cytometry analysis of the percentages of (A) CD8+ and (B) CD4+ and (C) the ratio of CD8+/CD4+ T cells in the scaffolds. The frequency of activated CD8+ T cells in the scaffolds was assessed by (D) CD44 and (E) GZMB, as well as by (F) co-expression of CD44 and GZMB T cells in freshly prepared (blue) or 6-month old (pink) full scaffolds. Full scaffold: Alginate-RGD scaffolds, loaded with aAPCs and CCL21, and post-conjugated with anti-CD3 and anti-CD28. Control Scaffold: Alginate-RGD scaffolds.

FIGS. 40A-40B show a series of graphs depicting the frequency of (A) activated CD44+GZMB+CD8+(B) Foxp3+CD25+CD4+ Tregs in tumors after being treated with fresh (blue) or 6-month old (pink) full scaffolds. Full scaffold: Alginate-RGD scaffolds, loaded with aAPCs and CCL21, and post-conjugated with anti-CD3 and anti-CD28. Control Scaffold: Alginate-RGD scaffolds.

FIGS. 41A-41D are schematics, photographs, and graphs demonstrating that engineered scaffolds not only can suppress the growth of local tumors, but they can also affect the distant tumors. (A) Schematics depict the timing of inoculation of primary and secondary tumors and follow up surgical implantation of the cell-free scaffolds. (B) Photographs show the growth of primary and secondary tumors in the control mouse. (C) Melanoma (B16-F10-Ova) tumor growth and (D) final tumor masses were measured 22 days after inoculation of primary tumors in wild-type mice treated with PBS (white), control scaffold (red), or full scaffold (blue) (n=4-7). Each point represents a mouse. Full scaffold: Alginate-RGD scaffolds, loaded with aAPCs and CCL21, and post-conjugated with anti-CD3 and anti-CD28. Control Scaffold: Alginate-RGD scaffolds.

FIGS. 42A-42B are graphs demonstrating that the percentage of tumor infiltrating CD8+ T cells was increased by more than two times in the contralateral tumor of the mice that received scaffold treatment. FIG. 42A shows the results of a representative flow cytometry study of CD8+ T cells present in the primary (left) and secondary (right) tumors after being treated with PBS (white), control scaffolds (red), or full scaffolds (blue). FIG. 42B shows the frequency of CD8+ T cells in primary (left) and secondary (right) tumors (n=4).

FIG. 43 shows SEM of tumor-associated CD8+ T cells, which were stained in primary (top) and secondary (bottom) tumors 22 days after tumor inoculation and treatment with full scaffolds (left) or PBS (right). Note: As 3 out of 7 mice treated with full scaffold formulation did not grow tumors, these representative sections were only found in the few mice with remaining tumors. Full scaffold: Alginate-RGD scaffolds, loaded with aAPCs and CCL21, and post-conjugated with anti-CD3 and anti-CD28. Control Scaffold: Alginate-RGD scaffolds.

FIG. 44 shows graphs depicting a flow cytometry study of PD-1+CD8+ T cells present in primary (left) and secondary (right) tumors after being treated with PBS (white), control scaffold (red), or full scaffold (blue) (n=4).

FIGS. 45A-45C show graphs depicting the results of a study of activated GranzymeB secreting CD8+ T cells. FIG. 45A shows the results of a flow cytometry study of GZMB+CD8+ T cell presence in primary (top) and secondary (bottom) tumors after being treated with PBS (white), or full scaffold (blue). The frequency (FIG. 45B) and MFI (FIG. 45C) of GZMB+CD8+ T cells in primary and secondary tumors (n=4) are shown for each group. Full scaffold: Alginate-RGD scaffolds, loaded with aAPCs and CCL21, and post-conjugated with anti-CD3 and anti-CD28. Control Scaffold: Alginate-RGD scaffolds.

FIG. 46 shows graphs demonstrating the results of a flow cytometry study of the frequency of CD44+GZMB+CD8+(activated) (top) and CD44+CD62L+CD8+(central memory) (bottom) T cells in primary (left) and secondary (right) tumors after being treated with PBS (white), control scaffold (red), or full scaffold (blue) (n=4). Full scaffold: Alginate-RGD scaffolds, loaded with aAPCs and CCL21, and post-conjugated with anti-CD3 and anti-CD28. Control Scaffold: Alginate-RGD scaffolds.

FIGS. 47A-47B show graphs depicting the results of a study of a flow cytometry study of CD44+KLRG-1+CD8+ T cell presence in primary and secondary tumors after being treated with Full or control Scaffolds. Representative FACS (FIG. 47A) and frequency (FIG. 47B) of CD44+KLRG-1+CD8+ T cells in primary and secondary tumors (n=4) are shown (PBS (white), control scaffold (red), or full scaffold (blue)). Full scaffold: Alginate-RGD scaffolds, loaded with aAPCs and CCL21, and post-conjugated with anti-CD3 and anti-CD28. Control Scaffold: Alginate-RGD scaffolds.

FIGS. 48A-48B are graphs depicting a study of the population of Tregs in both primary and secondary tumors. (A) Representative flow cytometry of Foxp3+CD25+CD4+ Tregs in primary and secondary tumors for mice treated with full scaffolds (blue) and PBS (black) is shown. (B) The quantified frequency of Foxp3+CD25+CD4+ Tregs in primary and secondary tumors is shown (PBS (white), control scaffold (red), or full scaffold (blue)). Full scaffold: Alginate-RGD scaffolds, loaded with aAPCs and CCL21, and post-conjugated with anti-CD3 and anti-CD28. Control Scaffold: Alginate-RGD scaffolds.

FIGS. 49A-49B are graphs depicting the results of a study of T cells recruited by scaffolds. (A) Flow cytometry study of CD8+ T cell presence in scaffolds implanted in tumors is shown (control scaffold (red), or full scaffold (blue)). (B) The frequency of CD8+ and CD4+ T cells as well as the CD8 to CD4 T cell ratios in control scaffolds (red; n=4) and full scaffolds (blue; n=7). Full scaffold: Alginate-RGD scaffolds, loaded with aAPCs and CCL21, and post-conjugated with anti-CD3 and anti-CD28. Control Scaffold: Alginate-RGD scaffolds.

FIGS. 50A-50B are graphs depicting the results of a flow cytometry study of CD44+CD8+ and GZMB+CD8+ T cell presence in scaffolds 17 days after being implanted in tumor-bearing mice. Representative FACS (A) and the frequency (B) of CD44+CD8+ and GZMB+CD8+ T cells as well as MFI of GZMB+CD8+ T cells in control (red; n=4) scaffolds and full (blue; n=7) scaffolds are shown. Full scaffold: Alginate-RGD scaffolds, loaded with aAPCs and CCL21, and post-conjugated with anti-CD3 and anti-CD28. Control Scaffold: Alginate-RGD scaffolds.

FIGS. 51A-51B are graphs depicting the results of a flow cytometry study of CD44+KLRG-1+CD8+ T cell presence in scaffolds 17 days after being implanted in tumor-bearing mice. Representative FACS (A) and the frequency (B) of CD44+KLRG-1+CD8+ T cells in control (red; n=4) and full (blue; n=7) scaffolds are shown. Full scaffold: Alginate-RGD scaffolds, loaded with aAPCs and CCL21, and post-conjugated with anti-CD3 and anti-CD28. Control Scaffold: Alginate-RGD scaffolds.

FIGS. 52A-52B are graphs depicting the results of a flow cytometry study of CD8+ T cell presence in draining lymph nodes of primary (left) and secondary (right) tumors after being treated with PBS (white/black), control (red; n=4) scaffolds, or with full (blue; n=7) scaffolds. Representative FACS graphs (A) and the frequency (B) of CD8+ T cells in draining lymph nodes of primary and secondary tumors are shown. Full scaffold: Alginate-RGD scaffolds, loaded with aAPCs and CCL21, and post-conjugated with anti-CD3 and anti-CD28. Control Scaffold: Alginate-RGD scaffolds.

FIGS. 53A-53B are graphs depicting the results of a flow cytometry study of GZMB+CD8+ T cell presence in draining lymph nodes of primary (left) and secondary (right) tumors after being treated with PBS (white/black), control (red; n=4) scaffolds, or with full (blue; n=7) scaffolds. Representative FACS graphs (A) and the frequency (B) of GZMB+CD8+ T cells in draining lymph nodes of primary (left) and secondary (right) tumors are shown. Full scaffold: Alginate-RGD scaffolds, loaded with aAPCs and CCL21, and post-conjugated with anti-CD3 and anti-CD28. Control Scaffold: Alginate-RGD scaffolds.

FIGS. 54A-54B are graphs depicting the results of a flow cytometry study of CD44+GZMB+CD8+(effector) T cell presence in draining lymph nodes of primary (left) and secondary (right) tumors after being treated with PBS (white/black), control (red; n=4) scaffolds, or with full (blue; n=7) scaffolds. Representative FACS graphs (A) and the frequency (B) of CD44+GZMB+CD8+ T cells in draining lymph nodes of primary and secondary tumors are shown. Full scaffold: Alginate-RGD scaffolds, loaded with aAPCs and CCL21, and post-conjugated with anti-CD3 and anti-CD28. Control Scaffold: Alginate-RGD scaffolds.

FIG. 55 shows graphs depicting the results of a flow cytometry study of the frequency of CD44+CD62L+CD8+(central memory) T cell presence in draining lymph nodes of primary (left) and secondary (right) tumors after being treated with PBS (white), control (red; n=4) scaffolds, or with full (blue; n=7) scaffolds. Full scaffold: Alginate-RGD scaffolds, loaded with aAPCs and CCL21, and post-conjugated with anti-CD3 and anti-CD28. Control Scaffold: Alginate-RGD scaffolds.

FIGS. 56A-56B show graphs depicting the results of a study on the population of Tregs. FIG. 56A shows representative flow cytometry of Foxp3+CD25+CD4+ Tregs in primary (top) and secondary (bottom) tumor draining lymph nodes for mice treated with full scaffolds (blue) or PBS (black). FIG. 56B shows the quantified frequency of Foxp3+CD25+CD4+ Tregs found in tumor draining lymph nodes for mice treated with PBS (white), control (red) scaffolds, or with full (blue) scaffolds. Full scaffold: Alginate-RGD scaffolds, loaded with aAPCs and CCL21, and post-conjugated with anti-CD3 and anti-CD28. Control Scaffold: Alginate-RGD scaffolds.

FIGS. 57A-57B show graphs depicting the results of a flow cytometry study of CD8+ T cell presence in the spleen of mice after being treated with PBS (white), control (red; n=4) scaffolds, or with full (blue; n=7) scaffolds. Representative FACS graphs (A) and the frequency (B) of CD8+ T cells are shown. Full scaffold: Alginate-RGD scaffolds, loaded with aAPCs and CCL21, and post-conjugated with anti-CD3 and anti-CD28. Control Scaffold: Alginate-RGD scaffolds.

FIGS. 58A-58C show graphs depicting the results of flow cytometry study of effector and memory T cells presence in the spleen of tumor bearing mice after being treated with PBS (white), control (red; n=4) scaffolds, or with full (blue; n=7) scaffolds. (A) Representative FACS graphs and frequency of GZMB+CD8+ T cells in the spleen are shown. The frequency of (B) CD44+GZMB+CD8+(effector) and (C) CD44+CD62L+CD8+(central memory) T cells in the spleen is also shown. Full scaffold: Alginate-RGD scaffolds, loaded with aAPCs and CCL21, and post-conjugated with anti-CD3 and anti-CD28. Control Scaffold: Alginate-RGD scaffolds.

FIGS. 59A-59F show schematics, photographs, and graphs demonstrating how engineered scaffolds can deliver tumor-reactive T cells and suppress growth of melanoma tumors. (A) The schematics demonstrate timing of tumor inoculation and follow up surgical implantation of the activated OTI cells-loaded biomaterial scaffold. (B) Photographs depict how the engineered device is surgically implanted in a B16-F10-ova bearing mice. (C) H&E staining showing connective tissue-like scaffolds is shown. (D) Representative photographic images of subdermal tumors from wild-type mice 22 days after tumor inoculation are shown. Alginate scaffolds carrying tumor reactive T cells and T cell-specific activator cues can eliminate melanoma tumors in mice. (E) Melanoma (B16-F10-Ova) tumor growth and (F) final tumor mass in wild-type mice with control (red) scaffolds, or with full (blue) scaffolds compared to PBS (n=5). Each point represents one mouse. Full scaffold: Alginate-RGD scaffolds, loaded with aAPCs and CCL21, and post-conjugated with anti-CD3 and anti-CD28. Control Scaffold: Alginate-RGD scaffolds. n=5 for each group.

FIG. 60 shows graphs and images depicting results of a study of tumor clearance. Top: Melanoma (B16-F10-Ova) tumor growth for groups with different treatments (left to right: PBS, intravenous (IV) injection of activated OT-I T cells, control scaffold to deliver OT-I T cells, full scaffold to deliver OT-I T cells). Each line represents the tumor size of a single mouse over time. Bottom: Histologic analysis of the tumor tissues via H&E stain for animals used as PBS control (left) vs. OT1-loaded full scaffolds (right).

FIGS. 61A-61C show graphs depicting results of a study of the presence of activated CD8+ T cells in tumors. The presence of tumor specific CD8+ T cells (OTI) as well as their level of cytokine secretion and PD-1 expression in tumors was studied 22 days after inoculation of tumor cells using flow cytometry (red=PBS; blue=IV injection; black=control scaffold; white=full scaffold). FIG. 61A depicts the presence of OTI and CD8+ T cells found in tumors. Frequency of CD8+ T cells with high expression of GZMB and PD-1 and mean fluorescence intensity (MFI) of T cells upregulating these two proteins were measured. FIG. 61B depicts the percentage of T cells with high co-expression of CD44 activation marker and GZMB effector cytokine were plotted. Representative flow cytometry graphs also presented. FIG. 61C depicts the frequency of Foxp3+CD25+CD4+ Tregs in tumor were studied. Representative flow cytometry graphs are shown for mice treated with indicated treatments. (n=5).

FIGS. 62A-62B are graphs comparing the presence of activated CD8+ T cells in tumors. Flow cytometry used to identify the presence of CD44 activated CD8+ T cells in tumors as shown (green=full scaffold/OT1; orange=control scaffold/OT1; aqua=OT1 IV injection; red=PBS control; black=activated OT1 T cell prior to loading). Shifts of the peak to the right indicate more expression of the target surface marker, here CD44, which means more activation of T cells.

FIG. 63 is a series of graphs depicting the presence of activated CD8+ T cells in tumors as shown (green=full scaffold/OT1; orange=control scaffold/OT1; aqua=OT1 IV injection; red=PBS control). Flow cytometry used to identify the presence of CD44+, Granzyme+, and PD-1+ cells gated on CD8+ T cells (upper panels) and gated on OTIs (lower panels) in tumors. OTI T cells were recognized by staining for Vα2 (V-alpha2) surface receptors. Shifts of the peak to the right mean more expression of the target surface (CD44 or PD-1) or intracellular cytokines (Granzyme B), which means more activation of T cells.

FIG. 64 is a series of confocal fluorescence microscopy showing the results of a terminal deoxynucleotidyl transferase dUTP nick end labeling (TUNEL) assay to observe DNA degradation as a measure of apoptotic tumor cells. Local delivery of tumor-reactive T cells (OT1) can promote tumor apoptosis. Cell apoptosis was detected using TUNEL staining for samples with various treatments (top: PBS control (left) and IV OTI (right); bottom: control scaffold+OTI (left) and full scaffold+OTI (right)).

FIGS. 65A-65C are a series of graphs depicting the results of a study on the presence of activated CD8+ T cells in tumor draining lymph nodes. Presence of tumor-antigen specific CD8+ T cells (OTI) as well as activation of CD8+ T cells in the tumor draining lymph nodes was studied 22 days after inoculation of tumor cells using flow cytometry (red=PBS; blue=IV injection; black=control scaffold; white=full scaffold). FIG. 65A shows the percentage of OTI and CD8+ T cells found in tumor draining lymph nodes. The frequency of CD8+ T cells with high expression of GZMB and PD-1 and mean fluorescence intensity (MFI) of T cells upregulating these two proteins were measured. FIG. 65B shows the percentage of T cells with high expression of CD44 activation marker and GZMB effector cytokine were plotted. Representative flow cytometry graphs are also presented. (n=5). FIG. 65C shows the frequency of Foxp3+CD25+CD4+ Tregs in tumors. Representative flow cytometry graphs are shown for mice treated with indicated treatments (n=5 for each group).

FIGS. 66A-66B are a series of graphs depicting the results of a study on the presence of activated CD8+ T cells in spleen. The presence of tumor-antigen specific CD8+ T cells (OTI) as well as activation of CD8+ T cells in the spleen of tumor bearing mice was studied 22 days after inoculation of tumor cells using flow cytometry (red=PBS; blue=IV injection; black=control scaffold; white=full scaffold). FIG. 66A show the percentage of OTI and CD8+ T cells found in spleen. Frequency of CD8+ T cells with high expression of GZMB and PD-1 and mean fluorescence intensity (MFI) of T cells upregulating these two proteins were measured. FIG. 66B shows the percentage of T cells with high expression of CD44 activation marker and GZMB effector cytokine as plotted. Representative flow cytometry graphs are also presented (n=5 for each group).

FIG. 67 is a schematic representation of one embodiment of scaffold components and fabrication steps.

FIGS. 68A-68L demonstrate immunoactive scaffold activates T cells and suppresses Tregs. FIG. 68A: Scanning electronic micrograph (SEM) images of macroporous 3D scaffolds. Images were taken from a region within the bulk of the scaffold. Scale bar is 200 μm.

FIG. 68B: Enlarged SEM images demonstrating association of T cells within the alginate-based scaffolds. Images were taken from a pore wall of the scaffold where T cells were aligned. Scale bar is 10 μm. FIG. 68C: To better assess pore sizes of the scaffold in vivo, scaffolds were implanted in wild-type non-tumor-bearing mice for 7 days, and H&E staining showed connective tissue-like scaffolds. Scale bar is 100 μm. FIG. 68D: Confocal fluorescence images showing interaction of primary T cells with the scaffold. Scale bars are 50 and 5 μm.

FIGS. 68E-68G show T cell activation and proliferation. FIG. 68E: Naïve CD4+ and CD8+ primary mouse T cells were co-cultured with 3D scaffolds. Scaffolds formulated by surface coating with stimulatory antibodies (anti-CD3 and anti-CD28) resulted in robust responses. Flow cytometry analysis of cell division (CFSE dilution) and expression of activation markers CD25 or CD44 assayed three days after the introduction of T cells into the biomaterial. % Proliferated is the percentage of T cells that divided at least once. FIG. 68F: Naïve CD4+ and CD8+ primary mouse T cells were co-cultured with 3D scaffolds. Scaffolds formulated by surface coating with stimulatory antibodies (anti-CD3 and anti-CD28) resulted in robust responses. Percentage of T cells expressing the effector cytokines IL-2, IFN-γ, or TNF-α. Each dot represents one independent experiment. FIG. 68G: Naïve CD4+ and CD8+ T cells were co-cultured with different formulations of 3D scaffolds. The starting number of T cells was 5×10⁶ cells. Absolute counts of viable T cells in scaffolds fabricated with stimulatory antibodies (anti-CD3 and anti-CD28) showed robust expansion of T cells. Each line represents an independent experiment. FIGS. 68H-68I show the effects of mechanical stiffness. FIG. 68H: Elastic modulus of the 3D alginate scaffold fabricated with three different concentrations of Ca²⁺. FIG. 68I: T cell proliferation and activation markers expression in scaffolds of various mechanical stiffness. FIGS. 68J-68L show the effects of TGF-β inhibition. FIG. 68J: Release of TGF-βi encapsulated PLGA nanoparticles embedded in the 3D alginate scaffold. The released TGF-βi was measured over time under gentle shaking (50 rpm) at 37° C. FIG. 68K: Flow cytometry analysis of Treg formation using antigen-presenting scaffolds in the presence (left) and absence (right) of TGF-βi releasing nanoparticles 4 days into culture. FIG. 68L: Inhibition of Treg development in 3D scaffold by soluble TGF-βi versus PLGA-encapsulated TGF-βi was quantified. The individual data are presented (n=5). The results were statistically analyzed using one-way ANOVA with post-hoc analysis.

FIGS. 69A-69J show engineered scaffolds that curb breast cancer. FIG. 69A: Schematic representation of the scaffold components. Sustained release of CCL21 helps recruit endogenous T cells, while the presentation of surface-conjugated activation cues (anti-CD3 and anti-CD28) and the sustained release of IL-2 activates recruited T cells. Sustained release of TGF-β inhibitor depletes Tregs in tumors. FIG. 69B: Schematic of 4T1 breast cancer experiment, along with photos of the injection of tumor cells into the mammary fat pad and the implantation of scaffolds into the mammary fat space. FIG. 69C: Bioluminescence imaging of luciferase-producing cancer cells in mice treated with immunoactive scaffolds or controls. FIG. 69D: Bioluminescence quantified over time across mice. FIG. 69E: Survival of mice after introduction of 4T1 breast cancer cells in treated and control groups. FIG. 69F: Tumor mass on day 15. FIG. 69G: T cells were more activated and enriched for cytotoxic T cells. FIG. 69H: Tregs were suppressed in mice treated with immunoactive scaffolds. FIGS. 69I-69J: Bioluminescence imaging on day 30 of head and upper limbs reveals metastatic disease distant from primary site. Quantified results of bioluminescence imaging of distant sites. Each point represents one mouse.

FIGS. 70A-70E show individual components cannot accomplish the same tumor suppression as full scaffolds. 4T1 breast cancer tumor growth (FIG. 70A) and final tumor mass (FIG. 70B) in wild-type mice implanted with either full or control scaffolds that were composed of only one individual component including aCD3/CD28 conjugation, IL-2 loading, CCL21 loading, or TGFbi loading as well as blank scaffolds. The treatment group that received intratumoral injection of all the components in the soluble format is also included here. Presence (FIG. 70C) and activation (FIG. 70D) of CD8+ T cells were studied. Frequency of Tregs (FIG. 70E) is also demonstrated in tumor tissues. (n=5). Each point represents one mouse. Please refer to Table 3 for the statistical comparisons.

FIGS. 71A-71K show engineered scaffolds can suppress the growth of local and distant breast tumors and create anti-cancer memory. FIG. 71A: Timing of i.v. injection of 4T1 breast cancer cells at 100 days post injection of primary tumors in mice who eliminated primary tumors (n=4). Naïve mice (same age) receiving i.v. injection of 4T1 breast cancer cells were used as a control. FIG. 71B: Representative bioluminescence imaging of luciferase-producing cancer cells at different times post injection of 4T1-Luc in mice treated with immunoactive scaffolds. FIG. 71C: Quantification of bioluminescence intensity of mice at different times post injection of 4T1-Luc cancer cells. FIG. 71D: Survival of mice after i.v. injection of 4T1 breast cancer cells in treated and control groups. FIG. 71E: Timing of inoculation of primary and secondary tumors and follow up surgical implantation of the cell-free scaffolds. Wildtype, immunocompetent syngeneic mice were implanted in the mammary fat pad with 5×10⁵ 4T1-luc breast cancer cells. After 5 days, the scaffolds were implanted and 3×10⁵ tumor cells were injected to the contralateral mammary fat pad. FIG. 71F: Bioluminescence imaging of luciferase-producing cancer cells in mice treated with immunoactive scaffolds or (PBS) controls at various timepoint. FIG. 71G: Growth of primary and secondary 4T1 tumors in mice as quantified over time using bioluminescence imaging. FIG. 71H: tumor masses were measured 15 days after inoculation of primary tumors in wild-type mice treated with immunoactive scaffolds. FIG. 71I: The frequency of CD8+ T cells and activated (GZMB+) CD8+ T cells in primary and secondary tumors. FIG. 71J: The quantified frequency of Foxp3+CD25+CD4+ Tregs in primary and secondary tumors. FIG. 71K: The frequency of central memory (CD44+CD62L+CD8+) T cells in primary and secondary tumors (n=4). Each point represents a mouse.

FIG. 72 shows secondary breast cancer tumor model, T cell activation in the draining lymph nodes, 15 and 21 days after inoculation of tumor cells. Percentage of CD8+ T cells found in tumor draining lymph nodes being treated with immunoactive scaffold or PBS control. Frequency of CD8+ T cells with high expression of GZMB and Foxp3+CD25+CD4+ Tregs were measured. (n=4).

FIG. 73 shows serum cytokine concentration. Concentration of IFN-γ and TNF-α inflammatory cytokines in mice peripheral blood 7 days post insertion of “Full” scaffolds (closed circles) compared to control mice injected with PBS (open circles).

FIGS. 74A-74H show engineered scaffolds that curb melanoma. FIG. 74A: Schematic of B16 experiment. FIG. 74B: Scaffold and its implantation into the subcutaneous space adjacent to the tumor. The engineered device was surgically implanted in a B16-F10-ova bearing mice. FIG. 74C: Representative image of tumors extracted from wild-type mice 22 days after tumor inoculation. FIG. 74D: Melanoma (B16-F10-Ova) tumor growth and final tumor mass in wild-type mice implanted with either full (n=7) or control scaffolds (n=4) compared to PBS (n=4). Each point represents one mouse. Survival of mice following treatment with either PBS, Blank Scaffold or immunoactive (Full) scaffolds. (n=5). FIG. 74E: Status of recruited T cells in tumors. Flow cytometry analysis of CD4+ and CD8+ T cells recruited and expanded in the tumors 22 days after subcutaneous injection of B16F10-ova cells. Activation of recruited CD8+ T cells was monitored by measuring surface expression of CD44 as well as intracellular measurement of Granzyme B expression. FIGS. 74F-74G: The frequency of Foxp3+CD25+CD4+ Tregs in tumors and tumor-draining lymph nodes. FIG. 74H: Activated CD8+ T cells inside scaffolds. Flow cytometry analysis of the percentage of CD8+ T cells in explanted scaffolds 17 days after subcutaneous implantation of scaffolds.

FIGS. 75A-75C show X-ray irradiation (sterilization) will not affect the functionality of IL-2 cytokine, CCL21 chemokine, and aCD3/aCD28 antibodies. FIG. 75A: Naïve CD8+ mouse T cells were cultured with control and irradiated IL-2 cytokine and aCD3/aCD28 antibodies. Flow cytometry analysis of cell division (CFSE dilution) was assayed three days after treatment of the T cells with the above mentioned formulations. FIG. 75B: the percentage of T cells that divided at least once. FIG. 75C: Assessment of CCL21 chemotaxis following irradiation. 5×10⁵ activated CD8+ T cells were loaded on top of the transwell chamber. Hydrogels containing fresh or irradiated recombinant CCL21 were placed in the bottom wells at the indicated concentrations. Viable cells that migrated to the lower chamber after 4 h were quantified after digesting the scaffold. Chemotactic Index: fold migration over background (empty scaffolds). 5 μm pore size was selected for Transwell migration assay.

FIGS. 76A-76I show engineered scaffolds can suppress the growth of local and distant tumors. FIG. 76A: Timing of inoculation of primary and secondary tumors and follow up surgical implantation of the cell-free scaffolds. FIG. 76B: Growth of primary and secondary tumors in the control mouse. FIG. 76C: Melanoma (B16-F10-Ova) tumor growth and final tumor masses (FIG. 76D) were measured 22 days after inoculation of primary tumors in wild-type mice treated with Full or control scaffolds (n=4-7). Each point represents a mouse. FIG. 76E: The frequency of CD8+ T cells in primary and secondary tumors (n=4). Flow cytometry study of the frequency of CD44+GZMB+CD8+(activated) (FIG. 76F) and CD44+CD62L+CD8+(central memory) T cells (FIG. 76G) in primary and secondary tumors (n=4). FIG. 76H: Representative flow cytometry of Foxp3+CD25+CD4+ Tregs in primary and secondary tumors of mice treated with Full Scaffolds (Blue) and PBS (Black). FIG. 76I: The quantified frequency of Foxp3+CD25+CD4+ Tregs in primary and secondary tumors.

FIGS. 77A-77J show engineered scaffolds deliver antigen-specific T cells, suppress Tregs, and control melanomas. FIG. 77A: Timing of tumor inoculation and implantation of the activated OTI cells-loaded scaffold. FIG. 77B: The engineered device is surgically implanted in a B16-F10-ova bearing mouse. FIG. 77C: H&E staining showing connective tissue-like scaffolds. FIG. 77D: Representative images of subdermal tumors from wild-type mice 22 days after tumor inoculation. Alginate scaffolds carrying tumor reactive T cells and T cell-specific activator cues can eliminate melanoma tumors in mice. FIG. 77E: Melanoma (B16-F10-Ova) tumor growth and final tumor mass (FIG. 77F) in wild-type mice with immunoactive or control scaffolds compared to PBS (n=5). Each point represents one mouse. FIG. 77G: Presence of tumor specific CD8+ T cells (OTI) in tumors was studied 22 days after inoculation of tumor cells using flow cytometry. FIG. 77H: Frequency of CD8+ T cells with high expression of GZMB effector cytokine. FIG. 77I: The frequency of Foxp3+CD25+CD4+ Tregs in tumor were studied (n=5). FIG. 77J: Tumor-cell apoptosis was detected using TUNEL staining after various treatments, examples of staining on the left and summarized data on the right.

FIG. 78 shows therapeutic advantage of T cell delivery in a more stablished melanoma tumor. Survival of mice after subcutaneous injection of either 5×10⁵ or 1×10⁶ B16F10-Ova melanoma cells following treatment with either PBS or immunoactive (Full) scaffolds in the presence or absence of loaded OT1 T cells.

FIG. 79 shows biosafety evaluation of Full scaffold. Histology analysis of the major organs of mice 7 days after surgical implantation of the Full scaffolds. Images are presented in two magnifications.

FIG. 80 shows change in the calcium content of alginate-based scaffolds following extended washing in PBS, and the subsequent incubation in T-cell media. At various times shown, the calcium content released from lysis of the scaffold were measured. Each data point represents an independent scaffold (n=5).

FIG. 81 shows the presence of BSA in the formulation does not affect T cell activation. Naïve OT1 CD8+ primary mouse T cells were co-cultured with Ovalbumin antigen (SIINFEKL) as a positive control or BSA (1 and 10 mg/ml) in the presence of LPS (100 ng/ml). Flow cytometry analysis of cell division (CFSE dilution) was assayed three days after treatment of T cells with the above mentioned formulations.

FIG. 82 shows population of tumor associated macrophages and dendric cells (DCs). Graphs showing the frequency of activated (CD86+) CD11b+F4/80+ macrophages and activated (CD86+MHCII+) CD11c+ DCs.

FIG. 83 shows the presence or absence of Luciferase (Luc) enzyme will not affect progression and response of 4T1 breast cancer cells in vivo. Tumor mass on day 15 after mammary fat pad injection of 5×105 4T1 or 4T1-Luc cancer cells. Frequency of CD8+ T cells and activated CD8+ T cells within tumor microenvironment. Suppression of Tregs in mice bearing 4T1 or 4T1-Luc cancer cells treated with either PBS control or immunoactive scaffolds were also evaluated. Each point represents one mouse.

FIG. 84 shows the presence or absence of Ova will not affect progression and response of B16F10 melanoma in vivo. Survival of mice after subcutaneous injection of 5×105 B16F10 or B16F10-Ova melanoma cells following treatment with either PBS, Blank Scaffold or immunoactive (Full) scaffolds.

FIG. 85 shows representative gating strategy for identifying T cell populations.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

In the following detailed description, numerous specific details are set forth in order to provide a thorough understanding of implantable scaffolds and microparticles, and the uses thereof. However, it will be understood by those skilled in the art that the production of these implantable scaffolds and microparticles and uses thereof may be practiced without these specific details. In other instances, well-known methods, procedures, and components have not been described in detail so as not to obscure their description.

Provided herein is a multifunctional biomaterial that is placed adjacent to a tumor and which attracts and potentiates cytotoxic T cells and suppresses local regulatory T cells. Together these activities allow for the much sought-after materials and methods for overcoming the immunosuppressive effects of the microenvironment of solid tumors.

Additionally, provided herein is a multifunctional biomaterial placed in a treatment area to deliver compositions treating localized symptoms of, for example, but not limited to, infectious and non-infectious medical conditions, injuries, damage, surgery, and transplant, where most needed in the treatment of localized conditions or symptoms, while avoiding systemic exposure to immunomodulatory agents.

Provided herein is a platform that holds the key to solve the above-mentioned challenges by offering a “synthetic lymph node” niche proximally to the tumor for supporting transferred T cells while enhancing their infiltration and cytotoxic capabilities. In some embodiments, this implantable, porous synthetic lymph node serves as a home for the recruitment of endogenous tumor resident T cells and provides them with the activation clues while fortifying them with necessary cytokines/chemokines at controlled rates. The mechanical stiffness of the biomaterial is optimized to mimic that of lymph nodes, including, but not limited to, serving as a home to T cells, e.g., for ACT purposes or for tumor resident T cells to obtain the required training against tumor cells, and facilitates their fight by increasing their number via proliferation signals and blocking the formation of suppressor T cells locally. This flexible platform holds high promises for localized immunomodulation and treatment of, e.g., cancers or other types of tumors.

Also provided herein is a platform for developed in situ lymphocyte (ISL) therapy, demonstrating potency in enhancing therapeutic efficacy of adoptive T cell therapy (ACT). ACT has been shown to hold high promises for many cancers including melanoma. Though its potency is limited by the inadequate T cell expansion in the tumor's suppressive microenvironment plus poor trafficking of tumor recognizing T cells to the tumor site. Thus, localization of trained T cells adjacent to the tumor while providing a niche that enhances their proliferation can overcome the main problems associated with ACT. Moreover, suppression of Treg in the tumor microenvironment can boost the therapeutic effects.

In some aspects, a porous scaffold is provided comprising at least one compound that regulates T cell immune response; and at least one compound that regulates induction of regulatory T cells (Tregs).

In some embodiments, the compound that regulates T cell immune response comprises a T cell immunostimulatory compound or a T cell immunosuppression compound.

In some embodiments, the at least one compound that regulates T cell immune response comprises a T cell immunostimulatory compound and the at least one compound that regulates induction of Tregs comprises a compound that suppresses induction of Tregs. In some embodiments, the T cell immunostimulatory compound is a T cell activator, a T cell attractant or a T cell adhesion compound. In some embodiments, the T cell immunostimulatory compound comprises a cytokine, a therapeutic or diagnostic protein, a growth factor, a chemokine, a therapeutic or diagnostic antibody or fragment thereof, an antigen-binding protein, a Fc fusion protein, an anticoagulant, an enzyme, a hormone, a thrombolytic, a peptide, an oligonucleotide, a nucleic acid, a chemokine ligand, or an anti-cluster of differentiation (anti-CD) antibody or fragment thereof. In some embodiments, the cytokine comprises an interleukin (IL). In some embodiments, the T cell immunostimulatory compound comprises interleukin-2 (IL-2), interleukin-4 (IL-4), interleukin-6 (IL-6), interleukin-7 (IL-7), interleukin-10 (IL-10), interleukin-12 (IL-12), interleukin-15 (IL-15), IL-2 superkine, chemokine (C-C motif) ligand 21 (CCL21), anti-cluster of differentiation 3 (anti-CD3), or anti-cluster of differentiation 28 (anti-CD28), a CpG oligonucleotide (including toll-like receptor 9 (TLR9) agonist adjuvants), poly (I:C), monophosphoryl lipid A (MPLA), imiquimod, or a cyclic dinucleotide, or any combination thereof.

In some embodiments, the at least one compound that regulates T cell immune response comprises a T cell immunosuppression compound and the at least one compound that regulates induction of Tregs comprises a compound that induces Tregs. In some embodiments, the T cell immunosuppression compound comprises stromal cell-derived factor 1a (SDF-1a). In some embodiments, the growth factor comprises transforming growth factor-beta (TGF-β), vascular endothelial growth factor (VEGF), or bone morphogenetic protein-2 (BMP-2). In some embodiments, the scaffolds comprises IL-2, IL-4 and TGF-β.

In some embodiments, the compound that suppresses induction of Tregs comprises a TGF-β inhibitor. In some embodiments, the TGF-β inhibitor is a TGF-β receptor inhibitor. In some embodiments, the TGF-β inhibitor is galinusertib (LY2157299) or SB505124. In other embodiments, the at least one compound that regulates induction of Tregs comprises a compound that induces Tregs. In some embodiments, the compound that induces Tregs is a TGF-β or an activator thereof.

Compounds that suppression induction of Tregs include, but are not limited to, inhibitors of transforming growth factor-beta (TGF-β), such as an inhibitor of the TGF-β receptor. Non-limiting examples of TGF-β receptor inhibitors include galinusertib (LY2157299), SB505124, small molecule inhibitors, antibodies, chemokines, apoptosis signals (e.g., cytotoxic T-lymphocyte-associated protein 4/programmed cell death protein 1 (CTLA-4/PD-1); Granzyme; tumor necrosis factor (TNF)-related apoptosis-inducing ligand (TRAIL); Fas/Fas-L, Galectin-9/transmembrane immunoglobulin and mucin domain 3 (TIM-3)). Compounds that induce Tregs include TGF-β and activators thereof (e.g., SB 431542, A 83-01, RepSox, LY 364947, D 4476, SB 525334, GW 788388, SD 208, R 268712, IN 1130, SM 16, A 77-01, AZ 12799734).

In some embodiments, the at least one compound that regulates induction of regulatory T cells is released slowly from the scaffold.

In some embodiments, the at least one compound that regulates induction of regulatory T cells comprises a compound that suppresses induction of regulatory T cells or a compound that induces regulatory T cells.

In some embodiments, the compound that suppresses induction of regulatory T cells is an inhibitor of transforming growth factor-beta (TGF-β), such as a TGF-β receptor inhibitor. In some embodiments, the inhibitor is galinusertib (LY2157299) or SB505124.

In another related aspect, one or more of the compounds comprises a therapeutic or diagnostic protein. In another related aspect, one or more of the compounds comprises a cytokine, a chemokine, a therapeutic or diagnostic antibody or fragment thereof, an antigen-binding protein, a Fc fusion protein, an anticoagulant, an enzyme, a hormone, or a thrombolytic. In another related aspect, the cytokine comprises an interleukin. In another related aspect, the interleukin comprises an IL-2, interleukin-4 (IL-4), interleukin-6 (IL-6), interleukin-7 (IL-7), interleukin-10 (IL-10), an IL-12, or an IL-15. In yet another related aspect, a cytokine may include a human cytokine. In still another related aspect, an IL-2 cytokine comprises an IL-2 superkine. In some embodiments, the IL-2 superkine comprises the sequence as set forth in SEQ ID NO: 3.

In some embodiments, the at least one compound that regulates T cell immune response is bound to heparin. In some embodiments, the heparin is bound to one or more microparticles embedded in the scaffold. In some embodiments, the one or more microparticles comprise one or more silica microparticles. In some embodiments, the heparin is provided at about 2 nanomols per milligram (nmol/mg) of silica. In some embodiments, the one or more silica microparticles are about 3 microns (μm) to about 25 microns (μm). In some embodiments, the silica is mesoporous silica. In some embodiments, the loading of the one or more silica microparticles by the at least one compound that regulates T cell immune response is increased by the bound heparin. In some embodiments, the release of the at least one compound that regulates T cell immune response from the one or more silica microparticles is reduced by the bound heparin. In some embodiments, the silica microparticles persist in vivo for at least 15-20 days.

In some embodiments, the porous scaffold further comprises one or more nanoparticles. In some embodiments, the nanoparticles comprise poly(lactic-co-glycolic acid) (PLGA). In some embodiments, the nanoparticles are bound to the at least one compound that regulates induction of regulatory T cells.

In some embodiments, the scaffold is biocompatible or biodegradable. In some embodiments, the scaffold comprises a polymer selected from alginate, hyaluronic acid and chitosan, or any combination thereof. In some embodiments, the polymer comprises an arginine-glycine-aspartate (RGD) peptide. In some embodiments, the porous scaffold comprises pores of from about 1 to about 7 nm.

In some embodiments, the scaffold is provided to be surgically implantable or injectable or administrable through a catheter. In some embodiments, the scaffold further comprises one or more immune cells. In some embodiments, the one or more immune cells are T cells. In some embodiments, the T cells comprise wild-type and transgenic, murine and human CD4+ and CD9* T cells. In some embodiments, the T cells are chimeric antigen receptor T cells (CAR-T cells). In some embodiments, anti-CD3 or anti-CD28 antibodies are covalently bound to the polymer.

In some embodiments, the porous scaffold comprises an alginate-RGD polymer comprising silica-heparin microparticles bound to IL-2, anti-CD3 and anti-CD28, PLGA nanoparticles comprising a TGF-β inhibitor, and anti-CD3 and anti-CD28 antibodies covalently bound to the alginate-RGD polymer.

In some aspects, a method is provided of regulating an immune response to a disease or medical condition or symptoms thereof, at a focus of interest in a subject in need, the method comprising providing a porous scaffold at a site at or near a site of the focus of interest, the porous scaffold comprising at least one compound that regulates T cell immune response and at least one compound that regulates induction of regulatory T cells (Tregs).

In some embodiments, the disease or medical condition comprises a tumor, a suspected tumor, or a resected tumor and the porous scaffold is provided at or adjacent to a focus of interest comprising the tumor, suspected tumor, or resected tumor.

In some embodiments, the tumor is a solid tumor. In some embodiments, the tumor, suspected tumor, or resected tumor comprises a cancerous, pre-cancerous, or non-cancerous tumor. In some embodiments, the tumor comprises a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor (for example, a triple-negative breast cancer), an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma, esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non-small cell lung cancer (NSCLC), squamous cell non-small cell lung cancer, breast cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16;16)(p13.1;q22); CBFB-MYH11; adult acute myeloid leukemia with t(8;21)(q22;q22); RUNX1-RUNX1T1; adult acute myeloid leukemia with t(9;11)(p22;q23); MLLT3-MLL; adult acute promyelocytic leukemia with t(15;17)(q22;q12); PML-RARA; alkylating agent-related acute myeloid leukemia, chronic lymphocytic leukemia, Richter's syndrome; Waldenstrom's macroglobulinemia, adult glioblastoma; adult gliosarcoma, recurrent glioblastoma, recurrent childhood rhabdomyosarcoma, recurrent Ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent Merkel cell carcinoma; stage III Merkel cell carcinoma; stage IV Merkel cell carcinoma, myelodysplastic syndrome and recurrent mycosis fungoides and Sezary syndrome. In some embodiments, at the site, T cells are stimulated to target the tumor, suspected tumor, or resected tumor, and the induction of Tregs is suppressed.

In some embodiments, said treating reduces the size of the tumor, eliminates the tumor, slows the growth or regrowth of the tumor, slows the growth or regrowth of a secondary tumor, or prolongs survival of said subject or any combination thereof.

In some embodiments, at the site, T cells are stimulated to target the focus of interest, and the induction of Tregs is suppressed. In other embodiments, at the site, T cells are suppressed at or near the focus of interest, and Tregs are induced.

In some embodiments, the disease or medical condition comprises an autoimmune disease, and the porous scaffold is provided at or adjacent to a focus of interest comprising an autoimmune-targeted or symptomatic focus of said autoimmune disease; the disease or medical condition comprises an allergic reaction or hypersensitivity reaction, and the porous scaffold is provided at or adjacent to a focus of interest comprising a reactive focus of said allergic reaction or hypersensitivity reaction; the disease or medical condition comprises a localized infection or an infectious disease, and the porous scaffold is provided at or adjacent to a focus of interest comprising a focus of infection or symptoms; the disease or medical condition comprises an injury or a site of chronic damage, and the porous scaffold is provided at or adjacent to a focus of interest comprising the injury or the site of chronic damage; the disease or medical condition comprises a surgical site, and the porous scaffold is provided at or adjacent to a focus of interest comprising the surgical site; the disease or medical condition comprises a transplanted organ, tissue, or cell, and the porous scaffold is provided at or adjacent to a focus of interest comprising a transplant site; or the disease or medical condition comprises a blood clot causing or at risk for causing a myocardial infarction, an ischemic stroke, or a pulmonary embolism, and the porous scaffold is provided at or adjacent to a focus of interest comprising the site of the blood clot. In some embodiments, said treating reduces or eliminates inflammation or another symptom of said autoimmune-targeted or symptomatic focus of said autoimmune disease, prolongs survival of said subject, or any combination thereof; reduces or eliminates inflammation or another symptom of allergic reaction or hypersensitivity reaction at said reactive focus of said allergic reaction or hypersensitivity reaction, prolongs survival of said subject, or any combination thereof; reduces or eliminates infection or symptoms at said focus of infection or symptoms of said localized infection or infectious disease, prolongs survival of said subject, or any combination thereof; reduces, eliminates, inhibits or prevents structural, organ, tissue, or cell damage, inflammation, infection, or another symptom at said site of injury or said site of chronic damage, improves structural, organ, tissue, or cell function at said site of injury or said site of chronic damage, improves mobility of said subject, prolongs survival of said subject, or any combination thereof; reduces, eliminates, inhibits, or prevents structural, organ, tissue, or cell damage, inflammation, infection, or another symptom at said surgical site, improves structural, organ, tissue, or cell function at said surgical site, improves mobility of said subject, prolongs survival of said subject, or any combination thereof; reduces, eliminates, inhibits or prevents transplanted organ, tissue, or cell damage or rejection, inflammation, infection or another symptom at said transplant site, improves mobility of said subject, prolongs survival of said transplanted organ, tissue, or cell, prolongs survival of said subject, or any combination thereof; or reduces or eliminates said blood clot causing or at risk for causing said myocardial infarction, said ischemic stroke, or said pulmonary embolism in said subject, improves function or survival of a heart, brain, or lung organ, tissue, or cell in said subject, reduces damage to a heart, brain, or lung organ, tissue, or cell in said subject, prolongs survival of a heart, brain, or lung organ, tissue, or cell in said subject, prolongs survival of said subject, or any combination thereof. In some embodiments, the disease or medical condition comprises a blood clot causing or at risk for causing a myocardial infarction, an ischemic stroke, or a pulmonary embolism, and the porous scaffold is provided at or adjacent to a focus of interest comprising the site of the blood clot together with angioplasty or another clot removal treatment.

In some aspects, a method is provided for stimulating T cells to target a solid tumor and for suppressing the induction of Tregs in a patient comprising providing the porous scaffold described herein at a site at or near a solid tumor, a suspected solid tumor or a resected solid tumor, the porous scaffold comprising at least one response cell immunostimulatory compound and at least one compound that suppresses induction of regulatory T cells (Tregs). In one embodiment, the tumor is an inoperable tumor.

In some aspects, a method is provided for regulating an immune response at a focus of interest in a subject in need, said method comprising providing a porous scaffold to the subject, at or near a site of the focus of interest, the porous scaffold comprising at least one compound that regulates T cell immune response; and at least one compound that regulates induction of regulatory T cells (Tregs), wherein regulating the immune response comprises increasing or decreasing proliferation of cytotoxic T cells; increasing or decreasing proliferation of helper T cells; maintaining, increasing, or decreasing the population of helper T cells at the site of said focus of interest; activating or suppressing cytotoxic T cells at the site of said focus of interest; or any combination thereof. In some aspects, a method is provided for treating a disease or medical condition, or alleviating symptoms thereof, at a focus of interest in a subject in need, said method comprising providing a porous scaffold at a site at or near a focus of interest, the porous scaffold comprising: at least one compound that regulates T cell immune response; and at least one compound that regulates induction of regulatory T cells (Tregs). In some embodiments, the compound that suppresses induction of Tregs comprises a TGF-β inhibitor. In some embodiments, the TGF-β inhibitor is a TGF-β receptor inhibitor. In some embodiments, the TGF-β inhibitor is galinusertib (LY2157299) or SB505124. In other embodiments, the at least one compound that regulates induction of Tregs comprises a compound that induces Tregs. In some embodiments, the compound that induces Tregs is a TGF-β or an activator thereof.

Compounds that suppression induction of Tregs include, but are not limited to, inhibitors of transforming growth factor-beta (TGF-β), such as an inhibitor of the TGF-β receptor. Non-limiting examples of TGF-β receptor inhibitors include galinusertib (LY2157299), SB505124, small molecule inhibitors, antibodies, chemokines, apoptosis signals (e.g., cytotoxic T-lymphocyte-associated protein 4/programmed cell death protein 1 (CTLA-4/PD-1); Granzyme; tumor necrosis factor (TNF)-related apoptosis-inducing ligand (TRAIL); Fas/Fas-L, Galectin-9/transmembrane immunoglobulin and mucin domain 3 (TIM-3)). Compounds that induce Tregs include TGF-β and activators thereof (e.g., SB 431542, A 83-01, RepSox, LY 364947, D 4476, SB 525334, GW 788388, SD 208, R 268712, IN 1130, SM 16, A 77-01, AZ 12799734).

In another aspect, a method is provided herein for making a porous biocompatible or biodegradable scaffold for regulating an immune response at a focus of interest in a subject in need, the method comprising: providing a porous scaffold comprising a polymer: embedding in the scaffold one or more microparticles or one or more nanoparticles, the one or more microparticles bound to heparin, and the heparin bound to at least one compound that regulates T cell immune response; or the one or more nanoparticles or microparticles bound to or encapsulate at least one compound that regulates induction of regulatory T cells (Tregs). In some embodiments, the porous biocompatible or biodegradable scaffold comprising a polymer comprising alginate, hyaluronic acid, chitosan, or a combination thereof, or an arginine-glycine-aspartate (RGD) peptide, or an alginate-RGD polymer; the one or more microparticles comprising silica-heparin; or the nanoparticles or microparticles comprising poly(lactic-co-glycolic acid) (PLGA). In some embodiments, the porous biocompatible or biodegradable scaffold further comprising one or more immune cells. In some embodiments, the porous biocompatible or biodegradable scaffold further comprising anti-CD3 or anti-CD28 antibodies covalently bound to the polymer. In some embodiments, the at least one compound that regulates T cell immune response comprising a T cell immunostimulatory compound comprising a cytokine, a therapeutic or diagnostic protein, a growth factor, a chemokine, a therapeutic or diagnostic antibody or fragment thereof, an antigen-binding protein, a Fc fusion protein, an anticoagulant, an enzyme, a hormone, a thrombolytic, a peptide, an oligonucleotide, a nucleic acid, a chemokine ligand, or an anti-cluster of differentiation (anti-CD) antibody or fragment thereof, interleukin-2 (IL-2), interleukin-4 (IL-4), interleukin-6 (IL-6), interleukin-7 (IL-7), interleukin-10 (IL-10), interleukin-12 (IL-12), interleukin-15 (IL-15), IL-2 superkine, chemokine (C-C motif) ligand 21 (CCL21), anti-CD3 or anti-CD28, or any combination thereof; or the at least one compound that regulates induction of regulatory T cells (Tregs) comprising a compound that suppresses induction of Tregs comprising galinusertib (LY2157299), SB505124, or another transforming growth factor-beta (TGF-β) inhibitor.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

In some embodiments, described throughout herein, are “porous scaffolds.” These scaffolds are able to provide T cell immunoregulatory compounds to a microenvironment within which they are implanted and located. The release of immunoregulatory compounds may be regulatable, providing targeted therapeutic biological molecule(s) or biological molecule(s) that in turn regulates a downstream therapeutic target. This regulatable production may in certain embodiments, reduce or eliminate systemic toxicity.

In some embodiments, described throughout herein, are “microparticles.” These microparticles are embedded in a scaffold. These microparticles may serve as a “platform” comprising at least one compound that regulates T cell immune response, providing an increased amount of a biomolecule or other compound that regulates T cell immune response, while retaining the regulatable aspects of the localized distribution. These “microparticles” may be targeted to a site of need by incorporating targeting molecules into an encapsulation coating. Additionally, at least one compound that regulates induction of T regulatory cells (Tregs) may in certain embodiments be incorporated into the microparticles. Additionally, the microparticles may further comprise an encapsulation coating (e.g., heparin), which may enhance the biophysical properties of the microparticles.

In some embodiments, “nanoparticles” can be used in place of microparticles, as described herein.

In some embodiments, described herein are uses of these “porous scaffolds” for treating cancer or other tumors. Use of these “scaffolds” in a therapeutic cancer or tumor treatment may in certain embodiments, prove advantageous as they may provide a regulatable expression system of a needed or advantageous biomolecule, such as a compound that regulates T cell immune response and/or a compound that regulates induction of regulatory T cells, within a localized treatment area that may further be targeted to T cells, which in turn could promote clearance of the cancer or tumor. These implantable scaffolds may further be biodegradable following implantation in a subject.

In some embodiments, described herein are uses of these “porous scaffolds” or “scaffolds” for treating a focus of interest of an autoimmune disease or an allergic reaction or hypersensitivity reaction, a localized site of an infection or infectious disease, a localized site of an injury or other damage, a transplant or other surgical site, a blood clot, or a symptom thereof, or a combination thereof. Use of these “factories” in the treatment of a focus of interest of an autoimmune disease or an allergic reaction or hypersensitivity reaction, a localized site of an infection or infectious disease, a localized site of an injury or other damage, a transplant or other surgical site, a blood clot, or a symptom thereof, or a combination thereof, may in certain embodiments, prove advantageous as they may provide a regulatable expression system of a needed or advantageous biomolecule, within a localized treatment area that may further be targeted to T cells, which could promote clearance of or alleviate localized symptoms of the autoimmune disease, allergic reaction or hypersensitivity reaction, infection or infectious disease, or blood clot, or could facilitate healing and/or prevent infection or rejection of a localized site of an injury or other damage, a transplant or other surgical site, or could alleviate localized symptoms thereof.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

The terms “subject,” “individual,” and “patient” are used interchangeably herein, and refer to an animal, for example a human, to whom treatment with a composition or formulation in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The term “higher vertebrates” is used herein and includes avians (birds) and mammals. The compositions described herein can be used to treat any suitable mammal, including primates, such as monkeys and humans, horses, cows, cats, dogs, rabbits, sheep, goats, pigs, and rodents such as rats and mice. In one embodiment, the mammal to be treated is human. The human can be any human of any age. In an embodiment, the human is an adult. In another embodiment, the human is a child. The human can be male, female, pregnant, middle-aged, adolescent, or elderly. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine, or porcine. In another embodiment, the subject is mammalian.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

As noted above, obstacles persist in developing and applying effective methods for activating cytotoxic T cells for cancer immunotherapy. As described herein, significant improvements have been made in the response of T cells to solid tumors despite their immunosuppressive tumor environment. TGF-β is known to be a potent component of the tumor microenvironment, which promotes cancer growth and metastasis and promotes the induction of Tregs from the helper T cells drawn to the tumor. Suppression of TGF-β could allow for a reduction in regulatory T cells and more effective CD8+ T cell killing, resulting in rapid clearance of solid tumors. Here we describe an approach to enable the local delivery of TGF-β inhibitor (TGF-βi) into the tumor environment for the enhancement of the immune responses during immunotherapy. An implantable scaffold is provided comprising means for local delivery of TGF-βi (e.g., in PLGA nanoparticles or microparticles embedded in the scaffold) and also one or more immunostimulatory compounds to attract and activate cytotoxic T cells to target the tumor (e.g., IL-2 on silica-heparin microparticles embedded in the scaffold). Systemic effects are avoided by employing local effects of the scaffold, which can induce a potent T cell response to a tumor or remaining tumor after resection, or even treat inoperable tumors, and then the scaffold can biodegrade over time.

As described herein, the studies described emphasize the local delivery of inhibitors and activators based in a biodegradable scaffold. Once administered, the scaffold can attract lymphocytes to the site of the tumor and allow simultaneous T cell stimulation and controlled release of the TGF-βi. The combined response of the immune system in the tumor microenvironment is then enhanced: Treg development is reduced in favor of effector T cell activation and tumor rejection is achieved by the activated T cells. This method provides an immunotherapy treatment that is more effective by directly altering the effects of the tumor microenvironment.

Details of each component of the scaffold are provided below. The implantable scaffold can be made of various biocompatible and biodegradable polymers. To further encourage cell trafficking within these structures, cell adhesion peptides such as but not limited to the chemokine CCL21, and immunostimulatory compounds such as IL-2, IL-4, IL-6, IL7, IL-10, IL-12, IL-15, or IL-2 superkine, or antibodies such as anti-CD3 and anti-CD28 are provided. To improve the resemblance of these 3D matrices to natural tissues techniques are used that create microscale pores within these structures that both allows for maximizing the loading capacity for delivering T cells and facilitates their expansion as well. The scaffolds are modified with anti-CD3/anti-CD28 antibodies and further comprise a TGF-βi, as well as IL-2 cytokine to provide activation signal for T cells and prevent formation of regulatory T cells.

### Scaffold

The scaffold may comprise a polymer such as but not limited to alginate, hyaluronic acid, or chitosan, or any combination thereof. It comprises one of more the components described below. The scaffold can be fabricated into a shape and size for facile insertion or implantation during a surgical or transdermal procedure. In one embodiment, the scaffold is about the shape and size of a pencil eraser. However, the shape and size can be configured for a particular application, for ease of insertion, and/or for retention at a particular site near a tumor or resected tumor site.

### Pores

Pores are created in the scaffold by freeze drying process such as that described in Biopolymer-Based Hydrogels As Scaffolds for Tissue Engineering Applications: A Review Biomacromolecules 2011, 12, 5, 1387-1408; https://pubs.acs.org/doi/abs/10.1021/bm200083n. In one embodiment, the pores are between about 1 and about 7 nm in size.

### Microparticles

In certain embodiments, disclosed herein are microparticles. In some embodiments, the microparticles comprise a polymer. In some embodiments, the polymer comprises a biocompatible polymer. In some embodiments, the biocompatible polymer comprises alginate, chitosan, or mesoporous silica. In some embodiments, the microparticles comprise silica microparticles. Silica microparticles such as mesoporous silica may be embedded in the scaffold. In some embodiments, the silica is bound to heparin. In some embodiments, about 2 nmol of heparin is bound per mg of silica. In some embodiments, the microparticle has a size comprising 1-1000 micrometers. In some embodiments, the particles are from about 3 to about 24 μm in diameter. In some embodiments, the microparticles comprise hyaluronic acid. In some embodiments, the microparticles comprise heparin.

In some embodiments, microparticles may be encapsulated by a coating. In some embodiments, coatings provide microparticles with enhanced biological characteristics, including interactions with cells, with compounds that regulate T cell immune response, with compounds that regulate induction of regulatory T cells, and with other biomolecules. In some embodiments, microparticles are encapsulated with a coating comprising heparin. In some embodiments, microparticles are encapsulated with alginate or alginate-heparin. In some embodiments, an alginate-heparin coating may be sulfated.

In some embodiments, microparticles may comprise a “coating” material. In some embodiments, these materials provide microparticles with enhanced biological characteristics, including interactions with cells and biomolecules. In some embodiments, microparticles are formed in the presence of a mix of alginate-heparin. In some embodiments, microparticles are formed in the presence of a mix of alginate. In some embodiments, an alginate may be sulfated.

A skilled artisan would appreciate that a description of a microparticle comprising an alginate or alginate-heparin coating may in certain embodiments, encompass a microparticle prepared in the presence of alginate or alginate and heparin, wherein these molecules and integral components of the microparticle synthesized.

In some embodiments, microparticles may be targeted to T cells. In some embodiments, a microparticle coat comprises biomolecules that recognize and bind cell surface markers on T cells. In some embodiments, cell surface markers on T cells include CD3 and CD28. In some embodiments, a biomolecule that recognized a T cell surface marker comprises an antibody or a fragment thereof.

Paramagnetic nanoparticles may be included in the microparticles, e.g., for purification or for ease of separation, and are commercially available (e.g., CHEMICELL™ GmbH). In some embodiments, the paramagnetic nanoparticles comprise superparamagnetic iron oxide nanoparticles (SPIONs). In some embodiments, a SPION comprises a particle having a size about 50-200 mm. This addition may in certain embodiments enhance purification of microparticles using methods well known in the art.

### Nanoparticles

In some embodiments, the scaffold comprises one or more nanoparticles. In some exemplary, but non-limiting, embodiments, the nanoparticle comprises a poly(lactic-co-glycolic acid) (PLGA, PLG), a copolymer, produced using methods known in the art. In some embodiments, the nanoparticle is sized between 1-100 nm. In some embodiments, the nanoparticle is biocompatible and/or biodegradable. This addition may in certain embodiments enhance purification of microparticles or nanoparticles using methods well known in the art.

In some embodiments, the nanoparticle is bound to at least one compound that regulates induction of regulatory T cells, as described herein.

### T Cells and Regulatory Compounds

In some embodiments, immune cells, for example T cell, are generated and expanded by the presence of cytokines in vivo. In some embodiments, cytokines that affect generation and maintenance to T-helper cells in vivo comprise IL-2, IL-4, IL-6, IL-7, IL-10, IL-12, IL-15, or IL-2 superkine. In some embodiments, T regulatory (Treg) cells are generated from naïve T cells by cytokine induction in vivo. In some embodiments, TGF-β and/or IL-2 play a role in differentiating naïve T cell to become Treg cells.

“Cytokines” are a category of small proteins (˜5-20 kDa) critical to cell signaling. Cytokines are peptides and usually are unable to cross the lipid bilayer of cells to enter the cytoplasm. Among other functions, cytokines may be involved in autocrine, paracrine and endocrine signaling as immunomodulating agents. Cytokines may be pro-inflammatory or anti-inflammatory. Cytokines include, but are not limited to, chemokines (cytokines with chemotactic activities), interferons, interleukins (ILs; cytokines made by one leukocyte and acting on one or more other leukocytes), lymphokines (produced by lymphocytes), monokines (produced by monocytes), and tumor necrosis factors. Cells producing cytokines include, but are not limited to, immune cells (e.g., macrophages, B lymphocytes, T lymphocytes and mast cells), as well as endothelial cells, fibroblasts, and various stromal cells. A particular cytokine may be produced by more than one cell type.

A skilled artisan would appreciate that the term “cytokine” may encompass cytokines beneficial to enhancing an immune response targeted against a cancer or a pre-cancerous or non-cancerous tumor or lesion. A skilled artisan would also appreciate that the term “cytokine” may encompass cytokines beneficial to enhancing an immune response against a disease or inflammation (e.g., resulting from surgery, an injury, or damage from an autoimmune response) or that the term “cytokine” may encompass cytokines beneficial to reducing an abnormal autoimmune response.

In some embodiments, a cytokine encoded by the nucleic acid expands and maintains T-helper cells (helper T cells). In some embodiments, a cytokine encoded by the nucleic acid expands T-helper cells. In some embodiments, a cytokine encoded by the nucleic acid maintains T-helper cells. In some embodiments, a cytokine encoded by the nucleic acid expands cytotoxic T cells (CTLs). In some embodiments, a cytokine encoded by the nucleic acid activates cytotoxic T cells. In some embodiments, a cytokine encoded by the nucleic acid expands and activates cytotoxic T cells. In some embodiments, a cytokine encoded by the nucleic acid increases proliferation of a T-helper cell population. In some embodiments, a cytokine encoded by the nucleic acid increases proliferation of a cytotoxic T cell population.

In some embodiments, the encoded cytokine comprises an interleukin (IL). A skilled artisan would appreciate that interleukins comprise a large family of molecules, including, but not limited to, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36.

In some embodiments, the encoded interleukin comprises an IL-2, IL-4, IL-6, IL-7, IL-10, IL-12, or an IL-15, or any combination thereof. In some embodiments, the encoded cytokine comprises an IL-2. In some embodiments, the encoded cytokine comprises an IL-12. In some embodiments, the encoded cytokine comprises an IL-15.

In some embodiments, the plasmid encodes any additional cytokine or polypeptide or peptide.

In some embodiments, the IL-2 cytokine comprises an IL-2 superkine (super IL-2 cytokine). IL-2 is a 133 amino acid glycoprotein with one intramolecular disulfide bond and variable glycosylation.

“IL-2 superkine” or “Super 2” (Fc) is an artificial variant of IL-2 containing mutations at positions L80F/R81D/L85V/I86V/I92F. These mutations are located in the molecule's core that acts to stabilize the structure and to give it a receptor-binding conformation mimicking native IL-2 bound to CD25. These mutations effectively eliminate the functional requirement of IL-2 for CD25 expression and elicit proliferation of T cells. Compared to IL-2, the IL-2 superkine induces superior expansion of cytotoxic T cells, leading to improved antitumor responses in vivo, and elicits proportionally less toxicity by lowering the expansion of T regulatory cells and reducing pulmonary edema. Examples of IL-2 superkine (Super2) deoxyribonucleic acid (DNA) and protein sequences can be found, e.g., in Table 1.

TABLE 1
IL-2 superkine (Super2) Sequence.

| Type of | Sequence |
|---|---|
| Sequence | (SEQ ID NO) |
| Super2 | GGAGCCATGGGAGAATTCGCACCTACTTCAAGTTCTACA |
| nucleo- | AAGAAAACACAGCTACAACTGGAGCATTTACTTCTGGAT |
| tide | TTACAGATGATTTTGAATGGAATTAATAATTACAAGAAT |
| sequence | CCCAAACTCACCAGGATGCTCACATTTAAGTTTTACATG |
| | CCCAAGAAGGCCACAGAACTGAAACATCTTCAGTGTCTA |
| | GAAGAAGAACTCAAACCTCTGGAGGAAGTGCTAAATTTA |
| | GCTCAGAGCAAAAACTTTCACTTCGATCCCAGGGACGTC |
| | GTCAGCAATATCAACGTATTCGTCCTGGAACTAAAGGGA |
| | TCTGAAACAACATTCATGTGTGAATATGCTGATGAGACA |
| | GCAACCATTGTAGAATTTCTGAACAGATGGATTACCTTT |
| | TGTCAAAGCATCATCTCAACACTAACTCAT |
| | (SEQ ID NO: 2) |
| Super2 | MGEFAPTSSSTKKTQLQLEHLLLDLQMILNGINNYKNPK |
| protein | LTRMLTFKFYMPKKATELKHLQCLEEELKPLEEVLNLAQ |
| sequence | SKNFHFDPRDVVSNINVFVLELKGSETTFMCEYADETAT |
| | IVEFLNRWITFCQSIISTLTH |
| | (SEQ ID NO: 3) |

A “T cell” is characterized and distinguished by the T cell receptor (TCR) on the surface. A T cell is a type of lymphocyte that arises from a precursor cell in the bone marrow before migrating to the thymus, where it differentiates into one of several kinds of T cells. Differentiation continues after a T cell has left the thymus. A “cytotoxic T cell” (CTL) is a CD8+ T cell able to kill, e.g., virus-infected cells or cancer cells. A “T helper cell” is a CD4+ T cell that interacts directly with other immune cells (e.g., regulatory B cells) and indirectly with other cells to recognize foreign cells to be killed. “Regulatory T cells” (T regulatory cells; Treg), also known as “suppressor T cells,” enable tolerance and prevent immune cells from inappropriately mounting an immune response against “self,” but may be co-opted by cancer or other cells. In autoimmune disease, “self-reactive T cells” mount an immune response against “self” that damages healthy, normal cells.

In some embodiments, the porous scaffold comprises a T cell immunostimulatory compound and/or a compound that suppresses induction of Tregs. In some embodiments, the porous scaffold comprises a T cell immunosuppression compound and/or a compound that induces Tregs.

One skilled in the art appreciates the many mechanisms of T cell immunostimulation and/or immunosuppression. Likewise, one skilled in the art appreciates the many mechanisms of Treg induction and/or suppression of Treg induction.

T cell immunostimulatory compounds include, but are not limited to, T cell activators, T cell attractants, or T cell adhesion compounds. T cell immunostimulatory compounds include, but are not limited to, cytokines, a therapeutic or diagnostic protein, a growth factor, a chemokine, a therapeutic or diagnostic antibody or fragment thereof, an antigen-binding protein, a Fc fusion protein, an anticoagulant, an enzyme, a hormone, a thrombolytic, a peptide, an oligonucleotide, a nucleic acid, chemokine ligands, and anti-CD antibodies or fragments thereof. Non-limiting examples include interleukins (e.g., IL-2, IL4, I-L6, IL-7, IL-10, IL-12, or IL-15, or an IL-2 superkine), chemokine ligands (e.g., CCL ligands, including CCL21), and anti-CD antibodies (e.g., anti-CD3 or anti-CD28) or fragments thereof, or any combination(s) thereof.

T cell immunosuppression compounds include, but are not limited to cytokines, chemokines, growth factors, or small molecule inhibitors.

Compounds that suppression induction of Tregs include, but are not limited to, inhibitors of transforming growth factor-beta (TGF-β), such as an inhibitor of the TGF-β receptor. Non-limiting examples of TGF-β receptor inhibitors include galinusertib (LY2157299) or SB505124. Compounds that induce Tregs include TGF-β and activators thereof (e.g., IL-2, IL-4).

As used herein, a “targeting agent,” or “affinity reagent,” is a molecule that binds to an antigen or receptor or other molecules. In some embodiments, a “targeting agent” is a molecule that specifically binds to an antigen or receptor or other molecule. In certain embodiments, some or all of a targeting agent is composed of amino acids (including natural, non-natural, and modified amino acids), nucleic acids, or saccharides. In certain embodiments, a “targeting agent” is a small molecule.

As used herein, the term “antibody” encompasses the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains VL and CL, and each heavy chain comprising immunoglobulin domains VH, C-gamma-1 (Cγ1), C-gamma-2 (Cγ2), and C-gamma-3 (Cγ3). In each pair, the light and heavy chain variable regions (VL and VH) are together responsible for binding to an antigen, and the constant regions (CL, Cγ1, Cγ2, and Cγ3, particularly Cγ2, and Cγ3) are responsible for antibody effector functions. In some mammals, for example in camels and llamas, full-length antibodies may consist of only two heavy chains, each heavy chain comprising immunoglobulin domains VH, Cγ2, and Cγ3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full-length antibodies, antibody fragments, and individual immunoglobulin domains including but not limited to VH, Cγ1, Cγ2, Cγ3, VL, and CL.

Depending on the amino acid sequence of the constant domain of their heavy chains, intact antibodies can be assigned to different “classes”. There are five-major classes (isotypes) of intact antibodies: IgA, IgD, IgE, IgG, and IgM, and several of these may be further divided into “subclasses”, e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known to one skilled in the art.

As used herein, the term “immunoglobulin G” or “IgG” refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. As used herein, the term “modified immunoglobulin G” refers to a molecule that is derived from an antibody of the “G” class. As used herein, the term “antibody” refers to a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA isotypes or classes, respectively.

The term “antibody” is meant to include full-length antibodies, and may refer to a natural antibody from any organism, an engineered antibody, or an antibody generated recombinantly for experimental, therapeutic, or other purposes as further defined below. Furthermore, full-length antibodies comprise conjugates as described and exemplified herein. As used herein, the term “antibody” comprises monoclonal and polyclonal antibodies. Antibodies can be antagonists, agonists, neutralizing, inhibitory, or stimulatory. Specifically included within the definition of “antibody” are full-length antibodies described and exemplified herein. By “full length antibody” herein is meant the structure that constitutes the natural biological form of an antibody, including variable and constant regions.

The “variable region” of an antibody contains the antigen binding determinants of the molecule, and thus determines the specificity of an antibody for its target antigen. The variable region is so named because it is the most distinct in sequence from other antibodies within the same isotype. The majority of sequence variability occurs in the complementarity determining regions (CDRs). There are 6 CDRs total, three each per heavy and light chain, designated VH CDR1, VH CDR2, VH CDR3, VL CDR1, VL CDR2, and VL CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen binding diversity (the CDRs) can be explored by the immune system to obtain specificity for a broad array of antigens.

Furthermore, antibodies may exist in a variety of other forms including, for example, Fv, Fab, and (Fab′)2, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986)).

The term “epitope” as used herein refers to a region of the antigen that binds to the antibody or antigen-binding fragment. It is the region of an antigen recognized by a first antibody wherein the binding of the first antibody to the region prevents binding of a second antibody or other bivalent molecule to the region. The region encompasses a particular core sequence or sequences selectively recognized by a class of antibodies. In general, epitopes are comprised by local surface structures that can be formed by contiguous or noncontiguous amino acid sequences.

As used herein, the terms “selectively recognizes”, “selectively bind” or “selectively recognized” mean that binding of the antibody, antigen-binding fragment or other bivalent molecule to an epitope is at least 2-fold greater, preferably 2-5 fold greater, and most preferably more than 5-fold greater than the binding of the molecule to an unrelated epitope or than the binding of an antibody, antigen-binding fragment or other bivalent molecule to the epitope, as determined by techniques known in the art and described herein, such as, for example, ELISA or cold displacement assays.

As used herein, the term “Fc domain” encompasses the constant region of an immunoglobulin molecule. The Fc region of an antibody interacts with a number of Fc receptors and ligands, imparting an array of important functional capabilities referred to as effector functions, as described herein. For IgG the Fc region comprises Ig domains CH2 and CH3. An important family of Fc receptors for the IgG isotype are the Fc gamma receptors (FcγRs). These receptors mediate communication between antibodies and the cellular arm of the immune system.

As used herein, the term “Fab domain” encompasses the region of an antibody that binds to antigens. The Fab region is composed of one constant and one variable domain of each of the heavy and the light chains.

In one embodiment, the term “antibody” or “antigen-binding fragment” respectively refer to intact molecules as well as functional fragments thereof, such as Fab, a scFv-Fc bivalent molecule, F(ab′)2, and Fv that are capable of specifically interacting with a desired target. In some embodiments, the antigen-binding fragments comprise:

(1) Fab, the fragment which contains a monovalent antigen-binding fragment of an antibody molecule, which can be produced by digestion of whole antibody with the enzyme papain to yield an intact light chain and a portion of one heavy chain;

(2) Fab′, the fragment of an antibody molecule that can be obtained by treating whole antibody with pepsin, followed by reduction, to yield an intact light chain and a portion of the heavy chain; two Fab′ fragments are obtained per antibody molecule;

(3) (Fab′)2, the fragment of the antibody that can be obtained by treating whole antibody with the enzyme pepsin without subsequent reduction; F(ab′)2 is a dimer of two Fab′ fragments held together by two disulfide bonds;

(4) Fv, a genetically engineered fragment containing the variable region of the light chain and the variable region of the heavy chain expressed as two chains; and

(5) Single chain antibody (“SCA”), a genetically engineered molecule containing the variable region of the light chain and the variable region of the heavy chain, linked by a suitable polypeptide linker as a genetically fused single chain molecule.

(6) scFv-Fc, is produced in one embodiment, by fusing single-chain Fv (scFv) with a hinge region from an immunoglobulin (Ig) such as an IgG, and Fc regions.

In some embodiments, an antibody provided herein is a monoclonal antibody. In some embodiments, the antigen-binding fragment provided herein is a single chain Fv (scFv), a diabody, a tri(a)body, a di- or tri-tandem scFv, a scFv-Fc bivalent molecule, an Fab, Fab′, Fv, F(ab′)2 or an antigen binding scaffold (e.g., affibody, monobody, anticalin, DARPin, Knottin, etc.). “Affibodies” are small proteins engineered to bind to a large number of target proteins or peptides with high affinity, often imitating monoclonal antibodies, and are antibody mimetics.

As used herein, the terms “bivalent molecule” or “By” refer to a molecule capable of binding to two separate targets at the same time. The bivalent molecule is not limited to having two and only two binding domains and can be a polyvalent molecule or a molecule comprised of linked monovalent molecules. The binding domains of the bivalent molecule can selectively recognize the same epitope or different epitopes located on the same target or located on a target that originates from different species. The binding domains can be linked in any of a number of ways including, but not limited to, disulfide bonds, peptide bridging, amide bonds, and other natural or synthetic linkages known in the art (Spatola et al., “Chemistry and Biochemistry of Amino Acids, Peptides and Proteins,” B. Weinstein, eds., Marcel Dekker, New York, p. 267 (1983); Morley, J. S., “Trends Pharm Sci.” (1980) pp. 463-468; Hudson et al., Int. J. Pept. Prot. Res. (1979) 14, 177-185; Spatola et al., Life Sci. (1986) 38, 1243-1249; Hann, M. M., J. Chem. Soc. Perkin Trans. I (1982) 307-314; Almquist et al., J. Med. Chem. (1980) 23, 1392-1398; Jennings-White et al., Tetrahedron Lett. (1982) 23, 2533; Szelke et al., European Application EP 45665; Chemical Abstracts 97, 39405 (1982); Holladay, et al., Tetrahedron Lett. (1983) 24, 4401-4404; and Hruby, V. J., Life Sci. (1982) 31, 189-199).

As used herein, the terms “binds” or “binding” or grammatical equivalents, refer to compositions having affinity for each other. “Specific binding” is where the binding is selective between two molecules. A particular example of specific binding is that which occurs between an antibody and an antigen. Typically, specific binding can be distinguished from non-specific when the dissociation constant (KD) is less than about 1×10-5 M or less than about 1×10-6 M or 1×10-7 M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

In addition to antibody sequences, an antibody according to the present invention may comprise other amino acids, e.g., forming a peptide or polypeptide, such as a folded domain, or to impart to the molecule another functional characteristic in addition to ability to bind antigen. For example, antibodies of the invention may carry a detectable label, such as fluorescent or radioactive label, or may be conjugated to a toxin (such as a holotoxin or a hemitoxin) or an enzyme, such as beta-galactosidase or alkaline phosphatase (e.g., via a peptidyl bond or linker).

In one embodiment, an antibody of the invention comprises a stabilized hinge region. The term “stabilized hinge region” will be understood to mean a hinge region that has been modified to reduce Fab arm exchange or the propensity to undergo Fab arm exchange or formation of a half-antibody or a propensity to form a half-antibody. “Fab arm exchange” refers to a type of protein modification for human immunoglobulin, in which a human immunoglobulin heavy chain and attached light chain (half-molecule) is swapped for a heavy-light chain pair from another human immunoglobulin molecule. Thus, human immunoglobulin molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half-antibody” forms when a human immunoglobulin antibody dissociates to form two molecules, each containing a single heavy chain and a single light chain. In one embodiment, the stabilized hinge region of human immunoglobulin comprises a substitution in the hinge region.

In one embodiment, the term “hinge region” as used herein refers to a proline-rich portion of an immunoglobulin heavy chain between the Fc and Fab regions that confers mobility on the two Fab arms of the antibody molecule. It is located between the first and second constant domains of the heavy chain. The hinge region includes cysteine residues which are involved in inter-heavy chain disulfide bonds. In one embodiment, the hinge region includes cysteine residues which are involved in inter-heavy chain disulfide bonds.

In one embodiment, the antibody or antigen-binding fragment binds its target with a KD of 0.1 nM-10 mM. In one embodiment, the antibody or antigen-binding fragment binds its target with a KD of 0.1 nM-1 mM. In one embodiment, the antibody or antigen-binding fragment binds its target with a KD within the 0.1 nM range. In one embodiment, the antibody or antigen-binding fragment binds its target with a KD of 0.1-2 nM. In another embodiment, the antibody or antigen-binding fragment binds its target with a KD of 0.1-1 nM. In another embodiment, the antibody or antigen-binding fragment binds its target with a KD of 0.05-1 nM. In another embodiment, the antibody or antigen-binding fragment binds its target with a KD of 0.1-0.5 nM. In another embodiment, the antibody or antigen-binding fragment binds its target with a KD of 0.1-0.2 nM.

In some embodiments, the antibody or antigen-binding fragment thereof provided herein comprises a modification. In another embodiment, the modification minimizes conformational changes during the shift from displayed to secreted forms of the antibody or antigen-binding fragment. It is to be understood by a skilled artisan that the modification can be a modification known in the art to impart a functional property that would not otherwise be present if it were not for the presence of the modification. Encompassed are antibodies which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

In some embodiments, the modification is one as further defined herein below. In some embodiments, the modification is a N-terminus modification. In some embodiments, the modification is a C-terminal modification. In some embodiments, the modification is an N-terminus biotinylation. In some embodiments, the modification is a C-terminus biotinylation. In some embodiments, the secretable form of the antibody or antigen-binding fragment comprises an N-terminal modification that allows binding to an Immunoglobulin (Ig) hinge region. In some embodiments, the Ig hinge region is from but is not limited to, an IgA hinge region. In some embodiments, the secretable form of the antibody or antigen-binding fragment comprises an N-terminal modification that allows binding to an enzymatically biotinylatable site. In some embodiments, the secretable form of the antibody or antigen-binding fragment comprises a C-terminal modification that allows binding to an enzymatically biotinylatable site. In some embodiments, biotinylation of said site functionalizes the site to bind to any surface coated with streptavidin, avidin, avidin-derived moieties, or a secondary reagent.

It will be appreciated that the term “modification” can encompass an amino acid modification such as an amino acid substitution, insertion, and/or deletion in a polypeptide sequence.

In one embodiment, a variety of radioactive isotopes are available for the production of radioconjugate antibodies and other proteins and can be of use in the methods and compositions provided herein. Examples include, but are not limited to, At211, Cu64, I131, I125, Y90, Re186, Re188, Sm153, Bi212, P32, Zr89 and radioactive isotopes of Lu. In a further embodiment, the amino acid sequences of the invention may be homologues, variants, isoforms, or fragments of the sequences presented. The term “homolog” as used herein refers to a polypeptide having a sequence homology of a certain amount, namely of at least 70%, e.g. at least 80%, 90%, 95%, 96%, 97%, 98%, 99% of the amino acid sequence it is referred to. Homology refers to the magnitude of identity between two sequences. Homolog sequences have the same or similar characteristics, in particular, have the same or similar property of the sequence as identified. The term ‘variant’ as used herein refers to a polypeptide wherein the amino acid sequence exhibits substantially 70, 80, 95, or 99% homology with the amino acid sequence as set forth in the sequence listing. It should be appreciated that the variant may result from a modification of the native amino acid sequences, or by modifications including insertion, substitution or deletion of one or more amino acids. The term “isoform” as used herein refers to variants of a polypeptide that are encoded by the same gene, but that differ in their isoelectric point (pI) or molecular weight (MW), or both. Such isoforms can differ in their amino acid composition (e.g. as a result of alternative splicing or limited proteolysis) and in addition, or in the alternative, may arise from differential post-translational modification (e.g., glycosylation, acylation, phosphorylation deamidation, or sulphation). As used herein, the term “isoform” also refers to a protein that exists in only a single form, i.e., it is not expressed as several variants. The term “fragment” as used herein refers to any portion of the full-length amino acid sequence of protein of a polypeptide of the invention which has less amino acids than the full-length amino acid sequence of a polypeptide of the invention. The fragment may or may not possess a functional activity of such polypeptides.

In an alternate embodiment, enzymatically active toxin or fragments thereof that can be used in the compositions and methods provided herein include, but are not limited, to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

A chemotherapeutic or other cytotoxic agent may be conjugated to the protein, according to the methods provided herein, as an active drug or as a prodrug. The term “prodrug” refers to a precursor or derivative form of a pharmaceutically active substance that is less cytotoxic to tumor cells compared to the parent drug and is capable of being enzymatically activated or converted into the more active parent form. (See, for example Wilman, 1986, Biochemical Society Transactions, 615th Meeting Belfast, 14:375-382; and Stella et al., “Prodrugs: A Chemical Approach to Targeted Drug Delivery,” Directed Drug Delivery, Borchardt et al., (ed.): 247-267, Humana Press, 1985.) The prodrugs that may find use with the compositions and methods as provided herein include but are not limited to phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use with the antibodies and Fc fusions of the compositions and methods as provided herein include but are not limited to any of the aforementioned chemotherapeutic.

Non-limiting examples of antibodies, antibody fragments and antigen-binding proteins include single-chain antibodies such as scFvs. A non-limiting example, a scFv that blocks PD-1 for the treatment of cancer or tumor, including in association with CAR-T therapy, wherein activation of scFv production can be directed at a particular site in the body, in one embodiment, at or near a tumor. Another non-limiting example includes brolucizumab, which targets VEGF-A and is used to treat wet age-related macular degeneration.

In another example, the therapeutic protein is an immune checkpoint inhibitor, such as an antibody fragment, or antigen-binding protein, that inhibits a checkpoint molecule such as but not limited to PD-1, PD-L1, CTLA-4, CTLA-4 receptor, PD1-L2, 4-1BB, OX40, LAG-3 and TIM-3. In one embodiment, a scFv that inhibits a checkpoint protein. In one embodiment, the porous scaffold is used in association with a cancer or tumor therapy, such as CAR-T therapy. Thus, the porous scaffold provides a similar therapeutic activity as antibodies to PD-1 and other checkpoint molecules.

### Cell Adhesion/Attraction Components

Any one or more cell adhesion and/or cell attraction and/or immunostimulatory and/or immunosuppression compounds or components may be included in or on the scaffold. In one embodiment, such components attract or activate T cells. Non-limiting examples include CCL21, anti-CD3 antibodies, anti-CD28 antibodies, or any combination thereof. In one embodiment, a combination of anti-CD3 and an anti-CD28 antibodies are used. Any one or more immunostimulatory components may be included in or on the scaffold. In some embodiments, components such as but not limited to IL-2, IL-4, IL-6, IL-7, IL-10, IL-12 IL-15, or IL-2 superkine are used, singly or in any combination. In one embodiment as described below, such components may be bound to mesoporous silica microparticles or to heparin-modified mesoporous silica microparticles comprising the scaffold. In another embodiment, post-modification of the scaffold is performed to conjugate anti-CD3 and anti-CD28 antibodies through EDC/NHS cross-linking reagents, to provide stronger T cell activation signals. In other embodiments, such compounds or components suppress T cell attraction or T cell activation. Additional embodiments are described elsewhere herein.

### Treg Regulators

Any one of various methods of regulating Treg induction and/or suppression of Treg induction may be used.

A TGF-β inhibitor (TGF-βi) such as a TGF-β receptor inhibitor may be used. Non-limiting examples include galinusertib (LY2157299) or SB505124. The compound is incorporated into the scaffold. In one embodiment, the TGF-βi suppresses the formation of induced Tregs and thus enhances the tumoricidal activity of T cells attracted to, activated, or delivered by the scaffolds described herein. In one embodiment the TGF-β inhibitor or inducer is slowly released from the scaffold. Alternatively, compounds that induce Tregs may be used. Non-limiting examples include TGF-β and activators thereof (e.g., IL-2, IL-4, etc.). Additional embodiments are described elsewhere herein.

### Methods of Making Scaffolds

Implantable scaffolds are made of various biocompatible and biodegradable polymers, such as alginate, hyaluronic acid, and chitosan. Microscale pores are created within the structures. To create scaffolds with stimulatory capability by this artificial niche, mesoporous silica microparticles are embedded in the scaffolds. These microparticles are loaded with at least one compound that regulates T cell immune response (e.g., with cytokines [e.g., IL-2, IL-4, IL-6, IL-7, IL-10, IL-12, IL-15, IL-2 superkine] to improve T cells' proliferation and effector functions).

The implantable scaffold can be made of various biocompatible and biodegradable polymers. To further encourage cell trafficking within these structures, cell adhesion peptides such as but not limited to the chemokine CCL21, and immunostimulatory compounds such as IL-2, IL-4, IL-6, IL-7, IL-10, IL-12 IL-15, or IL-2 superkine, or antibodies such as anti-CD3 and anti-CD28 are provided. To improve the resemblance of these 3D matrices to natural tissues techniques are used that create microscale pores within these structures that both allows for maximizing the loading capacity for delivering T cells and facilitates their expansion as well. The scaffolds are modified with anti-CD3/anti-CD28 antibodies and further comprise a TGF-βi, as well as IL-2 cytokine to provide activation signal for T cells and prevent formation of regulatory T cells.

### Methods of Using Scaffolds

The scaffolds described herein can be fabricated for various applications. In one aspect, the porous scaffold is provided at a site at or near a focus of interest in a subject in need. In one embodiment, one or more scaffolds are inserted surgically at or near the site of a tumor during resection or biopsy. In one embodiment the scaffold is implanted at or near the site of a tumor. In one embodiment the scaffold biodegrades. In some embodiment the mechanical properties of the scaffold as well as the degradation time can be modified for a particular use by changing the formulation.

In some embodiments, “treating” comprises therapeutic treatment including prophylactic or preventive measures, wherein the object is to prevent or lessen the targeted pathologic condition or disorder, for example to treat or prevent cancer. Thus, in some embodiments, treating may include directly affecting or curing, suppressing, inhibiting, preventing, reducing the severity of, delaying the onset of, reducing symptoms associated with cancer or a combination thereof. Thus, in other embodiments, treating may include directly affecting or curing, suppressing, inhibiting, preventing, reducing the severity of, delaying the onset of, reducing symptoms associated with a non-cancerous tumor or a combination thereof. Thus, in some embodiments, “treating,” “ameliorating,” and “alleviating” refer inter alia to delaying progression, expediting remission, inducing remission, augmenting remission, speeding recovery, increasing efficacy of or decreasing resistance to alternative therapeutics, or a combination thereof. In some embodiments, “preventing” refers, inter alia, to delaying the onset of symptoms, preventing relapse to a disease, decreasing the number or frequency of relapse episodes, increasing latency between symptomatic episodes, or a combination thereof. In some embodiments, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

A “cancer” is one of a group of diseases characterized by uncontrollable growth and having the ability to invade normal tissues and to metastasize to other parts of the body. Cancers have many causes, including, but not limited to, diet, alcohol consumption, tobacco use, environmental toxins, heredity, and viral infections. In most instances, multiple genetic changes are required for the development of a cancer cell. Progression from normal to cancerous cells involves a number of steps to produce typical characteristics of cancer including, e.g., cell growth and division in the absence of normal signals and/or continuous growth and division due to failure to respond to inhibitors thereof; loss of programmed cell death (apoptosis); unlimited numbers of cell divisions (in contrast to a finite number of divisions in normal cells); aberrant promotion of angiogenesis; and invasion of tissue and metastasis.

A “pre-cancerous” condition, lesion, or tumor is a condition, lesion, or tumor comprising abnormal cells associated with a risk of developing cancer. Non-limiting examples of pre-cancerous lesions include colon polyps (which can progress into colon cancer), cervical dysplasia (which can progress into cervical cancer), and monoclonal monopathy (which can progress into multiple myeloma). Premalignant lesions comprise morphologically atypical tissue which appears abnormal when viewed under the microscope, and which are more likely to progress to cancer than normal tissue.

A “non-cancerous tumor” or “benign tumor” is one in which the cells demonstrate normal growth, but are produced, e.g., more rapidly, giving rise to an “aberrant lump” or “compact mass,” which is typically self-contained and does not invade tissues or metastasize to other parts of the body. Nevertheless, a non-cancerous tumor can have devastating effects based upon its location (e.g., a non-cancerous abdominal tumor that prevents pregnancy or causes a ureter, urethral, or bowel blockage, or a benign brain tumor that is inaccessible to normal surgery and yet damages the brain due to unrelieved pressure as it grows).

In some embodiments, “treating” comprises therapeutic treatment including prophylactic or preventive measures, wherein the object is to prevent or lessen the targeted pathologic condition or disorder, for example to treat or prevent an autoimmune disease, an allergic reaction or hypersensitivity reaction, a localized infection or an infectious disease, an injury or other damage, a transplant or other surgical site, or a symptom thereof, or a combination thereof. Thus, in some embodiments, treating may include directly affecting or curing, suppressing, inhibiting, preventing, reducing the severity of, delaying the onset of, reducing symptoms associated with an autoimmune disease, an allergic reaction or hypersensitivity reaction, a localized infection or an infectious disease, an injury or other damage, a transplant or other surgical site, or a symptom thereof, or a combination thereof. Thus, in some embodiments, “treating,” “ameliorating,” and “alleviating” refer inter alia to delaying progression, expediting remission, inducing remission, augmenting remission, speeding recovery, increasing efficacy of or decreasing resistance to alternative therapeutics, or a combination thereof. In some embodiments, “preventing” refers, inter alia, to delaying the onset of symptoms, preventing relapse to a disease, decreasing the number or frequency of relapse episodes, increasing latency between symptomatic episodes, or a combination thereof. In some embodiments, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

A “focus of interest” a “localized environment,” or a “localized site” comprises a site in which the disease, reaction, infection, injury, or other medical condition is specific to one part or area of the body; in which a symptom or condition of the medical condition is specific to one part or area of the body; or in which treatment is desired for one part or area of the body (even if the disease, reaction, infection, injury, or other medical condition affects other parts or areas of the body or the body as a whole).

In some embodiments, the scaffold comprises a microparticle not comprising alginate, heparin, or a lipid coating. In some embodiments, the scaffold comprises a microparticle comprising alginate. In some embodiments, the scaffold comprises a microparticle comprising alginate-heparin. In certain embodiments, this scaffold can be administered via a catheter. In certain embodiments, this scaffold can be implanted or injected locally at the site of a tumor. Scaffolding comprising microparticles provides in some embodiments, further control over the release of the compound regulating T cell immune response and/or the compound regulating induction of Tregs, and also localizes the effects. In some embodiments, implantation, injection, or other administration of the scaffold provides a stronger cytokine gradient to boost up the therapeutic effects.

In some embodiments, application of the scaffold, or compositions thereof is for local use. This may, in certain embodiments, provide an advantage, wherein the controlled localized release of the compound regulating T cell immune response and/or the compound regulating induction of Tregs may provide a local immune effect thereby avoiding a toxic systemic effect of the cytokine. In one example, controlled release of IL-2 or an IL-2 superkine, may increase proliferation of cytotoxic T cells and or helper T cells in the area adjacent to the cancer or tumor, thereby promoting clearance of the cancer or tumor. In some embodiments, controlled release of IL-2 or an IL-2 superkine, may maintain a helper T cell population in the area adjacent to the tumor. In some embodiments, controlled release of IL-2 or an IL-2 superkine, may activate a cytotoxic T cell population in the area adjacent to the tumor. In some embodiments, controlled release of IL-2 or an IL-2 superkine, may lead to enhanced killing of tumor cells in the localized area at and adjacent to the tumor. In some embodiments, controlled release of IL-2 or an IL-2 superkine, provides enhanced clearance of a tumor. This technique may also be used for the treatment of other diseases, reactions, injuries, transplants, blood clots, and the like, recited herein.

As used herein, the terms “composition” and “pharmaceutical composition” may in some embodiments, be used interchangeably having all the same qualities and meanings. In some embodiments, disclosed herein is a pharmaceutical composition for the treatment of a cancer or tumor as described herein. In some embodiments, disclosed herein is a pharmaceutical composition for the treatment of cancer or tumor. In some embodiments, disclosed herein is a pharmaceutical composition for the use in methods locally regulating an immune response. In some embodiments, disclosed herein are pharmaceutical compositions for the treatment of an autoimmune disease, an allergic reaction or hypersensitivity reaction, a localized site of an infection or infectious disease, a localized site of an injury or other damage, a transplant or other surgical site, a blood clot causing or at risk for causing a myocardial infarction, an ischemic stroke, or a pulmonary embolism or a symptom thereof, or a combination thereof.

In some embodiments, a pharmaceutical composition comprises a porous scaffold, as described in detail above. In still another embodiment, a pharmaceutical composition for the treatment of a disease or medical condition, as described herein, comprises an effective amount of the compound regulating T cell immune response and/or the compound regulating induction of Tregs and a pharmaceutically acceptable excipient. In some embodiments, a composition comprising the porous scaffold comprising the compound regulating T cell immune response and/or the compound regulating induction of Tregs and a pharmaceutically acceptable excipient is used in methods for regulating an immune response.

### Tumors

In one embodiment, the subject for implantation of a scaffold as described herein has a solid tumor or cancer. In another embodiment, the tumor is a lymphatic tumor or cancer. In another embodiment, the tumor or cancer is any tumor or cancer. In another embodiment, the disease or medical condition comprises a tumor, a suspected tumor, or a resected tumor. In one embodiment, the tumor, suspected tumor, or resected tumor comprises a cancerous, pre-cancerous, or non-cancerous tumor. Non-limiting examples include a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor (for example, a triple-negative breast cancer), an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma, esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non-small cell lung cancer (NSCLC), squamous cell non-small cell lung cancer, breast cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16;16)(p13.1;q22); CBFB-MYH11; adult acute myeloid leukemia with t(8;21)(q22;q22); RUNX1-RUNX1T1; adult acute myeloid leukemia with t(9;11)(p22;q23); MLLT3-MLL; adult acute promyelocytic leukemia with t(15;17)(q22;q12); PML-RARA; alkylating agent-related acute myeloid leukemia, chronic lymphocytic leukemia, Richter's syndrome; Waldenstrom's macroglobulinemia, adult glioblastoma; adult gliosarcoma, recurrent glioblastoma, recurrent childhood rhabdomyosarcoma, recurrent Ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent Merkel cell carcinoma; stage III Merkel cell carcinoma; stage IV Merkel cell carcinoma, myelodysplastic syndrome and recurrent mycosis fungoides and Sezary syndrome. In another related aspect, the tumor or cancer comprises a metastasis of a tumor or cancer. In some embodiments, a solid tumor treated using a method described herein, originated as a blood tumor or diffuse tumor.

In some embodiments, a pharmaceutical composition comprises the porous scaffold, as described in detail above. In still another embodiment, a pharmaceutical composition for the treatment of cancer or tumor, as described herein, comprises an effective amount of the compound regulating T cell immune response and/or the compound regulating induction of Tregs and a pharmaceutically acceptable excipient. In some embodiments, a composition comprising the porous scaffold comprising the compound regulating T cell immune response and/or the compound regulating induction of Tregs and a pharmaceutically acceptable excipient is used in methods for regulating an immune response. In some embodiments, treating reduces the size of the tumor, eliminates the tumor, slows the growth or regrowth of the tumor, or prolongs the survival of the subject, or any combination thereof. In some embodiments, a composition comprising the porous scaffold is used in methods to reduce the size of a tumor. In some embodiments, a composition comprising the porous scaffold is used in methods to eliminate the tumor. In some embodiments, a composition comprising the porous scaffold is used in methods to slow the growth of a tumor. In some embodiments, a composition comprising the porous scaffold is used in methods to prolong the survival of the subject. In some embodiments, methods of treating described herein reduce the size of the tumor, eliminate said tumor, slow the growth or regrowth of the tumor, or prolong survival of said subject, or any combination thereof.

### Immune Response Stimulation or Suppression

In one embodiment, the scaffolds can be used to stimulate the immune response. In another embodiment, the scaffolds can be used to deliver signals to suppress the immune response. In a non-limiting example, by using TGF-β instead of a TGFβi in the formulation, the scaffolds will provide signals to promote formation of regulatory T cells (Tregs). These cells can contribute in modulating the immune response after organ transplantation.

In some embodiments, the disease or medical condition comprises an autoimmune disease, and the porous scaffold is provided at or adjacent to a focus of interest comprising an autoimmune-targeted or symptomatic focus of said autoimmune disease; the disease or medical condition comprises an allergic reaction or hypersensitivity reaction, and the porous scaffold is provided at or adjacent to a focus of interest comprising a reactive focus of said allergic reaction or hypersensitivity reaction; the disease or medical condition comprises a localized infection or an infectious disease, and the porous scaffold is provided at or adjacent to a focus of interest comprising a focus of infection or symptoms; the disease or medical condition comprises an injury or a site of chronic damage, and the porous scaffold is provided at or adjacent to a focus of interest comprising the injury or the site of chronic damage; the disease or medical condition comprises a surgical site, and the porous scaffold is provided at or adjacent to a focus of interest comprising the surgical site; the disease or medical condition comprises a transplanted organ, tissue, or cell, and the porous scaffold is provided at or adjacent to a focus of interest comprising a transplant site; or the disease or medical condition comprises a blood clot causing or at risk for causing a myocardial infarction, an ischemic stroke, or a pulmonary embolism, and the porous scaffold is provided at or adjacent to a focus of interest comprising the site of the blood clot.

In some embodiments, the autoimmune disease includes, for example, but is not limited to, rheumatoid arthritis, juvenile dermatomyositis, psoriasis, psoriatic arthritis, sarcoidosis, lupus, Crohn's disease, eczema, vasculitis, ulcerative colitis, multiple sclerosis, or type 1 diabetes, achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Baló disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatosis (EGPA), cicatricial pemphigoid, Cogan's syndrome, cold agglutinin disease, congenital hear block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis) giant cell myocarditis, glomerulonephritis, Goodpasture's syndrome, granulomatosis with polyangiitis, Grave's disease, Guillain-Barre syndrome, Hashimoto's thyroiditis, hemolytic anemia, Henoch-Schonlein purpura (HSP), Herpes gestationis or pemphigoid gestationis (PG), Hidradenitis suppurativa (HS; acne inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease, lupus, Lyme disease chronic, Menier's disease, microscopic polyangiitis (MPA), mixed connective tissue disease (MCTD), Mooren's ulcer, Mucha-Habermann disease, multifocoal motor neuropathy (MMN, MMNCB), multiple sclerosis, myasthenia gravis, myositis, narcolepsy, neonatallupus, neuromyelitis optica, neutropenia, ocular cicatricial pemphigoid, optic neuritis, palindromic rheumatism (PR), PANDAS, paraneoplasticcerebellar degeneration (PCD), paroxysmal nocturnal hemoglobinuria (PNH), Parry Romberg syndrome, Pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes types I-III, polymyalgia rheumatica, polymyositis, postmyocadial infarction syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy (RSD; complex regional pain syndrome [CRPS]), relapsing polychondritis, restless leg syndrome (RLS), retroperitoneal fibrosis, rheumatic fever, rheumatoid arthritis (RA), sarcoidosis, Schmidt syndrome, scleritis, scleroderma, Sjorgren's syndrome, sperm & testicular autoimmunity, stiff person syndrome (SPS), subacute bacterial endocarditis (SBE), Susac's syndrome, sympathetic ophthalmia (SO), Takayasu arteritis, temporal arteritis/giant cell arteritis, thrombocytopenic purpura (TTP), thyroid eye disease (TED), Tolosa-Hunt syndrome (THS), transverse myelitis, type 1 diabetes, ulcerative colitis (UC), undifferentiated connective tissue disease (UCTD), uveitis, vasculitis, vitiligo, or Vogt-Koyanagi-Harada disease.

Alternatively, protein production locally for autoimmune diseases targets the pathogenic antibodies in the disease, for example, a protein that breaks down antibodies in the vicinity (an IgG endopeptidase) or a protein that binds antibodies (a decoy of the antibody's autoimmune target).

In some embodiments, the allergic reaction includes, for example, but is not limited to, a localized allergic reaction or hypersensitivity reaction including a skin rash, hives, localized swelling (e.g., from an insect bite), or esophageal inflammation from food allergies or eosinophilic esophagitis, other enteric inflammation from food allergies or eosinophilic gastrointestinal disease, localized drug allergies when the drug treatment was local to a part of the body, or allergic conjunctivitis.

In some embodiments, the localized site of an infection or the localized site of an infectious disease includes, for example, but is not limited to, a fungal infection (e.g., aspergillus, coccidioidomycosis, tinea pedis (foot), tinea corporis (body), tinea cruris (groin), tinea capitis (scalp), and tinea unguium (nail)), a bacterial infection (e.g., methicillin-resistant Staphylococcus aureus [MRSA], localized skin infections, abscesses, necrotizing facsciitis, pulmonary bacterial infections [e.g., pneumonia], bacterial meningitis, bacterial sinus infections, bacterial cellulitis, such as due to Staphylococcus aureus (MRSA), bacterial vaginosis, gonorrhea, chlamydia, syphilis, Clostridium difficile (C. diff), tuberculosis, cholera, botulism, tetanus, anthrax, pneumococcal pneumonia, bacterial meningitis, Lyme disease), a viral infection (e.g., varicella-zoster/herpes zoster [shingles], Herpes simplex I [e.g., cold sores/fever blisters], Herpes simplex II [genital herpes], or human papilloma virus [e.g., cervical cancer, throat cancer, esophageal cancer, mouse cancer], Epstein-Barr virus [e.g., nasopharyngeal cancer], encephalitis viruses [e.g., brain inflammation], or hepatitis viruses [e.g., liver disease; hepatitis A, hepatitis B, hepatitis C, hepatitis D, hepatitis E, hepatitis F, hepatitis G] or COVID-19), a parasitic infection (e.g., an area infected by scabies, Chagas, Hypoderma tarandi, amoebae, roundworm, Toxoplasma gondii). In some embodiments, the injury or other damage includes, for example, but is not limited to traumatic injury (e.g., resulting from an accident or violence) or chronic injury (e.g., osteoarthritis). In some embodiments, the localized site of injury comprises a muscular-skeletal injury, a neurological injury, an eye or ear injury, an internal or external wound, or a localized abscess, an area of mucosa that is affected (e.g., conjunctiva, sinuses, esophagus), or an area of skin that is affected (e.g., infection, autoimmunity). In some embodiments, the transplant or other surgical site includes, for example, but is not limited to, the site and/or its local environment or surroundings of an organ, corneal, skin, limb, face, or other transplant, or a surgical site and/or its local environment or surroundings, for, e.g., but not limited to, treatment of surgical trauma, treatment of a condition related to the transplant or surgery, or prevention of infection. In some embodiments, the site is at or adjacent to a blood clot causing or at risk for causing a myocardial infarction, an ischemic stroke, or a pulmonary embolism. In some embodiments, the methods disclosed herein treat one or more symptoms of a disease, reaction, infection, injury, transplant, surgery, or blood clot. In some embodiments, the methods disclosed herein treat a combination thereof.

In some embodiments, a pharmaceutical composition comprises the compound regulating T cell immune response and/or the compound regulating induction of Tregs, as described in detail above. In still another embodiment, a pharmaceutical composition for the treatment of an autoimmune disease, an allergic reaction or hypersensitivity reaction, a localized site of an infection or infectious disease, a localized site of an injury or other damage, a transplant or other surgical site, a blood clot, or a symptom thereof of any one of these, or a combination thereof, as described herein, comprises an effective amount of the compound regulating T cell immune response and/or the compound regulating induction of Tregs and a pharmaceutically acceptable excipient. In some embodiments, a composition comprising the compound regulating T cell immune response and/or the compound regulating induction of Tregs and a pharmaceutically acceptable excipient is used in methods for regulating an immune response. In some embodiments, a composition comprising the compound regulating T cell immune response and/or the compound regulating induction of Tregs is used in methods for promoting clearance of or alleviating localized symptoms of the autoimmune disease, allergic reaction or hypersensitivity reaction, infection or infectious disease. In some embodiments, a composition comprising the compound regulating T cell immune response and/or the compound regulating induction of Tregs is used in methods for facilitating healing and/or preventing or inhibiting infection or rejection of a localized site of an injury or other damage, a transplant or other surgical site. In some embodiments, a composition comprising the compound regulating T cell immune response and/or the compound regulating induction of Tregs is used in methods for alleviating localized symptoms relating to an autoimmune disease, an allergic reaction or hypersensitivity reaction, a localized site of an infection or infectious disease, a localized site of an injury or other damage, a transplant or other surgical site, or a symptom thereof, or a combination thereof. In some embodiments, a composition comprising the compound regulating T cell immune response and/or the compound regulating induction of Tregs is used in methods to prolong the survival of the subject. In some embodiments, methods of treating described herein for promoting clearance of or alleviating localized symptoms of the autoimmune disease, allergic reaction or hypersensitivity reaction, infection or infectious disease; for facilitating healing and/or preventing or inhibiting infection or rejection of a localized site of an injury or other damage, a transplant or other surgical site; for reducing or eliminating a blood clot causing or at risk for causing a myocardial infarction, an ischemic stroke, or a pulmonary embolism; or for alleviating localized symptoms thereof; or for a combination thereof.

In some embodiments, a method of use of the porous scaffold comprising the compound regulating T cell immune response and/or the compound regulating induction of Tregs further comprises a step of administering activated T cells to said subject. Methods of preparing T cells are known in the art. In some embodiments, these cells may be administered prior to or after administering the porous scaffold comprising the compound regulating T cell immune response and/or the compound regulating induction of Tregs. In some embodiments, T cells are administered by intravenous (i.v.) injection. In some embodiments, administration of T cells enhances the therapeutic effect provided by the regulated, local administration of the compound regulating T cell immune response and/or the compound regulating induction of Tregs, as administered from the porous scaffold.

Treatment of the subject with the porous scaffolds may also be used in conjunction with other known treatments. In a non-limiting example, when the disease or medical condition comprises a blood clot causing or at risk for causing a myocardial infarction, an ischemic stroke, or a pulmonary embolism, and the porous scaffold may be provided at or adjacent to a focus of interest comprising the site of the blood clot together with angioplasty or another clot removal treatment.

In some embodiments, treating reduces or eliminates inflammation or another symptom of the autoimmune-targeted or symptomatic focus of an autoimmune disease, prolongs survival of the subject, or any combination thereof; reduces or eliminates inflammation or another symptom of allergic reaction or hypersensitivity reaction at the reactive focus of an allergic reaction or hypersensitivity reaction, prolongs survival of the subject, or any combination thereof; reduces or eliminates infection or symptoms at the focus of infection or symptoms of a localized infection or infectious disease, prolongs survival of the subject, or any combination thereof; reduces, eliminates, inhibits or prevents structural, organ, tissue, or cell damage, inflammation, infection, or another symptom at a site of injury or a site of chronic damage, improves structural, organ, tissue, or cell function at a site of injury or a site of chronic damage, improves mobility of the subject, prolongs survival of the subject, or any combination thereof; reduces, eliminates, inhibits, or prevents structural, organ, tissue, or cell damage, inflammation, infection, or another symptom at a surgical site, improves structural, organ, tissue, or cell function at a surgical site, improves mobility of the subject, prolongs survival of the subject, or any combination thereof; reduces, eliminates, inhibits or prevents transplanted organ, tissue, or cell damage or rejection, inflammation, infection or another symptom at a transplant site, improves mobility of the subject, prolongs survival of a transplanted organ, tissue, or cell, prolongs survival of the subject, or any combination thereof; or reduces or eliminates a blood clot causing or at risk for causing a myocardial infarction, an ischemic stroke, or a pulmonary embolism in the subject, improves function or survival of a heart, brain, or lung organ, tissue, or cell in the subject, reduces damage to a heart, brain, or lung organ, tissue, or cell in the subject, prolongs survival of a heart, brain, or lung organ, tissue, or cell in the subject, prolongs survival of the subject, or any combination thereof.

In some embodiments, at the site, T cells are stimulated to target the focus of interest, and the induction of Tregs is suppressed. In other embodiments, at the site, T cells are suppressed at or near the focus of interest, and Tregs are induced.

In some aspects, a method is provided for regulating an immune response at a focus of interest in a subject in need, said method comprising providing a porous scaffold to the subject, at or near a site of the focus of interest, the porous scaffold comprising at least one compound that regulates T cell immune response; and at least one compound that regulates induction of regulatory T cells (Tregs), wherein regulating the immune response comprises increasing or decreasing proliferation of cytotoxic T cells; increasing or decreasing proliferation of helper T cells; maintaining, increasing, or decreasing the population of helper T cells at the site of said focus of interest; activating or suppressing cytotoxic T cells at the site of said focus of interest; or any combination thereof.

Unless otherwise indicated, all numbers expressing quantities, ratios, and numerical properties of ingredients, reaction conditions, and so forth used in the specification and claims are to be understood as being modified in all instances by the term “about”. All parts, percentages, ratios, etc. herein are by weight unless indicated otherwise.

As used herein, the singular forms “a” or “an” or “the” are used interchangeably and intended to include the plural forms as well and fall within each meaning, unless expressly stated otherwise or unless the context clearly dictates otherwise. For example, the term “a compound” or “at least one compound” may include a plurality of compounds, including mixtures thereof.

Also as used herein, “at least one” is intended to mean “one or more” of the listed elements. Singular word forms are intended to include plural word forms and are likewise used herein interchangeably where appropriate and fall within each meaning, unless expressly stated otherwise. Except where noted otherwise, capitalized and non-capitalized forms of all terms fall within each meaning.

The terms “comprises”, “comprising”, “includes”, “including”, “having” and their conjugates encompass “including but not limited to”.

The term “about” or “approximately” means within an acceptable error range for the particular value as determined by one of ordinary skill in the art, which will depend in part on how the value is measured or determined. In some embodiments, the term “about” refers to a deviance of between 0.0001-5% from the indicated number or range of numbers. In some embodiments, the term “about” refers to a deviance of between 1-10% from the indicated number or range of numbers. In some embodiments, the term “about” refers to a deviance of up to 25% from the indicated number or range of numbers. In some embodiments, the term “about” refers to ±10%.

Throughout this application, various embodiments may be presented in a range format. It should be understood that the description in range format is merely for convenience and brevity and should not be construed as an inflexible limitation on the scope of certain embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Any patent, patent application publication, or scientific publication, cited herein, is incorporated by reference herein in its entirety.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Production of Scaffolds with Immunostimulatory Capability and Methods of Use

Implantable scaffolds are made of various biocompatible and biodegradable polymers, such as alginate, hyaluronic acid, and chitosan. Microscale pores are created within the structures. To create scaffolds with stimulatory capability by this artificial niche, mesoporous silica microparticles are embedded in the scaffolds. These microparticles are loaded with compounds that stimulate T cells (e.g., cytokines [e.g., IL-2, IL-4, IL-6, IL-7, IL-10, IL-12, IL-15, IL-2 superkine], chemokine ligands [e.g., CCL21], anti-CD antibodies [e.g., anti-CD3, anti-CD28]) to improve T cells' proliferation and/or effector functions. Nanoparticles or microparticles comprising compounds that suppress induction of Tregs (e.g., TGF-beta inhibitors) may also be included.

The T cell immunostimulatory compound and/or the compound that suppresses induction of Tregs are selected for the treatment of a disease or medical condition of interest, or for the alleviation of localized symptoms, or combinations thereof, in a subject. Alternatively, the T cell immunostimulatory compound and/or the compound that suppresses induction of Tregs are diagnostic compound(s) selected for detecting the presence of a disease or medical condition of interest, or a component or indicator thereof, in a subject.

The scaffold is administered at, adjacent to, or near the site of the focus of interest in the subject in need thereof. The T cell immunostimulatory compound and/or the compound that suppresses induction of Tregs at, adjacent to, or near the site of the focus of interest treat(s) a localized environment within the subject in need thereof. Alternatively, the T cell immunostimulatory compound and/or the compound that suppresses induction of Tregs detect(s) the presence of a disease or medical condition of interest or a component or indicator thereof, within the subject in need thereof.

### Example 2: Production of Scaffolds with Immunosuppression Capability and Methods of Use

Implantable scaffolds are made of various biocompatible and biodegradable polymers, such as alginate, hyaluronic acid, and chitosan. Microscale pores are created within the structures. To create scaffolds with immunosuppression capability by this artificial niche, mesoporous silica microparticles are embedded in the scaffolds. These microparticles are loaded with cytokines (e.g., IL-2, IL-4, TGF-beta) and other suppressors to inhibit T cells' proliferation and/or effector functions. Nanoparticles or microparticles comprising compounds that induce Tregs (e.g., TGF-beta and activators thereof) may also be included.

The T cell immunosuppression compound and/or the compound that induces Tregs are selected for the treatment of a disease or medical condition of interest, or for the alleviation of localized symptoms, or combinations thereof, in a subject. Alternatively, the T cell immunosuppression compound and/or the compound that induces Tregs are diagnostic compound(s) selected for detecting the presence of a disease or medical condition of interest, or a component or indicator thereof, in a subject.

The scaffold is administered at, adjacent to, or near the site of the focus of interest in the subject in need thereof. The T cell immunosuppression compound and/or the compound that induces Tregs at, adjacent to, or near the site of the focus of interest treat(s) a localized environment within the subject in need thereof. Alternatively, the T cell immunosuppression compound and/or the compound that induces Tregs detect(s) the presence of a disease or medical condition of interest or a component or indicator thereof, within the subject in need thereof.

### Example 3: Treatment of Cancerous, Pre-Cancerous, and Non-Cancerous Tumors with Porous Scaffolds

Implantable scaffolds are made of various biocompatible and biodegradable polymers, such as alginate, hyaluronic acid, and chitosan. Microscale pores are created within the structures. To create scaffolds with stimulatory capability by this artificial niche, mesoporous silica microparticles are embedded in the scaffolds. These microparticles are loaded with compounds that stimulate T cells (e.g., cytokines [e.g., IL-2, IL-4, IL-6, IL-7, IL-10, IL-12, IL-15, IL-2 superkine], chemokine ligands [e.g., CCL21], anti-CD antibodies [e.g., anti-CD3, anti-CD28]) to improve T cells' proliferation and/or effector functions. Nanoparticles or microparticles comprising compounds that suppress induction of Tregs (e.g., TGF-beta inhibitors) may also be included.

The T cell immunostimulatory compound and/or the compound that suppresses induction of Tregs are selected for the treatment of a cancerous, pre-cancerous, or non-cancerous tumor, or for the alleviation of localized symptoms, or combinations thereof, in a subject. The T cell immunostimulatory compound and/or the compound that suppresses induction of Tregs acts in concert with other proteins or cells to enhance a desired immune response for the treatment or reduction in size of the cancerous, pre-cancerous, or non-cancerous tumor. The T cell immunostimulatory compound and/or the compound that suppresses induction of Tregs is selected, e.g., to inhibit cell division and/or growth (e.g., a growth factor inhibitor), to inhibit angiogenesis (e.g., an angiogenic factor inhibitor), to promote cell death (e.g., an apoptosis-promoting cytokine or other protein of interest), or to regulate an immune response (e.g., increasing proliferation of cytotoxic T cells, increasing proliferation of helper T cells, maintaining the population of helper T cells, activating cytotoxic T cells, or a combination thereof), in the vicinity of the tumor. Optionally, the method further comprises a step of administering activated T cells to the subject.

Where the tumor is cancerous or pre-cancerous (e.g., a growth comprising cells with at least one pre-cancerous mutation), the T cell immunostimulatory compound and/or the compound that suppresses induction of Tregs may be selected based on the type(s) of cells comprising the tumor and, e.g., any cell surface proteins specific to the cancerous or pre-cancerous cells as compared with neighboring healthy tissue.

The scaffold is administered adjacent to the tumor within the subject in need thereof. Where the tumor is inoperable, it may be possible to use a guided catheter to administer the porous scaffold adjacent to the tumor.

The T cell immunostimulatory compound and/or the compound that suppresses induction of Tregs at, adjacent to, or near the site of the focus of interest treat(s) a localized environment comprising the tumor within the subject.

### Example 4: Treatment of an Autoimmune-Targeted Focus or of a Symptomatic Focus of an Autoimmune Disease with Porous Scaffolds

Implantable scaffolds are made of various biocompatible and biodegradable polymers, such as alginate, hyaluronic acid, and chitosan. Microscale pores are created within the structures. To create scaffolds with stimulatory capability by this artificial niche, mesoporous silica microparticles are embedded in the scaffolds. These microparticles are loaded with compounds that suppress T cells (e.g., cytokines, IL-2, or TGF-beta) to improve T cells' proliferation and/or effector functions. Nanoparticles or microparticles comprising compounds that induce Tregs (e.g., TGF-beta and activators thereof) may also be included. If, as a non-limiting example, the subject has rheumatoid arthritis, the porous scaffold treatment is administered at or adjacent to joints (e.g., in the hands or feet) particularly inflamed or damaged by the effects of rheumatoid arthritis. If, as a non-limiting example, the subject has psoriasis, the porous scaffold treatment is administered at or adjacent to an area of psoriatic rash (e.g., especially if the area is one in which psoriasis is potentially dangerous, such as in close proximity to an eye). If, as a non-limiting example, the subject has eczema, the porous scaffold treatment is administered at or adjacent to an area of eczema on the skin. If, as a non-limiting example, the subject has multiple sclerosis, the porous scaffold treatment is administered at or adjacent to a damaged myelin sheath.

The T cell immunosuppression compound and/or the compound that induces Tregs are selected for the treatment of a an autoimmune-targeted focus or symptomatic focus of an autoimmune disease, or for the alleviation of localized symptoms, or combinations thereof, in a subject. The T cell immunosuppression compound and/or the compound that induces Tregs acts in concert with other proteins or cells to enhance a desired immune response for the treatment or reduction in size of the autoimmune-targeted focus or symptomatic focus of an autoimmune disease. The cytokine or other protein of interest is selected, e.g., to inhibit or promote (as needed) cell division and/or growth (e.g., a growth factor inhibitor), to inhibit inflammation (e.g., anti-inflammatory), to inhibit or promote (as needed) cell death (e.g., an apoptosis-promoting cytokine or other protein of interest), or to regulate an immune response (e.g., decreasing proliferation of cytotoxic T cells, decreasing proliferation of helper T cells, reducing cytotoxic T cells, or a combination thereof, as well as increasing recognition of self), in the vicinity of the autoimmune-targeted or symptomatic focus of the autoimmune disease. Optionally, the method further comprises a step of administering activated T cells to the subject. Optionally, the method further comprises a step of administering activated T cells to the subject.

The scaffold is administered adjacent to the autoimmune-targeted focus or symptomatic focus of the autoimmune disease within the subject in need thereof. Where the site of the focus of interest is inoperable, it may be possible to use a guided catheter to administer the porous scaffold adjacent to autoimmune-targeted focus or symptomatic focus of the autoimmune disease within the subject.

The T cell immunostimulatory compound and/or the compound that suppresses induction of Tregs at, adjacent to, or near the site of the focus of interest treat(s) a localized environment comprising the autoimmune-targeted focus or symptomatic focus of the autoimmune disease within the subject.

### Example 5: Treatment of a Reactive Focus of an Allergic Reaction or Hypersensitivity Reaction with Porous Scaffolds

Implantable scaffolds are made of various biocompatible and biodegradable polymers, such as alginate, hyaluronic acid, and chitosan. Microscale pores are created within the structures. To create scaffolds with stimulatory capability by this artificial niche, mesoporous silica microparticles are embedded in the scaffolds. These microparticles are loaded with compounds that suppress T cells (e.g., cytokines, IL-2, or TGF-beta) to improve T cells' proliferation and/or effector functions. Nanoparticles or microparticles comprising compounds that induce Tregs (e.g., TGF-beta and activators thereof) may also be included.

The T cell immunosuppression compound and/or the compound that induces Tregs are selected for the treatment of a reactive focus of an allergic reaction or hypersensitivity reaction, or for the alleviation of localized symptoms, or combinations thereof, in a subject. Non-limiting examples of a reactive focus of an allergic reaction or hypersensitivity reaction in a subject include a skin rash, a hive or hives, or a localized swelling (e.g., from an insect or other bite).

The T cell immunosuppression compound and/or the compound that induces Tregs acts in concert with other proteins or cells to enhance a desired immune response for the treatment or reduction in size of the reactive focus of an allergic reaction or hypersensitivity reaction, or for the alleviation of localized symptoms. The cytokine or other protein of interest is selected, e.g., to inhibit or promote (as needed) cell division and/or growth (e.g., a growth factor inhibitor), to inhibit inflammation (e.g., anti-inflammatory), to inhibit or promote (as needed) cell death (e.g., an apoptosis-promoting cytokine or other protein of interest), or to regulate an immune response (e.g., decreasing production or accumulation of histamine, increasing proliferation of cytotoxic T cells, increasing proliferation of helper T cells, maintaining the population of helper T cells, activating cytotoxic T cells, or a combination thereof), in the vicinity of the reactive focus of the allergic reaction or hypersensitivity reaction. Optionally, the method further comprises a step of administering activated T cells to the subject.

The scaffold is administered adjacent to the reactive focus of an allergic reaction or hypersensitivity reaction within the subject in need thereof.

The T cell immunostimulatory compound and/or the compound that suppresses induction of Tregs at, adjacent to, or near the site of the focus of interest treat(s) a localized environment comprising the reactive focus of an allergic reaction or hypersensitivity reaction within the subject.

### Example 6: Treatment of a Focus of Infection or Symptoms of a Localized Infection or an Infectious Disease with Porous Scaffolds

Implantable scaffolds are made of various biocompatible and biodegradable polymers, such as alginate, hyaluronic acid, and chitosan. Microscale pores are created within the structures. To create scaffolds with stimulatory capability by this artificial niche, mesoporous silica microparticles are embedded in the scaffolds. These microparticles are loaded with compounds that stimulate T cells (e.g., cytokines [e.g., IL-2, IL-4, IL-6, IL-7, IL-10, IL-12, IL-15, IL-2 superkine], chemokine ligands [e.g., CCL-19, CCL21, SDF-1a], anti-CD antibodies [e.g., anti-CD3, anti-CD28]) to improve T cells' proliferation and/or effector functions. Nanoparticles or microparticles comprising compounds that suppress induction of Tregs (e.g., TGF-beta inhibitors) may also be included.

The T cell immunostimulatory compound and/or the compound that suppresses induction of Tregs are selected for the treatment of a focus of infection or symptoms of a localized infection or an infectious disease, or for the alleviation of localized symptoms, or combinations thereof, in a subject. The T cell immunostimulatory compound and/or the compound that suppresses induction of Tregs acts in concert with other proteins or cells to enhance a desired immune response for the treatment or reduction in the size/amount/severity of the focus of infection or symptoms of a localized infection or an infectious disease. If, as a non-limiting example, the subject has fungal infection (e.g., as described herein), a bacterial infection (e.g., methicillin-resistant Staphylococcus aureus [MRSA], etc.), a viral infection (e.g., a shingles rash from varicella-zoster/herpes zoster; a cold sore/fever blister from, e.g., Herpes simplex I; a genital wart or blister from, e.g., Herpes simplex II, etc.), a parasitic infection (e.g., an area infected by scabies, Chagas, Hypoderma tarandi, an amoeba, a roundworm, Toxoplasma gondii, etc.), the T cell immunostimulatory compound and/or the compound that suppresses induction of Tregs treatment is administered at or adjacent to the infection site, rash, lesion, cold sore, wart, etc., either to treat the infection (e.g., an wound or surgical site infected with MRSA), to contain it or reduce its spread within the subject, to reduce its transmissibility to other individuals (Herpes simplex I or Herpes simplex II), or to reduce a symptom of the infection at the focus of symptoms (e.g., pain associated with an outbreak of shingles). Optionally, the method further comprises a step of administering activated T cells to the subject.

The T cell immunostimulatory compound and/or the compound that suppresses induction of Tregs acts in concert with other proteins or cells to enhance a desired immune response for the treatment of a localized focus of an infection or infectious disease or of one or more localized symptoms of the infection or infectious disease. The cytokine or other protein of interest is selected, e.g., to inhibit or promote (as needed) cell division and/or growth (e.g., a growth factor inhibitor), to inhibit inflammation (e.g., anti-inflammatory), to promote analgesic activity, to inhibit or promote (as needed) cell death (e.g., an apoptosis-promoting cytokine or other protein of interest), or to regulate an immune response (e.g., increasing proliferation of cytotoxic T cells, increasing proliferation of helper T cells, maintaining the population of helper T cells, increasing cytotoxic T cells, or a combination thereof), in the vicinity of the focus of infection or symptoms of a localized infection or an infectious disease. Optionally, the method further comprises a step of administering activated T cells to the subject.

The scaffold is administered at or adjacent to the focus of infection or symptoms of the localized infection or infectious disease within the subject in need thereof.

The T cell immunostimulatory compound and/or the compound that suppresses induction of Tregs at, adjacent to, or near the site of the focus of interest treat(s) a localized environment comprising the focus of infection or symptoms of the localized infection or infectious disease within the subject.

### Example 7: Treatment of an Injury or a Site of Chronic Damage with Porous Scaffolds

Implantable scaffolds are made of various biocompatible and biodegradable polymers, such as alginate, hyaluronic acid, and chitosan. Microscale pores are created within the structures. To create scaffolds with stimulatory capability by this artificial niche, mesoporous silica microparticles are embedded in the scaffolds. These microparticles are loaded with compounds that stimulate T cells (e.g., cytokines [e.g., IL-2, IL-4, IL-6, IL-7, IL-10, IL-12, IL-15, IL-2 superkine], chemokine ligands [e.g., CCL19, CCL21, and SDF-1a], anti-CD antibodies [e.g., anti-CD3, anti-CD28]) to improve T cells' proliferation and/or effector functions. Nanoparticles or microparticles comprising compounds that suppress induction of Tregs (e.g., TGF-beta inhibitors) may also be included.

The T cell immunostimulatory compound and/or the compound that suppresses induction of Tregs are selected for the treatment of selected for the treatment of an injury (e.g., trauma, chemical or of a site of chronic damage (e.g., osteoarthritis, type 1 diabetes, rheumatoid arthritis, lupus) in the subject, or for the alleviation of localized symptoms, or combinations thereof, in a subject. The T cell immunostimulatory compound and/or the compound that suppresses induction of Tregs acts in concert with other proteins or cells to enhance a desired immune response for the treatment or reduction in the size/amount/severity of the focus of infection or symptoms of an injury or a site of chronic damage. The porous scaffold treatment is administered at or adjacent to the injury or to the site of chronic damage, either to treat, reduce, or alleviate the injury (e.g., to promote repair, to promote vascularization, etc.), to prevent infection or further damage (e.g., fungal, bacterial, viral, or parasitic infection; neuropathy; muscle wasting; etc.), or to reduce a symptom of the injury or of the chronic damage (e.g., pain, inflammation, etc.).

The T cell immunostimulatory compound and/or the compound that suppresses induction of Tregs is selected, e.g., to inhibit or promote (as needed) cell division and/or growth (e.g., a growth factor inhibitor), to inhibit inflammation (e.g., anti-inflammatory), to promote analgesic activity, to inhibit or promote (as needed) cell death (e.g., an apoptosis-promoting cytokine or other protein of interest), or to regulate an immune response (e.g., increasing proliferation of cytotoxic T cells, increasing proliferation of helper T cells, maintaining the population of helper T cells, increasing cytotoxic T cells, or a combination thereof), in the vicinity of the injury or the site of chronic damage. Optionally, the method further comprises a step of administering activated T cells to the subject.

The scaffold is administered at or adjacent to the focus of infection or symptoms of the localized infection or infectious disease within the subject in need thereof.

The T cell immunostimulatory compound and/or the compound that suppresses induction of Tregs at, adjacent to, or near the site of the focus of interest treat(s) a localized environment comprising the injury or site of chronic damage within the subject.

### Example 8: Treatment of a Surgical Site with Porous Scaffolds

Implantable scaffolds are made of various biocompatible and biodegradable polymers, such as alginate, hyaluronic acid, and chitosan. Microscale pores are created within the structures. To create scaffolds with stimulatory capability by this artificial niche, mesoporous silica microparticles are embedded in the scaffolds. These microparticles are loaded with compounds that stimulate T cells (e.g., cytokines [e.g., IL-2, IL-4, IL-6, IL-7, IL-10, IL-12, IL-15, IL-2 superkine], chemokine ligands [e.g., CCL19, CCL21, and SDF-1a], anti-CD antibodies [e.g., anti-CD3, anti-CD28]) to improve T cells' proliferation and/or effector functions. Nanoparticles or microparticles comprising compounds that suppress induction of Tregs (e.g., TGF-beta inhibitors) may also be included. Alternatively, these microparticles are loaded with compounds that suppress T cells (e.g., cytokines, growth factors, or chemokines) to improve T cells' proliferation and/or effector functions. Nanoparticles or microparticles comprising compounds that induce Tregs (e.g., TGF-beta, IL-2, and activators thereof) may also be included.

The compound that regulates T cell immune response and/or the compound that regulates induction of Tregs are selected for the treatment of selected for the treatment of a surgical site in the subject, or for the alleviation of localized symptoms, or combinations thereof, in a subject. The porous scaffold treatment is administered at or adjacent to the surgical site, either to treat, reduce, or alleviate the effects of surgery (e.g., to promote repair, to promote vascularization, etc.), to prevent infection or further damage (e.g., fungal, bacterial, viral, or parasitic infection; neuropathy; muscle wasting; etc.), or to reduce a symptom of the effects of surgery (e.g., pain, inflammation, etc.).

The compound that regulates T cell immune response and/or the compound that regulates induction of Tregs acts in concert with other proteins or cells to enhance a desired immune response for the treatment or reduction in the size/amount/severity of the surgical site and type of surgery, as well as of one or more localized symptoms of the associated effects of surgery. The compound that regulates T cell immune response and/or the compound that regulates induction of Tregs is selected, e.g., to inhibit or promote (as needed) cell division and/or growth (e.g., a growth factor inhibitor), to inhibit inflammation (e.g., anti-inflammatory), to promote analgesic activity, to inhibit or promote (as needed) cell death (e.g., an apoptosis-promoting cytokine or other protein of interest), or to regulate an immune response (e.g., increasing proliferation of cytotoxic T cells, increasing proliferation of helper T cells, maintaining the population of helper T cells, increasing cytotoxic T cells, or a combination thereof), in the vicinity of the surgical site. Optionally, the method further comprises a step of administering activated T cells to the subject.

The scaffold is administered at or adjacent to the surgical site within the subject in need thereof.

The T cell immunostimulatory compound and/or the compound that suppresses induction of Tregs at, adjacent to, or near the site of the focus of interest treat(s) a localized environment comprising the surgical site within the subject.

### Example 9: Treatment of a Transplant Site Associated with a Transplanted Organ, Tissue, or Cells with Porous Scaffolds

Implantable scaffolds are made of various biocompatible and biodegradable polymers, such as alginate, hyaluronic acid, and chitosan. Microscale pores are created within the structures. To create scaffolds with stimulatory capability by this artificial niche, mesoporous silica microparticles are embedded in the scaffolds. These microparticles are loaded with compounds that suppress T cells (e.g., cytokines, chemokines, or growth factors) to improve T cells' proliferation and/or effector functions. Nanoparticles or microparticles comprising compounds that induce Tregs (e.g., TGF-beta and activators thereof) may also be included.

The T cell immunosuppression compound and/or the compound that induces Tregs are selected for the treatment of a transplant site associated with a transplanted organ, tissue, or cells, or for the alleviation of localized symptoms, or combinations thereof, in a subject. The T cell immunosuppression compound and/or the compound that induces Tregs porous scaffold treatment is administered at or adjacent to the transplant site associated with a transplanted organ, tissue, or cells, either to treat, reduce, or alleviate the surgery related to the transplant (e.g., to promote repair, to promote vascularization, etc.), to prevent infection or damage (e.g., fungal, bacterial, viral, or parasitic infection; neuropathy; muscle wasting; to reduce the likelihood of rejection, or to reduce a symptom of the transplant or surgery related thereto (e.g., pain, inflammation, etc.).

The T cell immunosuppression compound and/or the compound that induces Tregs acts in concert with other proteins or cells to enhance a desired immune response for the treatment of the transplant site associated with a transplanted organ, tissue, or cells. The T cell immunosuppression compound and/or the compound that induces Tregs is selected, e.g., to inhibit or promote (as needed) cell division and/or growth (e.g., a growth factor inhibitor), to inhibit inflammation (e.g., anti-inflammatory), to promote analgesic activity, to inhibit or promote (as needed) cell death (e.g., an apoptosis-promoting cytokine or other protein of interest), or to regulate an immune response, such as suppression of rejection (e.g., decreasing proliferation of cytotoxic T cells, decreasing proliferation of helper T cells, decreasing cytotoxic T cells, or a combination thereof), in the vicinity of the injury or the site of chronic damage.

The scaffold is administered at or adjacent to the transplant site associated with a transplanted organ, tissue, or cells in the subject, thereby reducing the likelihood of rejection and/or one or more of its symptoms or effects, as well as the symptoms or effects of the transplant surgery.

The T cell immunostimulatory compound and/or the compound that suppresses induction of Tregs at, adjacent to, or near the site of the focus of interest treat(s) a localized environment comprising the transplant site associated with a transplanted organ, tissue, or cells within the subject.

### Example 10: Treatment of a Blood Clot Causing or at Risk for Causing a Myocardial Infarction, an Ischemic Stroke, or a Pulmonary Embolism with Porous Scaffolds

Implantable scaffolds are made of various biocompatible and biodegradable polymers, such as alginate, hyaluronic acid, and chitosan. Microscale pores are created within the structures. To create scaffolds with stimulatory capability by this artificial niche, mesoporous silica microparticles are embedded in the scaffolds. These microparticles are loaded with compounds that stimulate T cells (e.g., cytokines [e.g., IL-2, IL-4, IL-6, IL-7, IL-10, IL-12, IL-15, IL-2 superkine], chemokine ligands [e.g., CCL21], anti-CD antibodies [e.g., anti-CD3, anti-CD28]) to improve T cells' proliferation and/or effector functions. Nanoparticles or microparticles comprising compounds that suppress induction of Tregs (e.g., TGF-beta inhibitors) may also be included. Alternatively, these microparticles are loaded with compounds that suppress T cells (e.g., cytokines) to improve T cells' proliferation and/or effector functions. Nanoparticles or microparticles comprising compounds that induce Tregs (e.g., TGF-beta and activators thereof) may also be included.

The compound that regulates T cell immune response and/or the compound that regulates induction of Tregs (e.g., a cytokine, a thrombolytic, or another protein of interest) are selected for the treatment of selected for the treatment of a blood clot causing or at risk for causing a myocardial infarction, an ischemic stroke, or a pulmonary embolism in the subject, or for the alleviation of localized symptoms, or combinations thereof, in a subject. In a non-limiting example, thrombolytic (“clot buster”) treatment is administered at or adjacent to the blood clot to break up, reduce, or eliminate the blood clot in order to treat or prevent infarction of a blood vessel and thereby to treat or prevent, e.g., a myocardial infarction (heart attack), an ischemic stroke, or a pulmonary embolism. Non-limiting examples of thrombolytics include tissue plasminogen activator (tPA), tenecteplase, alteplase, urokinase, reteplase, and streptokinase.

The porous scaffold treatment is administered at or adjacent to the blood clot, either to treat, reduce, or alleviate the effects of surgery (e.g., to promote repair, to promote vascularization, etc.), to prevent infection or further damage (e.g., fungal, bacterial, viral, or parasitic infection; neuropathy; muscle wasting; etc.), or to reduce a symptom of the effects of surgery (e.g., pain, inflammation, etc.). It is noted that the location of the blood clot may not be in the heart, the brain, or a lung at the time of treatment, but rather in some other part of the subject's body (e.g., the lower limbs and extremities; the carotid artery; the site of an injury, surgery, or a transplant; or elsewhere).

The compound that regulates T cell immune response and/or the compound that regulates induction of Tregs acts in concert with other proteins or cells to enhance a desired response for the treatment of a blood clot. The compound that regulates T cell immune response and/or the compound that regulates induction of Tregs (e.g., cytokine, thrombolytic or other protein of interest) is selected, e.g., to inhibit angiogenesis, to promote reduction or elimination of clotting, to inhibit inflammation (e.g., anti-inflammatory), to promote analgesic activity, to inhibit or promote (as needed) cell death (e.g., an apoptosis-promoting cytokine or other protein of interest), or to regulate an immune response (e.g., increasing proliferation of cytotoxic T cells, increasing proliferation of helper T cells, maintaining the population of helper T cells, increasing cytotoxic T cells, or a combination thereof), in the vicinity of the blood clot. Optionally, the method further comprises a step of administering activated T cells to the subject.

The porous scaffold is administered at or adjacent to or near the blood clot. The porous scaffold may be administered via a guided catheter, which may facilitate access to, and treatment of, the blood clot. The porous scaffold may be administered, in a non-limiting example, together with angioplasty (e.g., a balloon catheter) or other clot removal treatment.

The T cell immunostimulatory compound and/or the compound that suppresses induction of Tregs at, adjacent to, or near the site of the focus of interest treat(s) a localized environment comprising the blood clot within the subject.

### Materials and Methods for Examples 11-25

### Chemicals and Biologicals

Unless noted otherwise, all chemicals were purchased from SIGMA-ALDRICH™, INC. (St. Louis, Mo.). All glassware was cleaned overnight using concentrated sulfuric acid and then thoroughly rinsed with MILLI-Q® water. All the other cell culture reagents, solutions, and dishes were obtained from THERMO FISHER SCIENTIFIC™ (Waltham, Mass.), except as indicated otherwise.

### Preparation and Characterization of Artificial APC Microparticles

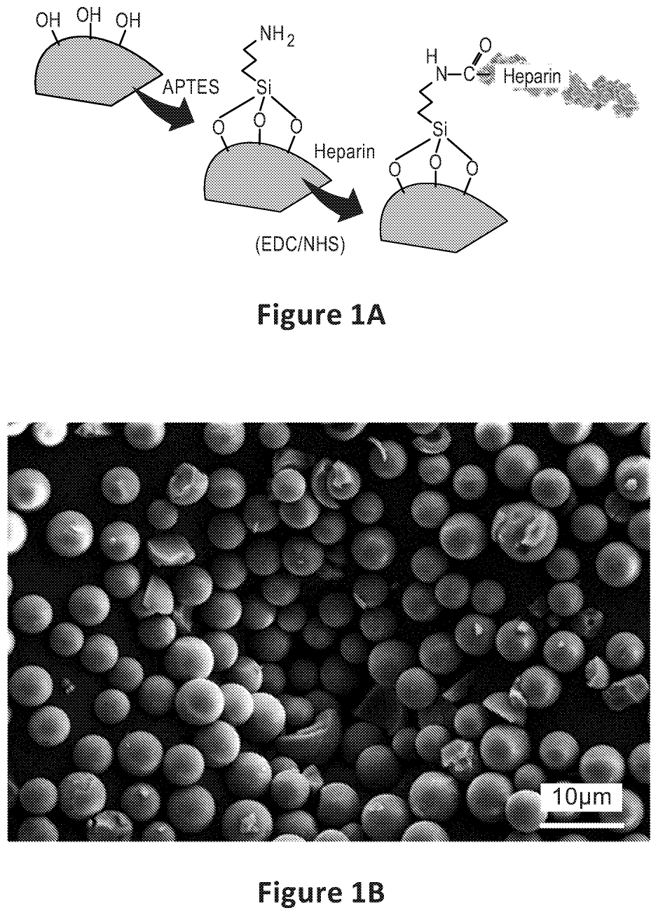

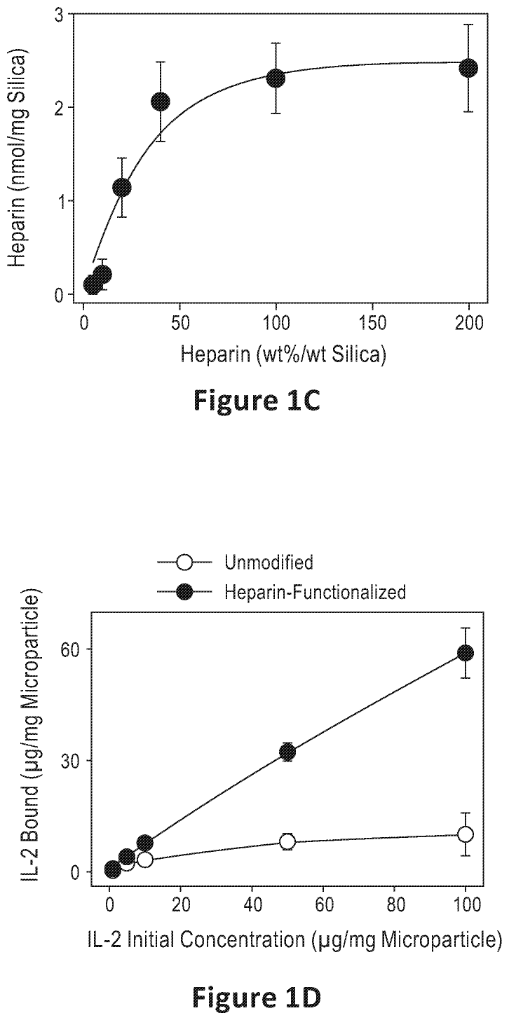

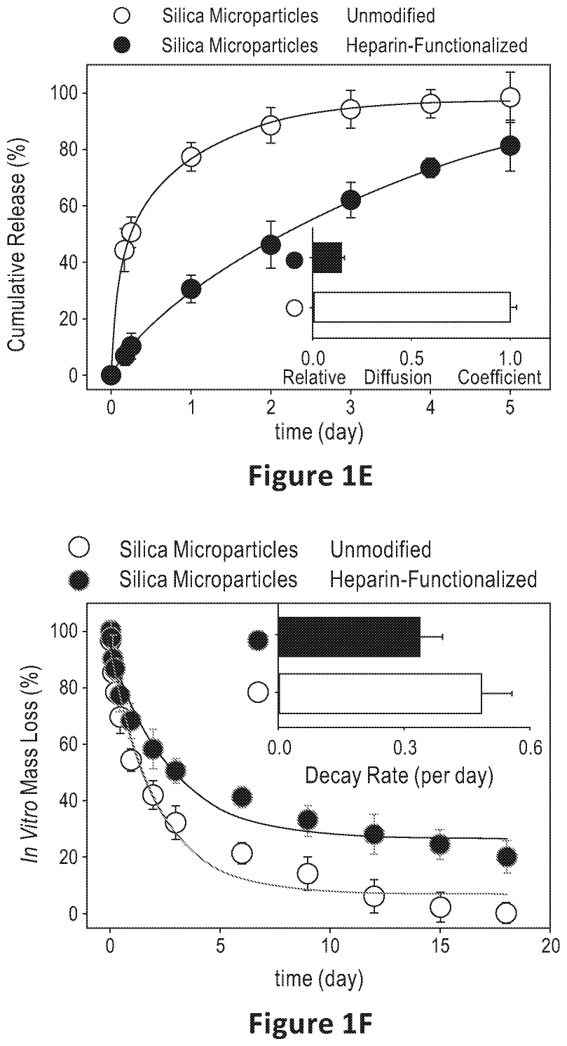

Monodisperse mesoporous silica microparticles (5 to 20 μm [micrometers/microns]) were formed using a microfluidic jet spray-drying route, using cetyltrimethylammonium bromide (CTAB) and/or Pluronic F127 as templating agents, and tetraethylorthosilicate (TEOS) for silica as reported before (see, e.g., Waldron, K. et al. Formation of monodisperse mesoporous silica microparticles via spray-drying. J. Colloid Interface Sci. (2014). doi:10.1016/j.jcis.2013.12.027; Liu, W., Chen, X. D. & Selomulya, C. On the spray drying of uniform functional microparticles. Particuology (2015). doi:10.1016/j.partic.2015.04.001). Carbodiimide chemistry (1-ethyl-3-(3-dimethylaminopropyl)carbodiimide hydrochloride [EDC]/N-hydroxysuccinimide [NHS]; EDC/NHS) was utilized to modify silica conjugates with heparin after treating the silica with (3-Aminopropyl)triethoxysilane (APTES) to provide primary amine groups (see FIG. 1A). Briefly, mesoporous silica microparticles (800 mg) was suspended in dehydrated Methanol (50 ml). Then, APTES (3 ml) was added and the suspension was stirred at room temperature overnight, and the final product was centrifuged (1500 rpm, 3 min) and washed with methanol five times, followed by drying under high vacuum. For the surface functionalization of the aminated-silica particles with heparin, heparin sodium salt (216 mg) was dissolved in deionized water (8 ml) and activated via successive addition of EDC (63 mg) and N-hydroxysulfosuccinimide (sulfo-NHS; 71.4 mg). After stirring for 5 min, the ethanolic solution of amino-functionalized silica (20 mg in 1.12 ml) was added to the reaction mixture and stirred for 12 hours (h) at room temperature. Afterwards the particles were separated by centrifugation and washed several times with deionized water and ethanol to remove unreacted reagents.

For the preparation of antibody-conjugated microparticles, anti-CD3 (clone 2C11; BIO-X-CELL™) and anti-CD28 (clone 37.51; BIO-X-CELL™) were covalently conjugated to the surface of particles using carbodiimide chemistry. After activation of antibodies' carboxylic groups for 10 min with EDC/NHS, microparticles were added and incubated under gentle stirring at 4° C. (degrees Celsius) overnight. The protein-functionalized microparticles (artificial antigen presenting cells, aAPCs) were then separated from the solution and washed several times. Unreacted functional groups were quenched by washing samples in Tris buffer (100 mM, pH 8) for 30 min. A 10-fold dilution of the conjugation density that is used in a conventional plate-bound stimulation method for T cell activation was selected as the final conjugation density for beads. Micro-bicinconinic acid (MICRO-BCA™) assay was used to quantify total amount of surface conjugated antibodies according to the manufacturer's protocol.

### Preparation and Characterization of Scaffolds

In some exemplary, but non-limiting, embodiments, to form the scaffolds, alginate (MW ˜250 kDa, high G blocks; Novamatrix UP MVG, FMC Biopolymer, Rockland, Me.) was oxidized with sodium periodate (1.5%), overnight at room temperature, then quenched the reaction by dropwise addition of ethylene glycol for 45 min. The solution (MWCO 3.5 kDa) was then dialyzed against deionized water for 3 days (d) followed by lyophilization. Afterward, the alginate was dissolved in 2-morpholin-4-ylethanesulfonic acid (MES) (MES 150 mM, NaCl 250 mM, pH 6.5) and covalently conjugated to RGD-containing peptide (GGGGRGDY [SEQ ID NO: 1]; GENSCRIPT™ USA Inc., Piscataway, N.J.) using carbodiimide chemistry (EDC/NHS). The reaction was continued for 24 h followed by dialysis (MWCO 20 kDa) and lyophilization. This alginate-RGD complex in phosphate buffered saline (PBS) was then cross-linked via calcium sulfate solution. The gels were casted in desired 24- or 96-well plates followed by two overnight washes to get rid of the extra calcium ions and then used as two-dimensional (2D) matrices. For three-dimensional (3D) structures these same scaffolds were frozen at −80° C., lyophilized for 3 days, and stored at 4° C. before cellular studies. To prepare aAPC loaded scaffolds, 20×10⁶ (20×106) aAPCs were mixed with 1 ml of alginate prior to crosslinking with CaSO4 (CaSO₄).

An array of different alginate formulations was then prepared by varying either the polymer content or the amount of crosslinker (here CaSO4). To measure the mechanical stiffness of the gels, an INSTRON™ Model 5542 mechanical tester was used, and all the samples were tested at a rate of 1 mm/min. The Young's modulus (YM; Young modulus) was then calculated from the slope of the linear region that corresponds with 0-10% strain. Here, the stiff gel comprised of alginate 2.5% with 40 mM CaSO4 was used.

X-ray irradiation (GULMAY MEDICAL™ RS320 X-ray unit) was used to irradiate the fabricated scaffolds before in vitro or in vivo functional assays, following ISO 11137-2:2013 recommended protocols. 16 A 25 kGy (2.5 Mrads) sterilization dose was used. Physical properties, including changes in morphology and mechanical stiffness of the scaffolds, or T cell activation property change after sterilization, were tested.

Scanning electron microscopy (SEM) images of the gels were taken to see the cross-sectional microstructure and porosity of the alginate-based scaffolds. The lyophilized scaffolds were freeze-fractured (using liquid nitrogen) for cross-sectional images. The scaffolds were sputtered with iridium (SOUTH BAY TECHNOLOGY™ Ion Beam Sputtering) prior to imaging with a ZEISS SUPRA™ 40VP scanning electron microscope (CARL ZEISS MICROSCOPY™ GmbH). The sizes of pores from different parts of the SEM images were then measured and analyzed using ImageJ software (NIH). For SEM imaging of cell-loaded scaffolds, the cell-laden hydrogels were fixed with 2.5% glutaraldehyde, followed by post-fixation in osmium tetroxide prior to serial dehydration in increasing concentrations of ethanol (25, 50, 75, 90, and 100%) for 15 min each, and iridium sputtering.

To immobilize anti-cluster of differentiation 3 (anti-CD3) and anti-cluster of differentiation 28 (anti-CD28) to the scaffolds, the freeze-dried scaffolds were activated with EDC/NHS or EDC/sulfo-NHS for 15 min. Then the scaffolds were washed twice with PBS (supplemented with 0.42 mM CaCl2) before addition of anti-CD3 and anti-CD28. Then they were incubated at 4° C. overnight. Unreacted functional groups were quenched by washing the scaffolds with Tris buffer (100 mM, pH 8) for 30 min. For T cell activation studies, 5×10⁶ (5×106) primary naïve T cells were added to the scaffolds and cultured for 3-5 days to study their effector functions.

To prepare IL-2 loaded aAPCs, microparticles were incubated with cytokine in PBS buffer containing bovine serum albumin (BSA; 0.1% w/v) and were gently shaken overnight at 4° C. The microparticles were then centrifuged and washed several times to remove unabsorbed cytokines. The concentration of IL-2 in the removed supernatant was measured using enzyme-linked immunosorbant assay (ELISA) to estimate the binding capacity of microparticles.

In vitro release of IL-2 from aAPCs or from aAPCs-loaded scaffolds as well as chemokine (C-C motif) ligand 21 (CCL21) release from the scaffolds were studied by incubating 20×10⁶ (20×106) microparticles or one scaffolds in 2 ml PBS (pH 7.4; supplemented with 1 mM CaCl2) at 37° C. At different time intervals, 500 μL (microliters) of the supernatant was collected and replaced with an equivalent volume of PBS. The concentration of released IL-2 was determined using a human IL-2 and murine CCL21 ELISA kits as a function of time.

TGF-β inhibitor, galunisertib (LY2157299) (CAYMAN CHEMICAL™), loaded poly(lactic-co-glycolic) acid (PLGA) nanoparticles (NPs) were prepared using a nanoprecipitation method as previously reported. RESOMER™ RG 503 PLGA (50:50; molecular weight: 28 kg/mol) was used in this study. LY2157299 (Cayman chemical https://www.caymanchem.com/product/15312/ly2157299) and PLGA were dissolved in 5 mL dichloromethane and sonicated into 1% poly vinyl alcohol (PVA) solution (50 ml) by probe sonicator (12 W) for 2 min. The resulting emulsification was then added to 100 ml of 0.5% PVA solution. The solution was agitated, and the dichloromethane was allowed to evaporate for 4 h. The solution was then centrifuged at 3000×g for 5 min to pellet out any non-nano dimensional materials. The supernatant was removed and ultracentrifuged and washed three times at 21,000 g for 20 min to wash away the PVA. The resulting nanoparticle solution was flash frozen in liquid nitrogen and lyophilized for 2 days prior to characterization and use. Hydrodynamic diameter and surface charge of formed PLGA NPs was studied using dynamic light scattering (DLS) and zeta potential measurements (ZETASIZER NANO™, Malvern, UK). To load these NPs into alginate-based scaffolds, LY2157299-loaded PLGA NPs were mixed with alginate prior to crosslinking via calcium. The concentration of released and LY2157299 from nanoparticles before and after loading into alginate scaffolds was determined by measuring the ultraviolet (UV) absorption of LY2157299.

### T Cell Isolation and Activation

All in vitro experiments were conducted in accordance with University of California at Los Angeles' (UCLA's; Los Angeles, Calif., USA) institutional policy on humane and ethical treatment of animals following protocols approved by the Animal Research Committee. Five- to eight-week-old wild-type or OT-I/OTII TCR transgenic mice (Jackson Labs) were used for all experiments.

Cell-culture media was RPMI supplemented with 10% heat-inactivated FBS, 1% penicillin/streptomycin, 1% sodium pyruvate, 1% HEPES buffer, 0.1% μM 2-mercaptoethanol. CD4+/CD8+ T cells were purified using EASYSEP™ immunomagnetic negative selection enrichment kits (STEM CELL TECHNOLOGIES™). (Per liter, RPMI 1640 medium is commercially available [see e.g., https://www.fishersci.com/shop/products/gibco-rpmi-1640-medium-41/p-4919923] and contains: glucose (2 g), pH indicator (phenol red, 5 mg), salts (6 g sodium chloride, 2 g sodium bicarbonate, 1.512 g disodium phosphate, 400 mg potassium chloride, 100 mg magnesium sulfate, and 100 mg calcium nitrate), amino acids (300 mg glutamine; 200 mg arginine; 50 mg each asparagine, cystine, leucine, and isoleucine; 40 mg lysine hydrochloride; 30 mg serine; 20 mg each aspartic acid, glutamic acid, hydroxyproline, proline, threonine, tyrosine, and valine; 15 mg each histidine, methionine, and phenylalanine; 10 mg glycine; 5 mg tryptophan; and 1 mg reduced glutathione), vitamins (35 mg i-inositol; 3 mg choline chloride; 1 mg each para-aminobenzoic acid, folic acid, nicotinamide, pyridoxine hydrochloride, and thiamine hydrochloride; 0.25 mg calcium pantothenate; 0.2 mg each biotin and riboflavin; and 0.005 mg cyanocobalamin)).

Control in vitro activation of CD4+/CD8+ T cells was performed by culturing 1×10⁶ (1×106) cells/mL in tissue culture-treated 24-well plates that were pre-coated with anti-CD3 (clone 2C11; BIO X CELL™) at a concentration of 10 μg/mL (micrograms/mL) plus addition of 2 μg/mL (micrograms/mL) soluble anti-CD28 (clone 37.51; BIO X CELL™). T cells were then collected from wells and allowed to proliferate in interleukin-2 (IL-2, BRB™ Preclinical Repository, NCI, NIH)-containing medium (50 U/mL), prior to being used for experiments.

For Treg formation experiments CD4+ T cells were purified from mouse spleen as mentioned above. Cells were then either activated on scaffolds or on anti-CD3e antibody (8 mg/ml) coated plates with the anti-CD28 antibody (2 mg/ml) supplemented medium. At the same time transforming growth factor-beta (TGF-beta; TGF-β) (15 ng/ml) was added to the media. After four days regulatory T cells were removed and stained with antibodies for flow cytometry analysis.

### Flow Cytometry

For flow cytometry analysis, antibodies to mouse antibodies, were purchased from EBIOSCIENCE™, BIOLEGEND™, or BD BIOSCIENCES™. To study proliferation behavior of T-cell responses during various treatments their expansion was measured by 5-(and-6)-carboxyfluorescein diacetate, succinimidyl ester (CFSE) dilution. For CFSE dilution experiments, 5×10⁵ (5×105) naive CD4+/CD8+ T cells were labeled with 2 μM CFSE for 13 min, followed by two washes and then incubation with splenocytes. Splenocytes were extracted from the spleen of wild type C57Bl/6 mice. Then the cells were incubated in ammonium-chloride-potassium (ACK) lysing buffer (GIBCO™) for 5 min at room temperature to remove red blood cells. The remaining cells were then treated with ova peptide as above to present to T cells. Trypan Blue was purchased from CALBIOCHEM™. Cells were analyzed on a CYTEK™ DxP10 flow cytometer using FLOWJO™ software (TREESTAR/BD™).

For intracellular staining of GranzymeB and Foxp3, the recommended protocol by EBIOSCIENCE™ Foxp3/Transcription Factor Staining Buffer Set was followed. The following antibodies were used for intracellular staining from BIOLEGEND™: Foxp3 (clone MF-14, AF647, Cat #126408); GZMB (clone GB11, AF647, Cat #515406), Mouse IgG1, kappa (κ) Isotype Ctrl (clone MOPC-21, AF647, Cat #400130).

### Migration Assay

The migration assay to evaluate the role of chemokines on recruitment of T cells and melanoma cancer in the presence and absence of magnetic particles was performed using regular Transwell migration (Majedi, F. S. et al. Cytokine Secreting Microparticles Engineer the Fate and the Effector Functions of T-Cells. Adv. Mater. 30, 1703178 (2018)). The number of migrated cells was evaluated after 4 h using an automatic cell counter.

### In Vivo Tumor Suppression Assay

2-5×10⁵ (2-5×105) B16F10-OVA tumor cells were subcutaneously injected into right or both (in the contralateral tumor model) right and left flanks of C57BL/6J WT mice (6-8 weeks old). These melanoma-derived cells are transfected to express chicken ovalbumin peptide (OVA)34. Five days after tumor cell injection, scaffolds were surgically implanted subcutaneously into the same approximate region of the tumors in both flanks. For cell-loaded studies, ex vivo activated OT-I T cells were transferred either intravenously using retro-orbital injections (100 microliters [μL] per animal) or implantable scaffolds at the same day. Tumor size was assessed over time using a digital caliber until day 22 at which animals were sacrificed and the tumor, draining lymph nodes, and spleen were extracted. Tumor mass was measured using a digital balance before digesting the tumor tissue for flow cytometry or fixing it for tissue sectioning. Tumors were digested by incubating in collagenase and DNase I (50 micrograms/mL [μg/mL]) at 37° C. for 15 min. These enzymes were inactivated with ethylenediamine tetra-acetic acid (EDTA) (20 microliters/mL [μL/mL] of solution). Tissues were then mechanically disaggregated and passed through a 0.7 micron [μm] cell strainer to obtain a single-cell suspension. Cells were then stained with the fluorochrome-conjugated antibodies on ice. For intracellular staining, cells were permeabilized with Granzyme B Fix/Perm buffer according to the manufacturer's instructions (BIOLEGEND™) before staining. Detection of apoptotic cells in tumor tissue was achieved using Terminal deoxynucleotidyl transferase-mediated dUTP nick-end labeling (TUNEL) staining following the manufacturer's directions. TUNEL-positive cells indicated as apoptotic melanoma cells. Tissue sections were imaged by a fluorescence microscope (KEYENCE™ BZ-X800, Osaka, Japan).

### Statistical Analysis

The Kruskal-Wallis rank sum test, one-way analysis of variance (ANOVA) and two-tailed Student's t-test were utilized as appropriate to analyze the data at a significance of alpha (α) or p<0.05. Quantitative data were expressed as mean±standard deviation (SD). To determine the number of specimens for the proposed experiments, power analysis was conducted based on our preliminary data.

### Example 11: Production of Scaffolds Having Microparticles with Enhanced Loading Capacity for Cytokines and Artificial Antigen Presenting Cells (aAPCs)

Implantable porous silica scaffolds were made as described herein. To create scaffolds with stimulatory capability, mesoporous silica microparticles were embedded in the pores of the scaffolds.

To enhance loading capacity of cytokines within these particles, their surfaces were modified with heparin (FIG. 1A). The resulting particles' diameter was characterized at 3-25 um with pore size 1-7 nm by scanning electron microscopy (FIG. 1B). The surface of the particles was fully heparinized with about 2 nmol heparin/mg silica (FIG. 1C).

To test the capacity of these heparin-conjugated particles, the key T cell growth factor IL-2 was loaded. Heparin modification improved loading by over 10-fold (FIG. 1D). IL-2 release was measured over five days. Heparin modification delayed the release kinetics significantly, and the resulting relative diffusion constant was 10-fold less for the heparin conjugated particles than those of silica alone (FIG. 1E).

To provide T cells with activation signals, the surfaces of these mesoporous silica microparticles were also decorated with antibodies that stimulate T cell activation (anti-CD3 and anti-CD28). The silica particles themselves are known to safely degrade over time. Testing of degradation of these enhanced silica microparticles demonstrated that the particles' masses are lost over 15-20 days (FIG. 1F).

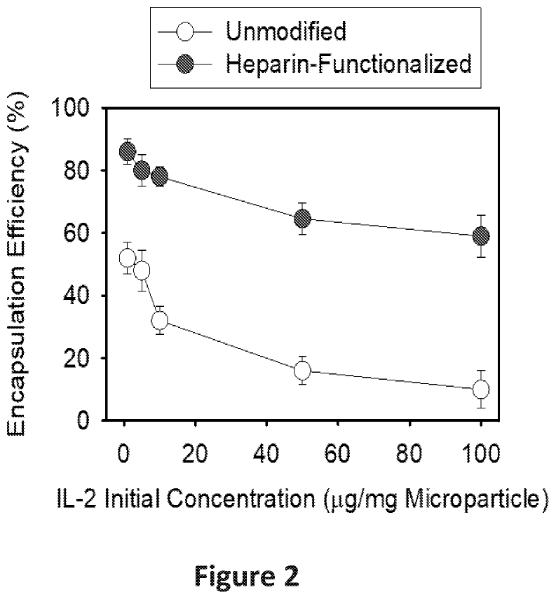

Loading efficiency on unmodified and heparin-functionalized silica microparticles was tested using IL-2 as the test compound. FIG. 2 shows the loading efficiency on unmodified and heparin-functionalized silica microparticles using IL-2 as the test compound. Heparin-functionalized silica microspheres provide greatly increased encapsulation.

The presence of heparin significantly increased the affinity of positively charged proteins, isoelectric point (pI)>7.5 (see, e.g., Majedi, F. S. et al. Cytokine Secreting Microparticles Engineer the Fate and the Effector Functions of T-Cells. Adv. Mater. 30, 1703178 (2018); Hasani-Sadrabadi, M. M. et al. Mechanobiological Mimicry of Helper T Lymphocytes to Evaluate Cell—Biomaterials Crosstalk. Adv. Mater. 30, 1-10 (2018)). To prepare IL-2 loaded silica microparticles, microparticles were incubated with cytokine in PBS buffer containing bovine serum albumin (BSA; 0.1% w/v) and were gently shaken overnight at 4° C. The microparticles were then centrifuged and washed several times to remove unabsorbed cytokines. The concentration of IL-2 in the removed supernatant was measured using enzyme-linked immunosorbant assay (ELISA) to estimate the binding capacity of microparticles. Here, heparin-functionalized mesoporous silica microparticles (5 μm [microns] in diameter) were synthesized and optimized to encapsulate and deliver IL-2 (FIG. 1). Monodisperse mesoporous silica microparticles (5 to 20 μm [microns]) were produced via a microfluidic jet spray-drying route, using cetyltrimethylammonium bromide (CTAB) and/or Pluronic F127 as templating agents, and tetraethylorthosilicate (TEOS) for silica (see, e.g., Waldron, K. et al. Formation of monodisperse mesoporous silica microparticles via spray-drying. J. Colloid Interface Sci. (2014). doi:10.1016/j.jcis.2013.12.027; Liu, W., Chen, X. D. & Selomulya, C. On the spray drying of uniform functional microparticles. Particuology (2015). doi:10.1016/j.partic.2015.04.001). Carbodiimide chemistry (NHS/EDC) was utilized to modify silica conjugates with heparin after treating the silica with (3-aminopropyl)triethoxysilane (APTES) to provide primary amine groups (FIG. 1A).

Change in physical properties of silica particles summarized in TABLE 2. Heparin-based conjugates (silica-heparin) was developed at several conjugation densities (FIG. 1C). Heparin presence provides enhanced efficiency and stability of cytokine binding that enables precise spatiotemporal control over the release profile of target proteins (here IL-2) (FIG. 1D, FIG. 2). Heparin modification has delayed the rate of release by about 5-fold compared to the unmodified silica (FIG. 1E). In vitro degradation of mesoporous silica (MES) beads indicates that these particles are mostly gone in two weeks and heparin modification accelerates the degradation as it will increase the hydrophilicity of particles (FIG. 1F).

TABLE 2
Change in physical characteristics of mesoporous silica microparticles
after surface functionalization with APTES and heparin.

| | Pore | | |
|---|---|---|---|
| Diameter | Diameter | Surface Area | Pore Volume |
| (μm) | (nm) | (m²/g) | (ml/g) |
| 5 | 11.5 | 384 | 1.1 |
| 15 | 9 | 430 | 0.97 |

| | Pore Diameter | Surface Area | |
|---|---|---|---|
| | (nm) | (m²/g) | Volume (ml/g) |
| Unmodified | 11.5 | 384 | 1.1 |
| Anime-modified | 10.4 | 250 | 0.65 |
| Heparin-functionalized | 7.9 | 119 | 0.23 |

### Example 12: Silica-Heparin Particles are Potent aAPCs for In Vitro T Cell Expansion

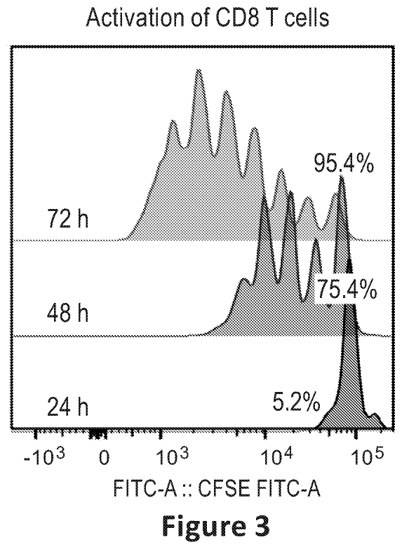

The activation of CD8 T cells following co-culturing with silica-based microparticles was studied. To serve as aAPCs the surfaces of IL-2 loaded silica-heparin beads were decorated with aCD3/aCD28 to provide the anchor for T cells through which they can engage with the beads and get activated as a result (FIG. 3). FIG. 3 shows increasing activation of CD8 T cells following co-culturing cells with silica-based microparticles.

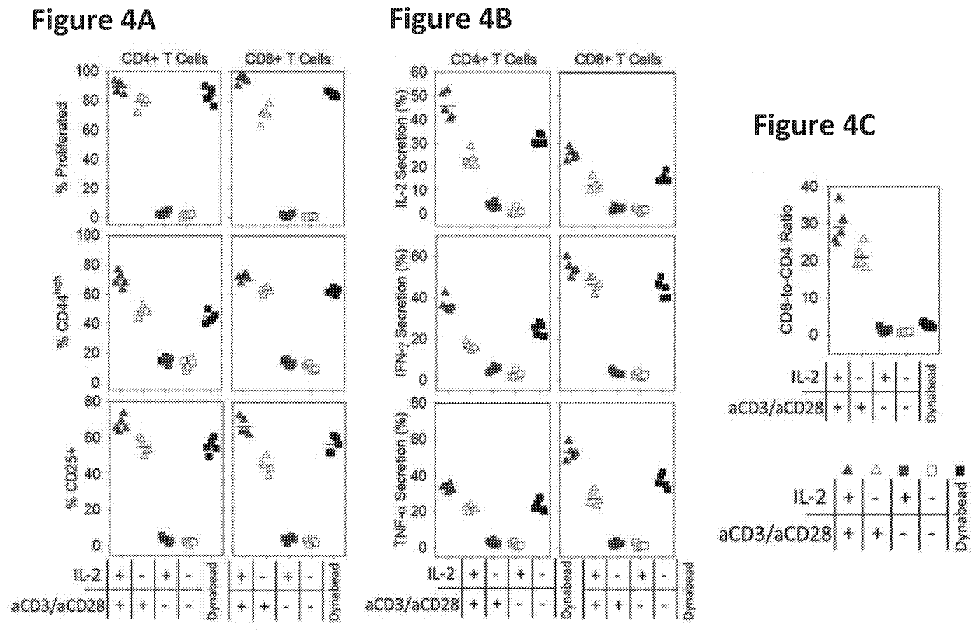

To evaluate the efficiency of these aAPCs in vitro, they were co-cultured with CD8+ and CD4+ T cells under various conditions. Plain silica-heparin particles, IL-2 loaded silica-heparin particles, aCD3/aCD28 decorated silica-heparin particles free of IL-2, and DYNABEADS™ supplemented with free IL-2 were compared with IL-2 loaded, aCD3/aCD28 decorated silica-heparin particles (FIG. 4). Antibody conjugated beads with or without IL-2 loading were tested as aAPCs, and T cell proliferation and activation were tracked upon a 3 day co-culture with beads (FIG. 4A).

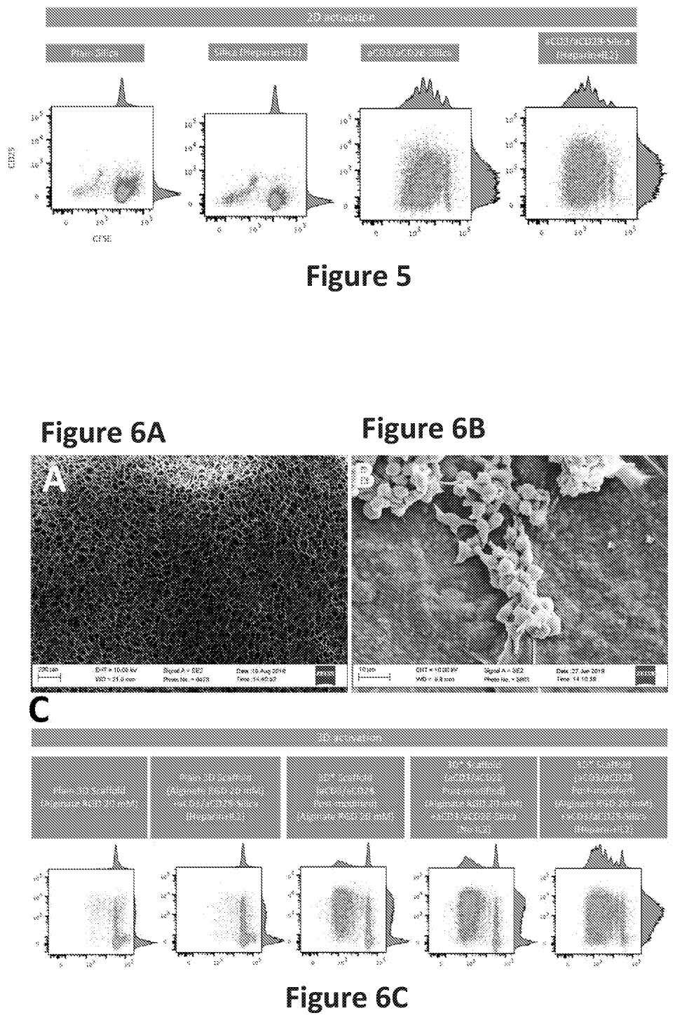

These particles strongly interacted with T cells and induced activation and proliferation of naive T cells. The presence of IL-2 helped reduce the population of undivided cells and resulted in an increased expression of activation markers such as CD25 on activated T cells (FIG. 4A, FIG. 5). It should be noted that IL-2 releasing, surface conjugated silica beads performed better than DYNABEADS™ supplemented with soluble IL-2 in terms of activation (FIG. 4A) and induction of cytokine secretion by T cells (FIG. 4B). Unique features of MES beads such as high loading capacities for cytokines and securing prolonged release while offering biodegradability renders them to be superior to DYNABEADS™. Prolonged release of IL-2 favors formation of effector cells (see, e.g., Majedi, F. S. et al. Cytokine Secreting Microparticles Engineer the Fate and the Effector Functions of T-Cells. Adv. Mater. 30, 1703178 (2018)). These engineered silica-based aAPCs are easier to produce at higher quantities (gram scale) compared to hydrogel-based aAPCs (see, e.g., Majedi, F. S. et al. Augmentation of T-Cell Activation by Oscillatory Forces and Engineered Antigen-Presenting Cells. Nano Lett. 580704 (2019). doi:10.1021/acs.nanolett.9b02252). Moreover, the biodegradability of these silica makes them superior compared to DYNABEAD™ for ex vivo and in vivo use. Increasing the strength of activation signal favored formation of CD8s in the case of co-culture of both CD8+ and CD4+ T cells with beads. The present beads favored CD8+ population by 30-folds compared to DYNABEADS™ making them suitable for in vivo cancer models (FIG. 4C).

### Example 13: 3D Scaffolds for T Cell Expansion Mimic Conditions of Lymph Nodes

These particles provided activation cues for cultured T cells, but in order to mimic the natural niche that T cells experience during their activation, the activation platform was transformed into a 3D matrix. Here, a biocompatible alginate scaffold was engineered, which was further decorated with 0.06 mole RGD per mg alginate RGD peptides to facilitate T cell attachment and trafficking. To achieve the optimal physical properties the stiffness of the hydrogel was engineered to be similar to the stiffness that T cells experience in lymph nodes during activation (see, e.g., Meng, K. P., Majedi, F. S., Thauland, T. J. & Butte, M. J. Mechanosensing through YAP controls T cell activation and metabolism. J. Exp. Med. 217, (2020)). Here, 40 mM calcium sulfate (CaSO4) was used as a crosslinker which resulted in relatively stiff (40 kPa) gels (see, e.g., Majedi, F. S. et al. T-cell activation is modulated by the 3D mechanical microenvironment. bioRxiv 580886 (2019)). The 3D porosity was then created by lyophilization to let the cells experience 3D trafficking while receiving the activation signals (FIG. 6A). Arrangement of the cells alongside the pore walls of the scaffolds was then confirmed by scanning electron microscopy (SEM) (FIG. 6B). To provide antigen presentation, developed aAPCs were embedded within the scaffolds and their activation capability were monitored after 5 days of seeding naive CD8+ T cells within them (FIG. 6C). Solely embedding the beads within the scaffolds failed to activate T cells in short term time periods. Antibody conjugated beads were possibly coated with a layer of alginate polymer, making them buried and unavailable for T cells to anchor to (FIG. 6C).

### Example 14: Post-Conjugation of 3D Scaffolds to Ensure Availability of Antibodies to T Cells for T Cell Expansion

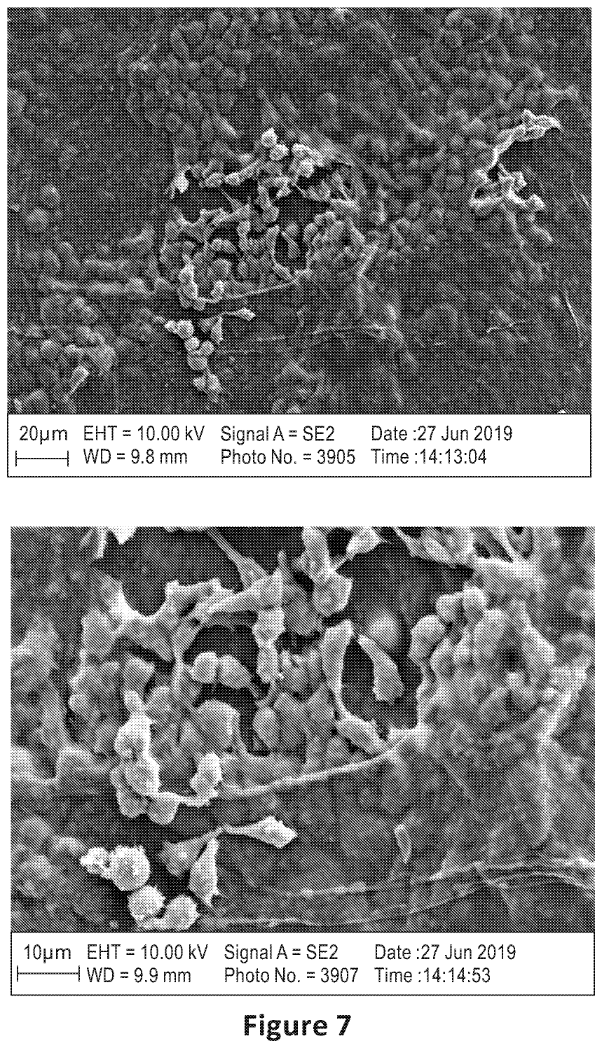

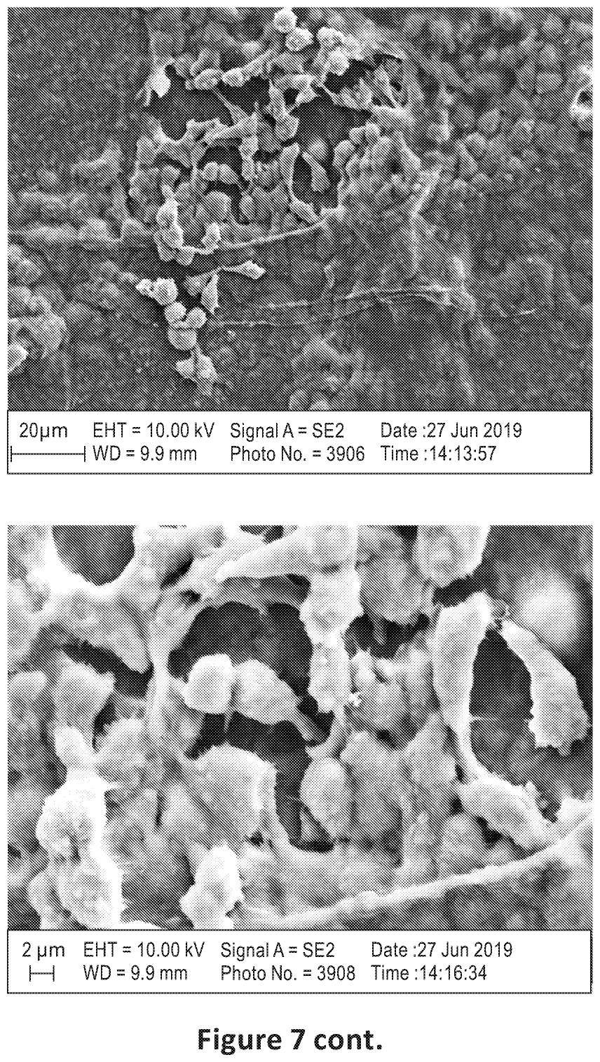

To overcome any unavailability, the 3D scaffolds were post-conjugated with anti-CD3/CD28 antibodies to ensure the availability of these antibodies to T cells. As for longer term in vivo treatments where the scaffold starts to degrade, embedded aAPC beads can also become available to cells (FIG. 7).

### Example 15: Proliferation, Activation, and Cytokine Secretion of Both CD8+ and CD4+ T Cells in the 3D Scaffolds for Improved T Cell Expansion

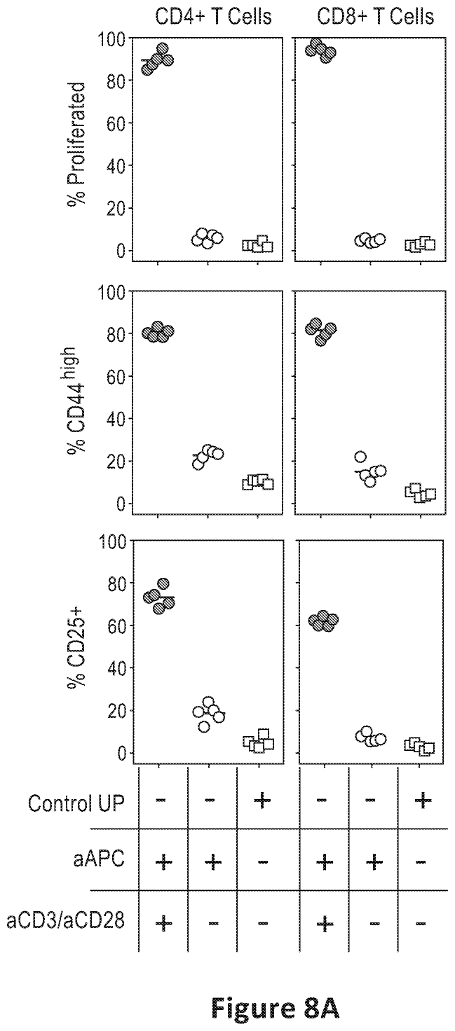

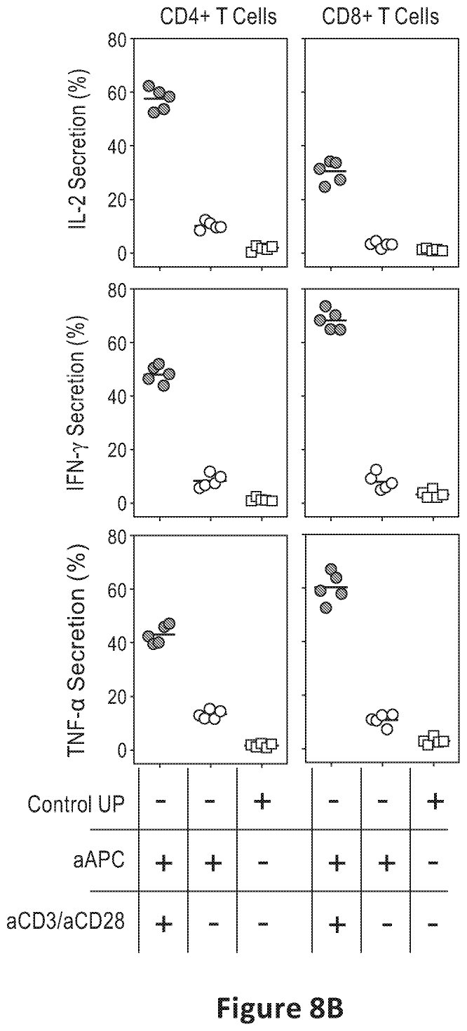

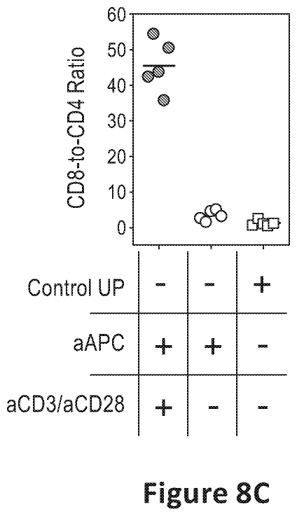

Proliferation, activation, and cytokine secretion of both CD8+ and CD4+ T cells was compared in the designed 3D scaffolds under different conditions (FIG. 8). To maintain consistent conditions for comparison, control particles were embedded within the scaffolds for IL-2 release so the release rate would not be a variant in the experiments (FIG. 8).

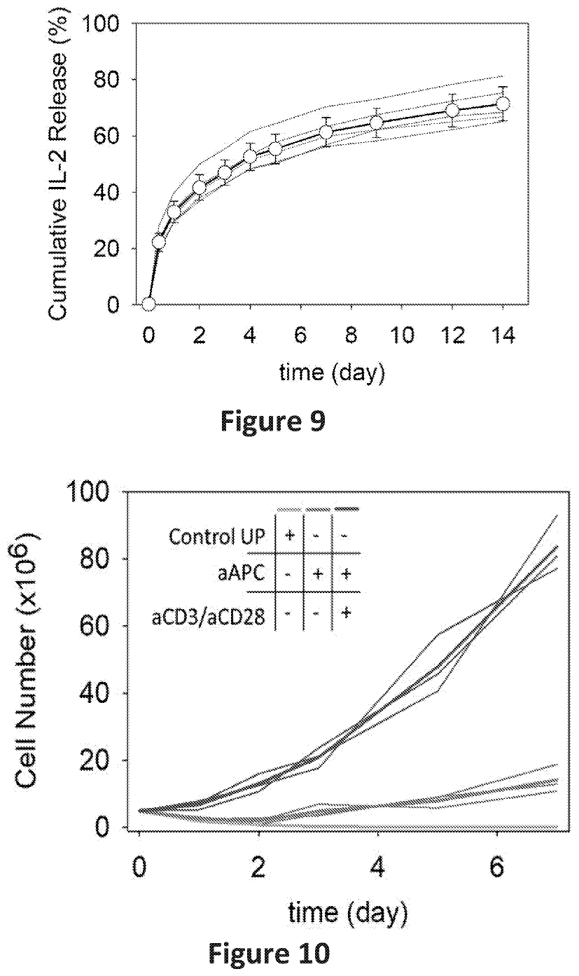

The release rate of IL-2 from the 3D alginate-RGD scaffold loaded with aAPCs was measured over time using ELISA (FIG. 9). Post conjugated 3D scaffolds loaded with antibody decorated, aAPC beads showed the highest proliferation and activation level (FIG. 8A) and similar to 2D upon co-seeding of both CD8+ and CD4+ T cells in the scaffolds CD8+ population was favored (FIG. 8C).

Due to the huge surface area that our microporous scaffold offers, T cell's expansion was improved by up to 9-fold upon scaffold post conjugation (FIG. 10).

### Example 16: Stiffness and Functionality Maintained in 3D Scaffolds Over Time

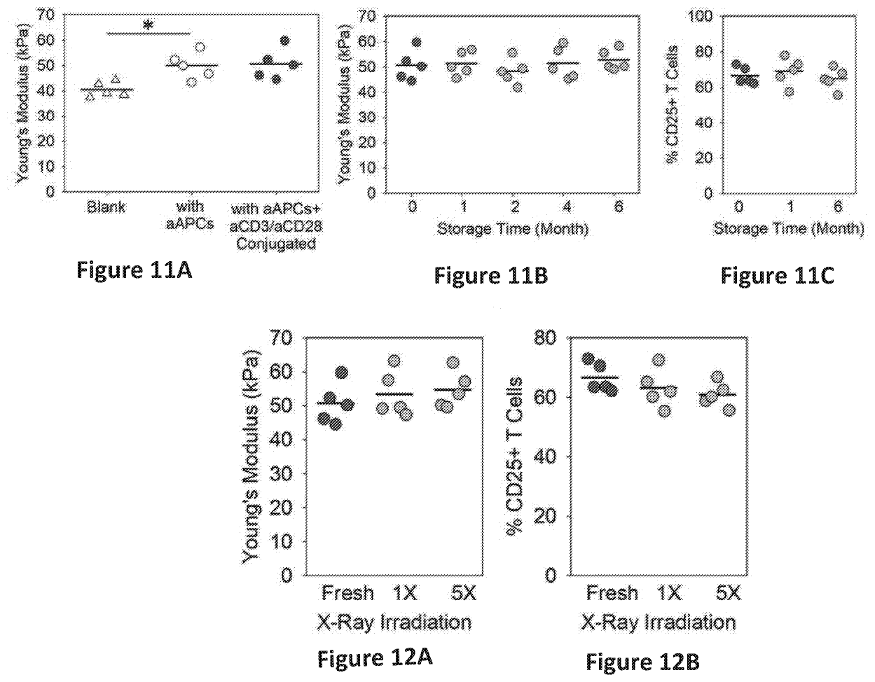

To optimize mechanical stiffness to be similar to that of lymph nodes, this feature was tested over time to determine the effects of particle dispersion within the scaffolds. While post-conjugation of scaffolds did not change their stiffness, loading of aAPCs within them slightly increased their stiffness (FIG. 11A) yet maintained stiffness within a reasonable range as compared with a lymph node's stiffness during an infection (see, e.g., Meng, K. P., Majedi, F. S., Thauland, T. J. & Butte, M. J. Mechanosensing through YAP controls T cell activation and metabolism. J. Exp. Med. 217, (2020); Experimental observations confirm the stiffening of lymph nodes in rodents from 4 kPa to 40 kPa upon viral infection with lymphocytic choriomeningitis virus (LCMV) ˜40 kPa.). Another feature tested was shelf-life of the scaffolds over time. Being able to store the scaffold for a long time without altering its properties is critical for clinical translation of such a product. No significant changes over time in the stiffness and functionality of the scaffolds were observed (FIGS. 11B-11C).

### Example 17: Functionality of 3D Scaffolds Maintained Following X-Ray Sterilization

For sterilization of scaffolds, X-ray irradiation (GULMAY MEDICAL™ RS320 x-ray unit) was used to irradiate the fabricated scaffolds before in vitro or in vivo functional tests, following ISO 11137-2:2013 recommended protocols (Corrigendum, T. Sterilization of health care products—Radiation—Part 2: Establishing the sterilization dose. Order A J. Theory Ordered Sets Its Appl. (2009); European Committee for Standardization. Sterilization of health care products—Radiation. BE EN ISO 11137-2:2013 (2013)). A sterilization dose of 25 kGy (2.5 Mrads) was used, because it has been reported that this dose does not alter the properties of pharmaceuticals (see, e.g., Abuhanoglu, G. & Ozer, A. Y. Radiation effects on pharmaceuticals. Fabad Journal of Pharmaceutical Sciences (2010)). Physical and biological properties, including changes in mechanical stiffness or change in T cell activation after sterilization process, were tested. Results showed non-significant changes in mechanical properties of scaffolds after receiving three cycles of 25 kGy sterilization dose (FIG. 12).

### Example 18: Localized Delivery In Vitro of Immunostimulants in 3D Scaffolds

Another hurdle ordinarily faced in the treatment of most solid tumors is the abundance of TGF-β which plays a key role in induction of Tregs in tumor microenvironment and leads to immune suppression (see, e.g., Park, J. et al. Combination delivery of TGF-β inhibitor and IL-2 by nanoscale liposomal polymeric gels enhances tumour immunotherapy. Nat. Mater. 11, 895-905 (2012)). TGF-β abundance and activity has been well documented in a number of murine tumor models (see, e.g., Park, J. et al. Combination delivery of TGF-β inhibitor and IL-2 by nanoscale liposomal polymeric gels enhances tumour immunotherapy. Nat. Mater. 11, 895-905 (2012); Gorelink, L. & Flavell, R. A. Immune-mediated eradication of tumors through the blockade of transforming growth factor-β signaling in T cells. Nat. Med. (2001). doi:10.1038/nm1001-1118; Liu, V. C. et al. Tumor Evasion of the Immune System by Converting CD4+CD25− T Cells into CD4+CD25+ T Regulatory Cells: Role of Tumor-Derived TGF-β. J. Immunol. (2007). doi:10.4049/jimmunol.178.5.2883; Yingling, J. M., Blanchard, K. L. & Sawyer, J. S. Development of TGF-β signalling inhibitors for cancer therapy. Nature Reviews Drug Discovery (2004). doi:10.1038/nrd1580) and may be a key counteracting player in IL-2 therapies where they seek to enhance CTLs activity. TGF-β in tumor cell growth and maintaining an immunologically cold tumor microenvironment plays a pivotal role (see, e.g., Yingling, J. M., Blanchard, K. L. & Sawyer, J. S. Development of TGF-β signalling inhibitors for cancer therapy. Nature Reviews Drug Discovery (2004). doi:10.1038/nrd1580; Kano, M. R. et al. Improvement of cancer-targeting therapy, using nanocarriers for intractable solid tumors by inhibition of TGF-β signaling. Proc. Natl. Acad. Sci. U.S.A (2007). doi:10.1073/pnas.0611660104).

While the exact source of TGF-β in a tumor microenvironment and the immunoprotective pathways behind its signaling blockade are not fully known, studies on combinatorial delivery of a TGF-β receptor-I inhibitor SB505124 plus an immunostimulant such as IL-2 has shown promising results in mouse melanoma model (see, e.g., Park, J. et al. Combination delivery of TGF-β inhibitor and IL-2 by nanoscale liposomal polymeric gels enhances tumour immunotherapy. Nat. Mater. 11, 895-905 (2012); Town, T. et al. Blocking TGF-β-Smad2/3 innate immune signaling mitigates Alzheimer-like pathology. Nat. Med. (2008). doi:10.1038/nm1781). However, the toxicity of systemic administration of immunostimulants, which block their therapeutic effects and use, has been widely reported (see, e.g., https://www.ncbi.nlm.nih.gov/pmc/articles/PMC3909428/).

The scaffold enables efficient, overtime, local delivery of these agents in the tumor bed (https://www.ncbi.nlm.nih.gov/pmc/articles/PMC3909428/).

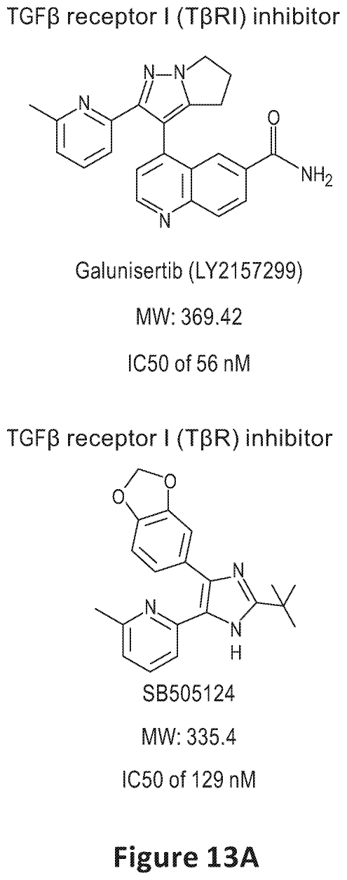

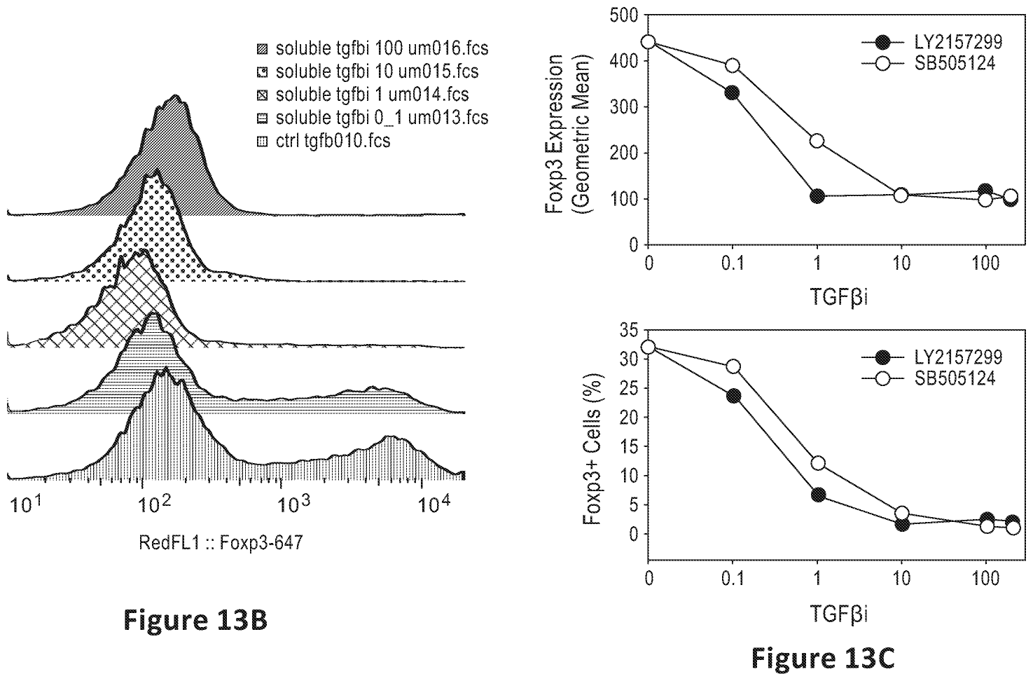

Here two of the commercially available TGF-β inhibitors, SB505124 and LY2157299 (Galunisertib) (see, e.g., Stauber, A. J., Credille, K. M., Truex, L. L., Ehlhardt, W. J. & Young, J. K. Nonclinical Safety Evaluation of a Transforming Growth Factor β Receptor I Kinase Inhibitor in Fischer 344 Rats and Beagle Dogs. J. Clin. Toxicol. 04, (2014); Rodón, J. et al. Pharmacokinetic, pharmacodynamic and biomarker evaluation of transforming growth factor-β receptor i kinase inhibitor, galunisertib, in phase 1 study in patients with advanced cancer. Invest. New Drugs (2015). doi:10.1007/s10637-014-0192-4; Yingling, J. M. et al. Preclinical assessment of galunisertib (LY2157299 monohydrate), a first-in-class transforming growth factor-β receptor type I inhibitor. Oncotarget (2018). doi:10.18632/oncotarget.23795; Can be tuned to be in the range of 2 weeks to 6 months), were tested at different doses (FIG. 13), as well as being tested for their potency in suppressing Treg formation. Galunisertib (LY2157299) is a TGF-beta receptor I (TβRI) inhibitor (molecular weight 369.42; IC50 56 nM) (top), while SB505124 is a TGF-beta receptor (TβR) inhibitor (molecular weight 335.4; IC50 129 nM) (bottom). At 1 nM concentrations, LY2157299 was found to be about twice as potent as SB505124 in suppressing Treg formation.

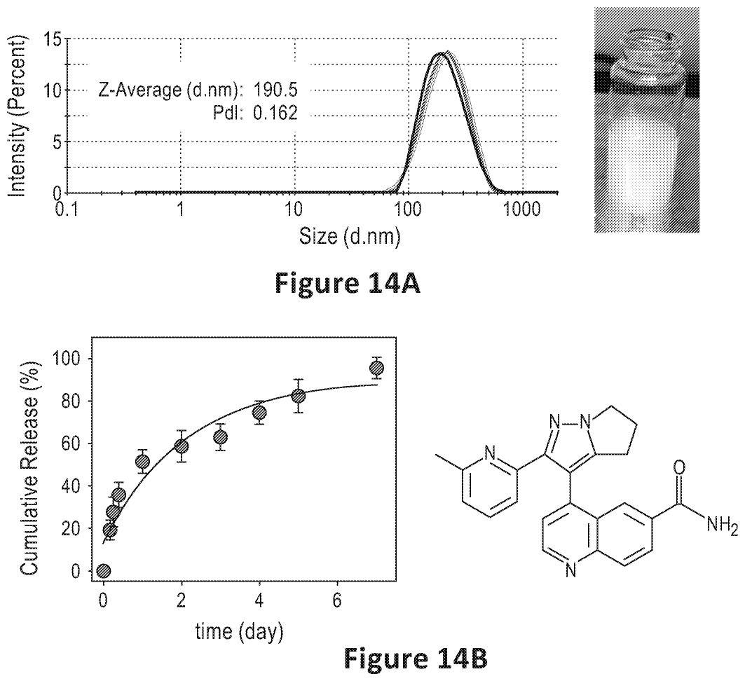

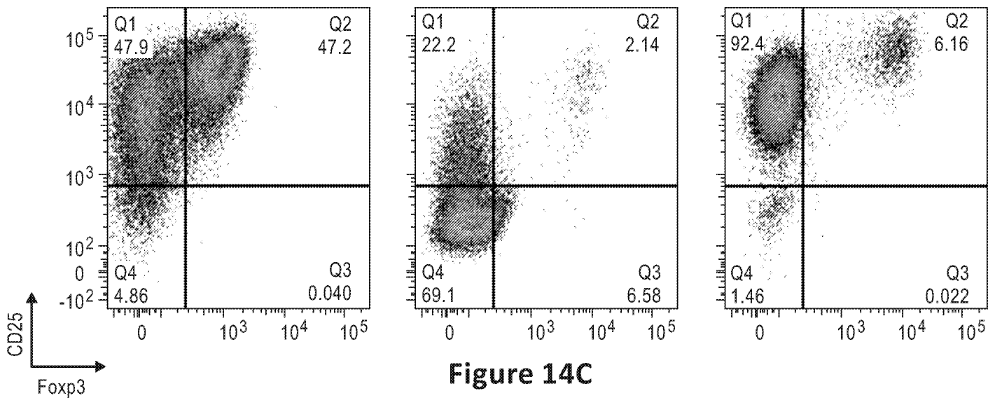

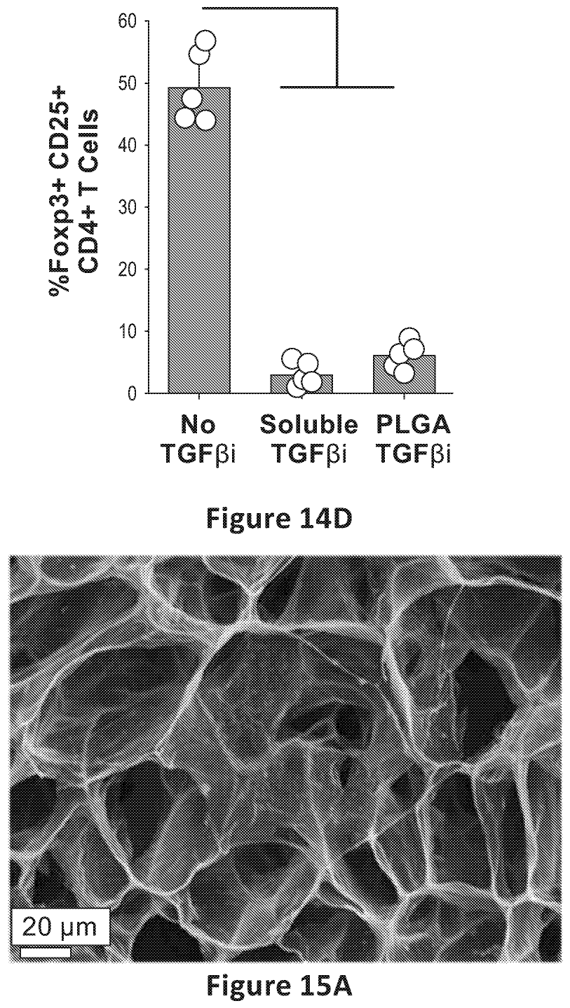

Due to the hydrophobic nature of this drug (LY2157299), poly(lactic-co-glycolic acid (PLGA) was selected as a carrier to load and release the selected TGF-β inhibitors. PLGA renders a slow, controlled biodegradation due to its compact structure. TGF-βi encapsulated PLGA nanoparticles with the size of about 200 nm were then fabricated and tested for their suppression capability against Treg formation (FIG. 14). Soluble TGF-βi was used as a control to check whether use of PLGA alters the activity of TGF-β inhibitors or not. Treg formation was suppressed by about 40 percent via both soluble administration of TGF-βi or upon co-culture with TGF-βi releasing PLGA nanoparticles (FIGS. 14C-14D).

### Example 19: Reduced Tregs in the Presence of 3D Scaffolds Due to Sustained Local Release

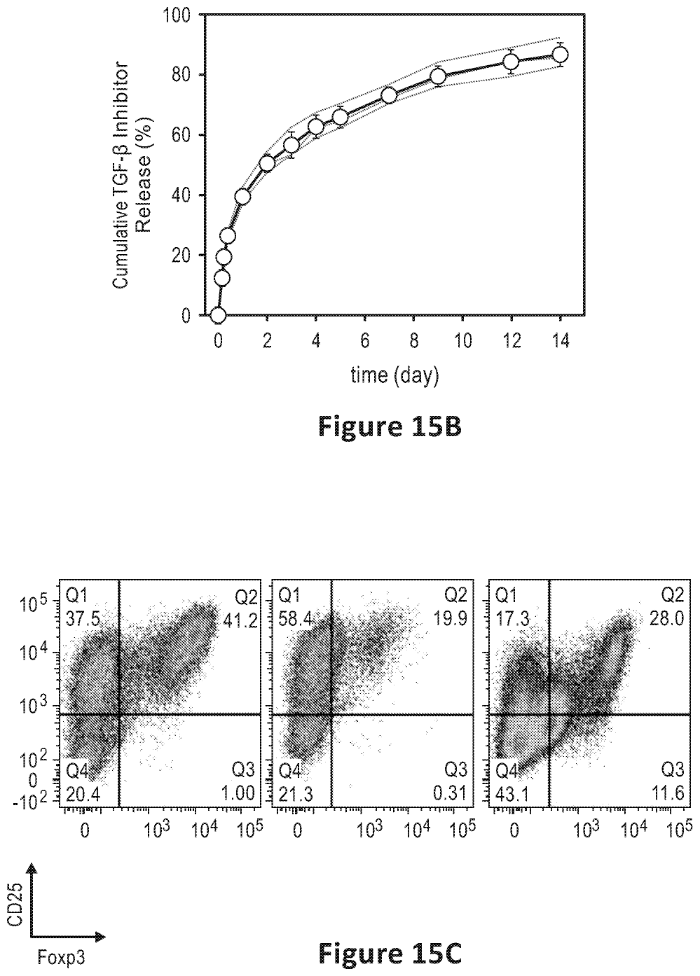

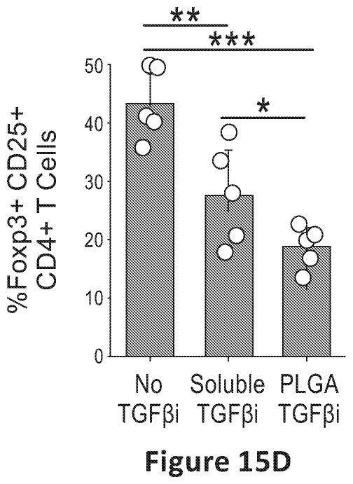

Once their functionality was confirmed in vitro, the particles with LY2157299 were loaded within the 3D scaffolds along with IL-2 releasing silica-heparin micro particles (FIG. 15). In each set of conditions, soluble TGF-β was supplemented in the media to induce formation of Tregs. In the 3D model, PLGA-loaded TGF-βi had a superior suppressive effect compared with its soluble administration (FIG. 15D). In the 3D formulation, Treg formation was inhibited by about 20 percent due to sustained, local release of TGF-βi on the adjacent cells.

### Example 20: 3D Scaffolds Enriched with Chemoattractant Tested for Both Active and Naïve T Cell Recruitment

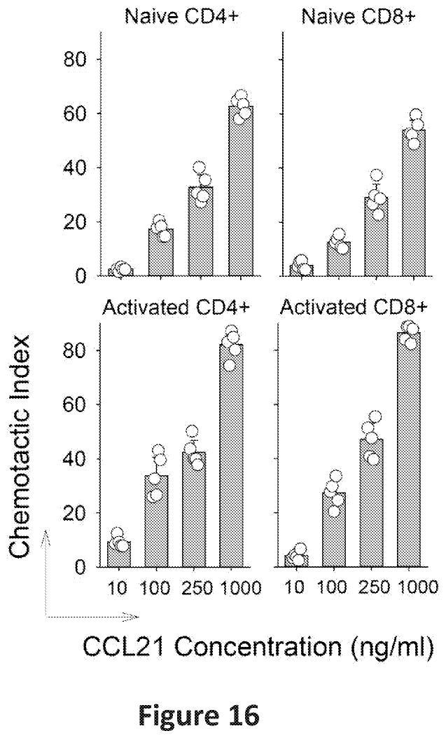

Once the capability of the 3D formulation for T cell activation, proliferation, and Treg suppression had been confirmed, the next step to make them suitable for in vivo functionality was to advertise them for the tissue resident T cells. To this end, the scaffolds were enriched with chemokine (C-C motif) ligand 21 (CCL21) as a chemoattractant to guide naive and active T cells (Weninger, W. et al. Naive T Cell Recruitment to Nonlymphoid Tissues: A Role for Endothelium-Expressed CC Chemokine Ligand 21 in Autoimmune Disease and Lymphoid Neogenesis. J. Immunol. (2003). doi:10.4049/jimmunol.170.9.4638; Liu, C. et al. The role of CCL21 in recruitment of T-precursor cells to fetal thymi. Blood (2005). doi:10.1182/blood-2004-04-1369) towards the synthetic lymph node. Different concentrations of CCL21 were mixed with alginate-RGD scaffold and were tested for both active and naive, CD8+ or CD4+ T cell recruitment using a transwell setup (FIG. 16).

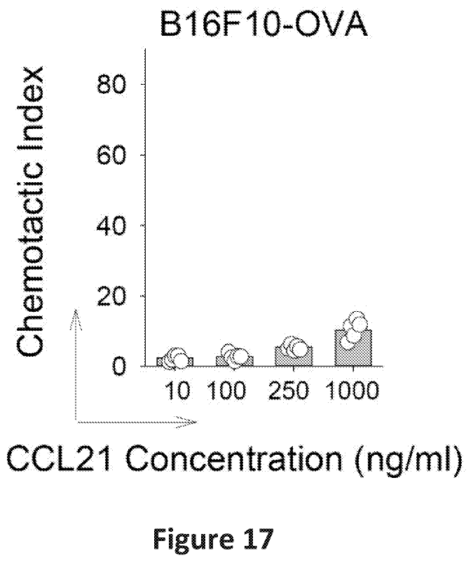

Because these scaffolds were designed to be implanted adjacent to the tumor tissue, recruitment of B16F10-OVA cells was also tested as a control, demonstrating that CCL21 have no significant effect on tested tumor cells (FIG. 17).

### Example 21: Implanting Synthetic Lymph Nodes for In Vivo T Cell Training

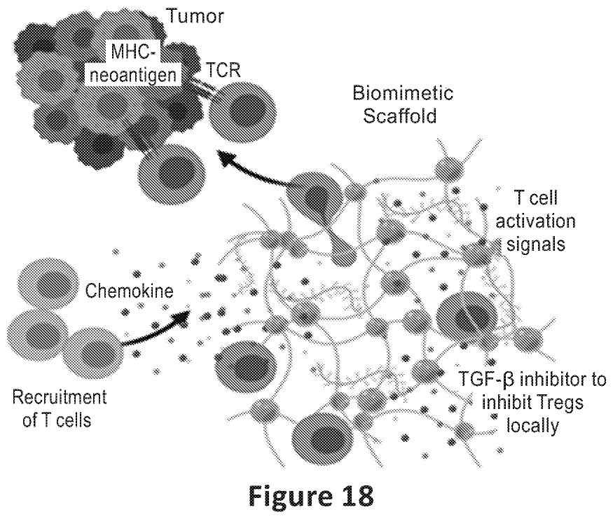

Upon demonstrating the scaffolds to be successful in vitro in terms of T cell recruitment, activation, expansion, and Treg suppression (FIG. 18), the scaffolds were then implanted in melanoma tumor-bearing wild-type C57/BL6 mice to evaluate their tumor clearance potency.

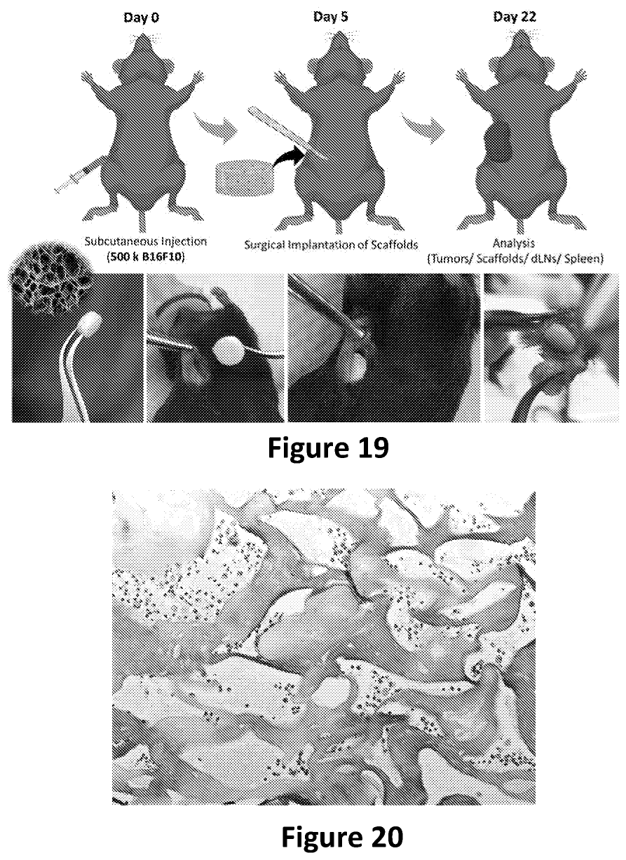

Typically, mice received subcutaneous injections of B16-F10 cells to their right flank followed by the scaffold implantation adjacent to the tumor once it was palpable. Without any further treatment animal's health was monitored and were euthanized 17 days afterwards. Implanted scaffolds, tumors, tumors' draining lymph nodes, and spleens were then retrieved for further studies (FIG. 19).

Hematoxylin and eosin (H&E) staining of the scaffold adjacent to the tumor showed successful tissue integration and recruitment of T cells via the implanted microporous scaffolds (FIG. 20).

### Example 22: Clearance of Tumors In Vivo

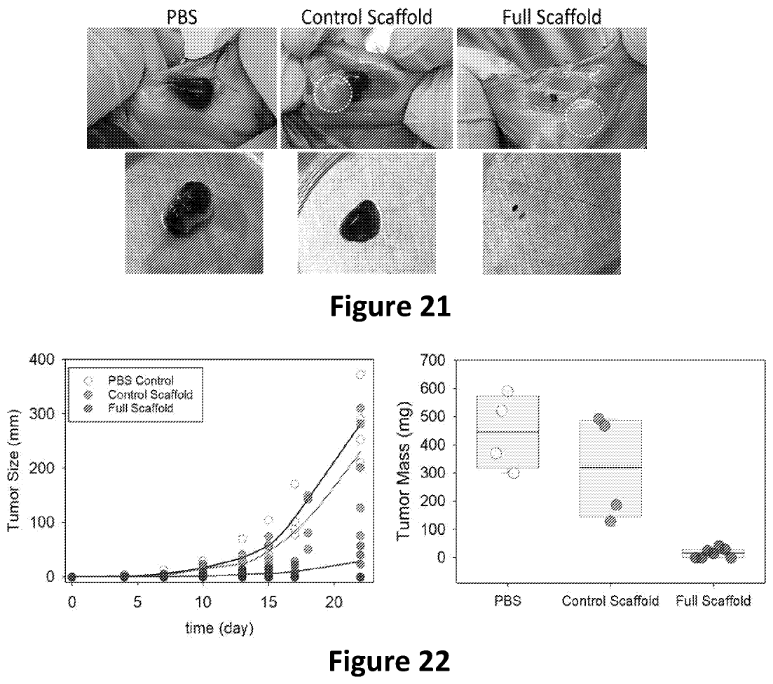

In this set of studies, blank scaffolds free of any particles or chemokines were used as controls along with PBS control. Tumor representative images were taken at the end of the experiments, their masses were measured (FIG. 21, FIG. 22) and tumor sizes were tracked overtime as an indicator of the tumor growth rate (FIG. 22). While for this aggressive tumor if left untreated (PBS control) or implanted control scaffold tumor will triple in size in 7 days, the full scaffold suppressed tumor growth drastically (FIG. 22). Full scaffold: Alginate-RGD scaffolds, loaded with aAPCs and CCL21, and post-conjugated with anti-CD3 and anti-CD28. Control Scaffold: Alginate-RGD scaffolds. Local recruitments and activation of endogenous T cells plus Treg suppression via the implanted alginate-based scaffold successfully eliminated the aggressive melanoma tumor in mice.

### Example 23: Status of Cells Recruited by Implanted Synthetic Lymph Nodes (ISL)

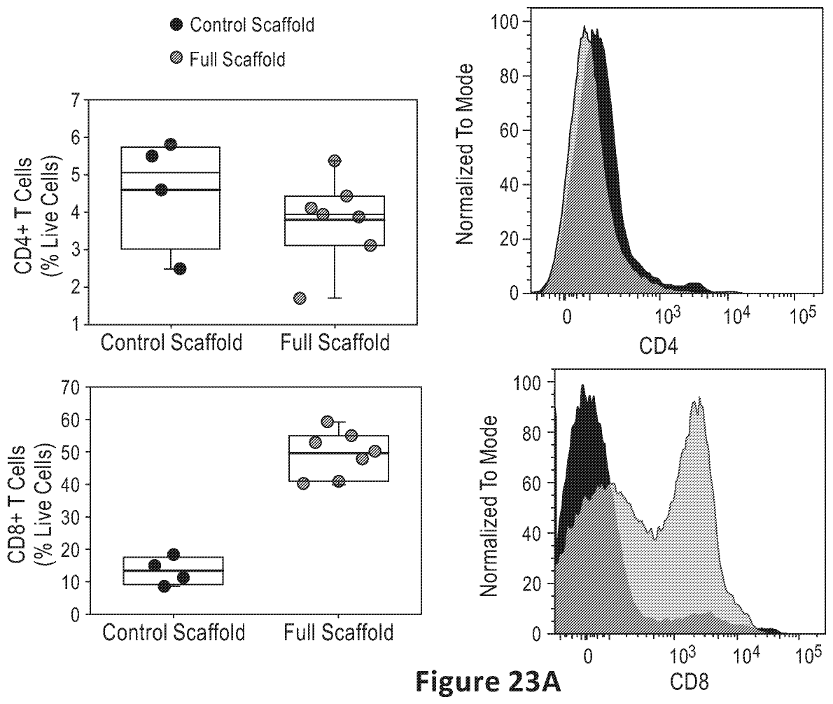

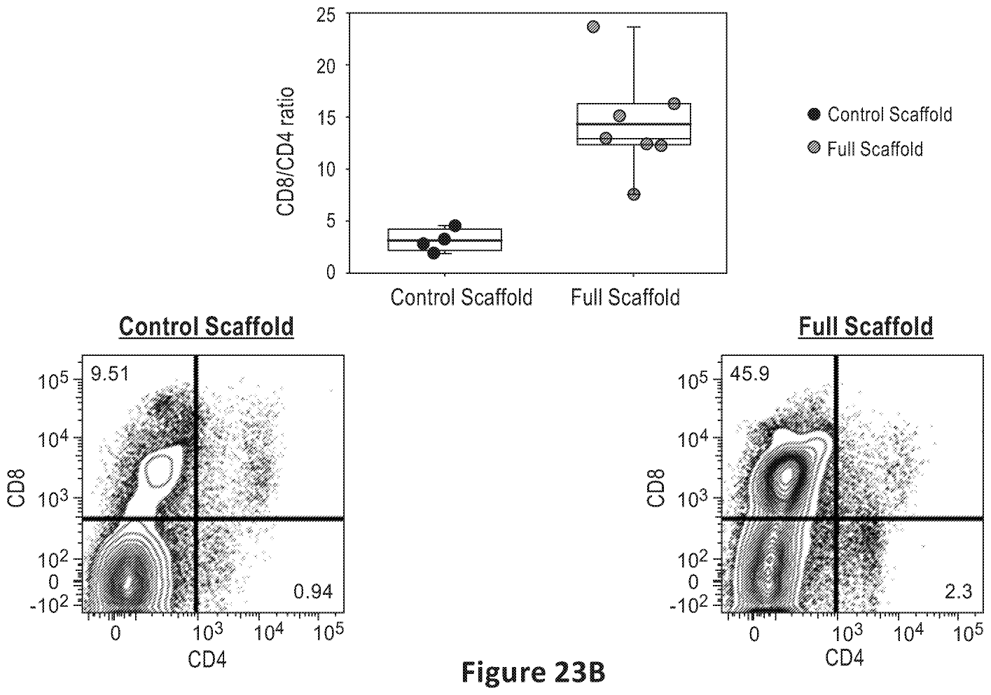

The status of recruited cells by our implanted synthetic lymph nodes (ISL) was assessed (FIG. 23). Due to the successful and prolonged release of the CCL21, T cells, CD8+ T cells in particular, constituted the majority of recruited lymphocytes in our full scaffolds (FIG. 23A) while no difference seemed to happen in the population of recruited CD4+ T cells in full vs. control scaffolds. Thus, CD8+ to CD4+ ratio of T cells were about 7 times higher in the full scaffold compared to the control one (FIG. 23B).

### Example 24: Activation of T Cells Recruited by the Implanted Synthetic Lymph Nodes

Tumor clearance potency of the implanted synthetic lymph nodes (ISL) was interesting as the scaffold offers polyclonal activation and expansion of endogenous T cells via conjugated anti-CD3/CD28 antibodies while providing IL-2 cytokine.

Despite the lack of tumor specific training, T cells that were recruited and trained in the ISL recognized the tumor and were capable of clearing it, suggesting that any changes might have happened to the population of endogenous tumor reactive T cells and that recruiting endogenous T cells in the ISL adjacent to the tumor allowed for dual exposure of them to both anti-CD3/CD28 antibodies which is provided by the ISL and antigens presented on the tumor simultaneously. As a result, either T cells had higher chances of recognizing tumor cells and killing them, or the ISL was recruiting and expanding tissue resident T cells which along the way results in activation and expansion of tumor-specific resident T cells and this plus suppression of Treg population is enough to suppress the tumor growth and clear it.

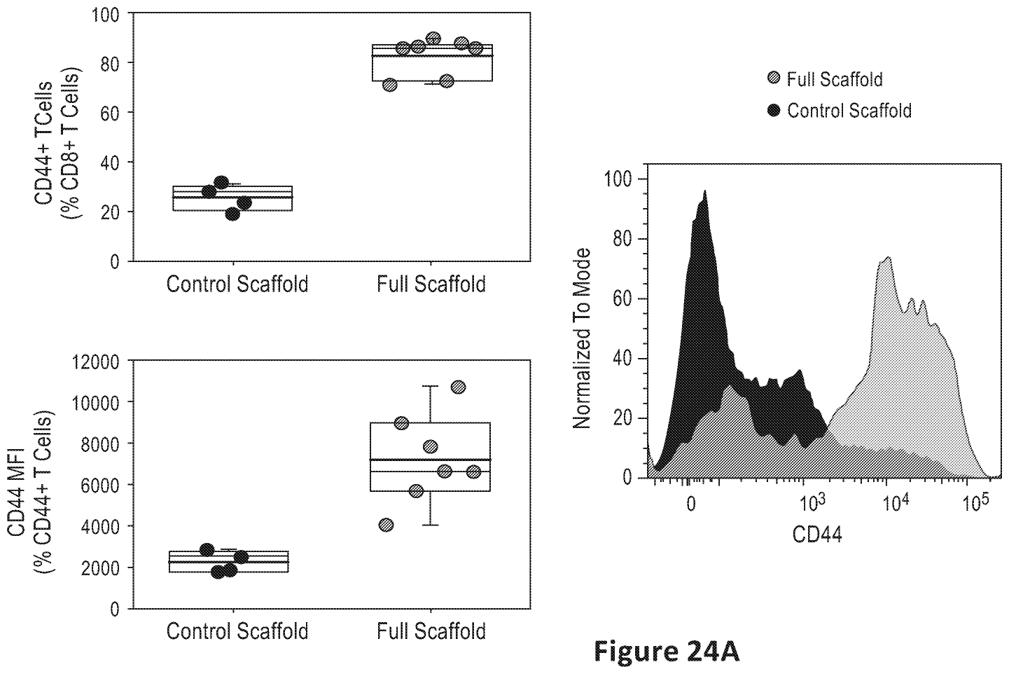

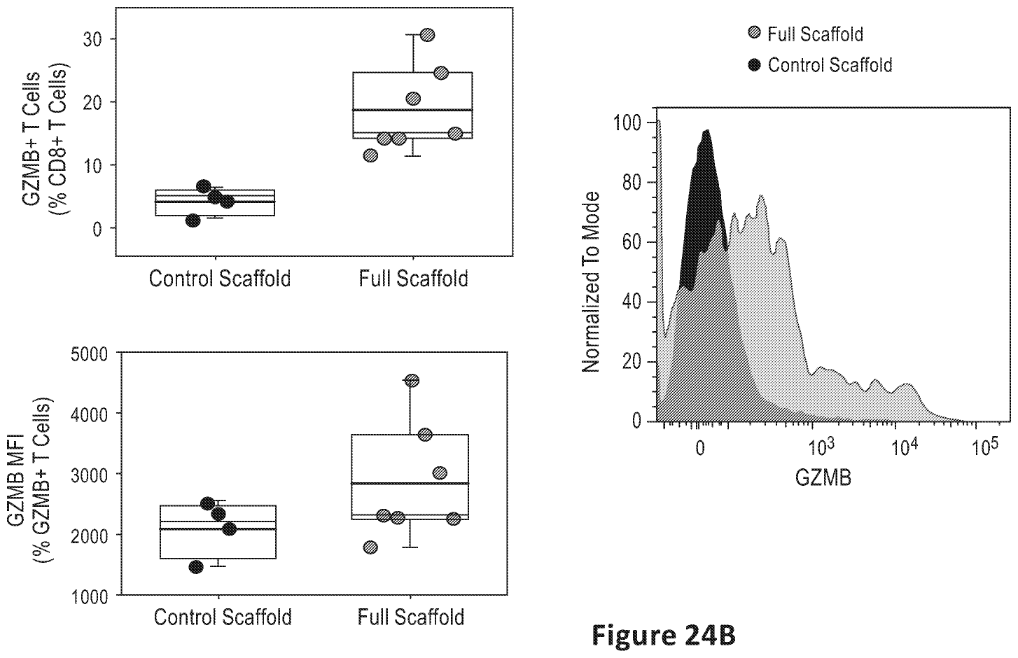

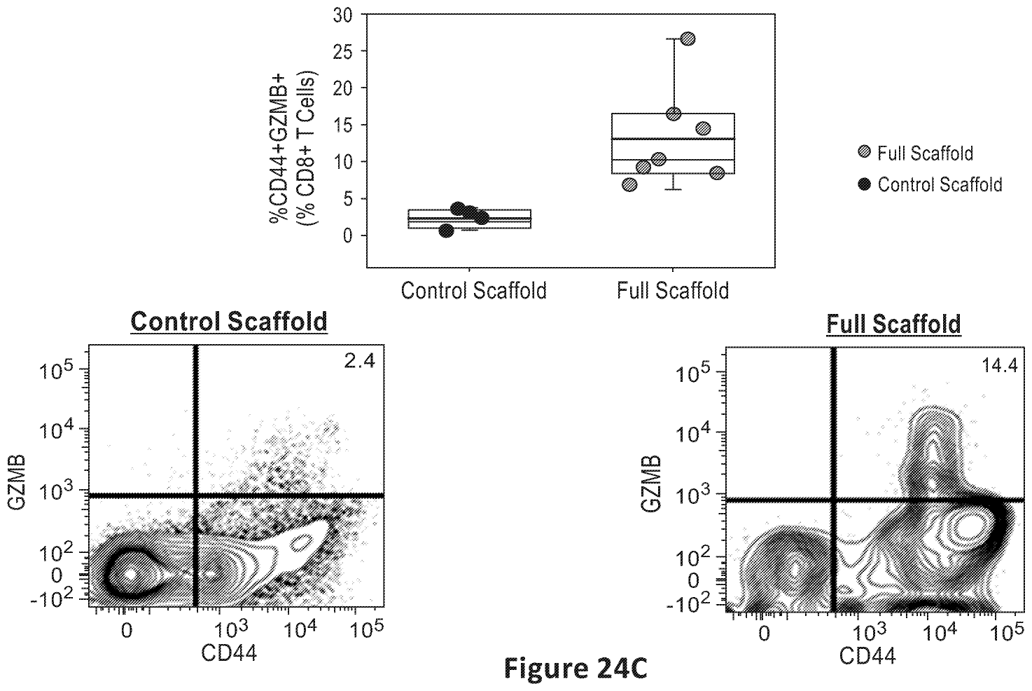

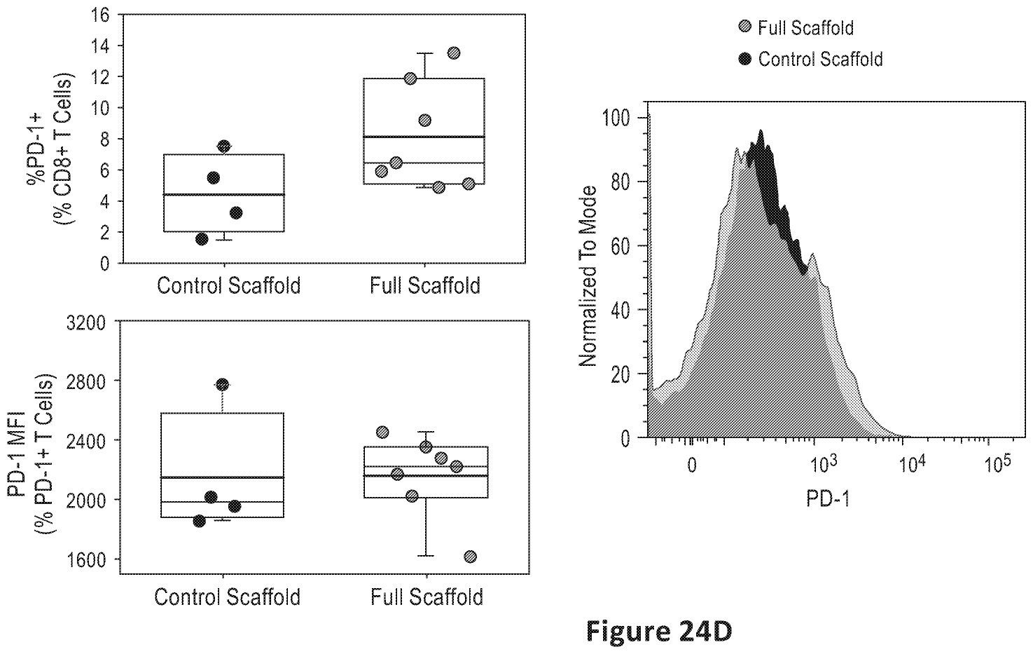

In order to confirm activation of the recruited T cells by the ISL the level of CD44 expression as an activation marker was assessed (FIG. 24A), and the GZMB expression was measured as an indicator of cytotoxicity tumor fighting T cells (FIG. 24B). Approximately 80 percent of the recruited CD8+ T cells were activated (FIG. 24A) from which 20% showed cytotoxic potency (FIG. 24B, FIG. 24C). No difference amongst the population of programmed-death-1 (PD-1) positive T cells was observed in our ISLs compared to control (FIG. 24D).

Moreover, the population of endogenous OTI T cells within the scaffold were no different from the control scaffold (FIG. 25).

### Example 25: Characterization of Tumor Infiltrated T Cells

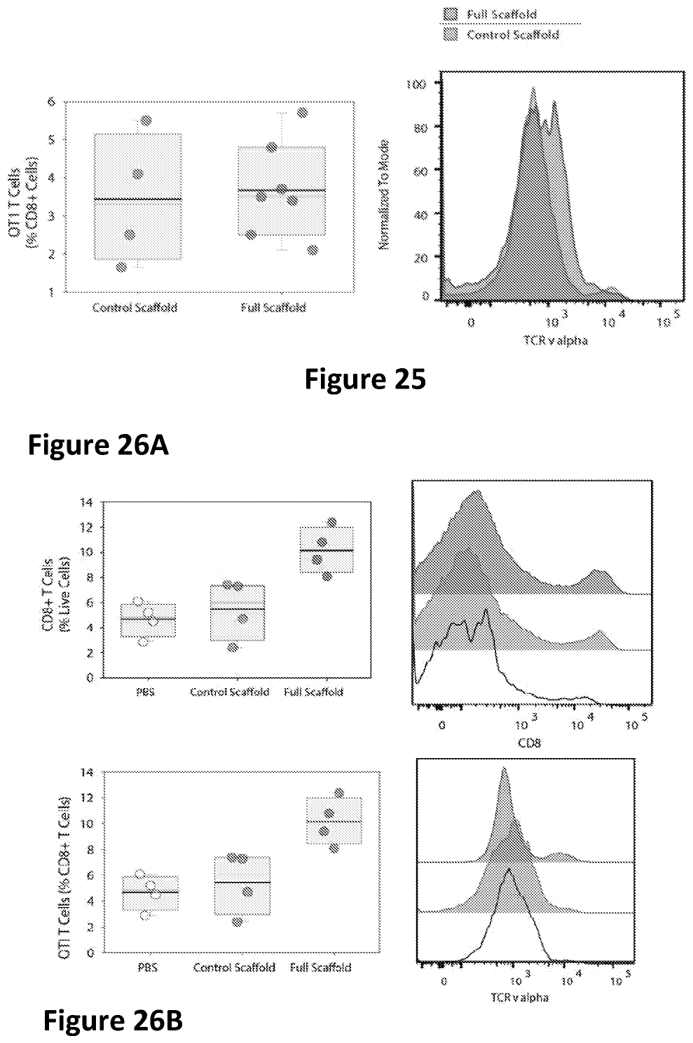

Mice bearing B16-F10-Ova tumors were euthanized 22 days after tumor injection. Three out of the seven mice that received the ISL had absolutely no tumor. Detectable tumors in the remainder of mice were then lysed and checked for the presence of polyclonal or tumor specific T cells (FIG. 26). The percentage of tumor infiltrated CD8+ T cells was increased significantly along with more than two times increase in the population of tumor specific OTIs (FIG. 26B) confirming the fact that the population of tumor specific T cells is improved in the ISL due to adjacency to tumor antigens plus a homing niche for activation and proliferation.

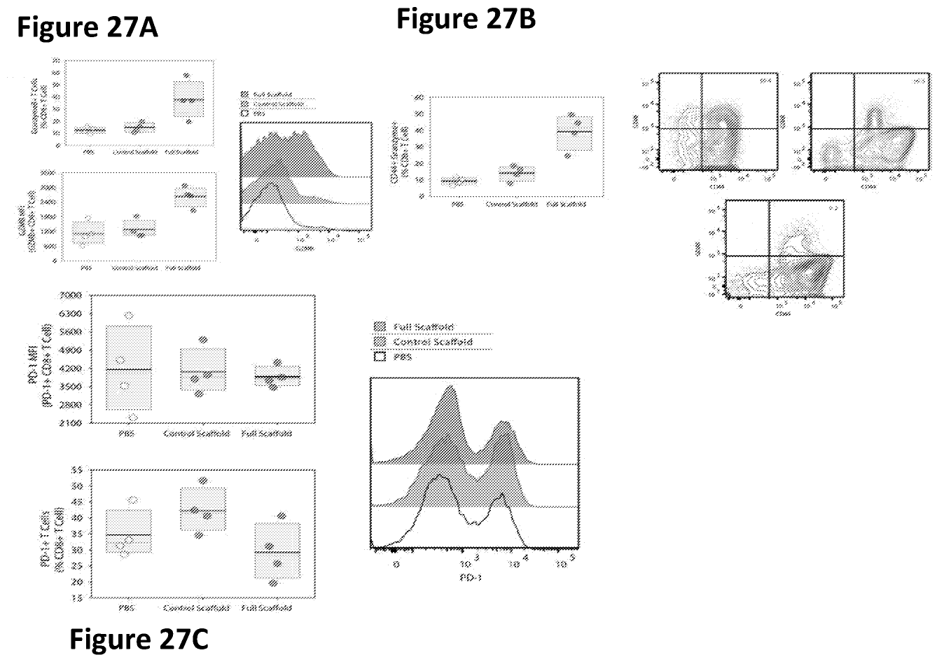

We then assessed the level of granzyme B (GZMB) expression of tumor infiltrating T cells in our ISL and we found a 40 percent increase in activated GZMB+ T cells (FIGS. 27A-27B). We found no significant difference in the levels of PD-1 expression on day 22 between the examined groups (FIG. 27C).

### Example 26: Reduction of Immunosuppression by Tumor Cells

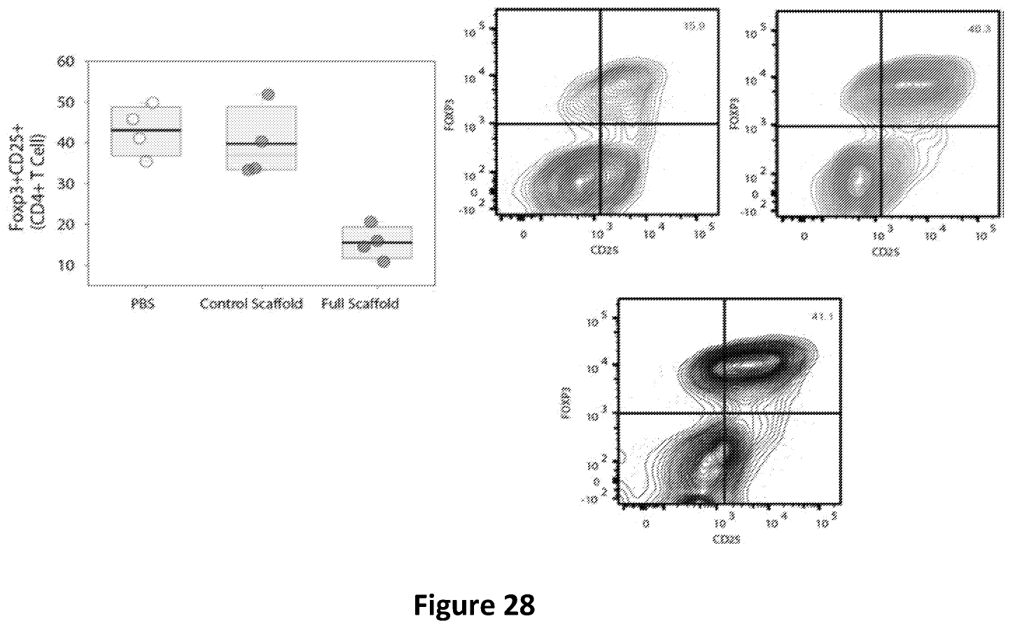

One of the major hurdles in most solid tumors, such as melanoma, is the immunosuppressive environment which tumor cells promote by inducing formation of Treg. In the scaffold platform, TGF-βi (LY2157299) releasing PLGA nanoparticles were designated to reverse tumor's immunosuppressive environment to an immunostimulant one to observe the effects of the ISL in rearranging T cell population around the tumor microenvironment. Treg population was observed to have been suppressed by about 30 percent with the scaffold formulation that carries TGF-βi (FIG. 28).

### Example 27: Effects of Scaffolds on CD8+ T Cells and OT-I Cells in Tumor Draining Lymph Nodes

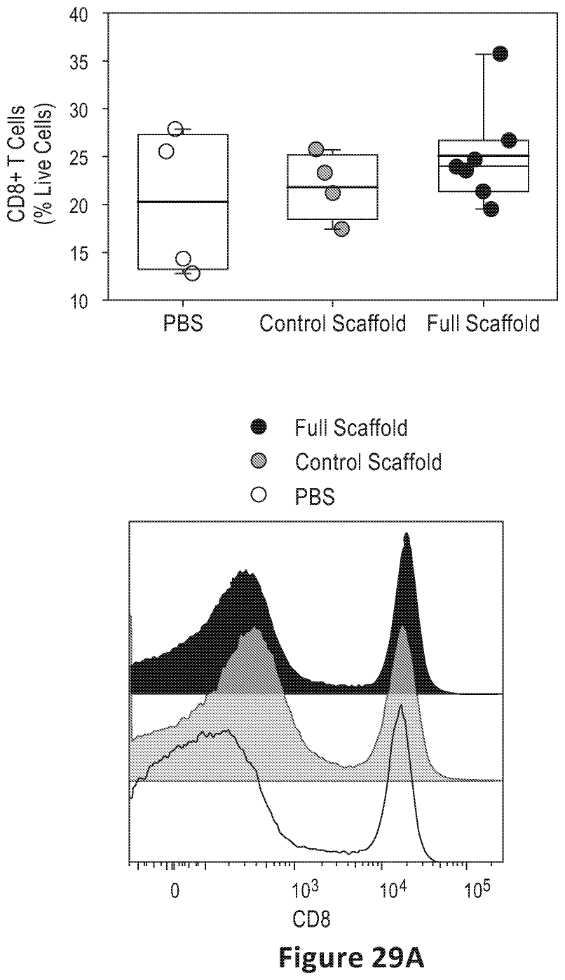

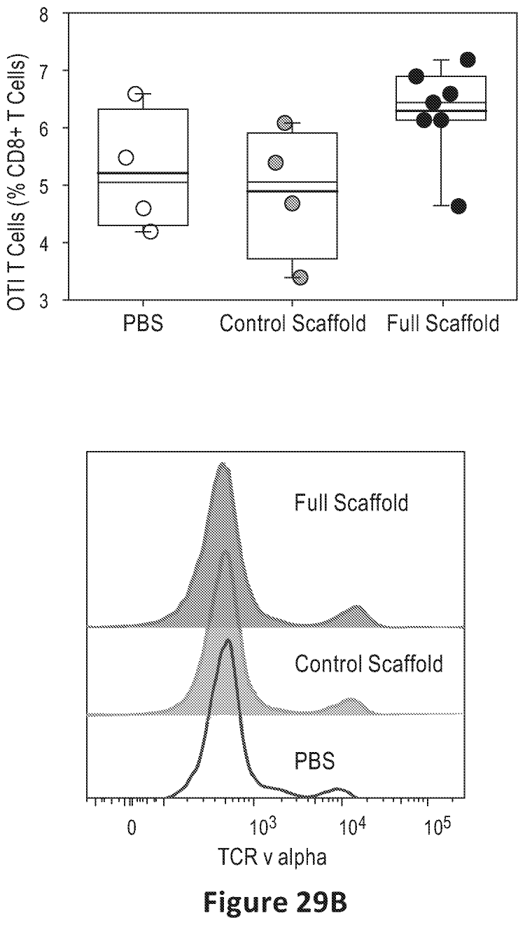

Furthermore, to determine whether local treatment caused any changes in the population of activated T cells elsewhere a study was performed to assess whether there were CD8+ T cells in the draining lymph node (FIG. 29). Results showed no significant difference in the population of activated CD8+ T cells (FIG. 29A) or OT-Is as representative sub populations in the tumor draining lymph nodes (FIG. 29B).

### Example 28: Observations of Scaffolds with Respect to Potential Side Effects or Autoimmune Reactions

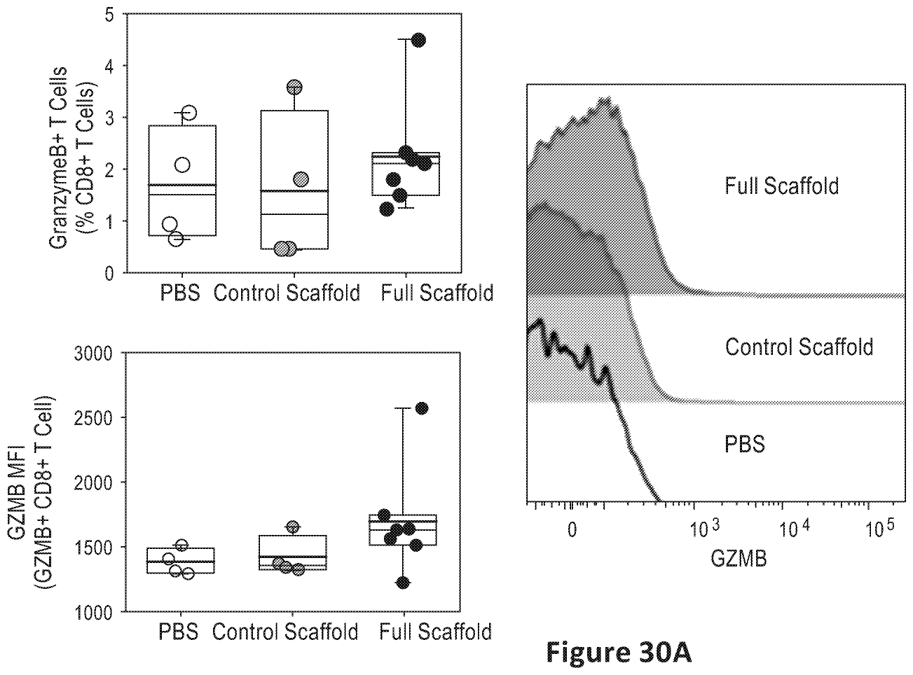

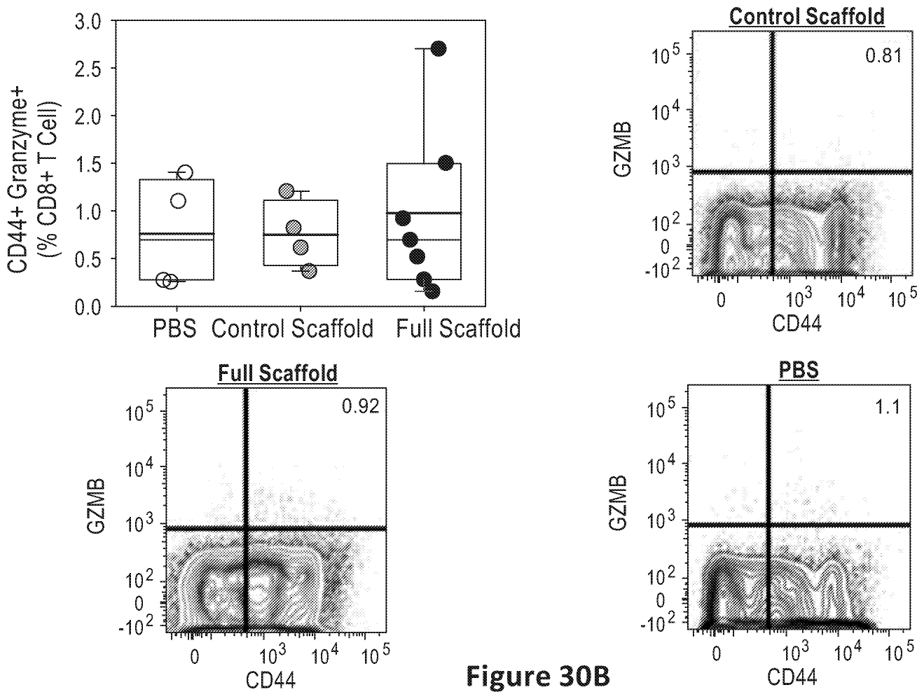

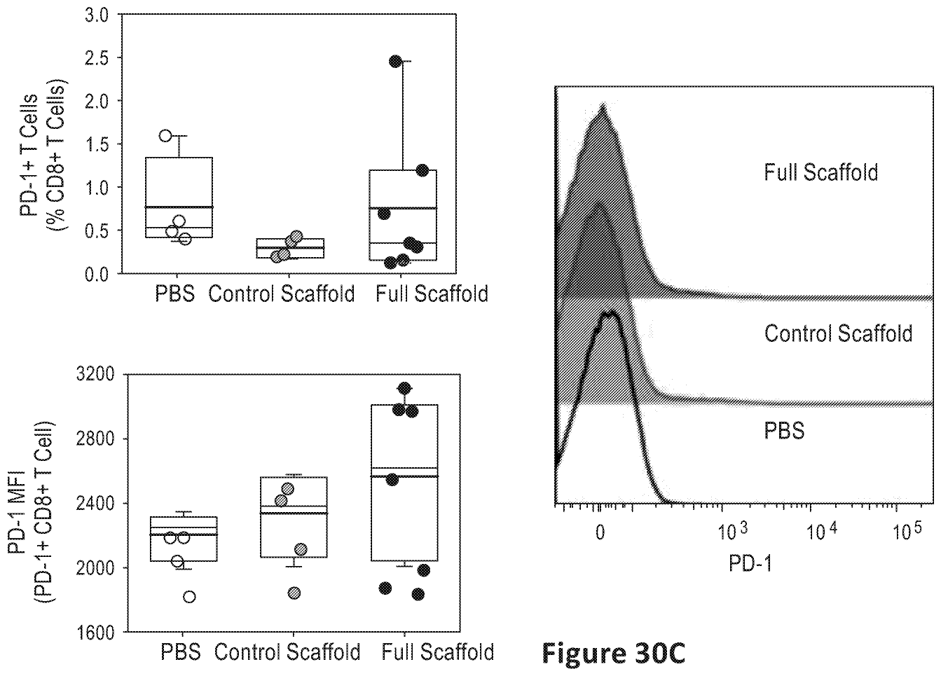

One of the major challenges that hampers the therapeutic efficacy of systemic administration of small molecules is the side effects that come with them due to their off-target distribution in other tissues. There was no observation of any meaningful changes in the percentages of GZMB+ or PD-1 expressing T cells in our ISL vs. control scaffold or PBS control (FIG. 30).

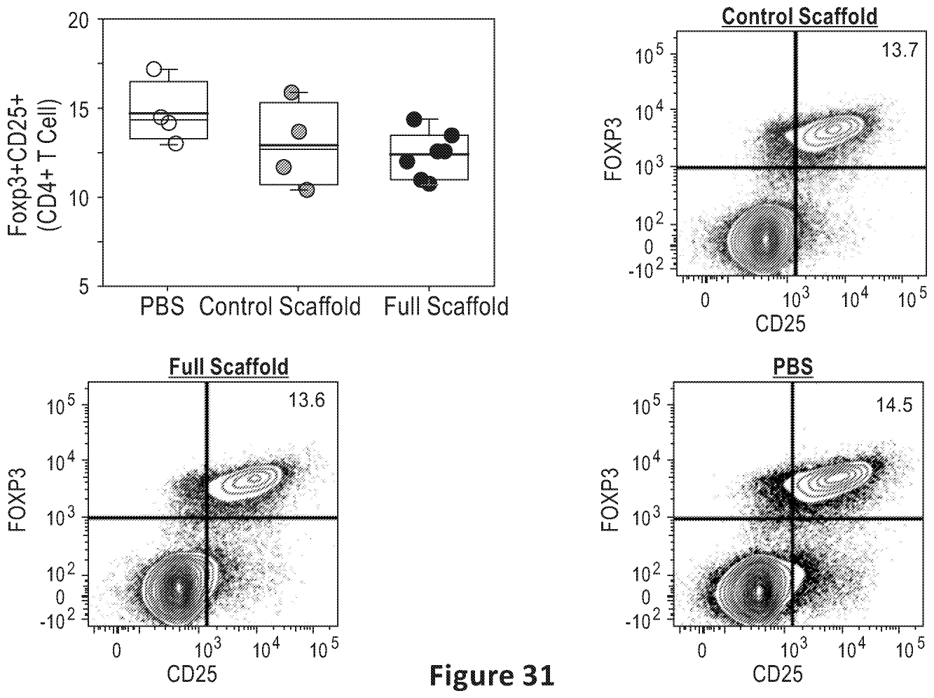

As systemic administration of TGF-βi can result in autoimmune disease (Wrzesinski, S. H., Wan, Y. Y. & Flavell, R. A. Transforming growth factor-β and the immune response: Implications for anticancer therapy. Clinical Cancer Research (2007). doi:10.1158/1078-0432.CCR-07-1157), the local release of TGF-βi adjacent to the tumor will result in suppression of Tregs in the draining lymph node was assessed. Results showed no significant changes in Treg populations in the draining lymph node (FIG. 31).

### Example 29: Effects of the Scaffolds on the Spleen

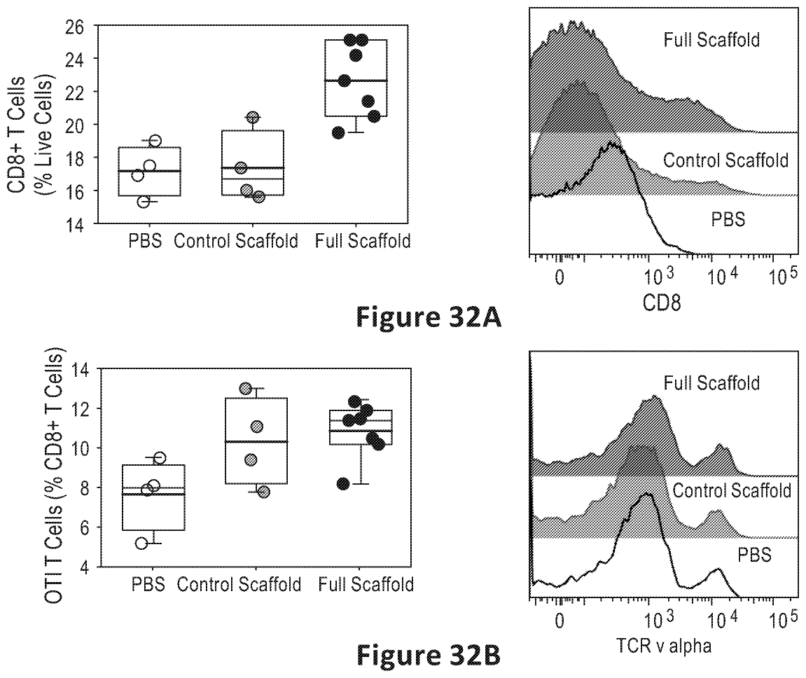

Additionally, changes in the population of T cells in the spleen were assessed, and it was found that activated CD8+ T cells were slightly (about 6%) increased using the ISL compared to control scaffold (FIG. 32A) while the changes in the population of TCR V-alpha positive T cells as a representative population was not meaningful (FIG. 32B).

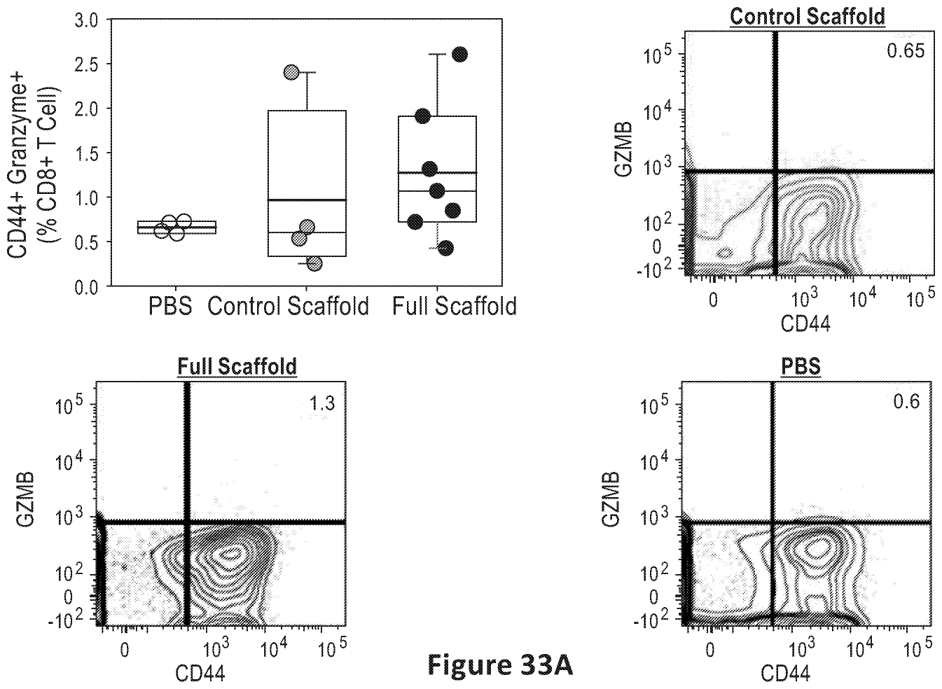

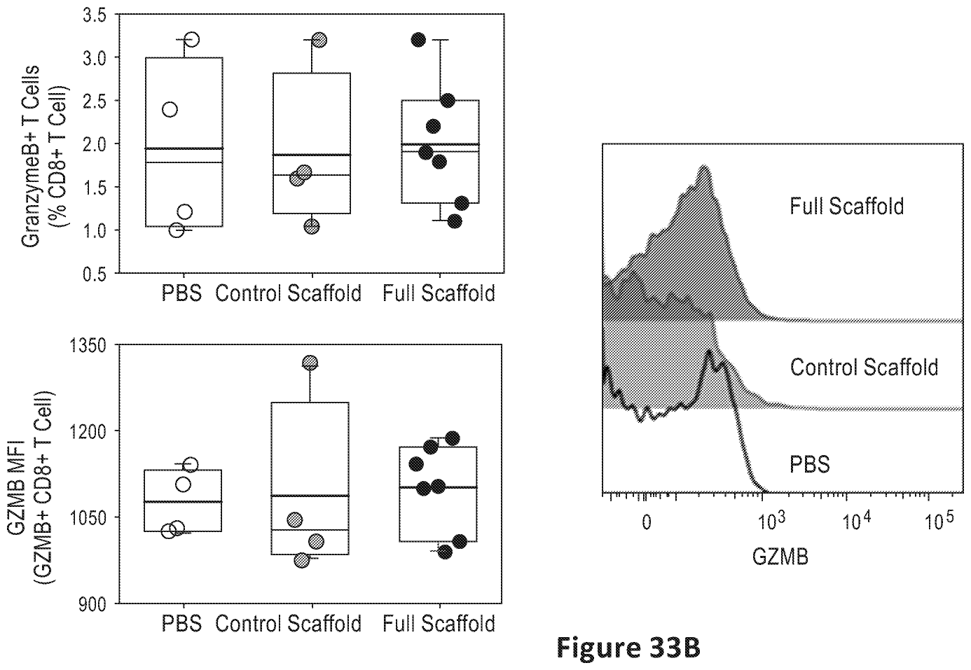

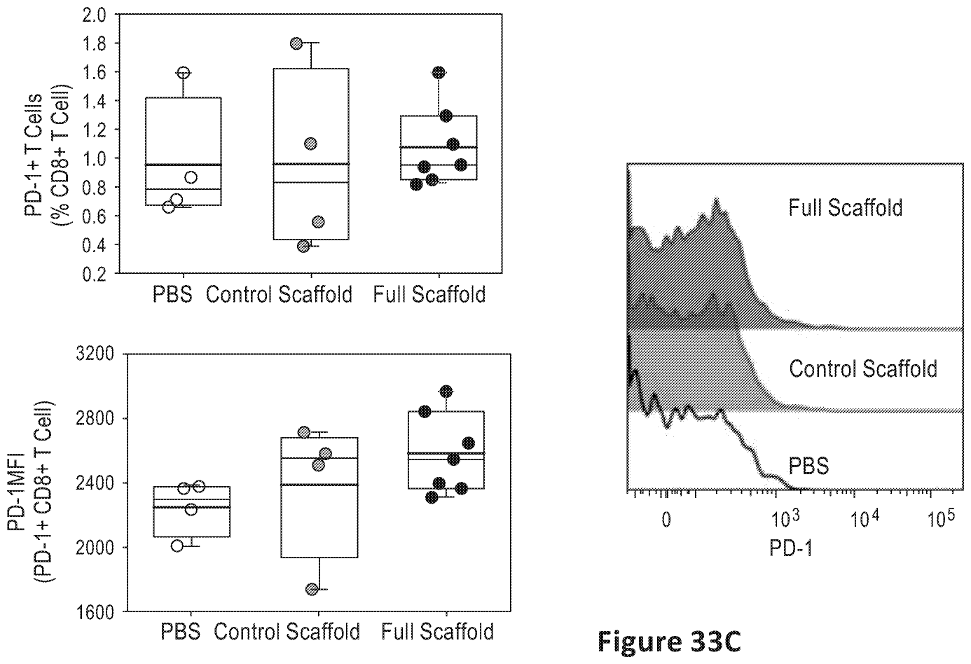

Moreover, no significant changes in the population of activated, GZMB+ T cells or PD-I expressing CD8+ T cells were observed in the spleen using the ISL vs. control conditions (FIG. 33).

### Example 30: Scaffolds with Chemokines Recruit T Cells

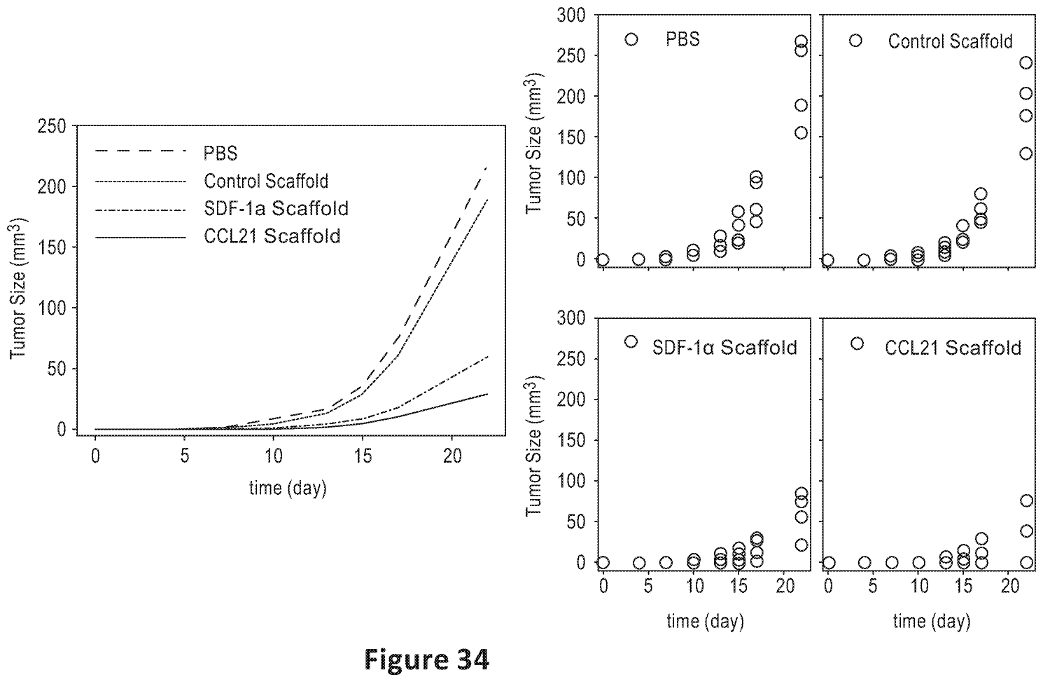

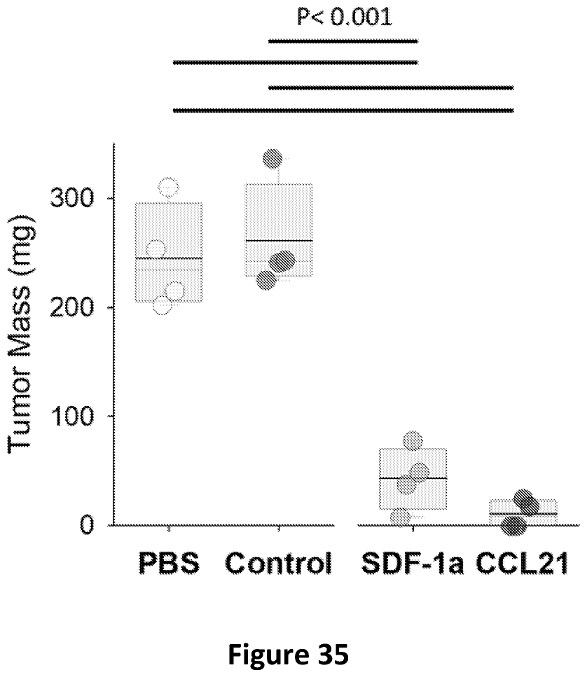

In order to advertise the scaffolds specifically for T cells, chemokine (C-C motif) ligand 21 (CCL21) was selected as a chemokine. CCL21, as one of the major ligands of C-C chemokine receptor type 7 (CCR7), is considered as the principal integrin activating chemokine. CCL21 has a possible role in recruitment of effector cells (Lin, Y., Sharma, S. & John, M. S. CCL21 cancer immunotherapy. Cancers (2014). doi:10.3390/cancers6021098; Novak, L., Igoucheva, O., Cho, S. & Alexeev, V. Characterization of the CCL21-mediated melanoma-specific immune responses and in situ melanoma eradication. Mol. Cancer Ther. (2007). doi:10.1158/1535-7163.MCT-06-0709). On the other hand, stromal cell-derived factor 1 alpha (SDF-1α) is another common chemokine known to regulate migration of many types of cells, especially progenitor cells (Cencioni, C., Capogrossi, M. C. & Napolitano, M. The SDF-1/CXCR4 axis in stem cell preconditioning. Cardiovasc. Res. 94, 400-407 (2012); Dunussi-Joannopoulos, K. et al. Efficacious immunomodulatory activity of the chemokine stromal cell—derived factor 1 (SDF-1): local secretion of SDF-1 at the tumor site serves as T-cell chemoattractant and mediates T-cell—dependent antitumor responses. Blood 100, 1551-1558 (2002)). To verify which chemokine serves our purpose best, all the components of the scaffolds were maintained identically except for the chemokine. As shown in FIG. 34 and FIG. 35 no significant difference in terms of tumor size or mass was noticed.

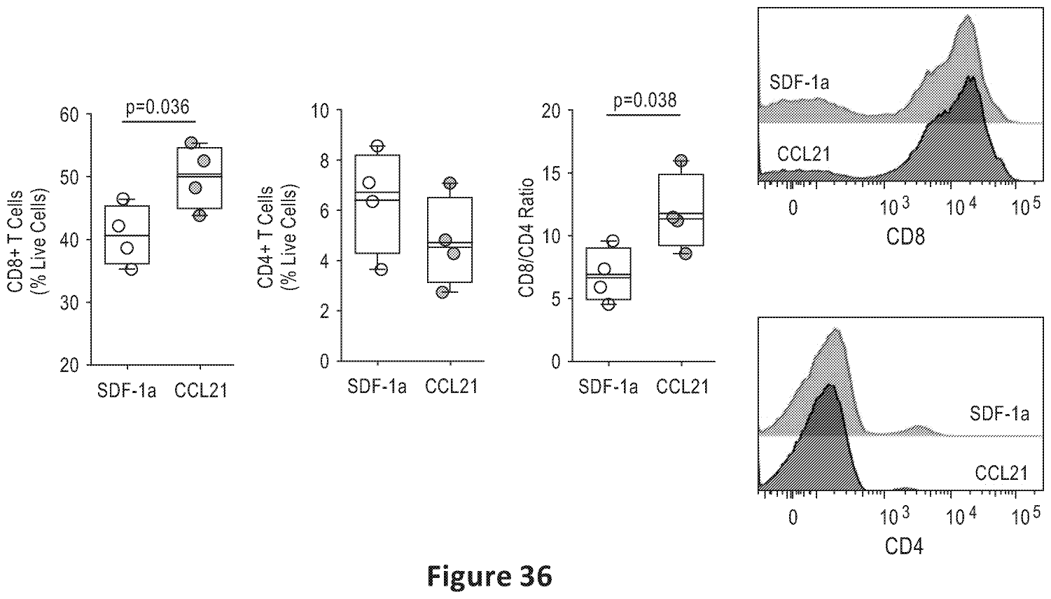

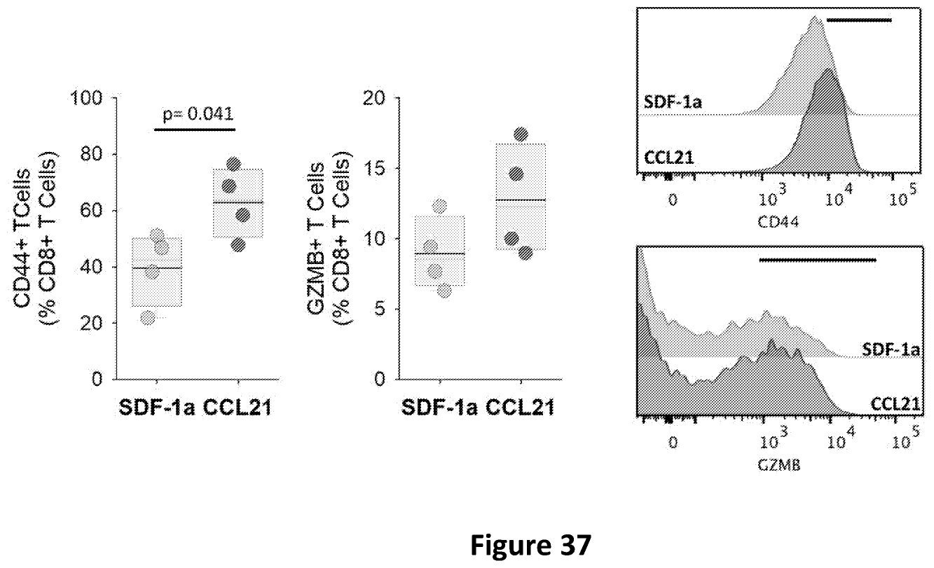

However, the percentage of recruited CD8+ via CCL21 was slightly higher and the population of non-CD8+ cells recruited in the scaffolds containing SDF-1a were visibly higher (FIG. 36). Additionally, more activated CD8+ T cells were found in scaffolds with CCL21 (FIG. 37), while GZMB secreting populations were roughly similar in both conditions. Together these data demonstrated that CCL21 favors recruitment of CD8+ T cells more than SDF-1α.

### Example 31: Stability of Stored Scaffolds Over Time

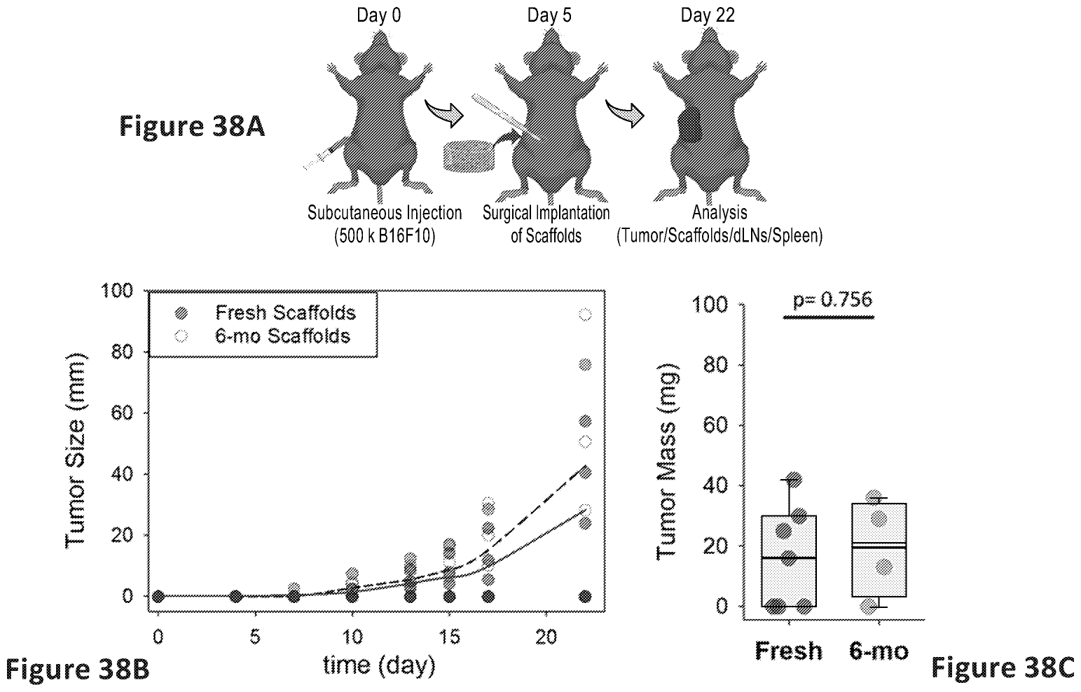

In order to test the stability and shelf-life of lyophilized scaffolds after 6-months in 4° C. both fresh and 6-month old scaffolds were implanted in mice and checked for their capability of T cell recruitment and activation (FIG. 38A). In terms of tumor suppression both the freshly made and 6-month old scaffolds performed well (FIG. 38).

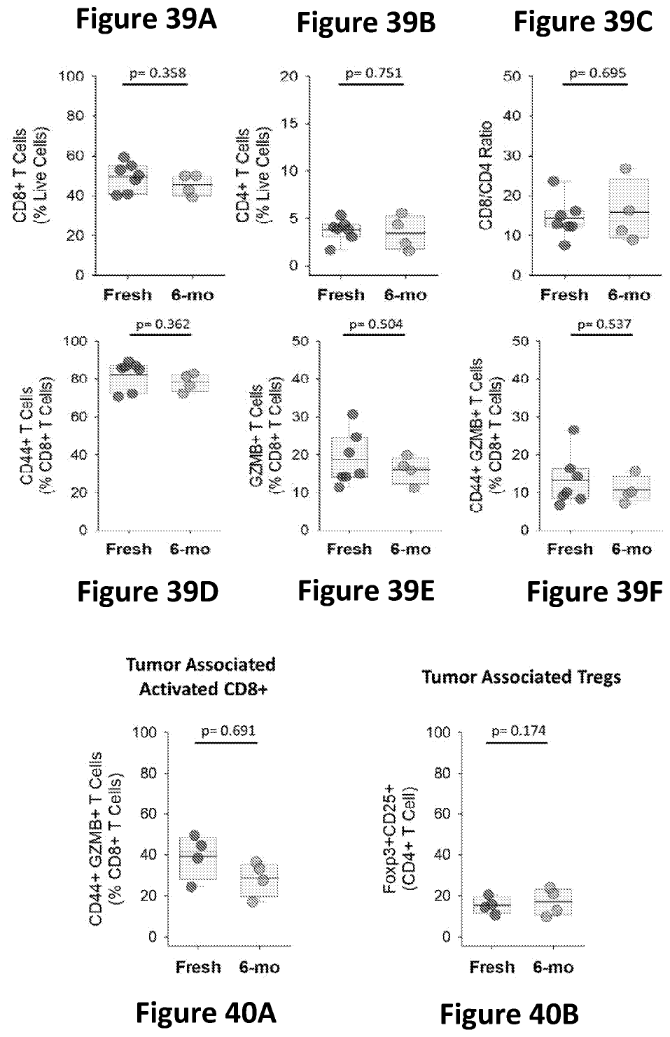

As shown in FIG. 39, the percentage of recruited, activated and GZMB+ T cells were comparable in old vs. fresh scaffolds.

The fact that the percentage of activated GZMB+ T cells and Tregs in the tumors were similar in old vs. fresh scaffolds confirms that the scaffolds preserve the functionality of loaded drugs and chemokines to a great extent (FIG. 40).

### Example 32: Treatment of the Primary Tumor with a Scaffold Suppresses a Secondary Tumor

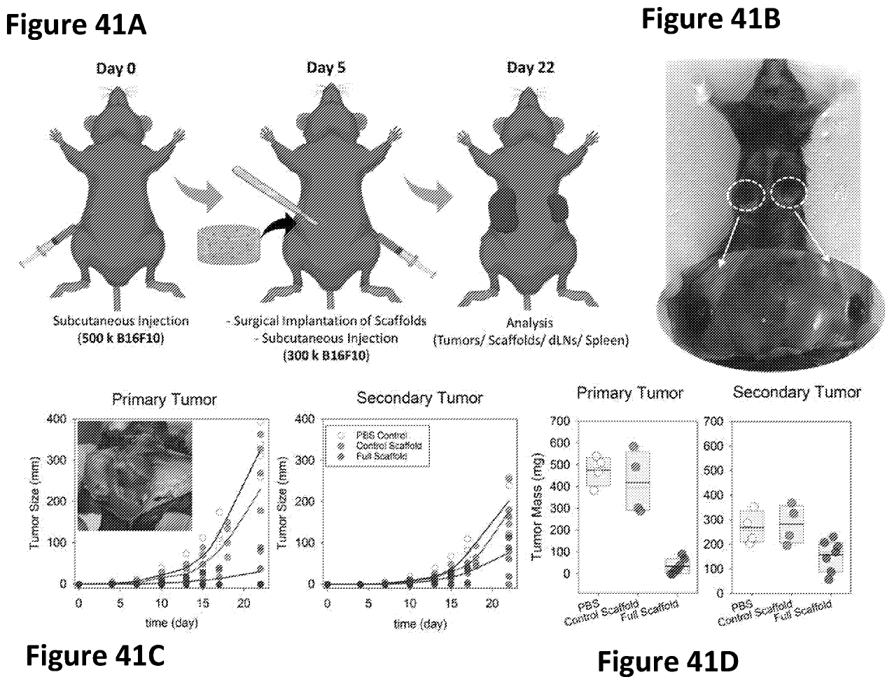

In order to assess the impact of local boosting of T cells adjacent to the primary tumor on formation of systemic immunity, mice were inoculated with a second tumor contralateral to the primary one on the same day on which the scaffolds were implanted (FIGS. 41A-41B). Tumor growth on both sides was monitored, and the tumor mass at the end of the experiments was measured (FIGS. 41C-41D).

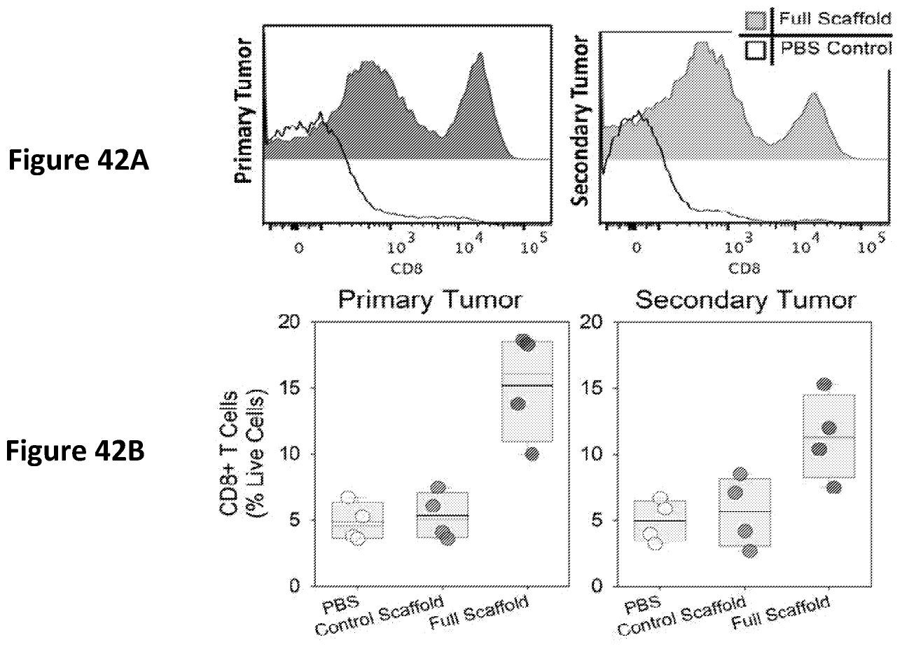

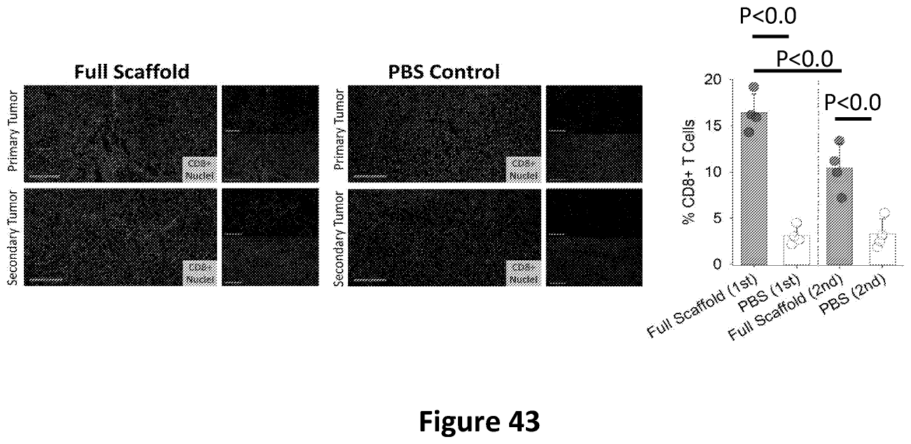

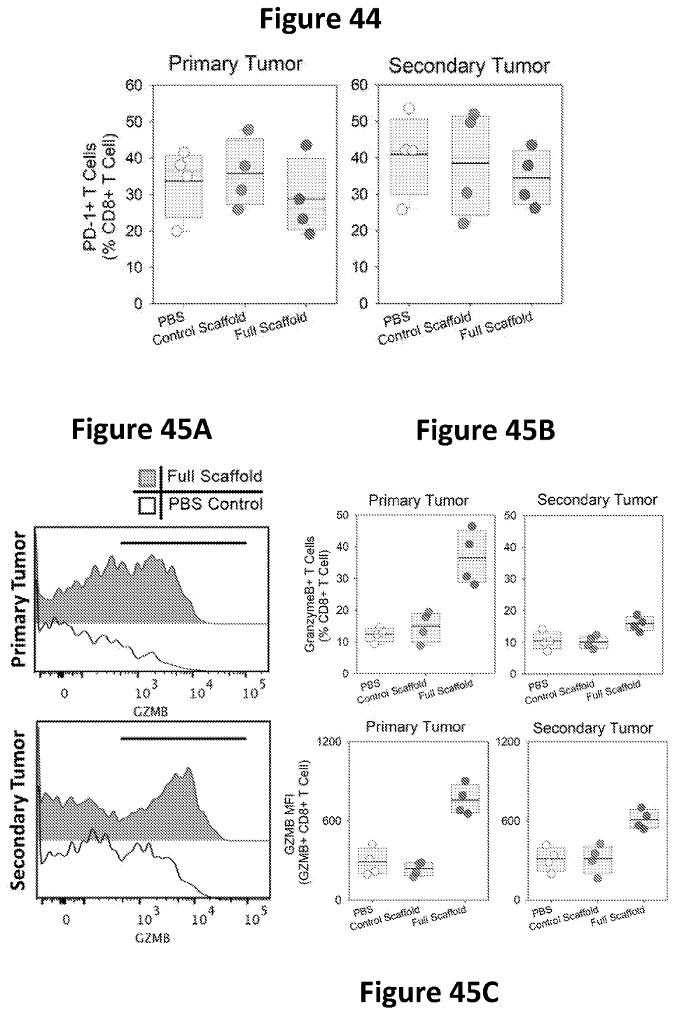

Strikingly, tumor growth in the secondary tumor was suppressed by about 40 percent upon local treatment of the primary tumor with scaffolds. Percentage of tumor-infiltrating CD8+ T cells was increased by more than two times in the contralateral tumor of the mice that received scaffold treatment (FIG. 42). Higher infiltration of CD8+ T cells in both primary and secondary tumors was also confirmed with immunofluorescence staining of tumor sections against CD8 antibodies (FIG. 43). No meaningful differences in the population of PD-1+ T cells were observed in either of the tumors (FIG. 44).

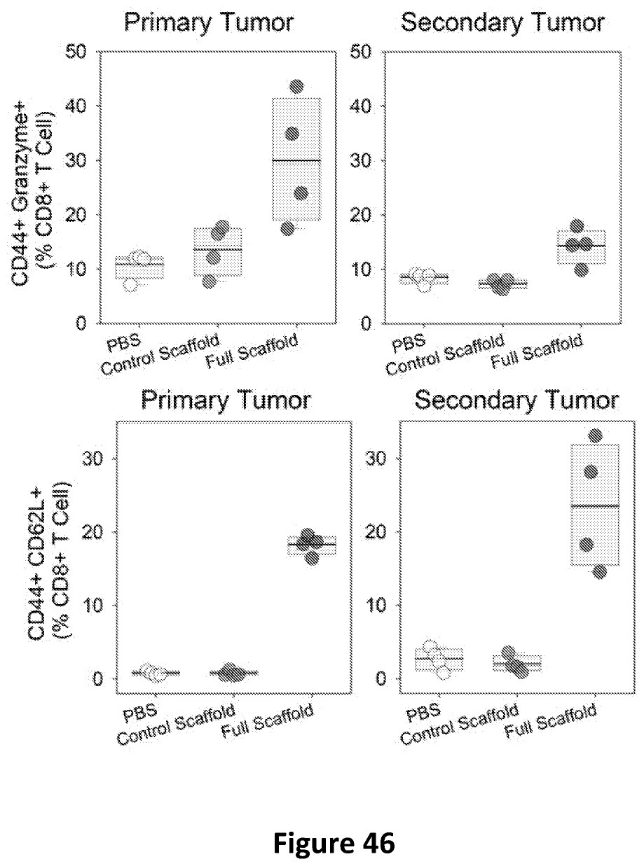

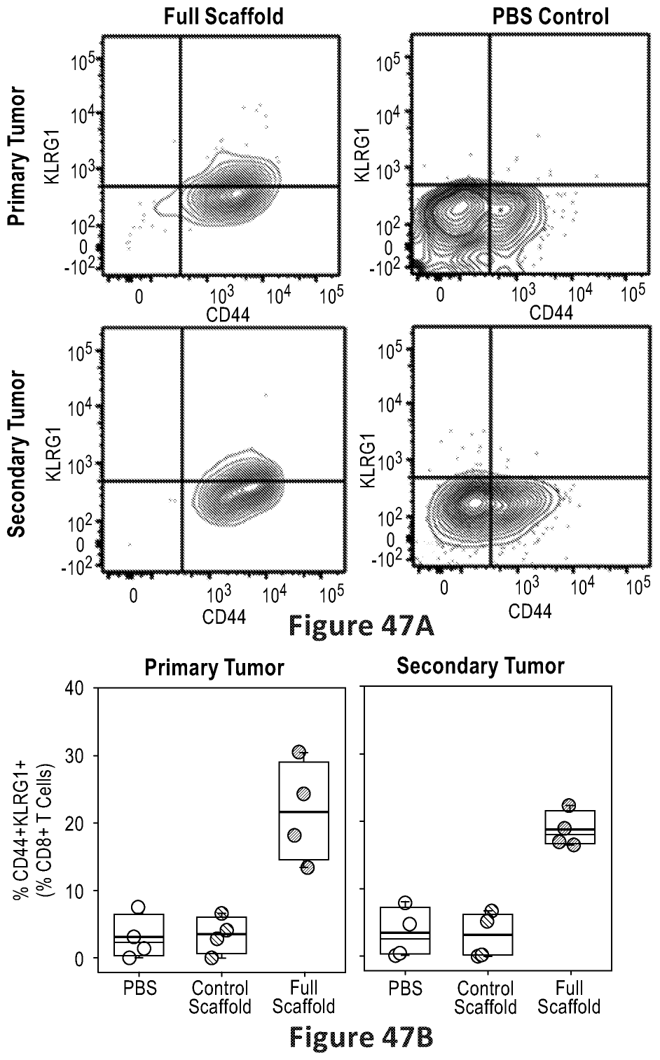

The population of activated GranzymeB secreting CD8+ T cells was considerably improved in the contralateral tumor, as well as primary tumor, which indicates that some of the tumor recognizing T cells that were trained adjacent to primary tumor were able to travel to the distant tumor (FIG. 45, FIG. 46). T memory response was induced in mice treated with full scaffolds as it was reflected in the drastic increase in the frequency of endogenous central memory (CD44+CD62L+CD8+) T cells (FIG. 46). Additionally, the population of short-lived effector CD8+ T cells (KLRG1+CD44+) was also considerably improved in both primary and secondary tumor (FIG. 47).

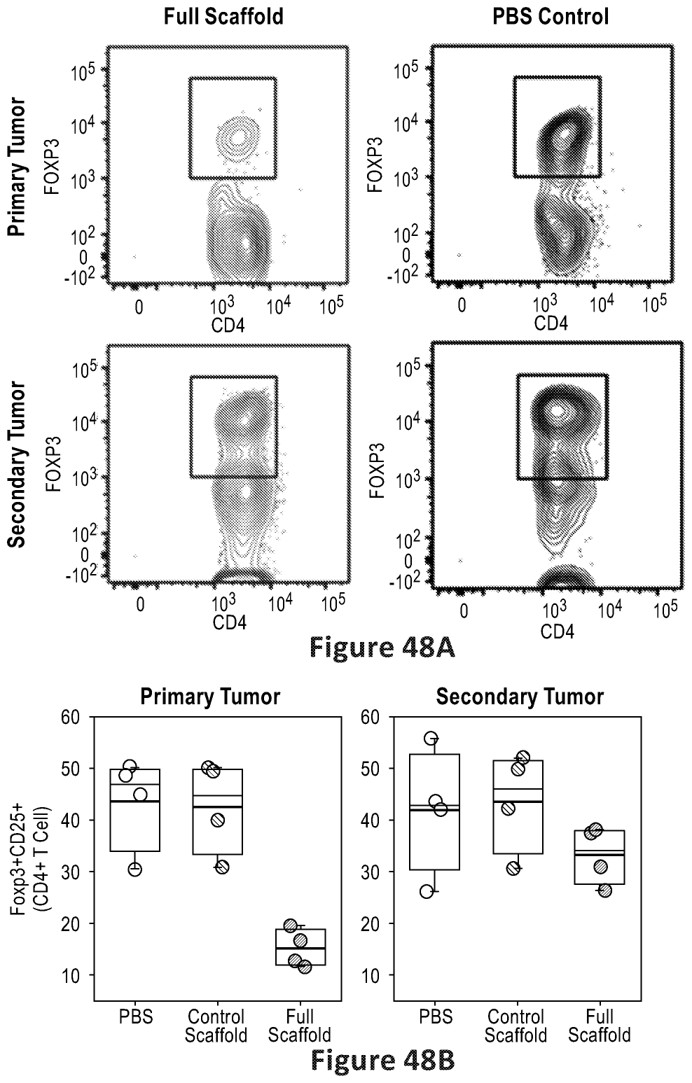

The population of Tregs in both tumors was also studied. Because the release of TGFβi is local to the primary tumor where the scaffold is implanted, suppression of regulatory T cells was only observed in the primary tumor, and no significant difference was noticed in the secondary tumor (FIG. 48). The fact that in the primary tumor scaffold is tackling tumor cells from two angles, one by enhancing the population of tumor reactive T cells and the other by suppressing Treg population, compared to secondary tumor where Treg population is undisturbed partially explains the lower suppression of tumor growth in the secondary tumor.

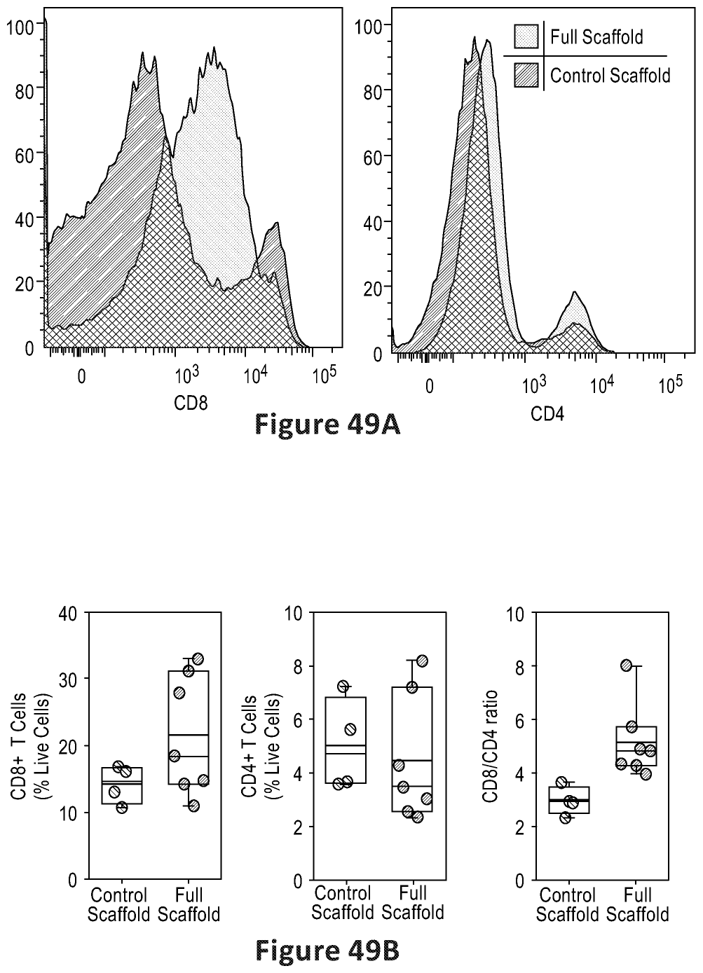

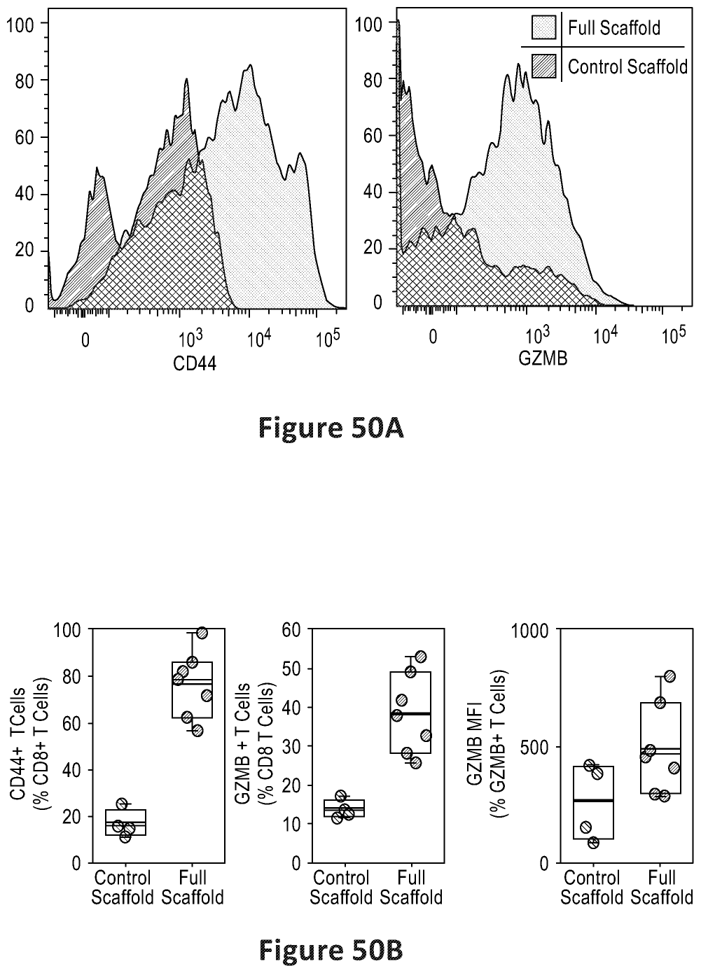

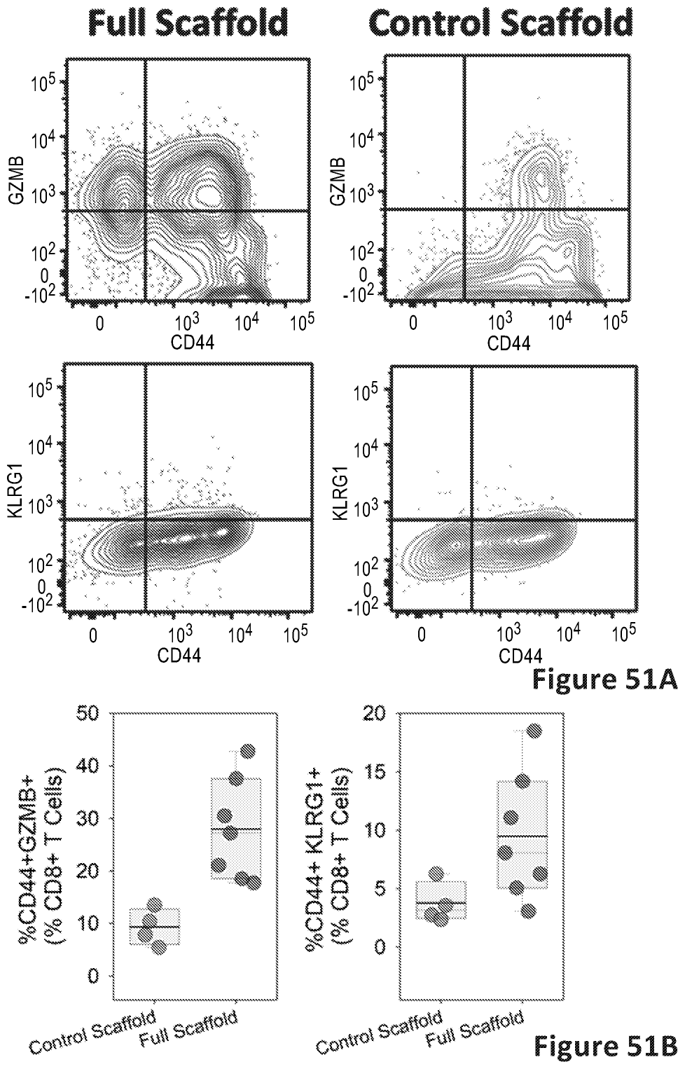

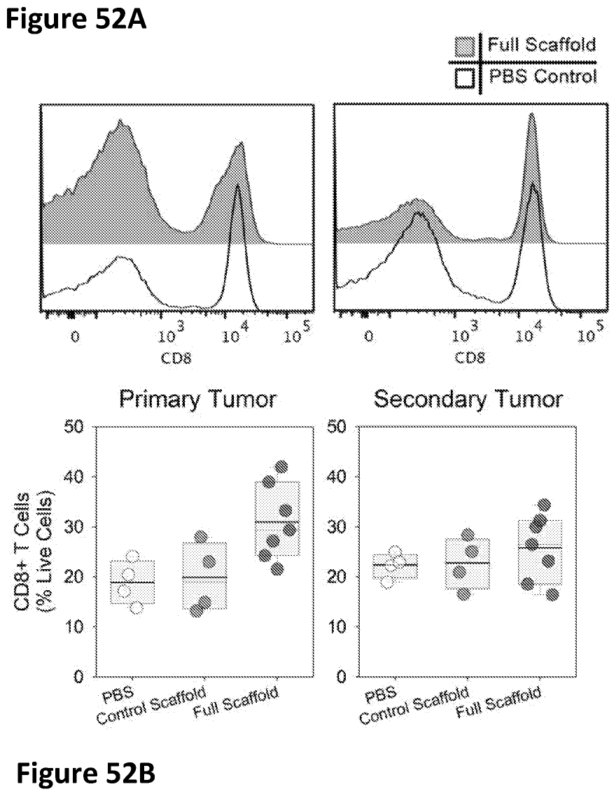

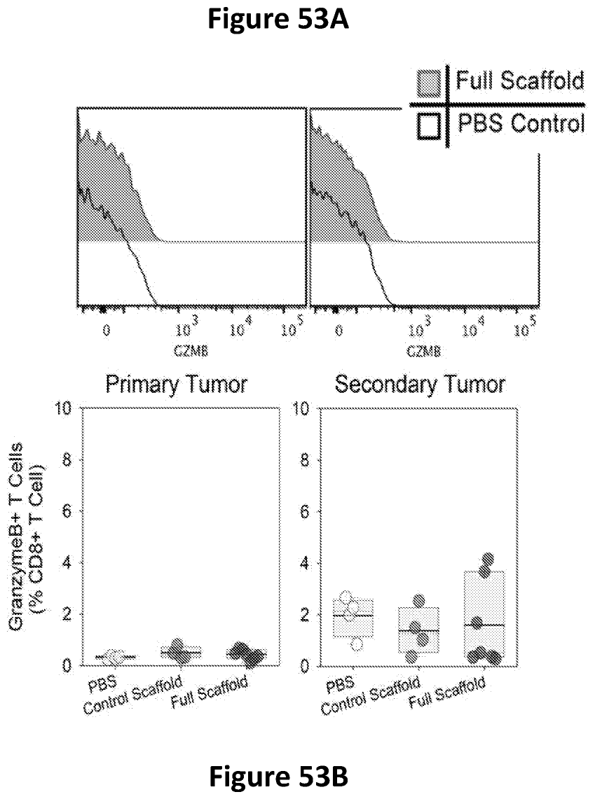

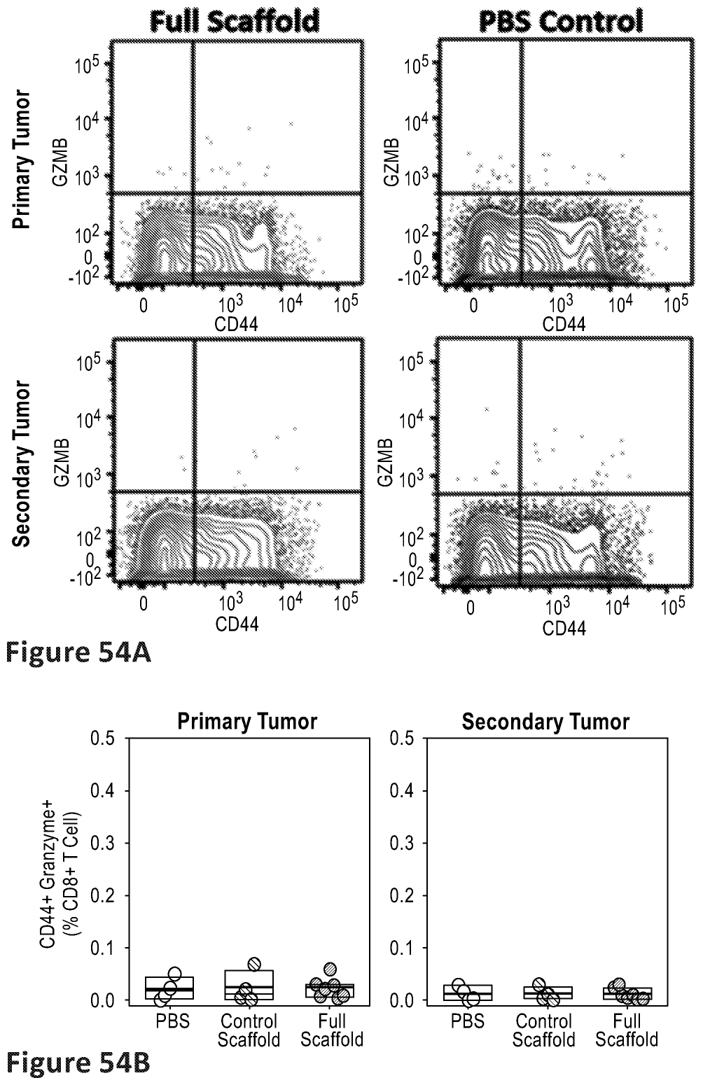

The T cells recruited by scaffolds were studied further (FIG. 49). Similar to previous results implanted scaffold favored CD8+ T cell recruitment and enhanced CD8 to CD4 ratio in the scaffolds (FIG. 49). The population of both activated and GZMB+CD8+ T cells was also noticeably improved (increased) in the full scaffold compared to the control (FIG. 50, FIG. 51). Additionally, short-lived effector T cells identified as CD44+KLRG1+ were also improved (increased) in the full scaffold (FIG. 51). Further, the draining lymph nodes of both primary and secondary tumors were studied for the population of CD8+ T cells and GZMB secreting T cells (FIGS. 52-54). Based on these results, local treatment seemed to put no effect on these populations in the draining lymph nodes.

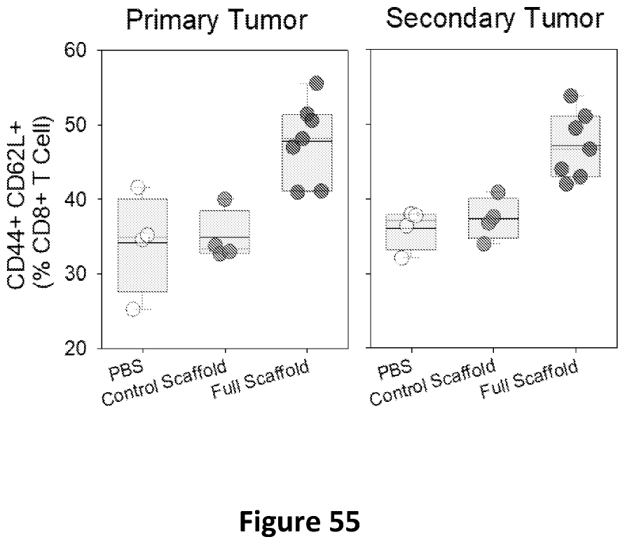

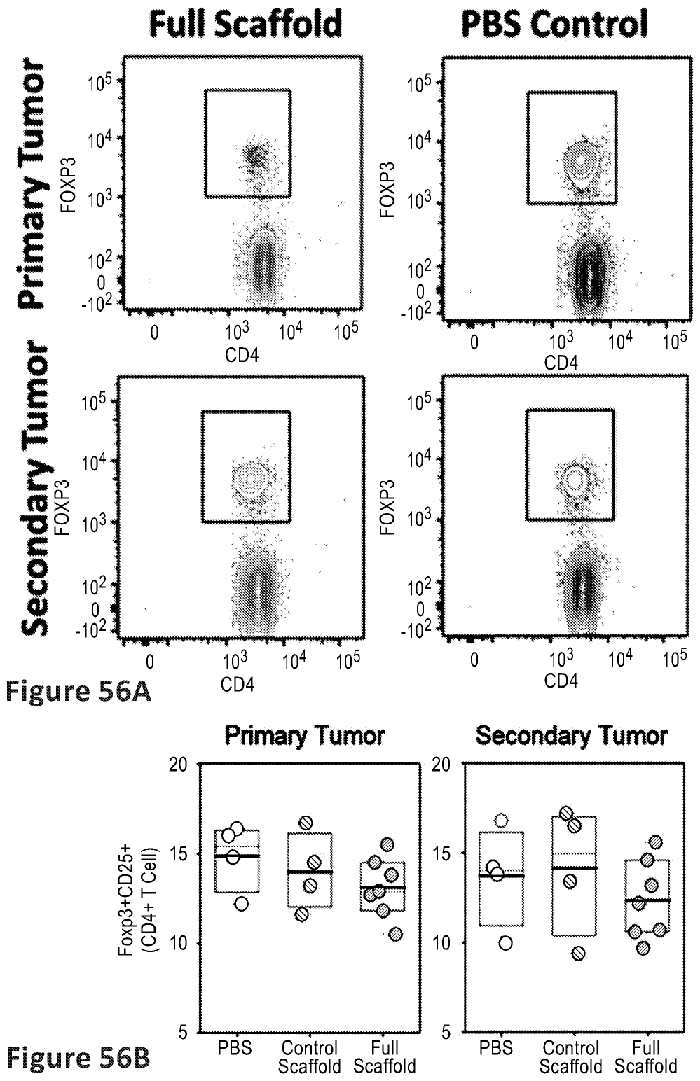

The populations of central memory T cells were also noticeably higher in the draining lymph nodes of both primary and secondary confirming the idea that local treatment has partially resulted in systemic immunization against the tumor (FIG. 55). As demonstrated before, the local release of TGFβi adjacent to the primary tumor showed no significant impact on the population of Tregs even in the draining lymph node of the primary tumor (FIG. 56).

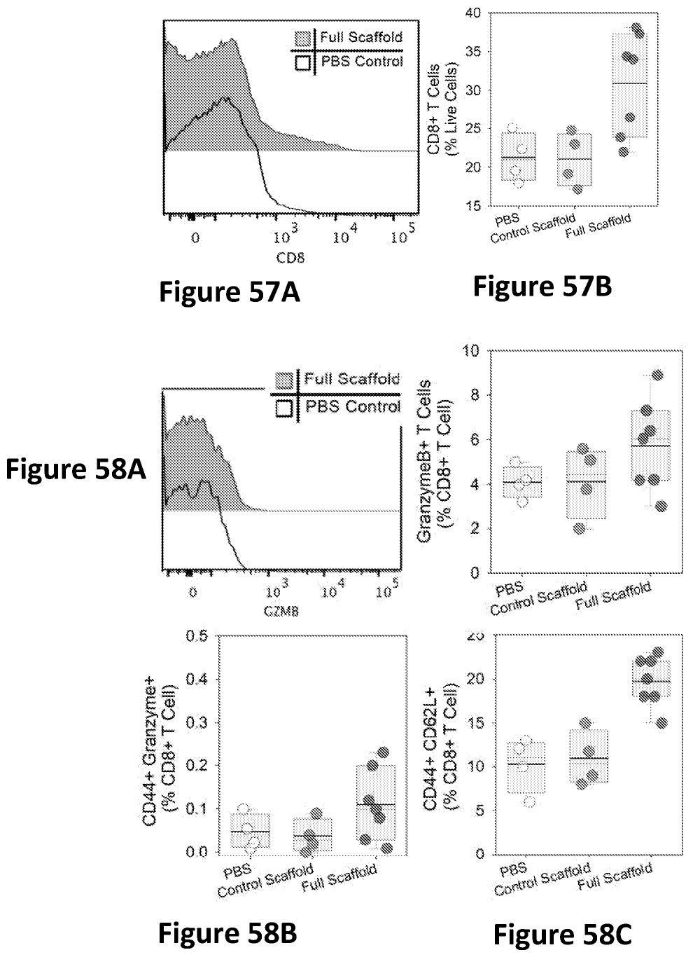

With respect to the spleen, while the population of CD8+ T cells were improved in the mice treated with full scaffolds (FIG. 57), the frequency of activated or GZMB+ T cells was not affected for the tumor-bearing mice receiving the full scaffold treatment. Again, as a results of full scaffold implantation increase in the population of central memory T cells were reflected in the spleen as well (FIG. 58).

### Example 33: ISL Boosts the Efficacy of Adoptive T Cell Therapy

ISL offers the capability of not only facilitating tumor infiltration by T cells and T cell expansion, but also renders the possibility of recruiting naïve tissue/tumor resident T cells and activating them while hampering the immunosuppressive microenvironment of the tumor.

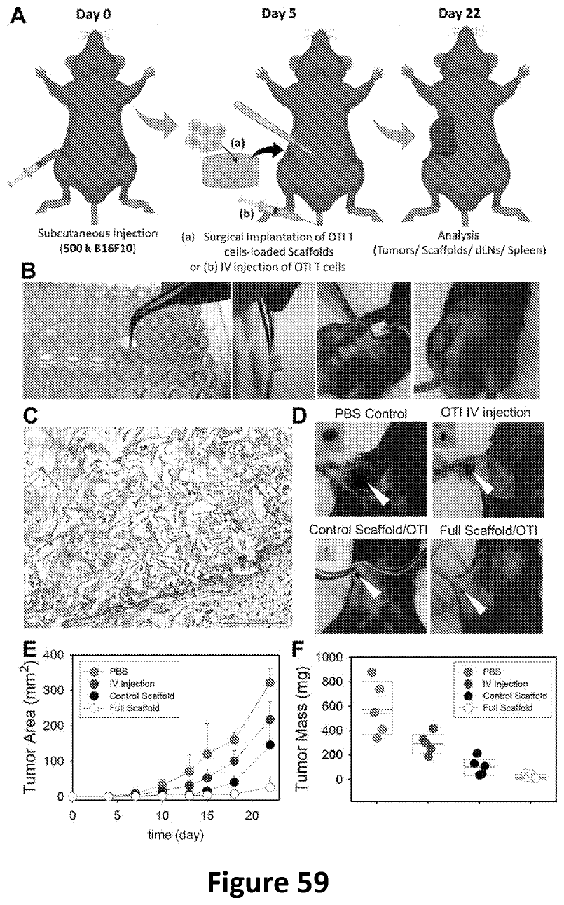

Melanoma model mice were injected subcutaneously with 2×10⁵ (2×105) Ova peptide expressing B16-F10 melanoma cells followed by OTI T cell-loaded ISLs once the tumor was palpable (day 5), when mice were randomized to four groups. Mice were sedated and received a small incision next to the just-palpable tumor and either a TGF-βi or plain Alg-RGD scaffold was inserted (FIG. 59B). The other two groups either received an intravenous (IV) injection of OT-1 cells or just PBS (this last group is still immunoreplete with endogenous T cells). ISLs were made in 96-well plates roughly about the size of a pencil eraser (FIGS. 59B-59C) and were then implanted adjacent to the tumor. H&E staining of the scaffolds adjacent to the tumor confirmed tissue engagement, successful delivery, and proliferation of OT-Is plus recruitment of endogenous T cells (FIG. 59C). (Mice were later euthanized 22 days afterwards for further analysis (FIG. 59A).)

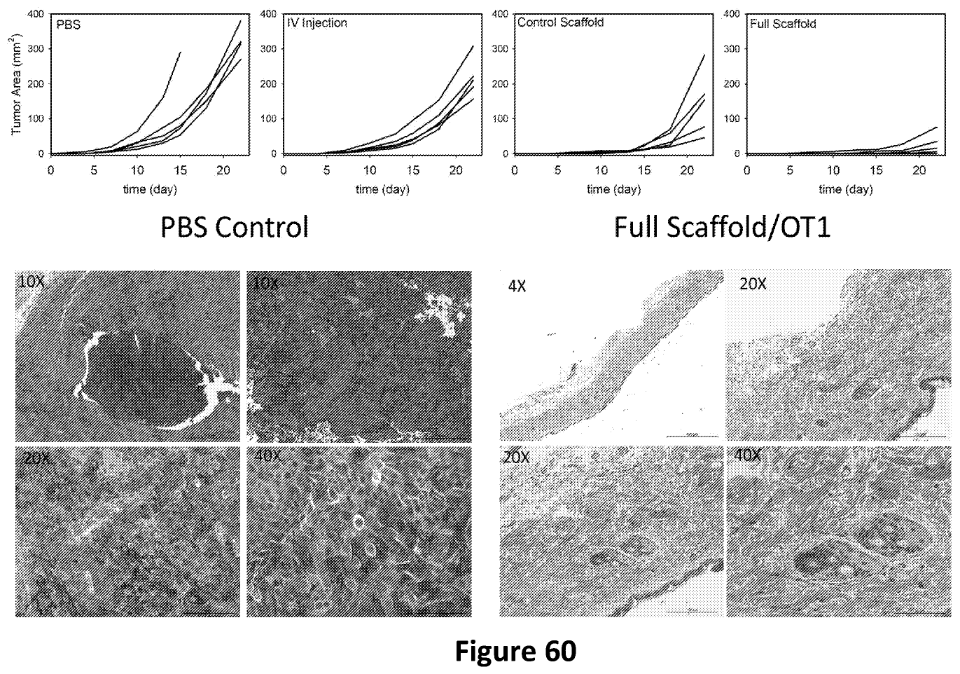

The area and mass of the tumors were then tracked while a blank scaffold (loaded with OT-I CD8+ T cells but free of any modification), IV injection of OT-I T cells, and PBS were used as controls (FIGS. 59D-59F). The OT-I loaded ISL suppressed tumor growth by about 16-fold compared to PBS control and improved growth rate by about 10-fold compared to IV injection of OT-Is (FIG. 59E). The IV injection control here represents the systemic injection of tumor recognizing T cells which due to the poor tumor infiltration loses the fight against cancer cells. On the other hand, control scaffolds used here overcome that issue by local delivery of trained T cells to the tumor but still fail to be as effective as full scaffolds, where besides local delivery of OT-1 s enhances ACT by supporting their expansion (FIG. 59A), recruitment of endogenous T cells and shutting down the induction of Tregs (FIGS. 59E-59F, FIG. 60). Histology images of the tumors treated with full scaffold platforms show significant tumor clearance (FIG. 60).

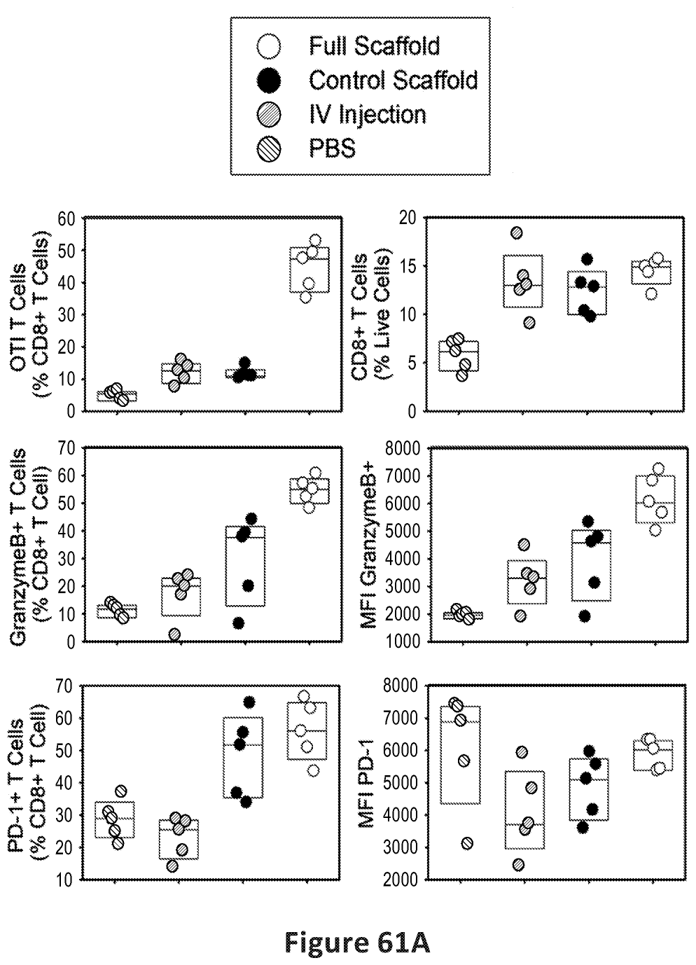

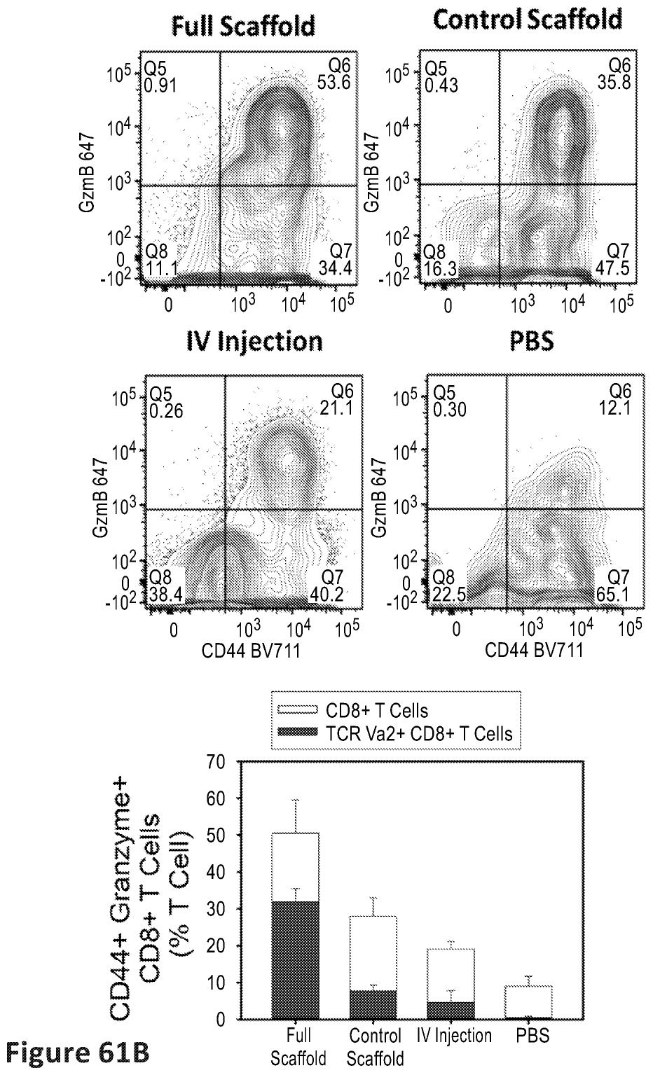

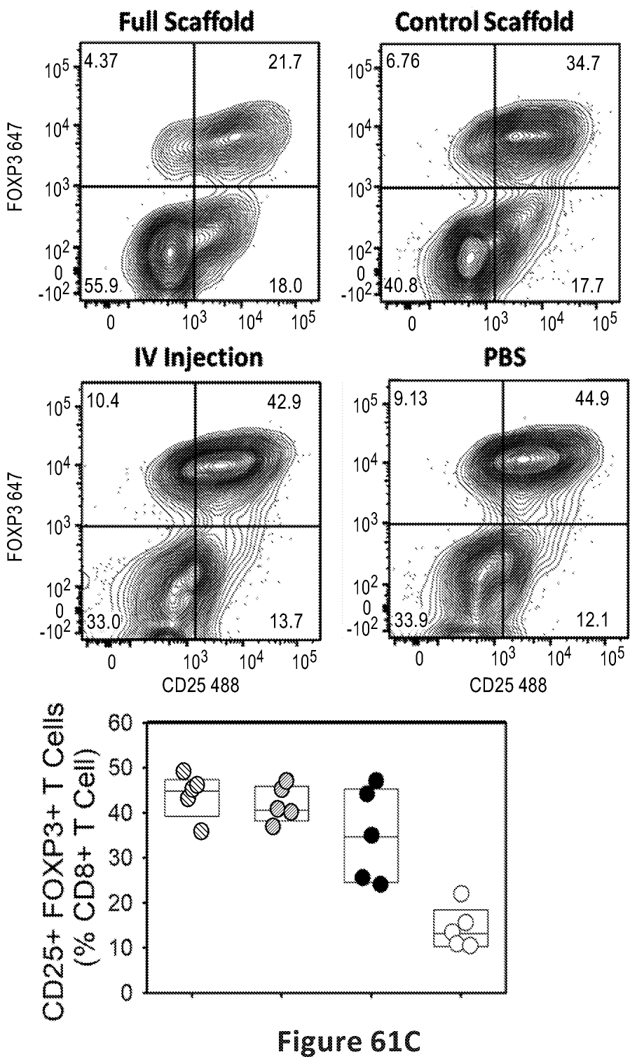

The populations of tumor infiltrating T cells were studied (FIG. 61). An approximately 40 percent increase in the population of tumor infiltrated OT-Is was observed in the full scaffold compared to the control scaffold or IV injection, possibly due to the higher proliferation of the delivered OT-Is along with suppression of Treg induction that allows for their higher tumor infiltration. Moreover, cytokine releasing GZMB+ T cell populations were also approximately 20 percent higher in the full scaffold vs. the control scaffold (FIGS. 61A-61B). The increased population of PD-I+CD8+ T cells in the control and full scaffold also indicates the fact that more T cells in these conditions have experienced tumor antigens. To study the differences amongst Treg populations, the presence of FOXP3+CD25+CD4+ T cells in the tumor was investigated (FIG. 61C). As expected, the Treg population was similar to the PBS control in mice that received an IV injection of OTIs or in control scaffolds where the scaffold served only as a cell transfer platform. On the other hand, regulatory T cells were suppressed by about 40 percent in the full scaffold due to efficient and sustained release of TGFβi. This result demonstrates the importance of tackling a tumor from the twin aspects of empowering tumor fighting T cells as well as weakening the immunosuppressors, promising for ACT therapies. Enhancing the infiltration and delivery of tumor specific T cells has been shown to be insufficient in many cases due to the high population of Tregs in the tumor. The scaffold platform addresses both needs.

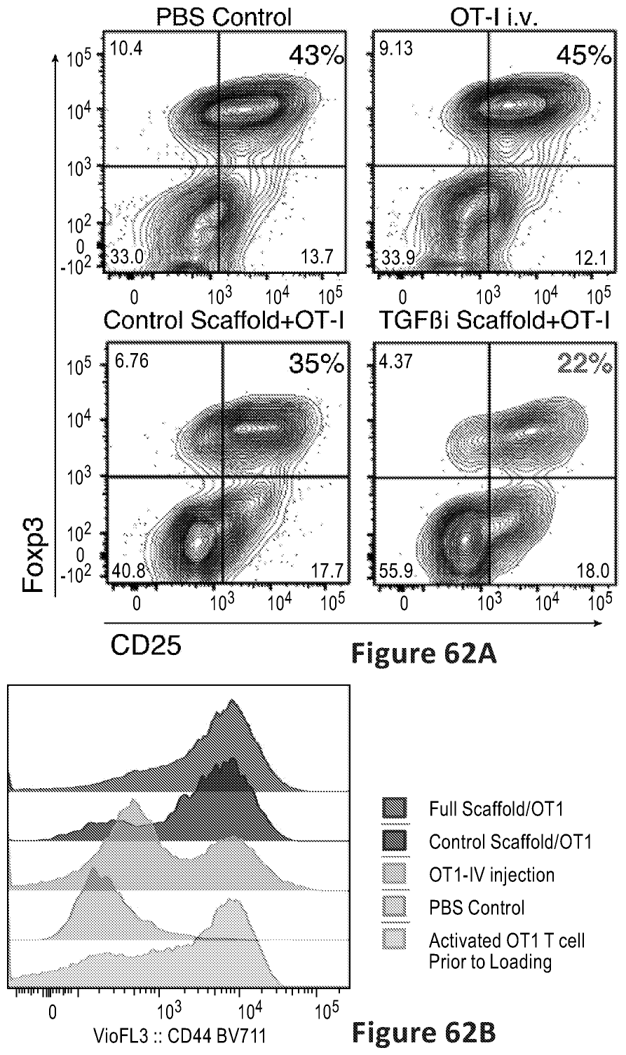

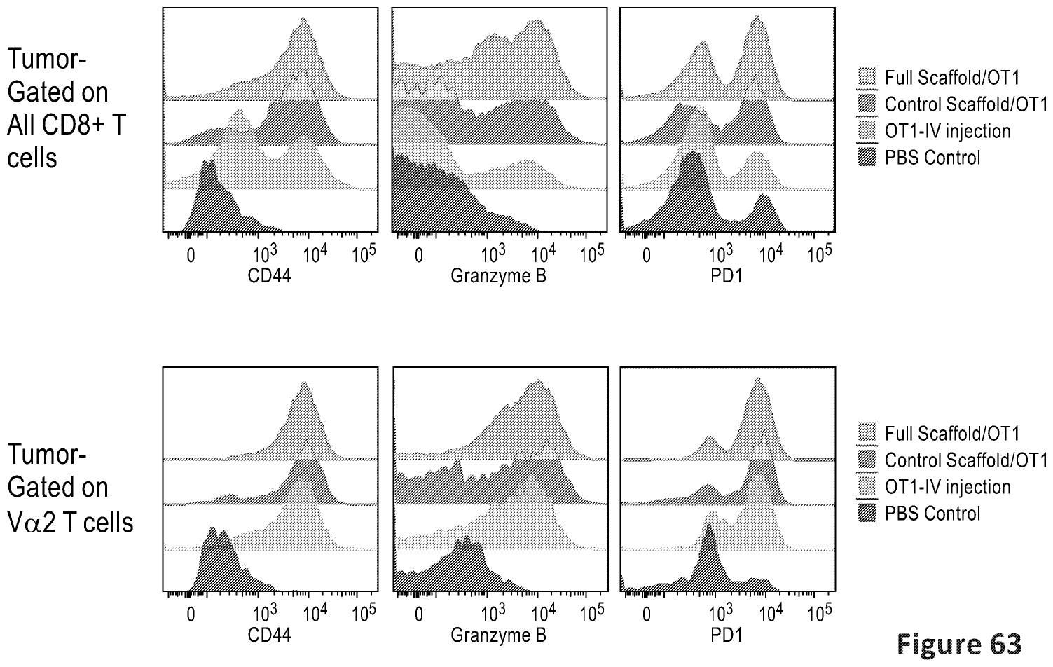

The population of activated tumor infiltrating CD8+ T cells was studied (FIGS. 62A-62B). FIGS. 62A-62B shows the level of CD44 expression as an indicator of activation status 22 days after treating the tumor implanted mice with various therapies. FIG. 63 shows the level of activation markers (CD44, granzymeB, and PD1) expression 22 days after treating the tumor implanted mice with various therapies.

The FACS representatives clearly demonstrated that a considerably smaller number of activated T cells reached and infiltrated tumors upon IV injection of OT1s compared to their local delivery within scaffolds. Treg in the tumors was reduced two-fold and no significant chance in Tregs in the proximal lymph node or spleen (FIG. 62A).

Interestingly, even when gated only on V-alpha2+(Vα2+) OTI T cells present in the tumor, a higher percentage of those preactivated OTI stayed active in the tumor upon local delivery via scaffolds compared to IV injection (FIG. 63). A similar pattern was recorded for GZMB+ T cells in each of those conditions confirming the superiority of local delivery in terms of encouraging higher and more efficient infiltration of tumor fighting T cells. Higher populations of antigen experienced T cells upon local delivery of OTIs were also confirmed by levels of PD-1 expression on T cells (FIG. 63). Activation of intratumoral CD8+OT-1 T cells was increased, as seen by activation markers and GranzymeB expression (FIG. 62, FIG. 63).

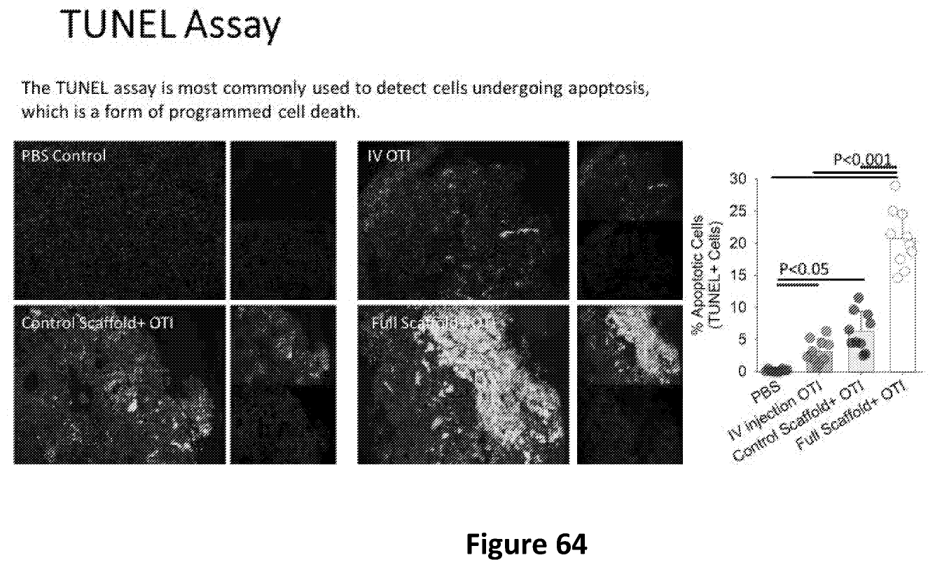

As a measure of tumor clearance, a terminal deoxynucleotidyl transferase dUTP nick end labeling (TUNEL) assay was used to observe DNA degradation in the groups. The TUNEL assay was used as a measure of apoptotic tumor cells where the microscopy images showed drastically higher percentage of tumor apoptosis in the full scaffold that delivered OTIs (FIG. 64). Full scaffold: Alginate-RGD scaffolds, loaded with aAPCs and CCL21, and post-conjugated with anti-CD3 and anti-CD28. Control Scaffold: Alginate-RGD scaffolds.

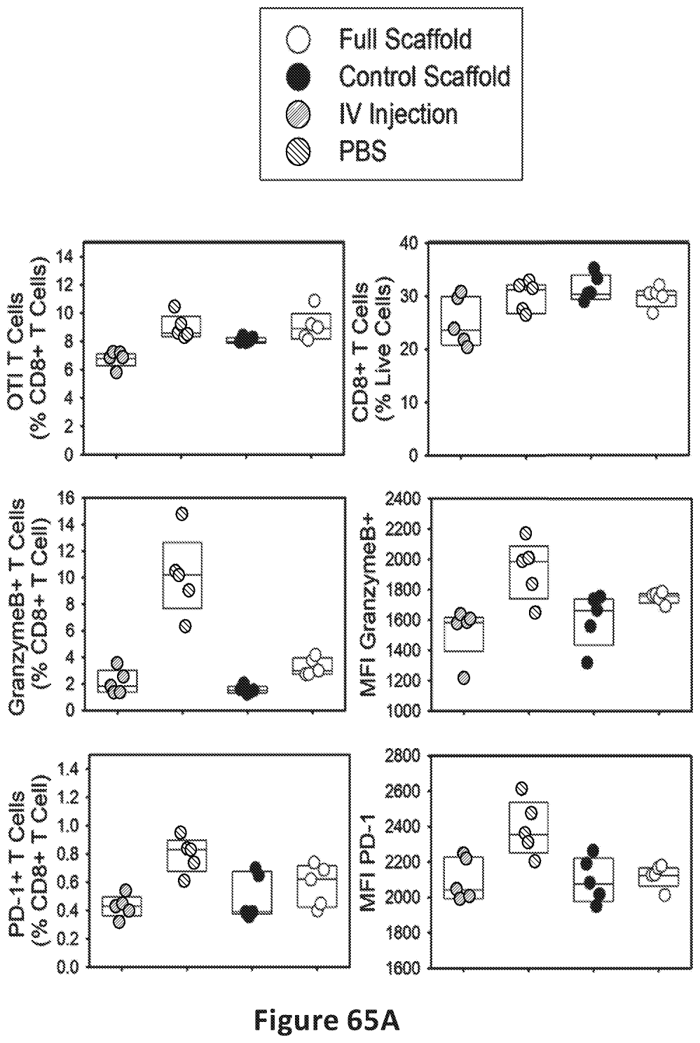

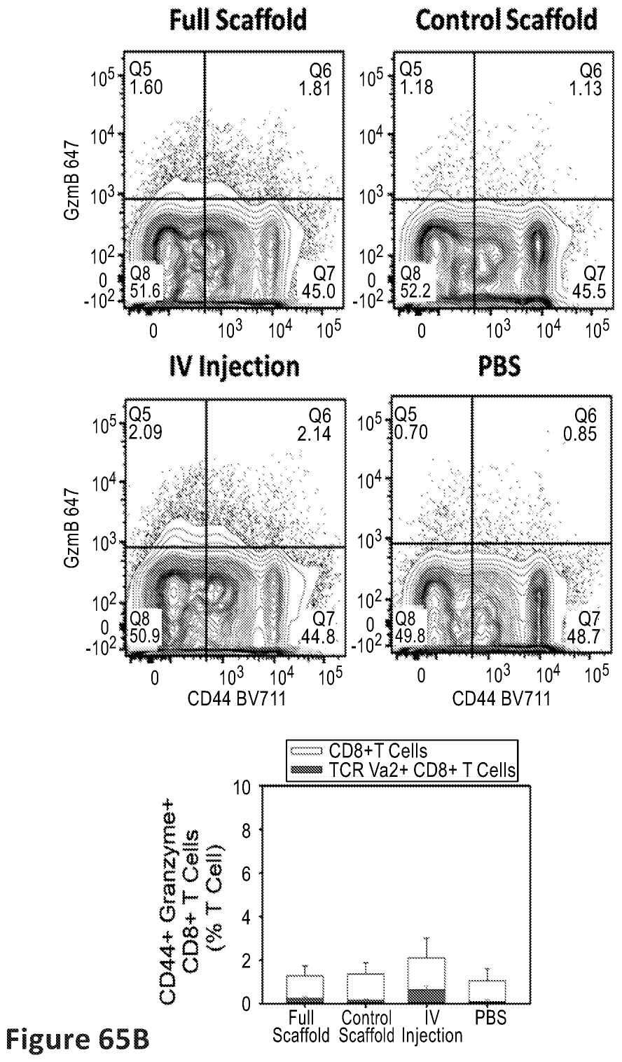

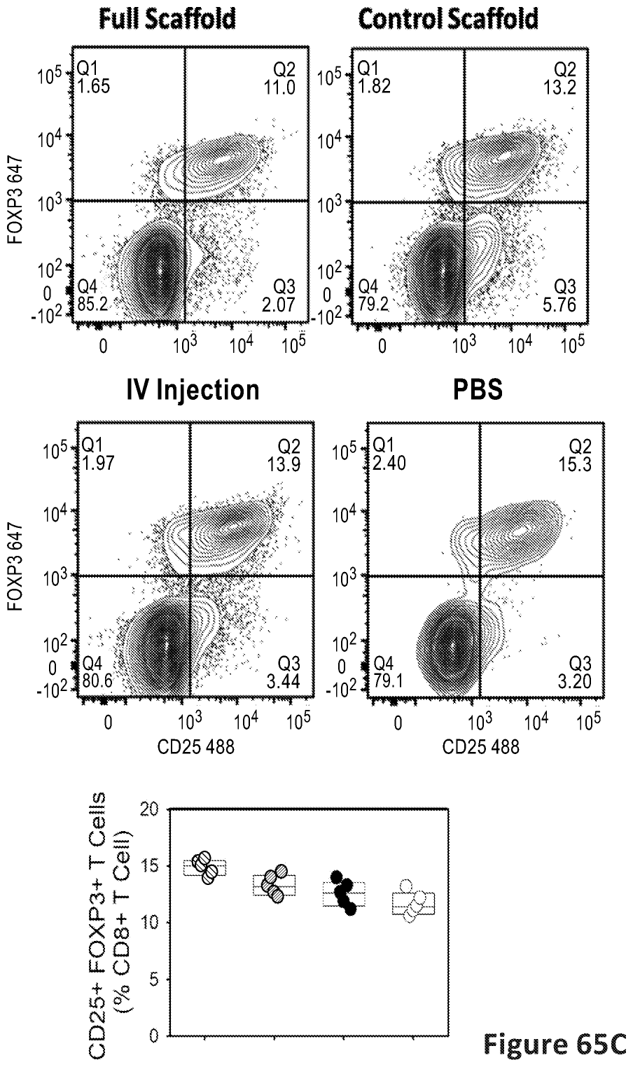

As a measure of the locality of the effects of the scaffolds, the tumor draining lymph node was studied further (FIG. 65). Results showed no difference in the population of OTI T cells or CD8+ T cells in general upon different treatments compared to PBS control. On the contrary, GZMB secreting T cells or PD-1+ population were higher in the case of IV injection of OTIs due to unspecific accumulation of OTIs in tissues other than tumor (FIG. 65A). This data proves the superiority of local delivery as opposed to systemic delivery which can cause several unwanted side effects. The population of CD44+GZMB+CD8+ T cells were also intact in the draining lymph node of scaffold treated mice (FIG. 65B). As another important cell population, important not to affect by local treatment, the percentage of FOXP3+CD25+CD4+ T cells as a measure of Treg population was compared (FIG. 65C). The data showed that the population of Tregs in the tumor draining lymph node was intact compared to PBS control, which eliminated the risk of autoimmune side effects that are normally associated with systemic delivery of TGF-βi drugs.

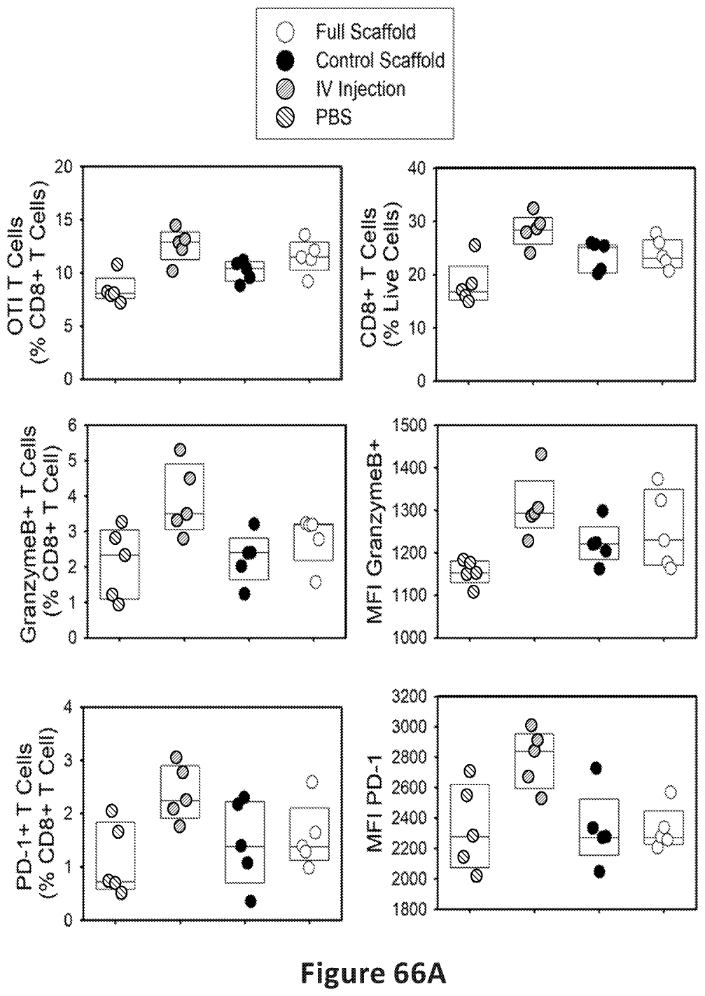

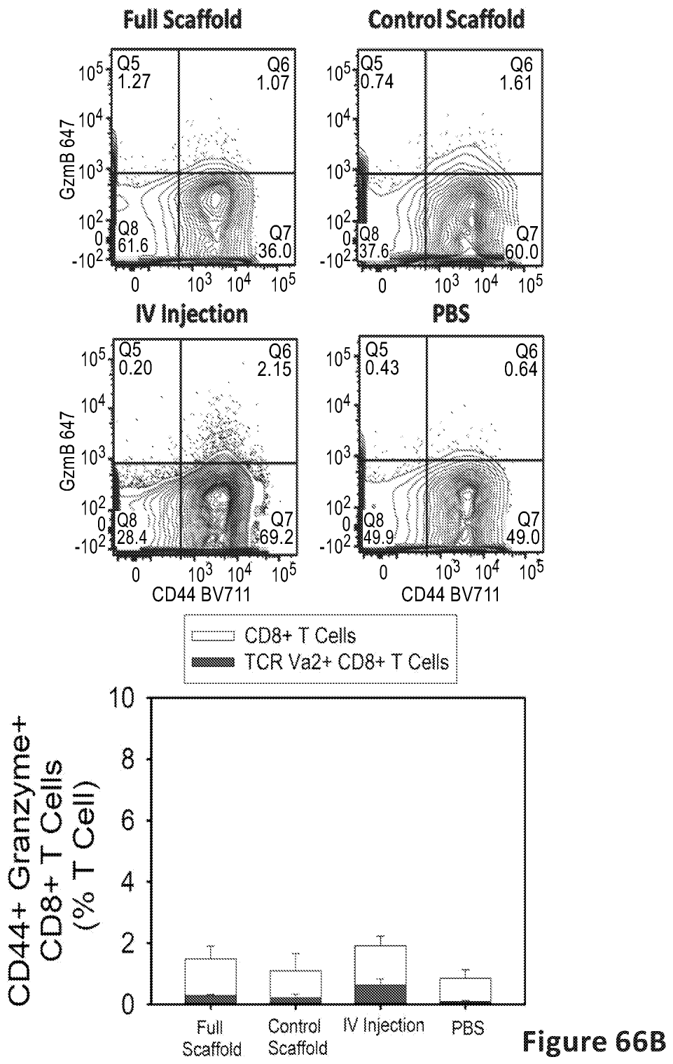

A similar trend was also observed in the spleens of mice treated with different formulations, where no meaningful differences were noted in the population of CD8+s, GZMB secreting T cells, and PD-1+ T cells in the mice treated with scaffolds, as compared to PBS controls, while IV injection of OTIs increased the population of GZMB+ T cells or PD-1 expressing T cells (FIG. 66).

These studies show that the full scaffold had effects on the tumor but no effects on a distal lymph node or spleen. Thus, the local and not systemic activity of the scaffold of the invention is demonstrated.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### Example 34: Engineered, Localized Depletion of Regulatory T Cells Produces Global Anti-Tumor Immunity

### Materials and Methods

### Chemicals and Biologicals

Unless noted otherwise, all chemicals were purchased from Sigma-Aldrich, Inc. (St. Louis, Mo.). All glassware was cleaned overnight using concentrated sulfuric acid and then thoroughly rinsed with Milli-Q water. All the other cell culture reagents, solutions, and dishes were obtained from ThermoFisher Scientific (Waltham, Mass.), except as indicated otherwise.

### Preparation and Characterization of Cytokine-Producing Microparticles

Monodisperse mesoporous silica microparticles (5 to 20 μm) were formed using a microfluidic jet spray-drying process, using cetyltrimethylammonium bromide (CTAB) and/or Pluronic F127 as templating agents, and tetraethylorthosilicate (TEOS) for silica as reported before. To conjugate heparin to the particles, amine groups were first added to the particles. Mesoporous silica microparticles (800 mg) were suspended in dehydrated methanol (50 mL), then (3-Aminopropyl)triethoxysilane (APTES) (3 mL) was added and stirred at room temperature overnight. The final product was centrifuged (1500 rpm, 3 min) and washed with methanol five times, followed by drying under high vacuum. To functionalize the aminated-silica particles with heparin, heparin sodium salt (216 mg) was dissolved in deionized water (8 mL) and activated via successive addition of EDC (63 mg) and sulfo-NHS (71.4 mg). After stirring for 5 min, the ethanolic solution of amino-functionalized silica (20 mg in 1.12 mL) was added to the reaction mixture and stirred for 12 h at room temperature. Afterwards the particles were separated by centrifugation and washed several times with deionized water and ethanol to remove unreacted reagents.

To incorporate IL-2 (Interleukine-2, BRB Preclinical Repository, NCI, NIH, Frederick, Md.) into these heparin-coated microparticles, the microparticles were incubated with different amounts of cytokine in PBS buffer containing bovine serum albumin (BSA; 0.1% w/v) and were gently shaken overnight at 4° C. The microparticles were then centrifuged and washed several times to remove unabsorbed cytokines.

To estimate the in vitro release of IL-2 from microparticles, 20×10⁶ microparticles were incubated in 2 mL PBS (pH 7.4; supplemented with 1 mM CaCl2) at 37° C. At different time intervals, 500 μL of the supernatant was collected, and replaced with an equivalent volume of PBS. The concentration of released IL-2 was determined using a human IL-2 ELISA kit (ThermoFisher). The final particles used for formulating scaffolds (below) employed 50 μg of IL-2 per mg of particles.

In some experiments, the silica particles were also covalently conjugated with T-cell activating antibodies: anti-CD3 (clone 2C11; Bio-X-Cell) and anti-CD28 (clone 37.51; Bio-X-Cell). One μg anti-CD3 and 0.25 μg anti-CD28 were used per 10⁶ silica particles. After activation of antibodies' carboxylic groups for 10 min with EDC/NHS, microparticles were added and incubated under gentle stirring at 4° C. overnight. The functionalized microparticles were then separated from the solution and washed several times. Unreacted functional groups were quenched by washing samples in Tris buffer (100 mM, pH 8) for 30 min. Micro-BCA assay was used to quantify total amount of surface conjugated antibodies according to the manufacturer's protocol.

### TGF-β Inhibitor Particles

Poly(lactic-co-glycolic) acid (PLGA) nanoparticles (NPs) were prepared to release the TGF-β inhibitor LY2157299 (also called galunisertib, Cayman Chemical). PLGA was prepared using a nanoprecipitation method as previously reported: briefly, Resomer RG 503 PLGA (50:50; molecular weight: 28 kg/mol) was used in this study. LY2157299 and PLGA were dissolved in 5 mL dichloromethane and sonicated into 1% poly vinyl alcohol (PVA) solution (50 mL) by probe sonicator (12 W) for 2 min. The resulting emulsification was then added to 100 mL of 0.5% PVA solution. The solution was agitated, and the dichloromethane was allowed to evaporate for 4 h. The solution was then centrifuged at 3000×g for 5 min to pellet out any non-nano size material. The supernatant was removed and ultracentrifuged and washed three times at 21,000 g for 20 min to wash away the PVA. The resulting nanoparticle solution was flash frozen in liquid nitrogen and lyophilized for 2 days prior to characterization and use. Hydrodynamic diameter and surface charge of formed PLGA NPs were studied using dynamic light scattering (DLS) and zeta potential measurements (Zetasizer Nano, Malvern, UK).

### Preparation and Characterization of Scaffolds

To form the scaffolds, the alginate (MW ˜250 kDa, high G blocks; Novamatrix UP MVG, FMC Biopolymer, Rockland, Me.) was first oxidized with sodium periodate (1.5%), overnight at room temperature, then the reaction was quenched by dropwise addition of ethylene glycol for 45 min. The solution was then dialyzed (MWCO 3.5 kDa) against deionized water for 3 days followed by lyophilization. Afterward, the alginate was dissolved in MES (MES 150 mM, NaCl 250 mM, pH 6.5) and covalently conjugated to RGD-containing peptide (GGGGRGDY; GenScript USA Inc., Piscataway, N.J.) using carbodiimide chemistry (NHS/EDC). The reaction was continued for 24 h followed by dialysis (MWCO 20 kDa) and lyophilization. This alginate-RGD complex in PBS was mixed with CCL21 protein (Peprotech Recombinant Murine Exodus-2, 250 ng per scaffold). To prepare scaffolds, 20×10⁶ IL-2-loaded heparin-functionalized silica microparticles were mixed with 1 mL of alginate. The alginate/chemokine/cytokine-particle/TGF-βi-NP mixture was cross-linked using calcium sulfate solution. Each resulting scaffold bore 400 IU of IL-2. The gels were casted in desired 24- or 96-well plates followed by two overnight washes to remove extra calcium ions. These scaffolds were frozen at −80° C., lyophilized for 3 days, and stored at 4° C. before use.

To load these NPs into alginate-based scaffolds, LY2157299-loaded PLGA NPs were mixed with alginate prior to crosslinking via calcium. The concentration of released LY2157299 from nanoparticles before and after loading into alginate scaffolds was determined by measuring the UV absorption of LY2157299.

An array of different alginate formulations was prepared by varying either the polymer content or the amount of crosslinker (here CaSO₄). To measure the mechanical stiffness of the gels, an Instron 5542 mechanical tester was used, and all the samples were tested at a rate of 1 mm/min. The Young's modulus was then calculated from the slope of the linear region that corresponds with 0-10% strain. The final alginate formulation employed for in vivo studies comprised alginate 2.5% with 40 mM CaSO₄.

Calcium measurement in the scaffold. Some calcium is sequestered into the alginate matrix for crosslinking, and some is extra. A 3-day wash (two overnights with change) was performed by immersing in PBS (PBS+0.42 mM CaCl₂). The calcium content of the scaffolds before and after washing was measured. The scaffolds were also immersed after washing into in vitro culture media (RPMI+10% FBS+antibiotics and amino acids) for 3 and 7 days. Then the calcium content of the scaffolds was measured. For all these measurements, scaffolds were lysed using EDTA (50 mM) and alginate lyase (porcine, SigmaAldrich, 3.4 mg/mL). Then dissociation of the EDTA-Ca2+ complex was triggered and released calcium ions for measurement by addition of acetic acid (0.05 vol %). Finally, the calcium content was measured using a Calcium Colorimetric Assay Kit (BioVision).

To immobilize anti-CD3 and anti-CD28 to the scaffolds, the freeze-dried scaffolds were activated with EDC/NHS for 15 min. Then the scaffolds were washed twice with PBS (supplemented with 0.42 mM CaCl₂) before addition of anti-CD3 and anti-CD28. Then they were incubated at 4° C. overnight. Unreacted functional groups were quenched by washing the scaffolds with Tris buffer (100 mM, pH 8) for 30 min. For T-cell activation studies, 5×10⁶ primary naïve T cells were added to the scaffolds and cultured for 3-5 days to study their effector functions.

CCL21 release from the scaffolds was measured by murine CCL21 ELISA kits as a function of time.

To sterilize the fabricated scaffolds before in vitro or in vivo functional assays, X-ray irradiation (Gulmay Medical RS320 X-ray unit) was employed as per ISO 11137-2:2013 recommended protocols. A dose of 25 kGy (2.5 Mrads) was used for sterilization. Physical properties, including changes in morphology and mechanical stiffness of the scaffolds, or T-cell activation property change after sterilization was tested.

The endotoxin level of digested scaffolds was measured using ToxinSensor™ Chromogenic LAL Endotoxin Assay Kit (Genscript). Each scaffold was dissolved in 1 mL of digestion buffer and the endotoxin level was calculated to be around 0.14±0.02 endotoxin units/ml. The FDA Center for Devices and Radiological Health has adopted the USP Endotoxin Reference Standard and limits the acceptable level of endotoxin contamination for medical devices to 0.5 endotoxin units/ml. Thus, our scaffolds contained a very low level of endotoxin at a level that is FDA-allowable.

Scanning electron microscopy (SEM) images of the gels were taken to see the cross-sectional microstructure and porosity of the alginate-based scaffolds. The lyophilized scaffolds were freeze-fractured (using liquid nitrogen) for cross-sectional images. The scaffolds were sputtered with iridium (South Bay Technology Ion Beam Sputtering) prior to imaging with a ZEISS Supra 40VP scanning electron microscope (Carl Zeiss Microscopy GmbH). The sizes of pores from different parts of the SEM images were then measured and analyzed using ImageJ software (NIH). For SEM imaging of cell-loaded scaffolds, the cell-laden hydrogels were fixed with 2.5% glutaraldehyde, followed by post-fixation in osmium tetroxide prior to serial dehydration in increasing concentrations of ethanol (25, 50, 75, 90, and 100%) for 15 min each, and iridium sputtering.

Confocal microscopy was used to study the association of T cells with the scaffolds. For these experiments, alginate was stained with amine-fluorescein prior to hydrogel formation. T cells were incubated with the scaffold in 8 well-plate Labtek II chambers for 30 min prior to fixation and staining. The supernatant was removed from Labtek chambers, and then scaffolds were fixed with 4% PFA. Then to permeabilize cells for intracellular staining, 0.1% Triton X-100 in PBS was incubated with cells for 5 min. To stain the cells, Alexa Fluor 568-conjugated phalloidin (Life Technologies) was used. After several washes, samples were covered with Fluoromount-G with DAPI (eBioscience) and stored at 4° C. before imaging with the Leica Confocal SP8-STED microscope. Images were analyzed using Leica software, Imaris, and ImageJ/Fiji.

### T-Cell Isolation and Activation

All in vitro experiments were conducted in accordance with UCLA's institutional policy on humane and ethical treatment of animals following protocols approved by the UCLA Animal Research Committee. Five- to eight-week-old wild-type or OT-I TCR transgenic mice (Jackson Labs) were used for all experiments.

T cells were purified from spleens using the EasySep immunomagnetic negative selection enrichment kit (Stem Cell Technologies). T cells were cultured in media comprising RPMI-1640 supplemented with 10% heat-inactivated FBS, 1% penicillin/streptomycin, 1% sodium pyruvate, 1% HEPES buffer, 0.1% μM 2-mercaptoethanol.

In vitro activation of T cells was performed by culturing 1×10⁶ cells/mL in tissue culture-treated 24-well plates that were pre-coated with anti-CD3 (clone 2C11; Bio X Cell) at a concentration of 10 μg/mL plus addition of 2 μg/mL soluble anti-CD28 (clone 37.51; Bio X Cell). T cells were then collected from wells and allowed to proliferate in interleukin-2 (IL-2, BRB Preclinical Repository, NCI, NIH)-containing medium (50 U/mL), prior to being used for experiments.

To study T cell proliferation and function, T cells were recovered from scaffolds at selected time points by digesting the scaffolds using EDTA (50 mM) and alginate lyase (porcine, SigmaAldrich, 3.4 mg/mL) for 20 min. The number of viable T cells recovered from the scaffolds was determined by Trypan Blue exclusion.

For Treg formation experiments CD4+ T cells were purified from mouse spleen as mentioned above. Cells were then either activated on scaffolds or on anti-CD3e antibody (8 μg/mL) coated plates with the anti-CD28 antibody (2 μg/mL) supplemented medium. At the same time TGF-β (15 ng/mL) was added to the media. After four days regulatory T cells were removed, fixed, permeabilized, and stained with antibodies for flow cytometric analysis of Foxp3 expression.

### Flow Cytometry

For flow cytometry analysis, antibodies to mouse antibodies were purchased from eBioscience, BioLegend, or BD Biosciences. To study proliferation behavior of T-cell responses during various treatments their expansion was measured by 5-(and-6)-carboxyfluorescein diacetate, succinimidyl ester (CFSE) dilution. For CFSE dilution experiments, 5×10⁵ naive CD4+/CD8+ T cells were labeled with 2 μM CFSE for 13 min, followed by two washes and then incubation with splenocytes. Splenocytes were extracted from the spleens of wild type C57Bl/6 mice. Then the cells were incubated in ACK lysis buffer (Gibco) for 5 min at room temperature to remove red blood cells. The remaining cells were then treated with ova peptide as above to present to T cells. Cells were analyzed on a Cytek DxP10 flow cytometer using FlowJo software (Treestar/BD).

For intracellular staining of GranzymeB and Foxp3, the manufacturer's protocol (Foxp3/Transcription Factor Staining Buffer Set, eBioscience) was followed. The following antibodies were used for intracellular staining from Biolegend: Foxp3 (clone MF-14, AF647, Cat #126408); GranzymeB (clone GB11, AF647, Cat #515406), Mouse IgG1, κ Isotype Ctrl (clone MOPC-21, AF647, Cat #400130).

### Migration Assay

The migration assay to evaluate the role of chemokines on recruitment of T cells and melanoma cells in the presence and absence of chemokine-functionalized scaffolds was performed using regular Transwell migration as reported. The number of migrated cells was evaluated after 4 h using an automatic cell counter.

### Breast Cancer Model

5×10⁵ 4T1-Luc or 4T1 cells (a triple-negative breast cancer cell line) were injected orthotopically into the mammary fat pad of female wild-type BALB/c mice (6-8 weeks old, Jackson Lab). The growth of tumors was monitored by measuring the bioluminescence signal with the IVIS Imaging System (Perkin Elmer) and/or using a digital caliber. Stable signals were determined by monitoring the photon count up to 30 min after intraperitoneal injection of luciferase substrate, D-luciferin (15 mg/mL diluted in PBS), into animals anesthetized with inhaled 2.5% isoflurane. Upper body metastases were also assessed in the same way on day 30 after covering the primary tumor sites. The Living Image software was used to analyze bioluminescence flux.

At 100 days post injection of primary tumor, surviving mice were rechallenged with retroorbital (i.v.) injection of 2×10⁵ 4T1-Luc tumor cells in 100 μL of HBSS. Development of metastases in lung was monitored by bioluminescence imaging.

The immunological abscopal effects were also tested for the breast cancer model. Immunoactive scaffolds and PBS control were prepared. Wildtype, immunocompetent syngeneic mice were implanted in the mammary fat pad with 5×10⁵ 4T1-luc breast cancer cells. After 5 days, the scaffolds were implanted and 3×10⁵ tumor cells were injected to the contralateral mammary fat pad. Tumor growth was then tracked using bioluminescence imaging.

### Melanoma Model

2-5×10⁵ B16F10-OVA or B16F10 tumor cells were subcutaneously injected into right or both (in the contralateral tumor model) right and left flanks of wild-type C57BL/6J WT mice (6-8 weeks old, Jackson Lab). These melanoma-derived cells are transfected to express chicken ovalbumin peptide (OVA). Five days after tumor cell injection, scaffolds were surgically implanted subcutaneously into the same approximate region of the tumors in both flanks. For cell-loaded studies, ex vivo activated OT-I T cells were transferred either intravenously using retro-orbital injections (100 μL per animal) or implantable scaffolds at the same day. 5×10⁶ OT-I T cells were given in all adoptive transfer experiments. Tumor size was assessed over time using a digital caliper until day 22 at which animals were sacrificed and the tumor, draining lymph nodes, and spleen were extracted.

The immunological abscopal effects were also tested for the melanoma model. Wildtype mice received a subcutaneous injection of 5×10⁵ B16F10-Ova cancer cells in the flank, and as before functionalized or unfunctionalized scaffolds were implanted subcutaneously after 5 days, when the tumors first became palpable. On the same day, we injected 3×10⁵ tumor cells contralaterally to primary one.

### Tumor Assays

Tumor masses were measured using a digital balance before digesting the tumor tissue for flow cytometry or fixing it for tissue sectioning. Tumors were digested by incubating in collagenase and DNase I (50 μg/mL) at 37° C. for 15 min. These enzymes were inactivated with EDTA (20 μL/mL of solution). Tissues were then mechanically disaggregated and passed through a 70 μm cell strainer to obtain a single-cell suspension. Cells were then stained with the fluorochrome-conjugated antibodies on ice. For intracellular staining, cells were permeabilized with Granzyme B Fix/Perm buffer according to the manufacturer's instructions (BioLegend) before staining. Detection of apoptotic cells in tumor tissue was achieved using Terminal deoxynucleotidyl transferase-mediated dUTP nick-end labeling (TUNEL) staining following the manufacturer's directions. TUNEL-positive cells indicated as apoptotic melanoma cells. Tissue sections were imaged by a fluorescence microscope (Keyence BZ-X800, Osaka, Japan).

Blood was collected 7 days post implantation of the scaffolds for cytokines analysis. Concentrations of IFN-γ and TNF-α in serum were determined by enzyme-linked immunosorbent assay (ELISA) (eBioscience).

### Hematology, Biochemistry, and Tissue Collection

After 7 days of treatment, all mice were sacrificed, and blood and major organs were collected for blood analysis and organ studies. Using a standard blood-collection technique, 1000 μL blood was drew into LTT-Lavender top EDTA anticoagulant collection tube for hematology analysis. The blood serums were also isolated using SST-serum Separator Tube containing clot activator with gel for blood chemistry analyses. Samples were analyzed by IDEXX BioAnalytics (West Sacramento, Calif.). Major organs as well as the scaffolds from these mice were also extracted, fixed and processed, and stained with hematoxylin and eosin. Pathology was examined using a digital microscope.

### Statistical Analysis

The Kruskal-Wallis rank sum test, one-way ANOVA and two-tailed Student's t-test were utilized as appropriate to analyze the data at a significance of α or p<0.05. Quantitative data were expressed as mean±standard deviation (SD). To determine the number of specimens for the proposed experiments, power analysis was conducted based on preliminary data.

### Results

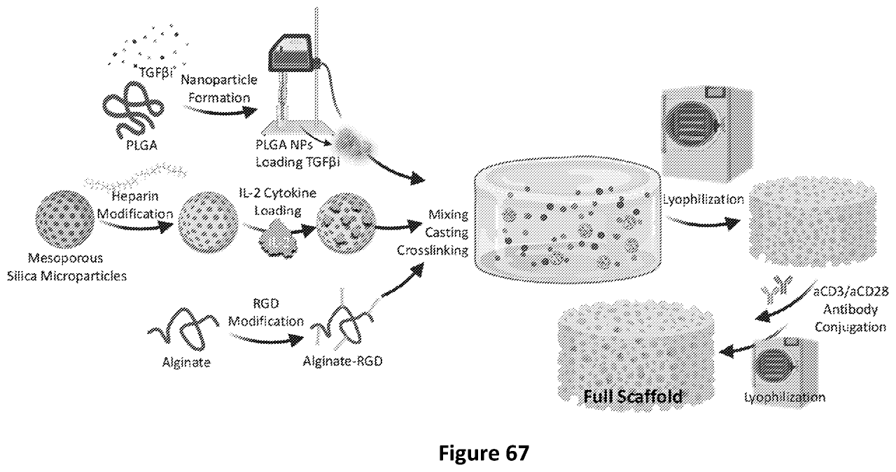

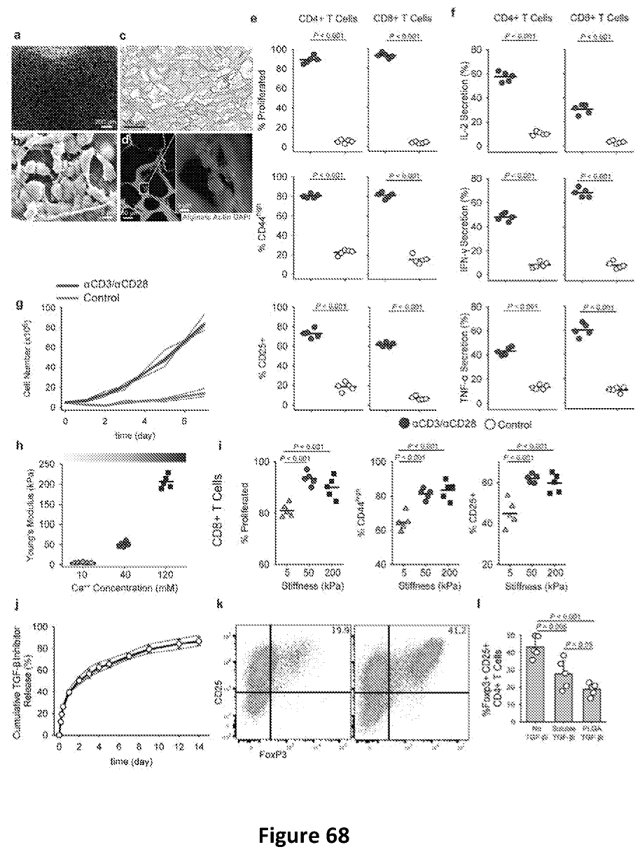

Scaffold fabrication. To enhance local immunity against tumors, the alginate-based scaffold disclosed herein combines multiple design considerations (FIG. 67). The scaffold facilities traffic of T cells throughout the structure by virtue of its microporous design (FIG. 68A). Naïve T cells pipetted onto the surface of the scaffold infiltrate throughout (FIG. 68B). Pore sizes of the scaffold were assessed in vivo 7 days after surgical implantation of scaffolds in wild-type, immunologically intact, non-tumor-bearing mice. As shown here, the pore size of the scaffolds after in vivo implantation (FIG. 68C) was quite similar to the pore size prior to implantation. Fluorescent microscopy showed that primary T cells crawled within the scaffold (FIG. 68D). Surfaces of the scaffold were covalently conjugated with stimulatory antibodies anti-CD3 and anti-CD28. To test the activation of T cells, naïve T cells were placed onto either the immunoactive scaffolds or control scaffolds that lacked all functionalization, and proliferation and cytokine production were measured by flow cytometry after 3 days. The T cells were quite activated, showed enhanced proliferation and cytokine production (FIGS. 68E-F), confirming that covalent conjugation of stimulatory antibodies to the surface area of the scaffold produced robust T cell activation. Over days, the scaffold allowed for a dramatic 20-fold increase in T cells (FIG. 68G). Because mechanical stiffness of the substrate can promote T-cell activation, the elastic modulus of the alginate 3D scaffold was tuned based on Ca²⁺ concentration (FIG. 68H) and found the resulting T-cell activation was maximized when the elastic modulus was ˜50 kPa (FIG. 68I). The design presented herein allowed for TGF-β inhibitor (TGF-βi) to gradually release from embedded poly(lactic-co-glycolic acid) (PLGA) nanoparticles (FIG. 68J), which suppressed the development of Foxp3+ Tregs from naïve CD4+ T cells being cultured with anti-CD3, anti-CD28, and TGF-β (FIGS. 68K-L). As shown herein, the interleukin 2 (IL-2) cytokine was gradually eluted from engineered mesoporous silica microparticles and from the scaffolds. Moreover, chemokines were also gradually released from alginate scaffolds resulting in robust recruitment of T cells. Taken together, these results show the scaffold described herein orchestrates suppression of Treg development and promotion of effector T-cell activation.

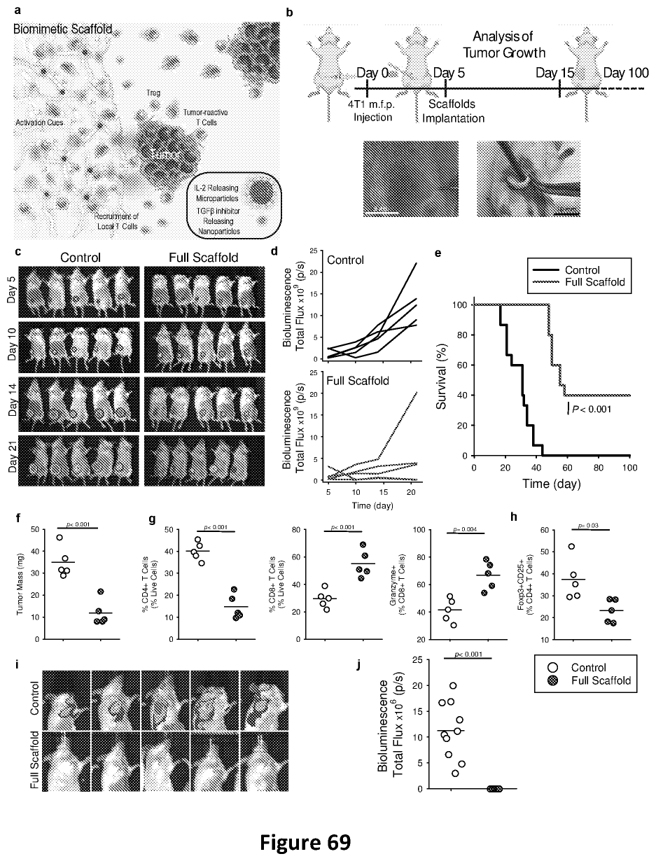

4T1 breast cancer model. To determine whether local depletion of Tregs could facilitate clearance of tumors, the scaffolds were tested in a murine breast cancer model. As described above, the mechanically tuned, 3D, macroporous, alginate scaffolds were coated with stimulatory antibodies and embedded with chemokine (C-C motif) ligand 21 (CCL21), IL-2-eluting mesoporous silicon microparticles, and nanoparticles that released TGF-β inhibitor (FIG. 69A). All mice received an injection of 4T1 breast cancer cells (a triple negative cell line) into the mammary fat pad (FIG. 69B). After 5 days, the tumors became palpable, and the scaffolds were implanted adjacent to the tumor in the intramammary fat space (FIG. 69B). Control mice were untreated. Tumor sizes were monitored twice weekly by luciferase imaging for the first three weeks. All mice were followed for survival and the experiment was ended on day 70. Four of five mice showed suppression of the growth of tumors in the mice treated with functionalized scaffolds, while all control mice showed growth of the tumors (FIGS. 69C-D). Survival was significantly improved in treated mice (FIG. 69E). These results show that the functionalized scaffolds controlled the growth of an aggressive breast cancer model in mice.

To assess the mechanism by which the scaffold allowed mice to control tumors, some mice were sacrificed on day 15 (10 days after scaffold implantation) and the tumors were examined by flow cytometry. The tumors in treated mice were much smaller (FIG. 69F). T cells were examined in the tumors, and there was a significant increase in infiltrating CD8+ T cells with a concomitant decrease in CD4+ T cells (FIG. 69G). Infiltrating CD8+ T cells showed an increase in their killing capability (FIG. 69G). It was found that the proportion of regulatory T cells in the tumor was reduced by half (FIG. 69H). These results confirm that the immunoactive scaffold depletes Tregs and enriches the presence of activated, cytotoxic T cells.

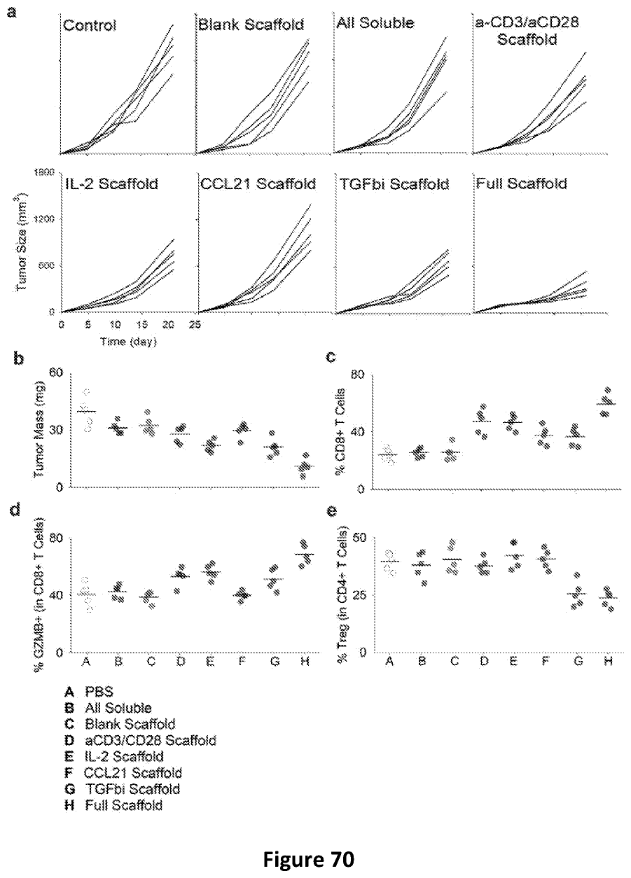

To highlight the contribution of each component of the scaffold, mice were injected with 4T1 breast cancer cells as above and were implanted alginate scaffolds comprising the individual ingredients. These experiments allowed us to examine the effects on tumor growth and on responding T cells within the tumor (FIG. 70). Scaffolds included ones with only: surface functionalization with anti-CD3 and anti-CD28; PLGA nanoparticles releasing of TGF-β inhibitor; release of the chemokine CCL21; and release of the cytokine IL-2. Injection of all these components intra/peritumorally was also tested. To control for the surgical implantation of the scaffold, unfunctionalized scaffolds were used. It was found that release of either TGF-β inhibitor or IL-2 significantly reduced tumor growth. The “full” scaffold combining all the components was the most potent at reducing tumor growth (see Table 3 for statistical analysis). The reduction of regulatory T cells in the tumor was only observed in the scaffold that secreted TGF-βi. The degree of Treg depletion by about 35% was comparable to the fully functionalized scaffolds. To understand which component(s) participated in the recruitment of cytotoxic T cells into the tumor, it was found that CD3/CD28 antibody coating of the scaffolds was the single most effective, even more than the CCL21 chemokine alone. Thus, local activation and expansion of cytotoxic T cells is important for their recruitment into the tumor. To understand which component(s) contributed to the elevated cytotoxicity of T cells (as measured by granzyme B expression), it was found that CD3/CD28 coating, release of IL-2, and release of TGF-βi were comparably potent. It is expected that offering more TCR stimulation to T cells would drive more cytotoxic function, and IL-2 is well-known to potentiate cytotoxicity as well. One can understand the finding that TGF-β inhibition increased cytotoxicity of effector T cells through both direct and indirect mechanisms. It is known that TGF-β signaling in T cells directly inhibits proliferation and cytotoxicity. In addition, global diminishment of regulatory T cells was shown to indirectly potentiate the cytotoxicity of intra-tumoral CD8+ T effectors. The results presented herein extended this finding to reveal that local diminishment of intratumoral Tregs evokes greater cytotoxicity in T effectors. Based on these observations, reducing intratumoral Tregs while activating and recruiting T effectors makes for the most potent therapy.

A dramatic extension of survival was found in the treated mice. To better understand the therapeutic impact by which survival was enabled, it was noted that in the 4T1 breast cancer model metastases develop spontaneously from the primary tumor, and the aggressive spread of metastases to the draining lymph nodes and other organs resembles human breast cancer. It was found that metastases in the brain and axillary lymph nodes were utterly eliminated in treated mice, whereas they were universally found in untreated mice (FIGS. 69I-J). These results show that the immunoactive scaffold described herein reduces both primary tumors and metastatic spread.

TABLE 3
One Way Analysis of Variance (ANOVA) was used for all pairwise
multiple comparison using Holm-Sidak method. All data were assessed
with Normality Test (Shapiro-Wilk) and Equal Variance Test (Brown-
Forsythe). Overall significance level is set to be 0.05.

| | | | | Tumor Treg |
|---|---|---|---|---|
| | | | Tumor | (Foxp3 + |
| | Tumor | | CD8 + | CD25 + |
| Comparison | Mass | Tumor CD8+ | GZMB+ | CD+) |
| PBS vs. All Sol | NS | NS | NS | NS |
| PBS vs. Blank | NS | NS | NS | NS |
| PBS vs. CD3/CD28 | 0.005 | <0.001 | 0.032 | NS |
| PBS vs. IL-2 | <0.001 | <0.001 | 0.005 | NS |
| PBS vs. CCL21 | 0.024 | 0.024 | NS | NS |
| PBS vs. TGFbi | <0.001 | 0.039 | NS | 0.001 |
| PBS vs. Full | <0.001 | <0.001 | <0.001 | <0.001 |
| All Sol vs. Blank | NS | NS | NS | NS |
| All Sol vs. | NS | <0.001 | NS | NS |
| CD3/CD28 | | | | |
| All Sol vs. IL-2 | 0.042 | <0.001 | 0.016 | NS |
| All Sol vs. CCL21 | NS | 0.049 | NS | NS |
| All Sol vs. TGFbi | 0.023 | NS | NS | 0.004 |
| All Sol vs. Full | <0.001 | <0.001 | <0.001 | <0.001 |
| Blank vs. | 0.017 | <0.001 | 0.009 | NS |
| CD3/CD28 | | | | |
| Blank vs. IL-2 | <0.001 | <0.001 | 0.001 | NS |
| Blank vs. CCL21 | NS | NS | NS | NS |
| Blank vs. TGFbi | <0.001 | 0.049 | 0.031 | <0.001 |
| Blank vs. Full | <0.001 | <0.001 | <0.001 | <0.001 |
| CD3/CD28 vs. IL-2 | NS | NS | NS | NS |
| CD3/CD28 vs. | NS | NS | 0.021 | NS |
| CCL21 | | | | |
| CD3/CD28 vs. | NS | NS | NS | 0.005 |
| TGFbi | | | | |
| CD3/CD28 vs. Full | <0.001 | 0.045 | 0.005 | 0.001 |
| IL-2 vs. CCL21 | NS | NS | 0.003 | NS |
| IL-2 vs. TGFbi | NS | NS | NS | <0.001 |
| IL-2 vs. Full | 0.013 | 0.029 | 0.036 | <0.001 |
| CCL21 vs. TGFbi | NS | NS | NS | <0.001 |
| CCL21 vs. Full | <0.001 | <0.001 | <0.001 | <0.001 |
| TGFbi vs. Full | 0.024 | <0.001 | 0.001 | NS |

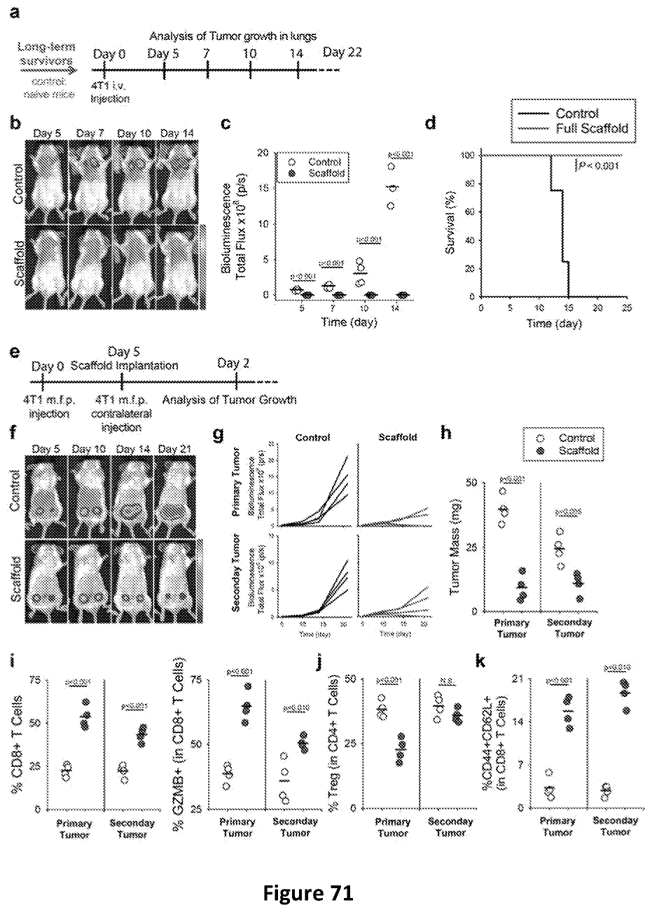

Next, it was sought to better understand whether the long-term survivors, which resisted both the primary tumor and metastases, had developed immunological memory that could protect them from recurrence. When the tumor-free mice were seen to survive to 100 days (˜40 days beyond the last non-surviving mouse), the survivors were rechallenged with an intravenous injection of 4T1 tumor cells, which deposits tumor cells into the lungs and emulates new metastases (FIG. 71A). To be sure this lung-metastatic model was performing as intended, and to directly compare the impact of immunological memory versus naïve T-cell responses, this intravenous injection of tumor cells was compared in naïve wild-type mice of same age. The lung metastasis for this group of survivors were examined by luciferase imaging (FIG. 71B). All (100%) the long-term survivors survived the rechallenge. They all utterly eliminated the development of detectable tumors in the lungs (FIG. 71C). In contrast, all the control mice developed lung metastases and died by or before day 14 (FIG. 71D). These results show that the immunomodulatory scaffolds engender durable memory that can protect against the recurrence of the cancer.

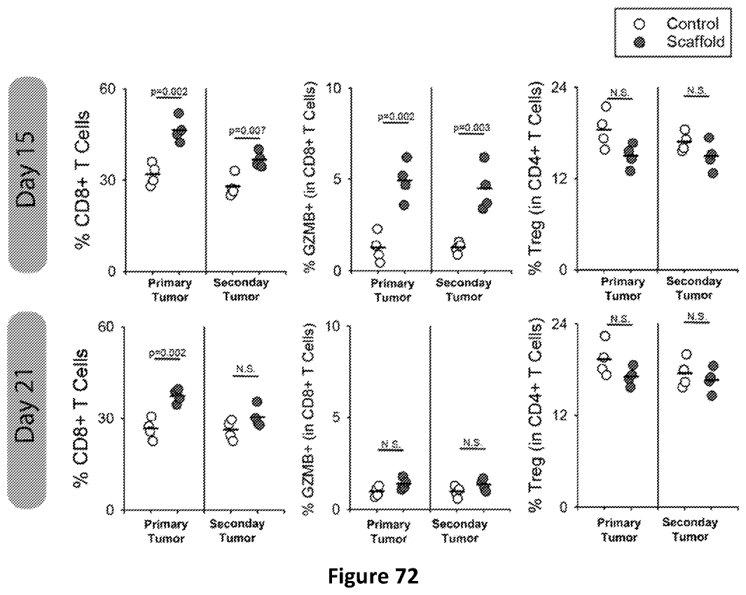

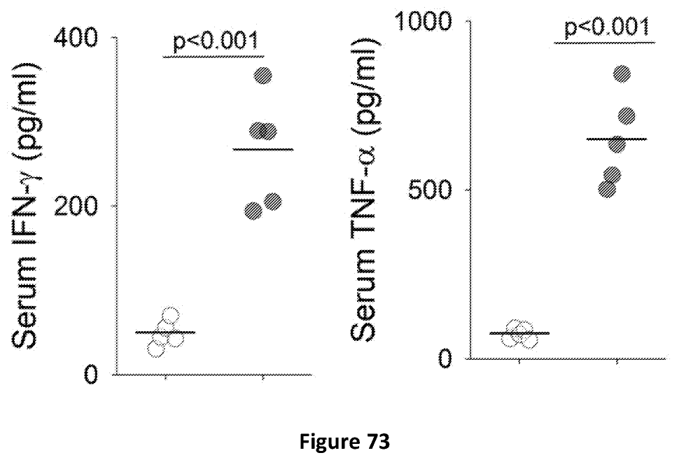

Distant Tumors. By the time many solid tumors are clinically detected, they have already spread regionally or distantly. It was next assessed whether local depletion of intratumoral Tregs in a primary tumor could alter the trajectory of the immune response in a distant secondary tumor. Wildtype mice received a mammary fat pad injection of 4T1-Luc cells, and as before immunoactive scaffolds were implanted subcutaneously after 5 days. On the same day, tumor cells were injected contralaterally to the primary one (FIG. 71E). Tumor growth was monitored on both sides using bioluminescence imaging (FIGS. 71F-71G). The growth and the mass of the secondary tumor were significantly suppressed upon local treatment of the primary tumor with immunoactive scaffolds but not control scaffolds (FIG. 71H). It was also noted an increase in the population of tumor-infiltrating, activated CD8+ T cells on both sides 15 days after the start of primary tumors (FIG. 71I). Activated T cells were also found in the draining lymph nodes, suggesting that local inhibition of Tregs allows widespread trafficking of activated effector T cells at earlier timepoints, but the effect diminished at later times (FIG. 72). It was also observed the expansion of central memory (CD62L+CD44+CD8+) T cells in the tumor (FIG. 71K). This T cell memory subset has been noted in mice and humans to be important for both clearance of tumors and prevention of their recurrence. Notably, the proportion of Tregs was markedly reduced only in the primary tumor (FIG. 71J) but not in the draining lymph nodes (FIG. 72). These results show that localized suppression of intratumoral Tregs resulted in both successful clearance of primary tumors as well as marked reduction of secondary tumors. The disproportionate increase in activated, cytotoxic CD8+ T cells in the distant lymph nodes and secondary tumor indicates that expansion of potent effector T cells in the primary tumor spreads to secondary sites. As further evidence of an immunological “abscopal” effect arising from the scaffold acting on the primary tumor, it was noted an elevated level of inflammatory serum cytokines in the circulation (FIG. 73). Thus, while the release of TGF-β inhibitor from the scaffold alters Tregs locally, there are global effects on effector T cells including clearing distant tumors and development of long-term protective memory.

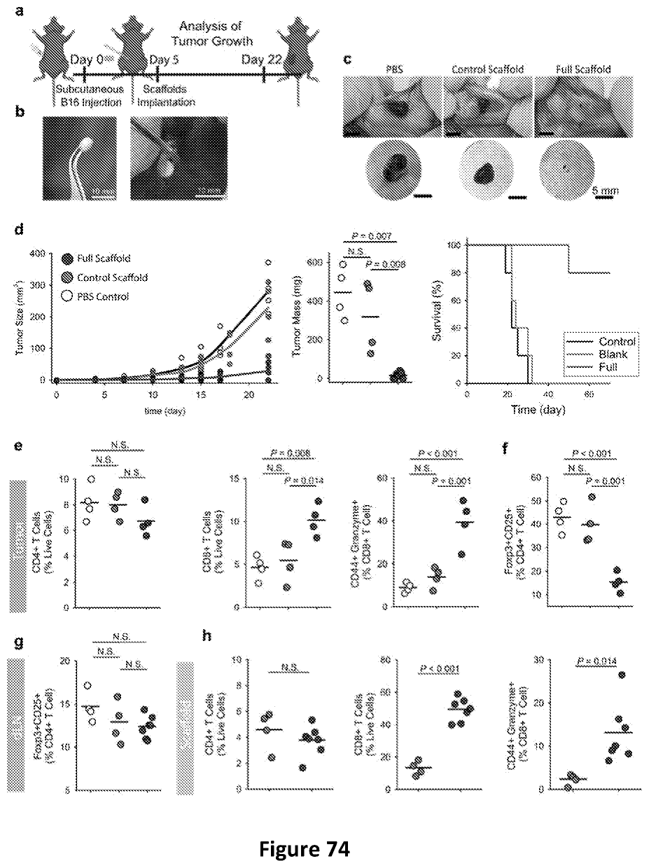

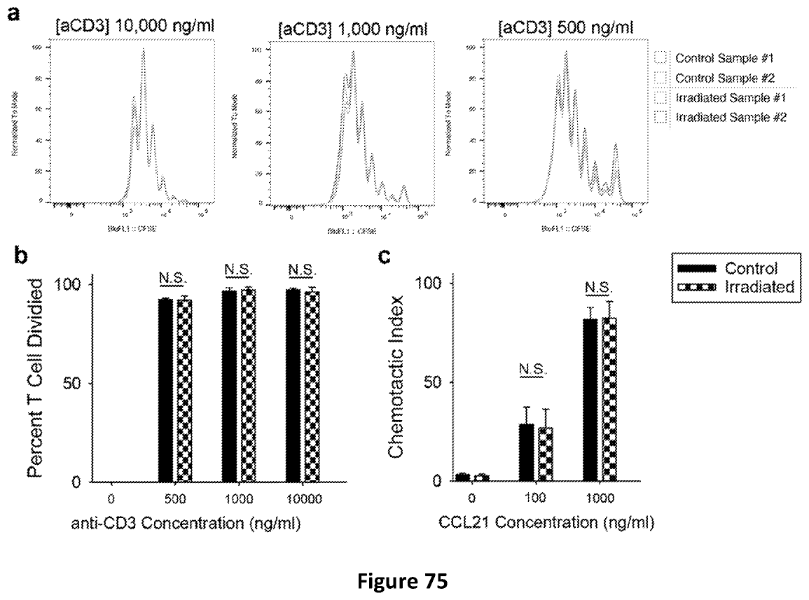

B16-F10 melanoma model. To determine whether local depletion of Tregs could facilitate clearance of a second tumor type, the scaffolds were tested in the murine melanoma model. Mice received a subcutaneous injection of B16-F10 cells to their right flank (FIG. 74A). The scaffolds were functionalized as described above (“full”) and control scaffolds lacked functionalization. After 5 days, when the tumors first became palpable, the scaffolds were implanted adjacent to the tumor in the subcutaneous space (FIGS. 74B-74C). Some mice remained entirely untreated. Unfunctionalized scaffolds served as controls for the surgery and wound healing. The experiment was ended for all mice on day 22, when the untreated mice required euthanasia for tumor size and morbidity. H&E staining of the tissue adjacent to the tumor and scaffold showed successful tissue integration and recruitment of T cells via the implanted microporous scaffolds. In all mice receiving the functionalized scaffold, tumor growth was suppressed (FIG. 74D) as compared with untreated mice or those receiving a control scaffold. The tumor size for treated mice was roughly one eighth the size of those receiving control scaffolds (FIG. 74D). For three (43%) of the treated mice, no residual tumor could be found at all (complete remission) (FIG. 74D). These results reveal that the immunomodulatory scaffold helps clear multiple types of solid tumors.

Tumors, implanted scaffolds, tumors' draining lymph nodes, and spleens were examined. The tumor-infiltrating T cells showed an increase in the proportion of CD8+ T cells, and they were highly activated and cytotoxic (FIG. 74E) in the tumors adjacent to functionalized scaffolds versus control scaffolds. These T cells were also significantly more activated and cytotoxic within the functionalized versus the control scaffolds (FIG. 74H). The proportions of total CD4+ T cells, on the other hand, were roughly consistent (FIGS. 74E-74H). The effect of recruitment and proliferation of the CD8+ T cells was local, as there was no increase in the proportion, activation, or cytotoxicity of CD8+ T cells in the draining lymph node at the late time point (22 days). These findings show that the functionalized scaffolds successfully recruited and locally activated T cells, promoted their entry into tumors, and promoted the clearance of even aggressive tumors.

To better understand the impact of local TGF-β inhibition on the melanoma model, various tissues were examined for the presence of Tregs. Tregs were depleted by about 60% in the tumors adjacent to functionalized scaffolds as compared with controls (FIG. 74F). As systemic administration of TGF-βi can result in autoimmune disease, it was examined whether the local release of TGF-βi adjacent to the tumor could suppress Tregs in distant parts. It was found no significant changes in Treg proportions in the draining lymph node (FIG. 74G). Exhaustion of tumor infiltrating lymphocytes is commonly noted, and there was no significant difference in PD-1^{hi} T cells.

To better understand the mechanism of these underlying findings, the T cells recruited to the scaffolds were examined. Previous work in melanomas overexpressing CCL21 showed recruitment of both CD4+ and CD8+ T cells, albeit with different tempos. The scaffolds above employed the chemokine CCL21 to recruit T cells into the milieu. It was compared whether ligation of two different chemokine receptors on T cells, CCR7 and CXCR4, by two different chemokines, CCL21 and CXCL12 (also called stromal cell-derived factor 1 (SDF-1a)), respectively, could explain the preferential recruitment of CD8+ T cells into the scaffold and tumor. It was found that both chemokines successfully recruited T cells and helped clear tumors compared to control scaffolds. It was found that CCL21 favored recruitment of CD8+ T cells and disfavored CD4+ T cells as compared with CXCL12. Despite the presence of identical activation antibodies CD3 and CD28 in the scaffold, co-ligating CCR7 with CCL21 also increased the cytotoxic state of the effector T cells. These results show that CCL21 improves recruitment of cytotoxic effectors into the tumor compared to CXCL12. Moreover, ligation of CCR7 improves the killing potential of CD8+ T cells. These results show that activation of chemokine receptors on T cells improves the therapeutic capability of the scaffold described herein.

An important detail in the therapeutic translation of this scaffold lies is its stability prior to use. Water-based hydrogels can have low stability because of spontaneous hydrolysis, which we sought to reduce by preparing our alginate-based scaffolds in a sterile, lyophilized, and dry state. To test whether the scaffolds could retain their functional ability after 6 months of cold storage, functionalized scaffolds were employed in the B16 melanoma mouse model and no difference was found between freshly fabricated scaffolds and older ones. Also, it was found sterilization through x-ray irradiation did not affect proteins within the scaffold.

These results showed that the immunoactive scaffold cleared tumors by recruiting and highly activating CD8+ T cells while depleting local Tregs within the tumor.

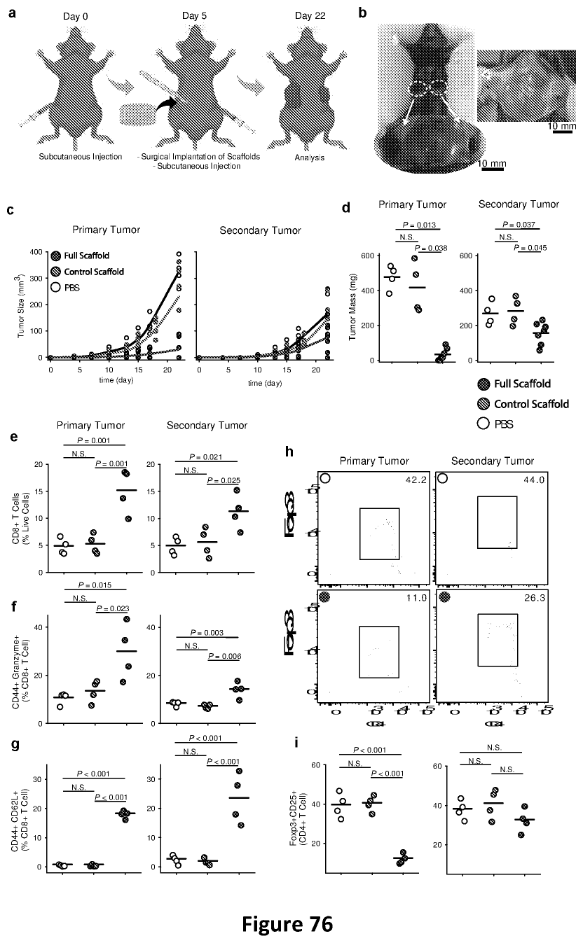

Distant Melanoma Tumors. It was next assessed whether local depletion of intratumoral Tregs in a primary tumor could alter the trajectory of the immune response in a distant secondary tumor. Wildtype mice received a subcutaneous injection of B16-F10 cells in the flank, and as before functionalized or unfunctionalized scaffolds were implanted subcutaneously after 5 days, when the tumors first became palpable. On the same day, tumor cells were injected contralaterally to primary one (FIG. 76A). Tumor growth was monitored on both sides and tumor masses measured. The growth of the secondary tumor was suppressed by about 40 percent upon local treatment of the primary tumor with immunoactive scaffolds but not control scaffolds (FIGS. 76B-76D). The proportion of tumor-infiltrating CD8+ T cells was increased by more than two-fold in the contralateral tumor of the mice that received functionalized scaffolds (FIG. 76E). Activated T cells were also found in the spleen and draining lymph nodes, suggesting that local inhibition of Tregs allows widespread trafficking of activated effector T cells. Those T cells were more activated and expressed more Granzyme B (FIG. 76F). It was noted the expansion at 22 days of an expanded population of CD62L+CD44+CD8+ T cells, suggesting the development of central memory T cells in the tumor. As before, the proportion of Tregs was markedly reduced in the primary tumor. Importantly, Tregs were also reduced in the secondary tumor, but to a lesser extent (FIGS. 76H-76I), and not in the lymph nodes. These results show that localized suppression of intratumoral Tregs resulted in both successful clearance of primary tumors as well as marked reduction of secondary tumors. The minor decrease in Tregs in the distant tumor supports the model that antigen-specific Tregs trained in the primary tumor may spread globally to facilitate distant tumors. By suppressing those Tregs in the primary tumor, Tregs were suppressed in the secondary tumors. However, the disproportionate increase in activated, cytotoxic CD8+ T cells in the secondary tumor despite the minor decrease in Tregs there indicates that expansion of potent effector T cells in the primary tumor spreads through the body (abscopal effect) and affect distant sites arising from the scaffold acting on the primary tumor as seen also in our 4T1 breast cancer data.

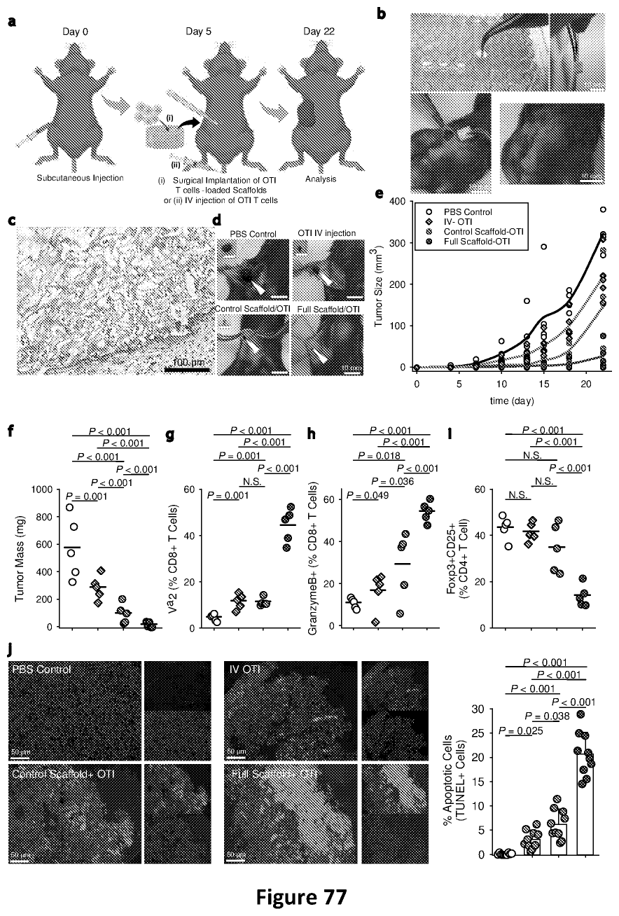

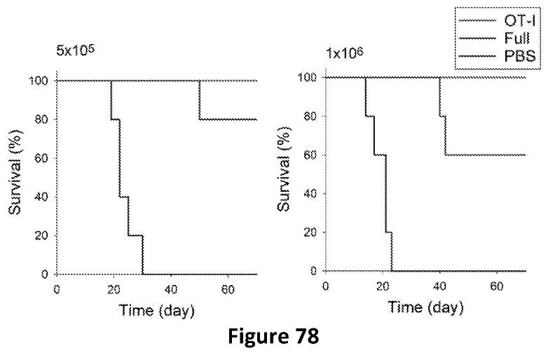

Adoptive T Cell Therapy. Delivery of engineered T cells like CAR-T cells is necessary for a variety of cancer therapies, but unlike in lymphomas, intravenous administration does not always allow for efficient delivery of tumor-specific T cells into solid tumors. Alginate-based scaffolds that lack structure have been used to deliver T cells in murine melanoma and breast cancer, where the matrix served as a T-cell depot to support their local expansion. We sought to employ the structural advantages of microporosity and mechanical rigidity of the scaffolds described herein to deliver T cells. Wildtype mice received a subcutaneous injection of B16-F10 cells in the flank, and as before functionalized or unfunctionalized scaffolds were implanted after 5 days, when the tumors first became palpable (FIGS. 77A-77D). These scaffolds were loaded just before implantation with 5×10⁶ activated OT-I T cells. Some mice instead received intravenous injection of either the same number (5×10⁶) of OT-I T cells or saline (FIGS. 77D-77F). Mice were euthanized at 22 days as before for further analysis.

H&E staining of the scaffolds adjacent to the tumor confirmed tissue engagement, successful delivery, and proliferation of OT-Is plus recruitment of endogenous T cells (FIG. 77C). The OT-I-loaded, functionalized scaffold significantly suppressed tumor growth compared to other treatments and controls, by ˜16-fold compared to saline control and ˜10-fold compared to IV injection of OT-I T cells (FIGS. 77E-77F). It was also observed significant improvement in survival of mice receiving the combination of full scaffolds and OT-1 T cells. All (100%) the mice receiving OT-I-loaded immunoactive scaffolds showed long-term survival (tracked up to 70 days), even when they had been injected with more B16F10-Ova cancer cells (1×10⁶ compared to 0.5×10⁶). These results show that scaffold-based delivery resulted in better tumor clearance than intravenous delivery of T cells.

Moreover, it was found that the accumulation and activation of antigen-specific T cells was far more effective with the immunoactive scaffolds than with delivery by the inert, control scaffold or intravenous delivery (FIGS. 77G-77I). Scaffold-based delivery of OT-I T cells dramatically outperformed intravenous delivery when looking at the resulting number of OT-I T cells in the tumor (FIG. 77G). Moreover, the scaffold-delivered T cells were more activated and showed greater cytotoxicity (FIG. 77H). Treg populations in the tumor showed significant differences between treatment groups. Regulatory T cells were suppressed by over 60 percent in the tumors adjacent to immunoactive scaffolds as compared to the control scaffold, intravenous delivery of T cells, or saline controls (FIG. 77I). TUNEL staining showed a significant increase in apoptotic tumor cells in the functionalized scaffold that delivered OT-I T cells versus controls (FIG. 77J). These results confirm that the immunoactive scaffold delivers highly cytotoxic, antigen-specific T cells to the tumor environment.

Tumor-draining lymph nodes showed a comparable number of OT-I T cells when they were delivered by the scaffold as compared with IV injection. On the other hand, highly activated OT-I cells were more present in the LN and spleens when mice received an IV injection versus when scaffolds delivered the T cells. As before, the proportion of Tregs in lymph nodes was comparable between groups. These results explain why intravenous injection of engineered, tumor-specific T cells so often fails: these activated T cells end up in the LNs and spleen rather than in the tumor. The direct access of effector T cells to the tumor microenvironment provided by the scaffold also bypasses the formidable barriers established by the tumor vasculature to the transport of tumor-specific T cells.

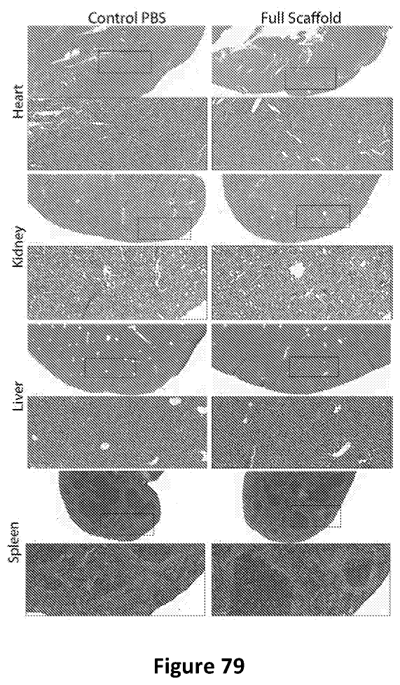

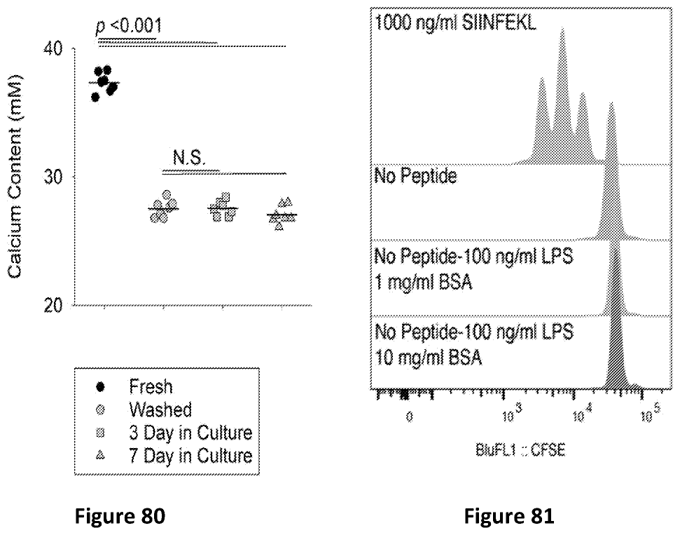

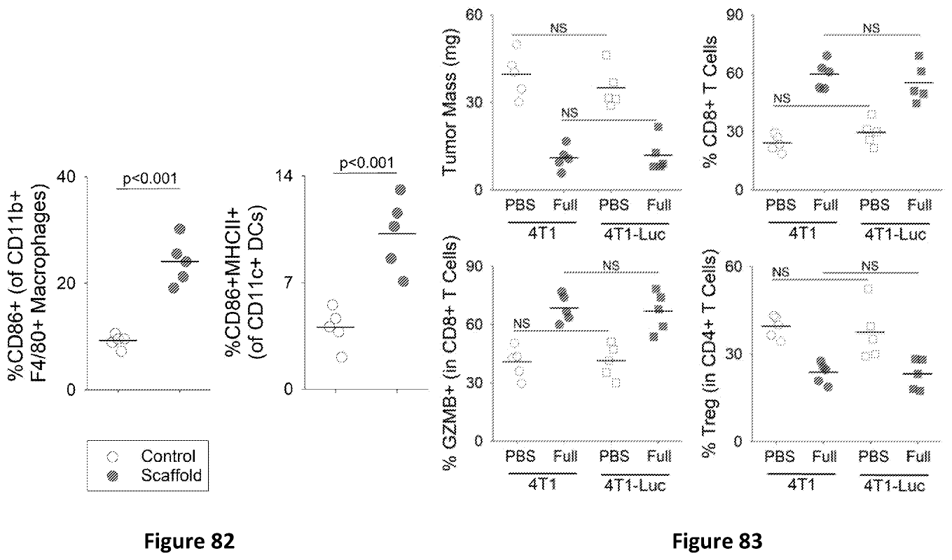

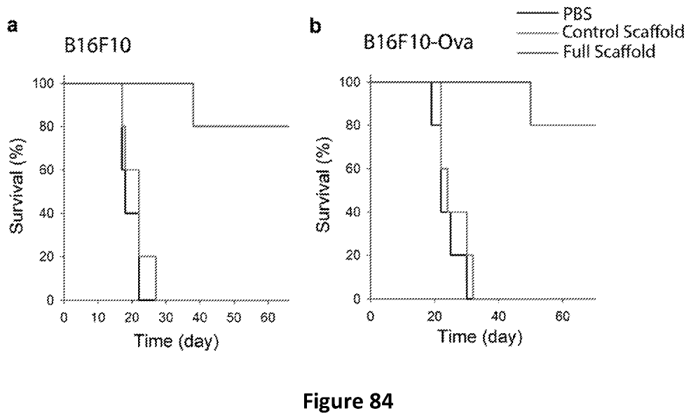

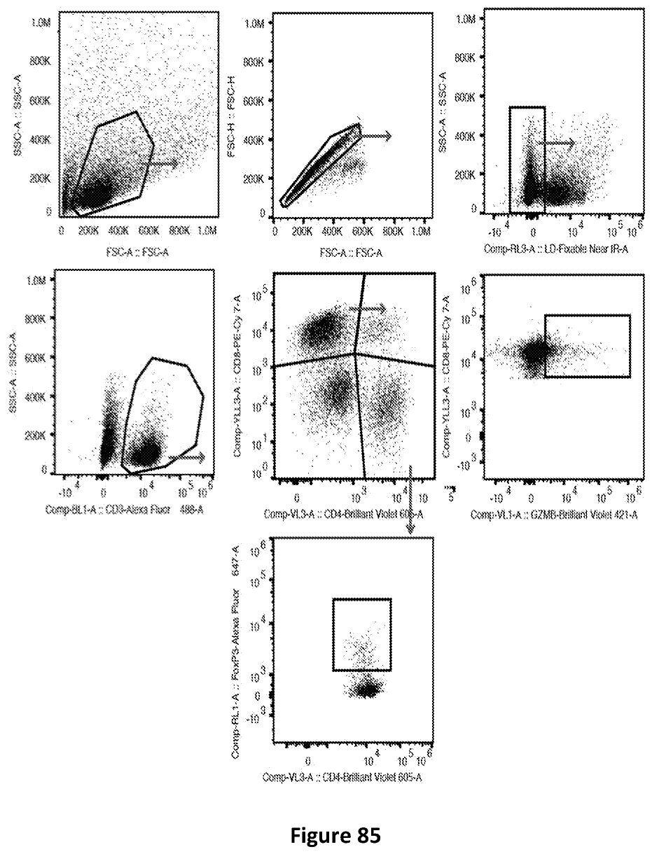

The toxicity of immunoactive scaffolds was also assessed one-week post-implantation in non-tumor bearing mice. It was found no significant changes in hematological counts (Table 4). Major organs including heart, liver, spleen, and kidney were also examined for histological alterations. No detectable abnormalities or lesions in these organs were noticed (FIG. 79).

TABLE 4
Biosafety Evaluation of Full Scaffold.
Blood chemistry and hematology for naïve mice treated
with either PBS injections (Control) or with implanted
Full scaffolds 7 days post-implantation.

| CLINICAL CHEMISTRY | Control | Full |
|---|---|---|
| ALP (U/L) | 110 ± 23 | 96 ± 18 |
| AST (U/L) | 189 ± 46 | 214 ± 64 |
| ALT (U/L) | 39 ± 17 | 47 ± 20 |
| Creatine kinase (U/L) | 1992 ± 723 | 2309 ± 672 |
| Albumin (g/dL) | 2.7 ± 0.4 | 2.8 ± 0.3 |
| Total Bilirubin (mg/dL) | 0.2 ± 0.03 | 0.2 ± 0.01 |
| Total Protein (g/dL) | 4.4 ± 0.3 | 4.5 ± 0.4 |
| Globulin (g/dL) | 1.6 ± 0.2 | 1.7 ± 0.3 |
| BUN (mg/dL) | 21 ± 4.3 | 23 ± 2.1 |
| Creatinine (mg/dL) | 0.2 ± 0.02 | 0.17 ± 0.03 |
| Cholesterol (mg/dL) | 72 ± 5 | 77 ± 6.9 |
| Glucose (mg/dL) | 152 ± 15 | 137 ± 20 |
| Calcium (mg/dL) | 8.3 ± 0.78 | 7.8 ± 0.5 |
| Phosphorus (mg/dL) | 13.5 ± 2 | 14.2 ± 1.3 |
| Bicarbonate TCO2 | 8.7 ± 1.1 | 9.6 ± 1.3 |
| (mmol/L) | | |
| Chloride (mmol/L) | 116 ± 5.6 | 114 ± 7 |
| Potassium (mmol/L) | 4.7 ± 0.9 | 5.3 ± 0.8 |
| ALB/GLOB ratio | 1.85 ± 0.26 | 1.65 ± 0.2 |
| Sodium (mmol/L) | 149 ± 5.1 | 154 ± 7.2 |
| NA/K Ratio | 31 ± 4.9 | 28 ± 6.2 |
| HEMATOLOGY | | |
| WBC (K/uL) | 1 ± 0.23 | 1.3 ± 0.33 |
| RBC (M/uL) | 8.78 ± 0.48 | 8.45 ± 0.56 |
| HGB (g/dL) | 14.1 ± 0.91 | 13.7 ± 1.8 |
| HCT (%) | 41.1 ± 3.9 | 38.4 ± 3.4 |
| MCV (fL) | 47 ± 4.7 | 48 ± 3.8 |
| MCH (pg) | 16.1 ± 0.6 | 17.3 ± 0.9 |
| MCHC (g/dL) | 34.8 ± 4.8 | 37.8 ± 5 |
| Platelet Count (K/uL) | 1018 ± 245 | 1209 ± 319 |
| Neutrophil (/uL) | 108 ± 48 | 128 ± 31 |
| Lymphocyte (/uL | 895 ± 254 | 1032 ± 301 |
| Monocyte (/uL) | 56 ± 21 | 78 ± 23 |
| Eosinophil (/uL) | 0 | 0 |
| Basophil (/uL) | 0 | 0 |

### DISCUSSION

To facilitate the immune response against solid tumors, a new multifunctional biomaterial scaffold was implanted adjacent to a tumor, which attracts cytotoxic T cells and enhances their function by suppressing tumor-resident Tregs. Together these activities allow for the much sought-after effect of overcoming the immunosuppressive effects of the microenvironment of solid tumors without global adverse effects. This flexible platform offers localized immunomodulation and treatment of multiple cancer types. The approach is general and could be adapted for localized immunoregulation in autoimmune diseases and infections as well.

Existing, clinical approaches to circumvent the immunosuppressive tumor microenvironment have serious downsides. For example, global inhibition of Tregs can lead to autoimmune side effects whereas systemic activation of T cells can lead to cytokine storms. Immunomodulatory solutions that act locally, thereby avoiding global side effects, would be attractive and may indeed be necessary. At the same time, intratumoral or intralymphatic injections of Treg-depleting therapies have shown limited promise because of the high oncotic pressure that rapidly dissipates the therapeutic effect. Another approach entails the release of cytokines to T cells via nanogel “backpacks,” which has been shown to enhance tumor clearance. However, the continual and uncontrolled release of cytokines risks systemic effects. Intravenous administration of collagen-binding domains fused to the cytokine IL-12 prolonged the release of cytokine into the tumor stroma; however, this approach increases risks of cardiovascular disease. Nanoparticles or microparticles that deliver cytokines to CD8+ T cells at carefully controlled rates have been developed and showed that prolonged exposure to IL-2 impacts their differentiation and effector function. Besides cytokines, antibodies that stimulate CD3 and CD28 are known to augment T-cell activation in situ. Intratumoral injection of stimulatory antibodies has been attempted but rapid transport away from the tumor environment has prevented potential clinical application. As it was demonstrated in the present disclosure, sustained release of cytokines and other immunomodulatory factors from a biomaterial into the local tumor microenvironment, while at the same time stably anchoring stimulatory antibodies near the tumor addresses these shortcomings.

In addition to biochemical cues, it has been shown in recent work that T cells sense the mechanical stiffness of 3D microenvironments and undergo augmented activation when the environment reaches an elastic modulus of ˜40 kPa. Mechanistically, it has been shown that the stiffness of the local environment affects T-cell activation by modulating their metabolic program. Others have employed biomechanically soft hydrogels in vivo to activate T cells, but it is now known that mechanical rigidity is important to maximize activation. The mechanical stiffness of the biomaterial disclosed herein was optimized to mimic that of activated lymph nodes.

Adoptive cell therapy of antigen-specific T cells, for example CAR-T cells, have shown tremendous success in non-solid tumors because intravenously delivered cells are preferentially trafficked to the lymph nodes and spleen where these cancers reside. On the other hand, in the context of solid tumors intravenous delivery of antigen-specific T cells has shown major inadequacies in delivery to the tumor. Regional/local injection of CAR-T cells has been superior to intravenous delivery for a variety of cancers. Delivery of CAR-T cells into the tumor microenvironment from a biopolymer device has been successfully demonstrated in mouse models of cancer, demonstrating the promise of this approach. These approaches required the activation of T cells ex vivo before delivery, which works well for CAR-T cells but does not work for enabling endogenous T-cell responses.

In summary, the present disclosure demonstrates that an approach orchestrating multiple immunological mechanisms simultaneously can overcome barriers imposed by solid tumors to allow endogenous or engineered T cells to infiltrate and eradicate otherwise aggressive cancers. Moreover, these T cells clear secondary tumors and metastases. Finally, the T cells retain long-term memory and protect from cancer recurrence.

1. A porous biocompatible or biodegradable scaffold for regulating an immune response in a subject in need thereof, the scaffold comprising
(i) one or more microparticles,
(ii) one or more nanoparticles, and
(iii) a polymer comprising one or more of alginate, hyaluronic acid, chitosan, and poly(lactic-co-glycolic acid), wherein said one or more microparticles comprise heparin, and the heparin is bound to at least one compound that regulates T cell immune response, wherein said one or more nanoparticles or microparticles comprise at least one compound that regulates induction of regulatory T cells (Tregs).

2. The porous scaffold of claim 1, wherein the alginate comprises one or more arginine-glycine-aspartate (RGD) peptides.

3. The porous scaffold of claim 1, wherein the one or more microparticles comprise one or more silica microparticles.

4. The porous scaffold of claim 1, wherein the one or more nanoparticles or microparticles comprise poly(lactic-co-glycolic acid) (PLGA).

5. The porous scaffold of claim 1, wherein the at least one compound that regulates T cell immune response comprises a cytokine, a growth factor, an immunostimulatory compound, a chemokine, or an antibody or fragment thereof.

6. The porous scaffold of claim 5, wherein the compound that regulates T cell immune response comprises one or more of interleukin-2 (IL-2), interleukin-4 (IL-4), interleukin-6 (IL-6), interleukin-7 (IL-7), interleukin-10 (IL-10), interleukin-12 (IL-12), interleukin-15 (IL-15), IL-2 superkine, chemokine (C-C motif) ligand 21 (CCL21), anti-CD3 antibodies, and anti-CD28 antibodies.

7. The porous scaffold of claim 5, wherein the antibody is covalently bound to the polymer.

8. The porous scaffold of claim 1, wherein the at least one compound that regulates induction of regulatory T cells is an inhibitor of transforming growth factor-beta (TGF-β).

9. The porous scaffold of claim 8, wherein the inhibitor of TGF-β is galinusertib (LY2157299) or SB505124.

10. The porous scaffold of claim 1, comprising one or more silica-heparin microparticles bound to IL-2; one or more PLGA nanoparticles or microparticles comprising a TGF-β inhibitor; and anti-CD3 and anti-CD28 antibodies covalently bound to an alginate-RGD polymer.

11. The porous scaffold of claim 1, wherein the scaffold further comprising one or more immune cells.

12. The porous scaffold of claim 11, wherein the immune cells comprise wild-type or transgenic T cells, murine or human T cells, CD4+ or CD8+ T cells, or chimeric antigen receptor T cells (CAR-T cells).

13. A method of treating a first tumor in a subject in need thereof, said method comprising providing the porous scaffold of claim 1 at a site at or near said first tumor, thereby treating said first tumor in said subject.

14. The method of claim 13, wherein said porous scaffold is surgically implanted or inserted at a site at or near said first tumor.

15. The method of claim 13, wherein said first tumor is a solid tumor.

16. The method of claim 13, wherein said porous scaffold comprises T cells that are stimulated to target the first tumor, and induction of Tregs is suppressed.

17. The method of claim 13, wherein said treating reduces or eliminates said first tumor, slows the growth or regrowth of the first tumor, prolongs survival of said subject, or any combination thereof.

18. The method of claim 13, wherein said treating slows or reduces metastasis of said first tumor.

19. The method of claim 13, wherein said treating induces immune memory that can protect against recurrence of the first tumor.

20. The method of claim 19 wherein said immune memory comprises inducing central memory (CD44+CD62L+CD8+) T cells.

21. The method of claim 13, wherein said treating induces immune responses against a second tumor at a site away from the site of said first tumor.

22. The method of claim 13, wherein the first tumor is breast cancer.

23. The method of claim 22, wherein the breast cancer is triple-negative breast cancer.

24. The method of claim 13, wherein the first tumor is melanoma.

25. The method of claim 21, where the second tumor is a metastasis of said first tumor.

26. The method of claim 25, wherein the metastasis is metastatic breast cancer or metastatic melanoma.

27. A method of preventing metastasis of a tumor in a subject in need thereof, said method comprising providing the porous scaffold of claim 1 at a site at or near said tumor, thereby preventing metastasis of said tumor in said subject.

28. The method of claim 27, wherein said porous scaffold is surgically implanted or inserted at a site at or near said tumor.

29. The method of claim 27, wherein said tumor is a solid tumor.

30. The method of claim 27, wherein said porous scaffold comprises T cells that are stimulated to target the tumor, and induction of Tregs is suppressed.

31. The method of claim 27, wherein said treating prolongs survival of said subject.

32. The method of claim 27, wherein the tumor is breast cancer.

33. The method of claim 32, wherein the breast cancer is triple-negative breast cancer.

34. The method of claim 27, wherein the tumor is melanoma.

35. The method of claim 27, wherein the metastasis is metastatic breast cancer or metastatic melanoma.


SEQUENCE LISTING (3 sequences)

SEQ ID NO: 1 - protein, 8 aa, synthetic construct
  REGION 1..8: note = Synthetic
GGGGRGDY

SEQ ID NO: 2 - DNA, 420 bp, synthetic construct
  misc_feature 1..420: note = Synthetic
  source 1..420: mol_type = other DNA
ggagccatgggagaattcgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcagagcaaaaactttcacttcgatcccagggacgtcgtcagcaatatcaacgtattcgtcctggaactaaagggatctgaaacaacattcatgtgtgaatatgctgatgagacagcaaccattgtagaatttctgaacagatggattaccttttgtcaaagcatcatctcaacactaactcat

SEQ ID NO: 3 - protein, 138 aa, synthetic construct
  REGION 1..138: note = Synthetic
MGEFAPTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLTH